(19) United States
(12) Patent Application Publication
(10) Pub. No.: US 2023/0348922 A1
(43) Pub. Date: Nov. 2, 2023

(54) METHODS AND COMPOSITIONS FOR ENHANCING YIELD AND DISEASE RESISTANCE

(71) Applicant: Pairwise Plants Services, Inc., Durham, NC (US)

(72) Inventors: Marisa Miller, Durham, NC (US); Devin Lee O'Connor, Hillsborough, NC (US); Lolita George Mathew, Cary, NC (US)

(21) Appl. No.: 18/309,891

(22) Filed: May 1, 2023

Related U.S. Application Data
(60) Provisional application No. 63/337,244, filed on May 2, 2022.

(51) Int. Cl. C12N 15/82; C12N 9/22; C12N 15/11

(52) U.S. Cl. CPC C12N 15/8213; C12N 9/22; C12N 15/11; C12N 15/8261; C12N 15/8271; C12N 15/8262; C12N 2310/20

(57) ABSTRACT

This invention relates to compositions and methods for modifying IDEAL PLANT ARCHITECTURE 1 (IPA1) genes, or orthologues thereof, encoding SQUAMOSA PROMOTER BINDING PROTEIN-LIKE (SPL) transcription factors in plants. The invention further relates to plants and/or parts thereof comprising one or more mutations in endogenous IPA1 genes, or orthologues thereof, produced using the methods and compositions of the invention.

### STATEMENT OF PRIORITY

This application claims the benefit, under 35 U.S.C. § 119 (e), of U.S. Provisional Application No. 63/337,244 filed on May 2, 2022, the entire contents of which is incorporated by reference herein.

### STATEMENT REGARDING ELECTRONIC FILING OF A SEQUENCE LISTING

A Sequence Listing in XML format, entitled 1499-101_ST26.xml, 776,947 bytes in size, generated on Apr. 27, 2023 and filed herewith, is hereby incorporated by reference into the specification for its disclosures.

### FIELD OF THE INVENTION

This invention relates to compositions and methods for modifying IDEAL PLANT ARCHITECTURE 1 (IPA1) genes or orthologues thereof, encoding SQUAMOSA PROMOTER BINDING PROTEIN-LIKE (SPL) transcription factors in plants. The invention further relates to plants comprising modified endogenous IPA1 genes and optionally having improved yield traits and/or disease resistance produced using the methods and compositions of the invention.

### BACKGROUND OF THE INVENTION

Evidence suggests that growth and defense pathways actively antagonize each other, with salicylic acid (SA)-mediated defense being the primary pathway counteracting growth (Butselaar et al. Trends Plant Sci 25, 566-576 (2020)). However, very few genes that actively regulate the growth-SA defense antagonism are characterized. Thus, optimization of plant performance for yield can lead to a penalty in defense. The converse is true in that selection for increased immunity can cause reductions in yield and growth.

Identification of genes that can be engineered to circumvent the antagonistic effects of yield and defense pathways will lead to improved plant health and performance. Novel strategies for modulating the expression of genes involved in improving yield traits and defense pathways are also needed for improvement of crop performance

### SUMMARY OF THE INVENTION

One aspect of the invention provides a plant or plant part thereof comprising at least one mutation in an endogenous IDEAL PLANT ARCHITECTURE 1 (IPA1) gene, or orthologue thereof, encoding a SQUAMOSA PROMOTER BINDING PROTEIN-LIKE (SPL) transcription factor, optionally wherein the endogenous IPA1 gene encoding a SPL transcription factor is an endogenous SQUAMOSA PROMOTER BINDING PROTEIN-LIKE 9 (SPL9) gene, an endogenous UNBRANCHED 2 (UB2) gene, or an endogenous UNBRANCHED 3 (UB3) gene, optionally wherein the at least one mutation may be a non-natural mutation.

A second aspect of the invention provides a plant cell comprising an editing system, the editing system comprising: (a) a CRISPR-Cas associated effector protein; and (b) a guide nucleic acid (e.g., gRNA, gDNA, crRNA, crDNA) comprising a spacer sequence with complementarity to an endogenous IDEAL PLANT ARCHITECTURE 1 (IPA1) target gene, or orthologue thereof, encoding a SQUAMOSA PROMOTER BINDING PROTEIN-LIKE (SPL) transcription factor.

A third aspect provides a plant cell comprising at least one mutation in one or more endogenous IDEAL PLANT ARCHITECTURE 1 (IPA1) genes, or orthologue thereof, encoding a SQUAMOSA PROMOTER BINDING PROTEIN-LIKE (SPL) transcription factor, wherein the at least one mutation is a substitution, insertion and/or a deletion that is introduced using an editing system that comprises a nucleic acid binding domain that binds to a target site in the one or more endogenous IPA1 genes, optionally wherein the at least one mutation may be a non-natural mutation.

In a fourth aspect, a method of providing a plurality of plants exhibiting altered plant architecture, improved yield traits and/or increased tolerance/resistance to abiotic and biotic stress is provided, the method comprising planting two or more plants of the invention in a growing area, thereby providing a plurality of plants exhibiting altered plant architecture, improved yield traits and/or increased tolerance/resistance to abiotic and biotic stress as compared to a plurality of control plants not comprising the at least one mutation, optionally wherein the plurality of plants exhibiting resistance to biotic stress exhibit increased disease resistance.

A fifth aspect provides a method of producing/breeding a transgene-free genome-edited (e.g., base-edited) plant, comprising: (a) crossing the plant of the invention with a transgene free plant, thereby introducing the mutation or modification into the plant that is transgene-free; and (b) selecting a progeny plant that comprises the mutation or modification but is transgene-free, thereby producing a transgene free genome-edited (e.g., base-edited) plant.

In a sixth aspect a method for editing a specific site in the genome of a plant cell is provided, the method comprising: cleaving, in a site-specific manner, a target site within an endogenous IPA1 gene in the plant cell, wherein the endogenous IPA1 gene is: (a) an SPL9 gene having at least 80% sequence identity to a nucleotide sequence of any one of SEQ ID NOs:72, 73, 75, 76, 78, 79, 81, 82, 143, 144, 182, 183, 222, 223, 255 or 256 and/or comprising a region having at least 80% sequence identity to a nucleotide sequence of any one of SEQ ID NOs:146-181, 185-221, 225-254 and/or 258-288, (b) a UB2 gene having at least 80% sequence identity to the nucleotide sequence of SEQ ID NO:84 or SEQ ID NO:85 and/or comprising a region having at least 80% sequence identity to a nucleotide sequence of any one of SEQ ID NOs:90-96 or 332-393; and/or (c) a UB3 gene having at least 80% sequence identity to the nucleotide sequence of SEQ ID NO:87 or SEQ ID NO:88 and/or comprising a region having at least 80% sequence identity to a nucleotide sequence of any one of SEQ ID NOs:90, 97-103 or 394-445, thereby generating an edit in the endogenous IPA1 gene of the plant cell.

A seventh aspect provides a method for making a plant, the method comprising: (a) contacting a population of plant cells that comprise an endogenous IPA1 gene with a nuclease targeted to the endogenous gene, wherein the nuclease is linked to a nucleic acid binding domain that binds to a target site in the endogenous gene, the endogenous IPA1 gene: (i) is an SPL9 gene comprising a nucleotide sequence having at least 80% sequence identity to the nucleotide sequence of any one of SEQ ID NOs:72, 73, 75, 76, 78, 79, 81, 82, 143, 144, 182, 183, 222, 223, 255 or 256, and/or comprising a region having at least 80% sequence identity to a nucleotide sequence of any one of SEQ ID NOs:146-181, 185-221, 225-254 and/or 258-288; (b) a UB2 gene having at least 80% sequence identity to the nucleotide sequence of SEQ ID NO:84 or SEQ ID NO:85, and/or comprising a region having at least 80% sequence identity to a nucleotide sequence of any one of SEQ ID NOs:90-96 or 332-393; and/or a UB3 gene having at least 80% sequence identity to the nucleotide sequence of SEQ ID NO:87 or SEQ ID NO:88, and/or comprising a region having at least 80% sequence identity to a nucleotide sequence of any one of SEQ ID NOs:90, 97-103 or 394-445 (b) selecting a plant cell from the population comprising a mutation in the endogenous IPA1 gene, wherein the mutation is a substitution and/or a deletion; and (c) growing the selected plant cell into a plant comprising the mutation in the endogenous IPA1 gene.

An eighth aspect of the invention provides a method for altering plant architecture, improving yield traits and/or increasing tolerance/resistance of a plant, comprising

(a) contacting a plant cell comprising an endogenous IPA1 gene with a nuclease targeted to the endogenous IPA1 gene, wherein the nuclease is linked to a nucleic acid binding domain that binds to a target site in the endogenous IPA1 gene, wherein the endogenous IPA1 gene is: (i) an SPL9 gene comprising a nucleotide sequence having at least 80% sequence identity to the nucleotide sequence of any one of SEQ ID NOs:72, 73, 75, 76, 78, 79, 81, 82, 143, 144, 182, 183, 222, 223, 255 or 256, and/or comprising a region having at least 80% sequence identity to a nucleotide sequence of any one of SEQ ID NOs:146-181, 185-221, 225-254 and/or 258-288; (ii) a UB2 gene having at least 80% sequence identity to the nucleotide sequence of SEQ ID NO:84 or SEQ ID NO:85, and/or comprising a region having at least 80% sequence identity to a nucleotide sequence of any one of SEQ ID NOs:90-96 or 332-393; and/or (iii) a UB3 gene having at least 80% sequence identity to the nucleotide sequence of SEQ ID NO:87 or SEQ ID NO:88, and/or comprising a region having at least 80% sequence identity to a nucleotide sequence of any one of SEQ ID NOs:90, 97-103 or 394-445; and (b) growing the plant cell into a plant, thereby altering plant architecture, improving yield traits and/or increasing tolerance/resistance of the plant.

A ninth aspect provides a method for producing a plant or part thereof comprising at least one cell having mutation in an endogenous IDEAL PLANT ARCHITECTURE 1 (IPA1) gene, or orthologue thereof, encoding a SQUAMOSA PROMOTER BINDING PROTEIN-LIKE (SPL) transcription factor, the method comprising contacting a target site in the endogenous IPA1 gene in the plant or plant part with a nuclease comprising a cleavage domain and a DNA-binding domain, wherein the DNA binding domain of the nuclease binds to a target site in the endogenous IPA1 gene, wherein the endogenous IPA1 gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NO:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UB3 gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89, thereby producing a plant or part thereof comprising at least one cell having a mutation in the endogenous IPA1 gene.

In a tenth aspect, a method of producing a plant or part thereof comprising a mutation in an endogenous IPA1 gene and having a phenotype of altered plant architecture, improved yield traits and/or increased tolerance/resistance to abiotic and biotic stress is provided, the method comprising contacting a target site in an endogenous IPA1 gene in the plant or plant part with a nuclease comprising a cleavage domain and a nucleic acid binding domain, wherein the nucleic acid binding domain of the nuclease binds to a target site in the endogenous IPA1 gene, wherein the endogenous IPA gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NO:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UB3 gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89, thereby producing a plant or part thereof having a mutated endogenous IPA1 gene and altered plant architecture, improved yield traits and/or increased tolerance/resistance to abiotic and biotic stress.

In an eleventh aspect, a guide nucleic acid that binds to a target site in an endogenous IPA1 gene is provided, wherein the endogenous IPA1 gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NO:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UNBRANCHED 3 (UB3) gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89.

In a twelfth aspect, a system comprising the guide nucleic acid of the invention and a CRISPR-Cas effector protein that associates with the guide nucleic acid.

A thirteenth aspect provides a gene editing system comprising a CR ISPR-Cas effector protein in association with a guide nucleic acid, wherein the guide nucleic acid comprises a spacer sequence that binds to a IPA1 gene.

A fourteenth aspect provides a complex comprising a CRISPR-Cas effector protein comprising a cleavage domain and a guide nucleic acid, wherein the guide nucleic acid binds to a target site in a IPA1 gene, wherein the IPA1 gene (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NO:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UNBRANCHED 3 (UB3) gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:349-445, and/or (iv) encoding a polypeptide sequence having at least 80% identity to the amino acid sequence of SEQ ID NO:89, wherein the cleavage domain cleaves a target strand in the IPA1 gene.

A fifteenth aspect provides an expression cassette comprising (a) a polynucleotide encoding CRISPR-Cas effector protein comprising a cleavage domain and (b) a guide nucleic acid that binds to a target site in an IPA1 gene, wherein the guide nucleic acid comprises a spacer sequence that is complementary to and binds to the target site in the IPA1 gene.

In a sixteenth aspect, a mutated nucleic acid encoding a SPL9 polypeptide is provided, the mutated nucleic acid comprising a sequence having at least 90% sequence identity to any one of SEQ ID NOs:389-300.

In a seventeenth aspect, a mutated nucleic acid encoding a UB2 polypeptide is provided, the mutated nucleic acid comprising a sequence having at least 90% sequence identity to any one of SEQ ID NOs:320, 322, or 324.

In an eighteenth aspect, a mutated nucleic acid encoding a UB3 polypeptide is provided, the mutated nucleic acid comprising a sequence having at least 90% sequence identity to any one of SEQ ID NOs:310, 312, 314, 316, or 318.

In a nineteenth aspect, a soybean plant or part thereof is provided comprising at least one mutation in at least one endogenous SLP9 gene having a gene identification number (gene ID) of Glyma_02G177500 (SPL9a), Glyma_09 G113800 (SPL9b), Glyma_03 g143100 (SPL9c), and/or Glyma_19 g146000 (SPL9d).

A twentieth aspect provides a guide nucleic acid that binds to a target nucleic acid in a SPL9 gene having a gene identification number (gene ID) of Glyma_02G177500 (SPL9a), Glyma_09 G113800 (SPL9b), Glyma_03 g143100 (SPL9c), and/or Glyma_19 g146000 (SPL9d).

A further aspect provides a mutated endogenous SPL9 gene in a plant cell, wherein the mutated endogenous SPL9 gene comprises a nucleic acid sequence having at least 90% identity to any one of SEQ ID NOs:389-300.

An additional aspect provides a mutated endogenous UNBRANCHED 2 (UB2) gene in a plant cell, wherein the mutated endogenous UB2 gene comprises a nucleic acid sequence having at least 90% identity to any one of SEQ ID NOs:320, 322 or 324, and/or a mutated endogenous UNBRANCHED 3 (UB3) gene in a plant cell, wherein the mutated endogenous UB3 gene comprises a nucleic acid sequence having at least 90% identity to any one of SEQ ID NOs:310, 312, 314, 316, or 318.

In a further aspect a mutated UNBRANCHED 2 (UB2) polypeptide in a plant cell is provided, the mutated UB2 polypeptide having at least 90% identity to any one of SEQ ID NOs:321, 323 or 325, and/or a mutated endogenous UNBRANCHED 3 (UB3) polypeptide in a plant cell, the mutated UB3 polypeptide having at least 90% identity to any one of SEQ ID NOs:311, 313, 315, 317, or 319.

Further provided are polypeptides, polynucleotides, nucleic acid constructs, expression cassettes and vectors for making a plant or part thereof of this invention.

These and other aspects of the invention are set forth in more detail in the description of the invention below.

### BRIEF DESCRIPTION OF THE SEQUENCES

SEQ ID NOs:1-17 are exemplary Cas12a amino acid sequences useful with this invention.

SEQ ID NOs:18-20 are exemplary Cas12a nucleotide sequences useful with this invention.

SEQ ID NO:21-22 are exemplary regulatory sequences encoding a promoter and intron. SEQ ID NOs:23-29 are exemplary cytosine deaminase sequences useful with this invention.

SEQ ID NOs:30-40 are exemplary adenine deaminase amino acid sequences useful with this invention.

SEQ ID NO:41 is an exemplary uracil-DNA glycosylase inhibitor (UGI) sequences useful with this invention.

SEQ ID NOs:42-44 provides an example of a protospacer adjacent motif position for a Type V CRISPR-Cas12a nuclease.

SEQ ID NOs:45-47 provide example peptide tags and affinity polypeptides useful with this invention.

SEQ ID NOs:48-58 provide example RNA recruiting motifs and corresponding affinity polypeptides useful with this invention.

SEQ ID NOs:59-60 are exemplary Cas9 polypeptide sequences useful with this invention.

SEQ ID NOs:61-71 are exemplary Cas9 polynucleotide sequences useful with this invention.

SEQ ID NO:72 is an example SPL9a genomic sequence.

SEQ ID NO:73 is an example SPL9a coding (cds) sequence.

SEQ ID NO:74 is an example SPL9a polypeptide sequence.

SEQ ID NO:75 is an example SPL9b genomic sequence.

SEQ ID NO:76 is an example SPL9b coding (cds) sequence.

SEQ ID NO:77 is an example SPL9b polypeptide sequence.

SEQ ID NO:78 is an example SPL9c genomic sequence.

SEQ ID NO:79 is an example SPL9c coding (cds) sequence.

SEQ ID NO:80 is an example SPL9c polypeptide sequence.

SEQ ID NO:81 is an example SPL9d genomic sequence.

SEQ ID NO:82 is an example SPL9d coding (cds) sequence.

SEQ ID NO:83 is an example SPL9d polypeptide sequence.

SEQ ID NO:84 is an example UB2 genomic sequence.

SEQ ID NO:85 is an example UB2 coding (cds) sequence.

SEQ ID NO:86 is an example UB2 polypeptide sequence.

SEQ ID NO:87 is an example UB3 genomic sequence.

SEQ ID NO:88 is an example UB3 coding (cds) sequence.

SEQ ID NO:89 is an example UB3 polypeptide sequence.

SEQ ID NO:90 is an example UB3 promoter region.

SEQ ID NO:91, SEQ ID NO:92 and SEQ ID NO:93 are example portions of a 5′ UTR region of a UB2 nucleic acid.

SEQ ID NO:94 and SEQ ID NO:95 are example intron regions of a UB2 nucleic acid.

SEQ ID NO:96 is an example portion of a 3′ UTR region of a UB2 nucleic acid.

SEQ ID NO:97 and SEQ ID NO:98 are example UB3 promoter regions.

SEQ ID NO:99 is an example portion of a 5′ UTR region of a UB3 nucleic acid.

SEQ ID NO:100 and SEQ ID NO:101 are example intron regions of a UB3 nucleic acid. SEQ ID NO:102 and SEQ ID NO:103 are example 3′ UTR regions of a UB3 nucleic acid.

SEQ ID NOs:104-124 and 301 are example spacer sequences for nucleic acid guides useful for targeting SPL9 nucleic acids.

SEQ ID NOs:125-142, 326 and 327 are example spacer sequences for nucleic acid guides useful for targeting UB2 and UB3 nucleic acids.

SEQ ID NO:144 is an example SPL9a genomic sequence.

SEQ ID NO:145 is an example SPL9a coding (cds) sequence.

SEQ ID NO:146 is an example SPL9a polypeptide sequence.

SEQ ID NO:182 is an example SPL9b genomic sequence.

SEQ ID NO:183 is an example SPL9b coding (cds) sequence.

SEQ ID NO:184 is an example SPL9b polypeptide sequence.

SEQ ID NO:222 is an example SPL9c genomic sequence.

SEQ ID NO:223 is an example SPL9c coding (cds) sequence.

SEQ ID NO:224 is an example SPL9c polypeptide sequence.

SEQ ID NO:255 is an example SPL9d genomic sequence.

SEQ ID NO:256 is an example SPL9d coding (cds) sequence.

SEQ ID NO:257 is an example SPL9d polypeptide sequence.

SEQ ID NOs:146-181, 185-221, 225-254 and 258-288 are example portions or regions of SPL9a, SPL9b, SPL9c and SPL9d genomic sequences.

SEQ ID NOs:289-300 are example SPL9 sequences edited/modified as described herein.

SEQ ID NOs:307-303 are example deleted portions of SPL9 sequences.

SEQ ID NOs:310, 312, 314, 316 and 318 are example UB3 gene sequences edited/modified as described herein.

SEQ ID NOs:311, 313, 315, 317 and 319 are example UB3 polypeptide sequences encoded by mutated UB3 gene sequences SEQ ID NOs:310, 312, 314, 316 and 318, respectively.

SEQ ID NOs:320, 322, and 324 are example UB2 gene sequences edited/modified as described herein.

SEQ ID NOs:321, 323, and 325 are example UB2 polypeptide sequences encoded by mutated UB3 gene sequences SEQ ID NOs:320, 322, and 324, respectively.

SEQ ID NOs:330-331 are example deleted portions of UB3/UB2 sequences.

SEQ ID NOs:332-445 are example portions or regions of UB2 and UB3 genomic sequences.

### DETAILED DESCRIPTION

The present invention now will be described hereinafter with reference to the examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control. A control plant is typically the same plant as the edited plant, but the control plant has not been similarly edited and therefore does not comprise (is devoid of) the mutation. A control plant maybe an isogenic plant and/or a wild type plant. Thus, a control plant can be the same breeding line, variety, or cultivar as the subject plant into which a mutation as described herein is introgressed, but the control breeding line, variety, or cultivar is free of the mutation. In some embodiments, a comparison between a plant of the invention and a control plant is made under the same growth conditions, e.g., the same environmental conditions (soil, hydration, light, heat, nutrients and the like).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence. A “heterologous” nucleotide/polypeptide may originate from a foreign species, or, if from the same species, is substantially modified from its native form in composition and/or genomic locus by deliberate human intervention.

A “native” or “wild type” nucleic acid, nucleotide sequence, polypeptide or amino acid sequence refers to a naturally occurring or endogenous nucleic acid, nucleotide sequence, polypeptide, or amino acid sequence. Thus, for example, a “wild type endogenous IDEAL PLANT ARCHITECTURE 1 (IPA1) gene” is an IPA2 gene that is naturally occurring in or endogenous to the reference organism, e.g., a plant. As a further examples, a SQUAMOSA PROMOTER BINDING PROTEIN-LIKE 9 (SPL9) gene is an SPL9 gene (e.g., SPL9a, SPL9b, SPL9c, SPL9d) that is naturally occurring in or endogenous to the reference organism, e.g., a plant, such as a soybean plant; and an endogenous UNBRANCHED2 (UB2) gene or endogenous UNBRANCHED3 (UB3) gene are UB2/UB3 genes that are naturally occurring in or endogenous to the reference organism, e.g., a plant, such as a corn plant.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “knock-out mutation” is a mutation that results in a non-functional protein, but which may have a detectable transcript or protein. A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wild type gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

A “gain-of-function” allele or mutation is a mutation that confers a new function on the encoded gene product and/or confers a new gene expression pattern. In some embodiments, a gain-of-function mutation may be dominant or semi-dominant.

As used herein, a “non-natural mutation” refers to a mutation that is generated though human intervention and differs from mutations found in the same gene that have occurred in nature (e.g., occurred naturally). A “non-natural” mutation as used herein does not include a mutation generated in a gene through human intervention, but which is the same mutation as a naturally occurring mutation in that gene.

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

A plant in which at least one orthologous IPA1 gene encoding an SPL transcription factor is modified as described herein (e.g., comprises a modification as described herein) may have altered plant architecture, improved yield traits and/or increased tolerance/resistance to abiotic and biotic stress as compared to a plant or plant part devoid of the same modification (e.g., mutation) in the at least one orthologous IPA1 gene. As used herein, “improved yield traits” refers to any plant trait associated with growth, for example, biomass, yield, nitrogen use efficiency (NUE), inflorescence size/weight, fruit yield, fruit quality, fruit size, seed size, seed number, foliar tissue weight, nodulation number, nodulation mass, nodulation activity, number of seed heads, number of tillers, number of branches, number of flowers, number of tubers, tuber mass, bulb mass, number of seeds, total seed mass, rate of leaf emergence, rate of tiller/branch emergence, rate of seedling emergence, length of roots, number of roots, size and/or weight of root mass, or any combination thereof. Thus, in some aspects, “improved yield traits” may include, but is not limited to, increased inflorescence production, increased fruit production (e.g., increased number, weight and/or size of fruit; e.g., increase number, weight, and/or size of ears for, e.g., maize), increased fruit quality, increased number, size and/or weight of roots, increased meristem size, increased seed size, increased biomass, increased leaf size, increased nitrogen use efficiency, increased height, increased internode number and/or increased internode length as compared to a control plant or part thereof (e.g., a plant that does not comprise/is devoid of a mutated endogenous IPA1 nucleic acid (e.g., a mutated IPA1 gene)). In some embodiments, a plant or part thereof of the invention may exhibit improved yield traits, wherein the improved yield traits include but are not limited to, one or more of the phenotype(s) of increased kernel row number, optionally without substantially reducing ear length, increased kernel size, increased ear length, decreased tiller number, decreased tassel branch number, reduced time to flowering, increased seed number per plant, increased pods per node and/or per plant, and/or increased seed weight, in any combination. Improved yield traits can also result from increased planting density of plants of the invention. Thus, in some aspects, a plant of the invention is capable of being planted at an increased density (as a consequence of altered plant architecture resulting from the endogenous mutation), which results in improved yield traits as compared to a control plant that is planted at the same density. In some aspects, improved yield traits can be expressed as quantity of grain produced per area of land (e.g., bushels per acre of land).

As used herein a “control plant” means a plant that does not contain an edited IPA1 gene or genes as described herein that imparts an enhanced/improved trait (e.g., yield trait) or altered phenotype. A control plant is used to identify and select a plant edited as described herein and that has an enhanced trait or altered phenotype as compared to the control plant. A suitable control plant can be a plant of the parental line used to generate a plant comprising a mutated IPA1 gene(s), for example, a wild type plant devoid of an edit in an endogenous IPA1 gene as described herein. A suitable control plant can also be a plant that contains recombinant nucleic acids that impart other traits, for example, a transgenic plant having enhanced herbicide tolerance. A suitable control plant can in some cases be a progeny of a heterozygous or hemizygous transgenic plant line that is devoid of the mutated IPA1 gene as described herein, known as a negative segregant, or a negative isogenic line.

An enhanced trait may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant. An altered phenotype may be, for example, plant height, biomass, canopy area, anthocyanin content, chlorophyll content, water applied, water content, and water use efficiency.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. However, any technique can be used to measure the amount of, the comparative level of, or the difference in any selected chemical compound or macromolecule in the transgenic plants.

As used herein an “enhanced trait” means a characteristic of a plant resulting from mutations in an IPA1 gene(s) as described herein. Such traits include, but are not limited to, an enhanced agronomic trait characterized by enhanced plant morphology, physiology, growth and development, yield, nutritional enhancement, disease or pest resistance, or environmental or chemical tolerance. In some embodiments, an enhanced trait/altered phenotype may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, drought tolerance, increased water use efficiency, cold tolerance, increased nitrogen use efficiency, and increased yield. In some embodiments, a trait is increased yield under nonstress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, ear size, ear tip filling, kernel abortion, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), flowering time and duration, ear number, ear size, ear weight, seed number per ear or pod, seed size, composition of seed (starch, oil, protein) and characteristics of seed fill.

Also used herein, the term “trait modification” encompasses altering the naturally occurring trait by producing a detectable difference in a characteristic in a plant comprising a mutation in an endogenous IPA1 gene encoding an SPL transcription factor as described herein relative to a plant not comprising the mutation, such as a wild-type plant, or a negative segregant. In some cases, the trait modification can be evaluated quantitatively. For example, the trait modification can entail an increase or decrease in an observed trait characteristics or phenotype as compared to a control plant. It is known that there can be natural variations in a modified trait. Therefore, the trait modification observed entails a change of the normal distribution and magnitude of the trait characteristics or phenotype in the plants as compared to a control plant.

The present disclosure relates to a plant with improved economically important characteristics, more specifically increased yield. More specifically the present disclosure relates to a plant comprising a mutation(s) in an IPA1 gene(s) as described herein, wherein the plant has increased yield as compared to a control plant devoid of said mutation(s). In some embodiments, plants produced as described herein exhibit increased yield or improved yield trait components as compared to a control plant. In some embodiments, a plant of the present disclosure exhibits an improved trait that is related to yield, including but not limited to increased nitrogen use efficiency, increased nitrogen stress tolerance, increased water use efficiency and increased drought tolerance, as defined and discussed infra.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, e.g., increased root biomass, steeper root angle and/or longer roots, and the like), flowering time and duration, grain fill period. Root architecture and development, photosynthetic efficiency, nutrient uptake, stress tolerance, early vigor, delayed senescence and functional stay green phenotypes may be factors in determining yield. Optimizing the above-mentioned factors can therefore contribute to increasing crop yield.

Reference herein to an increase/improvement in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive organs, and/or to propagules (such as seeds) of that plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination.

“Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor, for example, can be a combination of the ability of seeds to germinate and emerge after planting and the ability of the young plants to grow and develop after emergence. Plants having early vigor also show increased seedling survival and better establishment of the crop, which often results in highly uniform fields with the majority of the plants reaching the various stages of development at substantially the same time, which often results in increased yield. Therefore, early vigor can be determined by measuring various factors, such as kernel weight, percentage germination, percentage emergence, seedling growth, seedling height, root length, root and shoot biomass, canopy size and color and others.

Further, increased yield can also manifest as increased total seed yield, which may result from one or more of an increase in seed biomass (seed weight) due to an increase in the seed weight on a per plant and/or on an individual seed basis an increased number of, for example, flowers/panicles per plant; an increased number of pods; an increased number of nodes; an increased number of flowers (“florets”) per panicle/plant; increased seed fill rate; an increased number of filled seeds; increased seed size (length, width, area, perimeter), which can also influence the composition of seeds; and/or increased seed volume, which can also influence the composition of seeds. In one embodiment, increased yield can be increased seed yield, for example, increased seed weight; increased number of filled seeds; and increased harvest index.

Increased yield can also result in modified architecture, or can occur because of modified plant architecture.

Increased yield can also manifest as increased harvest index, which is expressed as a ratio of the yield of harvestable parts, such as seeds, over the total biomass

The disclosure also extends to harvestable parts of a plant such as, but not limited to, seeds, leaves, fruits, flowers, bolls, pods, siliques, nuts, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example seeds, or weight of seeds, per acre, pounds per acre, bushels per acre, tones per acre, tons per acre, kilo per hectare.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, retranslocation (within the plant) and use of nitrogen by the plant.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Typically, plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein, “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein, “water deficit” refers to the conditions or environments that provide less than optimal amounts of water needed for adequate/successful growth and development of plants.

As used herein, “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the World Intellectual Property Organization (WIPO) Standard ST.26. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82)/0, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a “portion” of a wild type CRISPR-Cas repeat sequence (e.g., a wild Type CRISPR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment may comprise, consist essentially of or consist of about 20, 30, 40, 50, 100, 150, 200, 250, 300, 350, 400, 450, 500, 550, 600, 650, 700, 750, 800, 850, 900, 1000, 1050, 1100, 1150, 1200, 1250, 1300, 1350, 1400, 1450 1500, 1550, 1600, 1650, 1700, 1750, 1800, 1850, 1900, 1950, 2000, 2500, 3000, 3500, 4000, 4500, 5000, 5500, 6000, 6500, 7000, or more consecutive nucleotides or any range or value therein of an endogenous IDEAL PLANT ARCHITECTURE 1 (IPA1) gene, or orthologue thereof, encoding a SQUAMOSA PROMOTER BINDING PROTEIN-LIKE (SPL) transcription factor (e.g., endogenous SQUAMOSA PROMOTER BINDING PROTEIN-LIKE 9 (SPL9) genes, n endogenous UNBRANCHED 2 (UB2) genes, and/or endogenous UNBRANCHED 3 (UB3) genes).

In some embodiments, a “sequence-specific nucleic acid binding domain” (e.g., sequence-specific DNA binding domain) may bind to one or more fragments or portions of IPA1 nucleic acids (e.g., IPA1 and/or orthologues thereof) encoding SPL transcription factors as described herein (e.g., SEQ ID NOs:146-181, 185-221, 225-254 and/or 258-288).

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 260, 270, 280, 290, or 300, or more consecutive amino acids of a reference polypeptide. In some embodiments, a polypeptide fragment may comprise, consist essentially of, or consist of about 10, 11, 12, 13, 14, 15, 16, 17, 18, 19, 20, 25, 30, 35, 40, 45, 50, 60, 70, 80, 90, 100, 125, 150, 175, 200, 225, 250, 275, 300, 325, 340, 341, 342, 343, 345, 350, 351, 352, 353, 354, 355, 356, 357, 358, 359, 360, 365, 366, 367, 368, 369, 370, 372, or 373, or more consecutive amino acid residues, or any range or value therein, of a polypeptide encoded by an endogenous IPA1 gene or orthologue thereof (e.g., a fragment or a portion of SEQ ID NO:74, SEQ ID NO:77, SEQ ID NO:80, SEQ ID NO:83, SEQ ID NO:86, SEQ ID NO:89, SEQ ID NO:145, SEQ ID NO:184, SEQ ID NO:224 and/or SEQ ID NO:257). In some embodiments, a deletion may be an in-frame deletion. In some embodiments, such a deletion may be a null mutation, a dominant negative mutation, a semi-dominant mutation, a hypermorphic mutation, or a weak loss-of-function mutation (e.g., hypermorphic mutation), which when comprised in a plant can result in the plant exhibiting improved yield traits without a reduction in disease resistance, a plant exhibiting improved yield traits and increased disease resistance, and/or a plant having increased disease resistance. In some embodiments, the at least one mutation in an endogenous IPA1 gene in a plant may result in the plant having altered plant architecture, improved yield traits and/or increased tolerance/resistance to abiotic and biotic stress as compared to a plant or plant part devoid of the same mutation. Improved yield traits can include, but are not limited to, increased yield (bu/acre), increased kernel row number, increased ear length, ears exhibiting increased kernel row number without a substantial reduction in ear length, increased kernel size, increased branch number, increased flower number, increased node number, increased biomass, increased tassel branch number (TBN), decreased tiller number, decreased tassel branch number (TBN), increased seed number, increased seed size/weight, increased number of pods per node, increase number of pods per plant, and the like, as compared to a plant not comprising said deletion. In some embodiments, improved yield traits in a plant or part thereof of the invention can comprise one or more of the phenotype(s) of increased kernel row number, increased kernel size, increased ear length, decreased tiller number, decreased tassel branch number, reduced time to flowering, increased seed number per plant, increased pods per node and/or per plant, and/or increased seed weight, in any combination.

An IPA1 gene, or orthologue thereof, may be edited in more than one location, thereby providing an IPA1 gene or orthologue thereof comprising more than one mutation. A plant may comprise more than one IPA1 gene, or orthologue thereof, and one or more than one IPA1 gene, or orthologue thereof, in the plant may be edited.

In some embodiments, a “portion” or “region” in reference to a nucleic acid means at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more consecutive nucleotides from a gene (e.g., a IPA1 gene or orthologue thereof, e.g., SPL9, UB2/UB3). In some embodiments, a portion or region of an IPA1 gene, or orthologue thereof, may be about 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 75, 80, 81, 82, 83, 84, 85, 90, 91, 92, 93, 94, 95, 96, 97, 98, 99, 100, 101, 102, 103, 104, 105, 110, 111, 12, 113, 114, 115, 116, 117, 118, 119, 120, 121, 122, 123, 124, 125, 126, 127, 128, 129, 130, 131, 132, 133, 134, 135, 140, 141, 142, 143, 144, 145, 146, 147, 148, 149, 150, 151, 152, 153, 154, 155, 156, 157,158, 159, 160, 161, 162, 163, 164, 165, 170, 171, 172, 173, 174, 175, 180, 185, 186, 187, 188, 189, 190, 191, 192, 193, 194, 195, 196, 197, 198, 199, 200, 205, 206, 207, 208, 209, 211, 212, 213, 214, 215, 216, 217, 218, 219, 220, 221, 222, 223, 224, 225, 230, 235, 240, 241, 242, 243, 244, 245, 246, 247, 248, 249, 250, 251, 252, 253, 254, 255, 260, 265, 270, 275, 276, 277, 278, 279, 280, 281, 282, 283, 284, 285, 286, 287, 288, 289, 290, 300, 325, 330, 335, 340, 345, 346, 347, 348, 349, 350, 351, 352, 353, 354, 355, 356, 357, 358, 359, 360, 365, 370, 371, 372, 737, 374, 375, 380, 390, 400, 420, 440, 441, 442, 443, 444, 445, 446, 447, 448, 449, or 450, or more consecutive nucleotides, or any range or value therein (e.g., from about nucleotide 2001 to about nucleotide 2364 with reference to the nucleotide numbering of SEQ ID NO:72, from about nucleotide 1 to about nucleotide 364 with reference to the nucleotide numbering of SEQ ID NO:73, from about nucleotide 2001 to about nucleotide 2370 with reference to the nucleotide numbering of SEQ ID NO:75, from about nucleotide 1 to about nucleotide 370 with reference to the nucleotide numbering of SEQ ID NO:76, from about nucleotide 2001 to about nucleotide 2347 with reference to the nucleotide numbering of SEQ ID NO:78, from about nucleotide 1 to about nucleotide 347 with reference to the nucleotide numbering of SEQ ID NO:79, from about nucleotide 2001 to about nucleotide 2349 with reference to the nucleotide numbering of SEQ ID NO:81, or from about nucleotide 1 to about nucleotide 349 with reference to the nucleotide numbering of SEQ ID NO:82; e.g., SEQ ID NOs:146-181, 185-221, 225-254 and/or 258-288). In some embodiments, a “portion” or “region” in reference to a polypeptide means at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 70, 75, 80, 85, 90, 95, 100, 110, 120, 130, 140, 150, 175, 200, 225, 250, 275, 300, 325, or 350, or more consecutive amino acid residues from a polypeptide (e.g., a SQUAMOSA PROMOTER BINDING PROTEIN-LIKE (SPL) transcription factor).

In some embodiments, a “sequence-specific nucleic acid binding domain” may bind to one or more fragments or portions of nucleotide sequences encoding SPL transcription factors as described herein.

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene. Frameshift mutations in a gene can result in the production of a polypeptide that is longer, shorter or the same length as the wild type polypeptide depending on when the first stop codon occurs following the mutated region of the gene.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82)/0, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40, 50, 60, 70, or 80 nucleotides or more).

In some embodiments of the invention, the substantial identity exists over a region of consecutive amino acid residues of a polypeptide of the invention that is about 3 amino acid residues to about 20 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8 consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids in length or more consecutive amino acid residues). In some embodiments, two or more SPL polypeptides may be identical or substantially identical (e.g., at least 70% to 99.9% identical; e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% identical or any range or value therein).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2×(or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific nucleic acid binding domain (e.g., a sequence-specific nucleic acid binding domain from a polynucleotide-guided endonuclease, a zinc finger nuclease, a transcription activator-like effector nuclease (TALEN), an Argonaute protein, and/or a CR1SPR-Cas endonuclease (e.g., CR1SPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CR1SPR-Cas endonuclease (e.g., CR1SPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus and/or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a nucleic acid binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA2-δ promoter from Arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific nucleic acid (e.g., DNA) binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific nucleic acid binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter or they may be different promoters. Thus, a polynucleotide encoding a sequence specific nucleic acid binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific nucleic acid binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific nucleic acid binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally, included are shuttle vectors by which is meant a DNA vehicle capable, naturally or by design, of replication in two different host organisms, which may be selected from actinomycetes and related species, bacteria and eukaryotic (e.g., higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific nucleic acid binding protein, the reverse transcriptase and/or the deaminase are expressed and the sequence-specific nucleic acid binding protein binds to the target nucleic acid, and the reverse transcriptase and/or deaminase may be fused to either the sequence-specific nucleic acid binding protein or recruited to the sequence-specific nucleic acid binding protein (via, for example, a peptide tag fused to the sequence-specific nucleic acid binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

The present invention provides methods and compositions for altering plant architecture, improving yield traits in plants and/or increasing plant tolerance/resistance to abiotic and biotic stress, optionally wherein yield traits are improved without loss of defense responses, that is, optimizing plant performance for yield without a penalty in defense against abiotic and biotic stresses. An example of a gene that regulates the growth-defense tradeoff is rice IDEAL PLANT ARCHITECTURE 1 (IPA1), which encodes a SQUAMOSA PROMOTER BINDING PROTEIN-LIKE (SPL) transcription factor that is regulated by microRNA 156 (miR156) (Jiao et al. Nat Genet 42, 541-544 (2010)). IPA1 was shown to have distinct binding affinities depending on the immune status of the plant (Wang et al. Science 361, 1026-1028 (2018)). During pathogen infection, IPA1 is phosphorylated and preferentially activates expression of a key regulator of SA-mediated defenses, WRKY45. In the absence of an immune response, IPA1 is not phosphorylated and activates genes crucial for growth and yield like DENSE AND ERECT PANICLE 1 (DEP1). In rice plants harboring the ipa1-1D allele, a mutation in the miR156 binding site elevates transcript and protein levels of IPA1 in both infected and uninfected plants, thereby leading to quantitative improvements to both yield and defense.

In soy, there are four IPA1 orthologs that are SPL9 family transcription factors, SPL9a-d. These four GmSPL9 genes are negatively regulated by GmmiR156b (Bao, A. et al. BMC Plant Biol 19, 131 (2019); Cao et al. Plant Mol Biol 89, 353-363 (2015)). Corn orthologs of IPA1 are unbranched 2 and unbranched 3 (UB2, UB3) (Chuck et al. Proc National Acad Sci 111, 18775-18780 (2014)). While decreasing SPL activity may increase the activity of stem cell identity genes resulting in increased meristem size, kernel row number, and yield, the context under which SPLs are misregulated is important. This is because strong SPL loss of function in corn results in dramatic changes to plant architecture including de-repressed leaves, homeotic organ identity changes, increased root number, and increased tiller number, which traits are not desirable for increased yield (Chuck et al. Nat Genet 39, 544-549 (2007)). How any of these developmental phenotypes may determine disease susceptibility, or if corn SPLs are involved in disease resistance is not known.

Accordingly, as described herein, editing technology is used to target IPA1 genes in plants to generate plants with improved yield traits without loss in or antagonism of disease resistance. Mutations that may be useful for production of such plants include, for example, substitutions, deletions and insertions, optionally a point mutation. In some aspects, a mutation generated by the editing technology can result in a dominant negative mutation, a semi-dominant mutation, a hypomorphic mutation, a weak loss-of-function mutation, a hypermorphic mutation, or a null allele, optionally, wherein the mutation results in a null allele.

In some embodiments, the invention provides a plant or plant part thereof, the plant or plant part comprising at least one (e.g., one or more) mutation (e.g., 1, 2, 3, 4, 5, or more mutations) in an endogenous IDEAL PLANT ARCHITECTURE 1 (IPA1) gene, or orthologue thereof, encoding a SQUAMOSA PROMOTER BINDING PROTEIN-LIKE (SPL) transcription factor. An endogenous IPA1 gene encoding a SPL transcription factor may be an endogenous SQUAMOSA PROMOTER BINDING PROTEIN-LIKE 9 (SPL9) gene, an endogenous UNBRANCHED 2 (UB2) gene, or an endogenous UNBRANCHED 3 (UB3) gene. An SPL9 gene includes, for example, an SPL9a gene, a SPL9b gene, a SPL9c gene and/or a SPL9d gene. In some embodiments, an endogenous gene IPA1 gene, or orthologue thereof, may be regulated by miR156, optionally, wherein the miR156 includes, but is not limited to, miR156a, miR156b, miR156c, miR156d, miR156e. In some embodiments, the miR156 is miR156b.

In some embodiments, an endogenous IPA1 gene may be an endogenous SPL9 gene, optionally wherein the endogenous SPL9 gene is an SPL9a gene, a SPL9b gene, a SPL9c gene and/or a SPL9d gene, optionally the SPL9 gene is present in the plant or part thereof as two paralogous pairs (a) an SPL9a gene and a SPL9b gene and/or (b) a SPL9c gene and a SPL9d gene. Example SPL9 genes useful with the invention include, but are not limited to, those that (a) comprise a nucleotide sequence having at least 80% sequence identity to the nucleotide sequence of any one of SEQ ID NOs:72, 75, 78, 81, 143, 182, 222, or 255, (b) comprise a coding sequence having at least 80% sequence identity to the nucleotide sequence of any one of SEQ ID NOs:73, 76, 79, 82, 144, 183, 223, or 256, (c) comprise a region having least 80% sequence identity to the nucleotide sequence of any one of SEQ ID NOs:146-181, 185-221, 225-254 and/or 258-288; and/or (d) encode a polypeptide sequence having at least 80% identity to the amino acid sequence of any one of SEQ ID NO:74, 77, 80, 83, 145, 184, 224, or 257.

In some embodiments, an endogenous IPA1 gene may be an endogenous UB2 gene and/or an endogenous UB3 gene. Example UB2 genes useful with the invention (a) comprise a nucleotide sequence having at least 80% sequence identity to the nucleotide sequence of SEQ ID NO:84, (b) comprise a coding sequence having at least 80% sequence identity to the nucleotide sequence of SEQ ID NO:85, (c) comprise a region having least 80% sequence identity to the nucleotide sequence of any one of SEQ ID NOs:90-96 or 332-393; and/or (d) encode a polypeptide sequence having at least 80% identity to the amino acid sequence of SEQ ID NO:86. Example UB3 genes useful with the invention (a) comprise a nucleotide sequence having at least 80% sequence identity to the nucleotide sequence of SEQ ID NO:87, (b) comprise a coding sequence having at least 80% sequence identity to the nucleotide sequence of SEQ ID NO:88, (c) comprise a region having least 80% sequence identity to the nucleotide sequence of any one of SEQ ID NOs:90, 97-103 or 394-445; and/or (d) encode a polypeptide sequence having at least 80% identity to the amino acid sequence of SEQ ID NO:89.

A mutation in an endogenous IPA1 gene, or orthologue thereof, in a plant, plant part thereof or a plant cell may be any type of mutation that results in a plant having altered plant architecture, improved yield traits and/or increased tolerance/resistance to abiotic and biotic stress as compared to a plant or plant part devoid of the same mutation, optionally wherein these modifications in phenotype occur without loss in disease response. Such mutations include a base substitution, a base deletion and/or a base insertion. In some embodiments, a mutation may comprise a base substitution to an A, a T, a G, or a C. In some embodiments, the at least one mutation may be a base substitution to from a C to a T (C>T). In some embodiments, a mutation may be a deletion of one or more base pairs (e.g., 1, 2, 3, 4, 5, 6, 7, 8, 9, or 10 base pairs to about 50, 60, 70, 80, 90 or 100 or more base pairs; e.g., 1 base pair to about 100 base pairs or any value or range therein) or an insertion of one or more base pairs. In some embodiments, a deletion or insertion may be an in-frame deletion, an in-frame insertion, an out-of-frame deletion or an out-of-frame insertion. In some embodiments, a mutation in an IPA1 gene as described herein can result in a dominant negative mutation, a semi-dominant mutation, a hypomorphic mutation, a weak loss-of-function mutation, a hypermorphic mutation, or a null allele, optionally where the mutation may result in a null allele. In some embodiments, the mutation in an IPA1 gene as described herein results in a hypomorphic mutation. In some embodiments, a mutation may be a null allele and a hypomorphic mutation. In some embodiments, a mutation in an IPA1 gene as described herein may be a non-natural mutation.

In some embodiments, a mutation (e.g., at least one mutation, e.g., one or more mutations) in an endogenous IPA1 gene encoding a SPL transcription factor may be in the first exon of the endogenous IPA1 gene encoding a SPL transcription factor, optionally resulting in a premature stop codon and a null allele. In some embodiments, the mutation is in an SPL9 gene and may be present in at least one (e.g., 1, 2, 3, or 4) of the SPL9a gene, the SPL9b gene, the SPL9c gene, and/or the SPL9d gene, wherein the at least one mutation is in the first exon, optionally resulting in a premature stop codon and a null allele. In some embodiments, the mutation is in an SPL9 gene and may be present in at least two (e.g., 2, 3, or 4) of the SPL9a gene, the SPL9b gene, the SPL9c gene, and/or the SPL9d gene, in any combination, optionally wherein the at least one mutation is in the first exon of the SPL9a gene, the SPL9b gene, the SPL9c gene, and/or the SPL9d, optionally resulting in a premature stop codon and a null allele. In some embodiments, the mutation is present in each of the SPL9a gene, the SPL9b gene, the SPL9c gene and the SPL9d gene. In some embodiments, the first exon of an SPL9a gene can be located from about nucleotide 2001 to about nucleotide 2364 with reference to the nucleotide numbering of SEQ ID NO:72 and/or from about nucleotide 1 to about nucleotide 364 with reference to the nucleotide numbering of SEQ ID NO:73. In some embodiments, the first exon of an SPL9b gene can be located from about nucleotide 2001 to about nucleotide 2370 with reference to the nucleotide numbering of SEQ ID NO:75 and/or from about nucleotide 1 to about nucleotide 370 with reference to the nucleotide numbering of SEQ ID NO:76. In some embodiments, the first exon of the SPL9c gene can be located from about nucleotide 2001 to about nucleotide 2347 with reference to the nucleotide numbering of SEQ ID NO:78 and/or from about nucleotide 1 to about nucleotide 347 with reference to the nucleotide numbering of SEQ ID NO:79. In some embodiments, the first exon of the SPL9d gene can be located from about nucleotide 2001 to about nucleotide 2349 with reference to the nucleotide numbering of SEQ ID NO:81 and/or from about nucleotide 1 to about nucleotide 349 with reference to the nucleotide numbering of SEQ ID NO:82.

In some embodiments, at least one mutation in an SPL9 gene can be (a) in a region of the first exon of the SPL9a gene located from about nucleotide 2053 to about nucleotide 2115 with reference to the nucleotide numbering of SEQ ID NO:72 or SEQ ID NO:75, (b) in a region of the first exon of the SPL9b gene located from about nucleotide 2015 to about nucleotide 2077 with reference to the nucleotide numbering of SEQ ID NO:78 or SEQ ID NO:81(c) in a region of the first exon of the SPL9c gene located from about nucleotide 1 to about nucleotide 115 with reference to the nucleotide numbering of SEQ ID NO:73 or SEQ ID NO:76 and/or (d) a region of the first exon of the SPL9d gene located from about nucleotide 1 to about nucleotide 77 with reference to the nucleotide numbering of SEQ ID NO:79 or SEQ ID NO:82. In some embodiments, at least one mutation in an SPL9 gene can be located (a) in a region of the first exon of an SPL9a gene having at least 80% sequence identity to the nucleotide sequence of SEQ ID NOs:161-177, (b) in a region of the first exon of an SPL9b gene having least 80% sequence identity to the nucleotide sequence of any one of SEQ ID NOs:201-217, (c) in a region of the first exon of an SPL9c gene having least 80% sequence identity to the nucleotide sequence of any one of SEQ ID NOs:240-250, or (d) in a region of the first exon of an SPL9d gene having least 80% sequence identity to the nucleotide sequence of any one of SEQ ID NOs:274-284.

In some embodiments, at least one mutation can be located in a region of an SPL9a gene having at least 80% sequence identity to the nucleotide sequence of any one of SEQ ID NOs:146-181. In some embodiments, at least one mutation can be located in a region of an SPL9b gene having at least 80% sequence identity to the nucleotide sequence of any one of SEQ ID NOs:185-221. In some embodiments, at least one mutation can be located in a region of an SPL9c gene having at least 80% sequence identity to the nucleotide sequence of any one of SEQ ID NOs:225-254. In some embodiments, at least one mutation can be located in a region of an SPL9d gene having at least 80% sequence identity to the nucleotide sequence of any one of SEQ ID NOs:258-288.

In some embodiments, a mutation (e.g., at least one mutation, e.g., one or more mutations) in an endogenous IPA1 gene encoding a SPL transcription factor can be in the first exon of the endogenous IPA1 gene encoding a SPL transcription factor, wherein IPA1 gene is a UB2 gene or a UB3 gene. In some embodiments, a mutation in a UB2 gene may be within the third exon of the endogenous UB2 gene (see e.g., third exon of SEQ ID NO:84; e.g., SEQ ID NOs: 358-376, optionally SEQ ID NOs:373-376). In some embodiments, a mutation in a UB3 gene may be within the third exon of the endogenous UB3 gene (see e.g., third exon of SEQ ID NO:87; e.g., SEQ ID NOs:408-426, optionally SEQ ID NOs: 415-416. In some embodiments, the mutation in the first exon or the third exon results in a premature stop codon and a null allele, optionally resulting in a hypomorphic or knockout mutation.

In some embodiments, a mutation (e.g., at least one mutation, e.g., one or more mutations) in an endogenous IPA1 gene encoding a SPL transcription factor may be in a miR156 binding site of the endogenous IPA1 gene, optionally wherein the endogenous IPA1 gene is an SPL9 gene, a UB2 gene and/or a UB3 gene. In some embodiments, the endogenous IPA1 gene can be (a) an SPL9a gene and the miR156 binding site can be located in a region from about nucleotide 6569 to about nucleotide 6588 with reference to the nucleotide numbering of SEQ ID NO:72, from about nucleotide 758 to about nucleotide 777 with reference to the nucleotide numbering of SEQ ID NO:73, and/or from about nucleotide 6624 to about nucleotide 6847 with reference to the nucleotide numbering of SEQ ID NO:143, (b) an SPL9b gene and the miR156 binding site is from about nucleotide 6269 to about nucleotide 6288 with reference to the nucleotide numbering of SEQ ID NO:75, from about nucleotide 760 to about nucleotide 780 with reference to the nucleotide numbering of SEQ ID NO:76, and/or from about nucleotide 6265 to about nucleotide 6488 with reference to the nucleotide numbering of SEQ ID NO:182, (c) an SPL9c gene and the miR156 binding site is from about nucleotide 5388 to about nucleotide 5407 with reference to the nucleotide numbering of SEQ ID NO:78, from about nucleotide 761 to about nucleotide 780 with reference to the nucleotide numbering of SEQ ID NO:79, and/or from about nucleotide 5665 to about nucleotide 5887 with reference to the nucleotide numbering of SEQ ID NO:222, and/or (d) an SPL9d gene and the miR156 binding site is from about nucleotide 5798 to about nucleotide 5817 with reference to the nucleotide numbering of SEQ ID NO:81, from about nucleotide 737 to about nucleotide 756 with reference to the nucleotide numbering of SEQ ID NO:82, and/or from about nucleotide 6120 to about nucleotide 6342 with reference to the nucleotide numbering of SEQ ID NO:255.

In some embodiments, (a) a mutation (e.g., at least one mutation, e.g., one or more mutations) in a miR156 binding site of an endogenous SPL9a gene can be located in a region of the SPL9a gene from about nucleotide 6549 to about nucleotide 6608 with reference to the nucleotide numbering of SEQ ID NO:72 and/or from about nucleotide 738 to about nucleotide 797 with reference to the nucleotide numbering of SEQ ID NO:73, optionally in a region having about 80% sequence identity to any one of the nucleotide sequences of SEQ ID NO:178-181; (b) a mutation in a miR156 binding site of an endogenous SPL9b gene can be located in a region of the endogenous SPL9b gene from about nucleotide 6250 to about nucleotide 6308 with reference to the nucleotide numbering of SEQ ID NO:75 and/or from about nucleotide 741 to about nucleotide 800 with reference to the nucleotide numbering of SEQ ID NO:76, optionally in a region having about 80% sequence identity to any one of the nucleotide sequences of SEQ ID NO:218-221, (c) a mutation in a miR156 binding site of an endogenous SPL9c gene can be located in a region of the endogenous SPL9c gene from about nucleotide 5368 to about nucleotide 5427 with reference to the nucleotide numbering of SEQ ID NO:78 and/or from about nucleotide 742 to about nucleotide 800 with reference to the nucleotide numbering of SEQ ID NO:79, optionally in a region having about 80% sequence identity to any one of the nucleotide sequences of SEQ ID NO:251-254, and/or (d) a mutation in a miR156 binding site of an endogenous SPL9d gene can be located in a region of the of the endogenous SPL9d gene from about nucleotide 5778 to about nucleotide 5837 with reference to the nucleotide numbering of SEQ ID NO:81 and/or from about nucleotide 718 to about nucleotide 775 with reference to the nucleotide numbering of SEQ ID NO:82, optionally in a region having about 80% sequence identity to any one of the nucleotide sequences of SEQ ID NO:285-288.

In some embodiments, the endogenous IPA1 gene can be UB2 gene and the miR156 binding site in the UB2 gene can be located in a region from about nucleotide 4928 to about nucleotide 4947 with reference to the nucleotide numbering of SEQ ID NO:84 and/or from about nucleotide 815 to about nucleotide 834 with reference to the nucleotide numbering of SEQ ID NO:85, and/or the endogenous IPA1 gene is a UB3 gene and the miR156 binding site in the UB3 gene is located in a region from about nucleotide 5301 to about nucleotide 5320 with reference to the nucleotide numbering of SEQ ID NO:87 and/or from about nucleotide 848 to about nucleotide 866 with reference to the nucleotide numbering of SEQ ID NO:88.

In some embodiments, a mutation in a miR156 binding site of an endogenous UB2 gene can be located in a region of the UB2 gene that is from about nucleotide 4894 to about nucleotide 4967 with reference to the nucleotide numbering of SEQ ID NO:84 and/or from about nucleotide 781 to about nucleotide 854 with reference to the nucleotide numbering of SEQ ID NO:85, and/or a mutation in a miR156 binding site of an endogenous UB3 gene can be located in a region of the UB3 gene that is from about nucleotide 5267 to about nucleotide 5339 with reference to the nucleotide numbering of SEQ ID NO:87 and/or from about nucleotide 814 to about nucleotide 887 with reference to the nucleotide numbering of SEQ ID NO:88.

In some embodiments at least one mutation in the miR156 binding site can be a substitution or deletion, optionally an in-frame deletion or an out-of-frame deletion. In some embodiments, the at least one mutation in the miR156 binding site is a point mutation, optionally a silent point mutation. In some embodiments, the point mutation can be a substitution, optionally wherein the substitution is a C>A, T or G, optionally a C>A. In some embodiments, the at least one mutation may be a non-natural mutation.

In some embodiments, a mutation in a miR156 binding site as described herein can upregulate the expression of the endogenous IPA1 gene, e.g., the mutation results in upregulation of the endogenous SPL9a gene, the endogenous SPL9b gene, the endogenous SPL9c gene, the endogenous SPL9d gene, the endogenous UNBRANCHED 2 (UB2) gene, and/or the endogenous UNBRANCHED 3 (UB3) gene.

In some embodiment, a mutation (e.g., at least one mutation, optionally a non-natural mutation) can be a base substitution in a region of the endogenous UB2 gene or the endogenous UB3 gene that is associated with increased kernel row number (KRN), optionally without a substantial decrease in ear length, and/or increased tassel branch number (TBN). A region of an endogenous UB2 gene associated with increased KRN can be located, for example, from about nucleotide 4379 to about nucleotide 4800 with reference to the nucleotide numbering of SEQ ID NO:84 and/or from about nucleotide 626 to about nucleotide 688 with reference to the nucleotide numbering of SEQ ID NO:85. In some embodiments, a region of an endogenous UB3 gene associated with increased KRN can be located, for example, from about nucleotide 5094 to about nucleotide 5157 with reference to the nucleotide numbering of SEQ ID NO:87 and/or from about nucleotide 641 to about nucleotide 703 with reference to the nucleotide numbering of SEQ ID NO:88. In some embodiments, an edit in an endogenous UB3 gene that is associated with increased KRN can be located from about nucleotide 5108 to about nucleotide 5110 with reference to the nucleotide numbering of SEQ ID NO:87 and/or from about nucleotide 655 to about nucleotide 657 with reference to the nucleotide numbering of SEQ ID NO:88.

In some embodiments, a region of a UB2 gene or a UB3 gene that is associated with increased tassel branch number (TBN) can be targeted for modification as described herein. In some embodiments, the region of an endogenous UB2 gene associated with increased TBN can be from about nucleotide 4834 to about nucleotide 4896 with reference to the nucleotide numbering of SEQ ID NO:84 and/or from about nucleotide 721 to about nucleotide 783 with reference to the nucleotide numbering of SEQ ID NO:85. In some embodiments, an edit in an endogenous UB2 gene that is associated with increased TBN can be located from about nucleotide 4864 to about nucleotide 4866 with reference to the nucleotide numbering of SEQ ID NO:84 and/or from about nucleotide 751 to about nucleotide 753 with reference to the nucleotide numbering of SEQ ID NO:85. In some embodiments, the region of an endogenous UB3 gene associated with increased TBN can be from about nucleotide 5204 to about nucleotide 5266 with reference to the nucleotide numbering of SEQ ID NO:87 or from about nucleotide 751 to about nucleotide 813 with reference to the nucleotide numbering of SEQ ID NO:88. In some embodiments, an edit in an endogenous UB3 gene that is associated with increased TBN can be located from about nucleotide 5231 to about nucleotide 5233 with reference to the nucleotide numbering of SEQ ID NO:87 and/or from about nucleotide 778 to about nucleotide 790 with reference to the nucleotide numbering of SEQ ID NO:88.

In some embodiments, a mutation (e.g., at least one mutation, e.g., one or more mutations, optionally wherein the mutation(s) are non-natural mutation) in an endogenous IPA1 gene encoding a SPL transcription factor may be a mutation in a 5′ untranslated region (UTR) and/or 3′ UTR of the endogenous IPA1 gene, optionally wherein the endogenous IPA1 gene is an SPL9 gene (e.g., SPL9a, SPL9b, SPL9c, SPL9d), a UB2 gene and/or a UB3 gene.

In some embodiments, an endogenous IPA1 gene can be (a) an SPL9a gene and the at least one mutation may be in a region of the 5′ UTR located from about nucleotide 1826 to about nucleotide 1981 and/or from about nucleotide 1846 to about nucleotide 1961 with reference to the nucleotide numbering of SEQ ID NO:72, optionally in a region having at least 80% sequence identity to a nucleotide sequence of any one of SEQ ID NOs:146-160; (b) an SPL9b gene and the at least one mutation may be in a region of the 5′ UTR located from about nucleotide 1804 to about nucleotide 1973 and/or from about nucleotide 1824 to about nucleotide 1953 with reference to the nucleotide numbering of SEQ ID NO:75, optionally in a region having at least 80% sequence identity to a nucleotide sequence of any one of SEQ ID NOs:185-200; (c) an SPL9c gene and the at least one mutation may be in a region of the 5′ UTR located from about nucleotide 1593 to about nucleotide 1783 and/or from about nucleotide 1613 to about nucleotide 1764 with reference to the nucleotide numbering of SEQ ID NO:78, optionally in a region having at least 80% sequence identity to a nucleotide sequence of any one of SEQ ID NOs:225-239; and/or (d) an SPL9d gene and the at least one mutation may be in a region of the 5′ UTR located from about nucleotide 1555 to about nucleotide 1740 and/or from about nucleotide 1574 to about nucleotide 1720 with reference to the nucleotide numbering of SEQ ID NO:81, optionally a region having at least 80% sequence identity to a nucleotide sequence of any one of SEQ ID NOs:258-273. In some embodiments, the at least one mutation may be a non-natural mutation.

In some embodiments, an endogenous IPA1 gene can be (a) a UB2 gene and the at least one mutation may be in a region of the 5′ UTR located from about nucleotide 1414 to about nucleotide 1860, from about nucleotide 1414 to about nucleotide 1522, from about nucleotide 1454 to about nucleotide 1481, from about nucleotide 1553 to about nucleotide 1582, from about nucleotide 1597 to about nucleotide 1633, and/or from about nucleotide 1767 to about nucleotide 1819 with reference to the nucleotide numbering of SEQ ID NO:84, optionally wherein the region of the 5′ UTR is a promoter or is in a promoter, optionally in a region having at least 80% sequence identity to a nucleotide sequence of any one of SEQ ID NOs:133-136; and/or (b) a UB3 gene and the at least one mutation may be in a region of the 5′ UTR located from about nucleotide 1327 to about nucleotide 1646, from about nucleotide 1439 to about nucleotide 1467, from about nucleotide 1368 to about nucleotide 1394, from about nucleotide 1549 to about nucleotide 1606, from about nucleotide 1787 to about nucleotide 1855, and/or from about nucleotide 1747 to about nucleotide 1920 with reference to the nucleotide numbering of SEQ ID NO:87, optionally wherein the region of the 5′ UTR is a promoter or is in a promoter, optionally in a region having at least 80% sequence identity to a nucleotide sequence of any one of SEQ ID NOs:129-132. In some embodiments, the at least one mutation may be a non-natural mutation.

In some embodiments, an endogenous IPA1 gene can be (a) (a) a UB2 gene and the at least one mutation may be in a region of the 3′ UTR located from about nucleotide 5701 to about nucleotide 5882, and/or from about nucleotide 5742 to about nucleotide 5842 with reference to the nucleotide numbering of SEQ ID NO:84, optionally in a region having at least 80% sequence identity to a nucleotide sequence of any one of SEQ ID NOs:140-142; and/or (b) a UB3 gene and the at least one mutation may be in a region of the 3′ UTR located from about nucleotide 5940 to about nucleotide 6109, from about nucleotide 5980 to about nucleotide 6069, from about nucleotide 6516 to about nucleotide 6643, and/or from about nucleotide 6556 to about nucleotide 6603 with reference to the nucleotide numbering of SEQ ID NO:87, optionally in a region having at least 80% sequence identity to a nucleotide sequence of any one of SEQ ID NOs:137-139. In some embodiments, the at least one mutation may be a non-natural mutation.

In some embodiments, a mutation (e.g., at least one mutation, e.g., one or more mutations) in an endogenous IPA1 gene encoding a SPL transcription factor may be a mutation in an intron of the endogenous IPA1 gene, optionally wherein the endogenous IPA1 gene is an SPL9 gene (e.g., SPL9a, SPL9b, SPL9c, SPL9d), a UB2 gene and/or a UB3 gene, optionally wherein the at least one mutation may be a non-natural mutation.

In some embodiments, an endogenous IPA1 gene can be (a) a UB2 gene and the at least one mutation (optionally a non-natural mutation) may be in a region of the intron located from about nucleotide 2856 to about nucleotide 2971, from about nucleotide 2896 to about nucleotide 2931, from about nucleotide 3753 to about nucleotide 3893, and/or from about nucleotide 3793 to about nucleotide 3853 with reference to the nucleotide numbering of SEQ ID NO:84; and/or (b) a UB3 gene and the at least one mutation (optionally a non-natural mutation) may be in a region of the intron located from about nucleotide 2666 to about nucleotide 2784, from about nucleotide 2706 to about nucleotide 2744, from about nucleotide 4017 to about nucleotide 4147, and/or from about nucleotide 4057 to about nucleotide 4107 with reference to the nucleotide numbering of SEQ ID NO:87.

In some embodiments, a mutation (e.g., at least one mutation, e.g., one or more mutations) in a first exon, a third exon, in a miR156 binding site, in a 5′ UTR, in a 3′ UTR, in an intron, or in a region of an IPA1 gene encoding a SPL transcription factor associated with plant architecture, increased tolerance/resistance to abiotic and biotic stress and/or yield traits may be a dominant negative mutation, a semi-dominant mutation, a hypermorphic mutation, a hypomorphic mutation, a weak loss-of-function mutation, or a null allele, optionally wherein the mutation may be null allele. In some embodiments, the at least one mutation may be a non-natural mutation.

A mutation in an endogenous IPA1 gene encoding a SPL transcription factor as described herein can result in a plant that exhibits, for example, altered plant architecture, improved yield traits and/or increased tolerance/resistance to abiotic and biotic stress as compared to a plant or plant part devoid of the same mutation. In some embodiments, improved yield traits can include, but is not limited to, one or more of the phenotype(s) of increased kernel row number, increase kernel size, increase ear length, increased kernel row number without a substantial decrease in ear length, decreased tiller number, decreased tassel branch number, reduced time to flowering, increased seed number per plant, increased pods per node and/or per plant, and/or increased seed weight as compared to a plant or plant part devoid of the same mutation.

In some embodiments, a plant cell comprising an editing system is provided, the editing system comprising: (a) a CRISPR-Cas associated effector protein; and (b) a guide nucleic acid (e.g., gRNA, gDNA, crRNA, crDNA) comprising a spacer sequence with complementarity to an endogenous IDEAL PLANT ARCHITECTURE 1 (IPA1) target gene, or orthologue thereof, encoding a SQUAMOSA PROMOTER BINDING PROTEIN-LIKE (SPL) transcription factor, optionally wherein the IPA1 gene is a SQUAMOSA PROMOTER BINDING PROTEIN-LIKE 9 (SPL9) gene, an endogenous UNBRANCHED 2 (UB2) gene, or an endogenous UNBRANCHED 3 (UB3) gene, optionally wherein the SPL9 gene is a SPL9a gene, a SPL9b gene, a SPL9c gene, or a SPL9d gene. In some embodiments, the endogenous IPA1 target gene: (a) is a SLP9 gene that (i) comprises a nucleotide sequence having at least 80% sequence identity to the nucleotide sequence of any one of SEQ ID NOs:72, 75, 78, 81, 143, 182, 222, or 255; (ii) comprises a coding sequence having at least 80% sequence identity to the nucleotide sequence of any one of SEQ ID NOs:73, 76, 79, 82, 144, 183, 223, or 256; (iii) comprises a region having least 80% sequence identity to the nucleotide sequence of any one of SEQ ID NOs:146-181, 185-221, 225-254 and/or 258-288; and/or (iv) encodes a polypeptide sequence having at least 80% identity to the amino acid sequence of any one of SEQ ID NO:74, 77, 80, 83, 145, 184, 224, or 257; (b) a UB2 gene having at least 80% sequence identity to the nucleotide sequence of SEQ ID NO:84, (ii) comprises a coding sequence having at least 80% sequence identity to the nucleotide sequence of SEQ ID NO:85; (iii) comprises a region having least 80% sequence identity to the nucleotide sequence of any one of SEQ ID NOs:90-96 or 332-393; and/or (iv) encodes a polypeptide sequence having at least 80% identity to the amino acid sequence of SEQ ID NO:86; and/or (c) a UB3 gene having at least 80% sequence identity to the nucleotide sequence of SEQ ID NO:87, (ii) comprises a coding sequence having at least 80% sequence identity to the nucleotide sequence of SEQ ID NO:88; (iii) comprises a region having least 80% sequence identity to the nucleotide sequence of any one of SEQ ID NOs:90, 97-103 or 394-445; and/or (iv) encodes a polypeptide sequence having at least 80% identity to the amino acid sequence of SEQ ID NO:89. In some embodiments, the guide nucleic acid may comprises a nucleotide sequence (e.g., a spacer sequence) of any one of SEQ ID NOs:104-142, 301, 326, and/or 327. In some embodiments, the plant cell is a corn plant cell or a soybean plant cell.

In some embodiments, a plant cell is provided that comprises at least one mutation in one or more endogenous IDEAL PLANT ARCHITECTURE 1 (IPA1) genes, or orthologue thereof, encoding a SQUAMOSA PROMOTER BINDING PROTEIN-LIKE (SPL) transcription factor, wherein the at least one mutations is a substitution, insertion and/or a deletion that is introduced using an editing system that comprises a nucleic acid binding domain that binds to a target site in the one or more endogenous IPA1 genes, optionally wherein the plant cell is from corn or soybean. In some embodiments, the one or more endogenous IPA1 genes may be a SQUAMOSA PROMOTER BINDING PROTEIN-LIKE 9 (SPL9) gene, endogenous UNBRANCHED 2 (UB2) gene, or an endogenous UNBRANCHED 3 (UB3) gene, optionally wherein the SPL9 gene is a SPL9a gene, a SPL9b gene, a SPL9c gene, or a SPL9d gene. In some embodiments, the at least one mutation is a null allele, a knockout mutation, or a hypomorphic mutation. In some embodiments, the target site may be within a region of one or more endogenous SPL9 genes, the region having at least 80% sequence identity to any one of SEQ ID NOs:146-181, 185-221, 225-254 and/or 258-288. In some embodiments, the editing system may further comprise a nuclease, and the target site to which the nucleic acid binding domain binds may be: (a) in an SPL9 gene having at least 80% sequence identity to a nucleotide sequence of any one of SEQ ID NOs:72, 73, 75, 76, 78, 79, 81, 82, 143, 144, 182, 183, 222, 223, 255 or 256; and/or in a region of an SPL9 gene having at least 80% sequence identity to a nucleotide sequence of any one of SEQ ID NOs:146-181, 185-221, 225-254 and/or 258-288; (b) in a UB2 gene having at least 80% sequence identity to the nucleotide sequence of SEQ ID NO:84 or SEQ ID NO:85 and/or in a region of an UB2 gene having at least 80% sequence identity to a nucleotide sequence of any one of SEQ ID NOs:90-96 or 332-393; and/or (c) in a UB3 gene having at least 80% sequence identity to the nucleotide sequence of SEQ ID NO:87 or SEQ ID NO:88 and/or in a region of an UB2 gene having at least 80% sequence identity to a nucleotide sequence of any one of SEQ ID NOs:90, 97-103 or 394-445; and the at least one mutation within the endogenous IPA1 gene is made following cleavage by the nuclease. In some embodiments, the nuclease may be a zinc finger nuclease, transcription activator-like effector nucleases (TALEN), endonuclease (e.g., Fok1) or a CRISPR-Cas effector protein and/or the nucleic acid binding domain of the editing system may be from a polynucleotide-guided endonuclease, a CR1SPR-Cas endonuclease (e.g.; CR1SPR-Cas effector protein), a zinc finger nuclease, a transcription activator-like effector nuclease (TALEN) and/or an Argonaute protein. In some embodiments, the at least one mutation within the one or more endogenous IPA1 gene is an insertion and/or a deletion, optionally a point mutation. In some embodiments, the at least one mutation is an out-of-frame insertion or an out-of-frame deletion, optionally wherein the insertion and/or a deletion result in a premature stop codon and/or a truncated protein. In some embodiments, the at least one mutation may be a non-natural mutation.

In some embodiments, a plant may be regenerated from a plant part or the plant cell of the invention, optionally wherein the regenerated plant exhibits a phenotype of one or more of altered plant architecture, improved yield traits and/or increased tolerance/resistance to abiotic and biotic stress as compared to a control plant or part thereof that is devoid of the at least one mutation.

Further provided is a method of providing a plurality of plants exhibiting altered plant architecture, improved yield traits and/or increased tolerance/resistance to abiotic and biotic stress is provided, the method comprising planting two or more plants of the invention (e.g., 2, 3, 4, 5, 6, 7, 8, 9, 10, 20, 30, 40, 50, 100, 200, 300, 400, 500, 1000, 2000, 3000, 400, 5000, or 10,000 or more plants of the invention) in a growing area, thereby providing a plurality of plants exhibiting altered plant architecture, improved yield traits and/or increased tolerance/resistance to abiotic and biotic stress as compared to a plurality of control plants not comprising the at least one mutation, optionally wherein the plurality of plants exhibiting resistance to biotic stress exhibit increased disease resistance.

In some embodiments, a method of producing/breeding a transgene-free genome-edited (e.g., base-edited) plant is provided, the method comprising: (a) crossing the plant of the invention with a transgene free plant, thereby introducing the mutation or modification into the plant that is transgene-free; and (b) selecting a progeny plant that comprises the mutation or modification but is transgene-free, thereby producing a transgene free genome-edited (e.g., base-edited) plant.

In some embodiments, a method of creating a mutation in an endogenous IPA1 gene in a plant is provided, comprising: (a) targeting a gene editing system to a portion of the IPA1 gene, the portion having: (i) at least 80% sequence identity to any one of SEQ ID NOs:146-181, 185-221, 225-254 and/or 258-288; (ii) at least 80% sequence identity to any one of SEQ ID NOs:90-96 or 332-393; and/or (iii) at least 80% sequence identity to any one of SEQ ID NOs:90, 97-103 or 394-445; and (b) selecting a plant that comprises a modification located in a region of the IPA1 gene having: (i) at least 80% sequence identity to any one of SEQ ID NOs:146-181, 185-221, 225-254 and/or 258-288; (ii) at least 80% sequence identity to any one of SEQ ID NOs:90-96 or 332-393; and/or (iii) at least 80% sequence identity to any one of SEQ ID NOs:90, 97-103 or 394-445.

In some embodiments, a method of generating variation in a IPA1 polypeptide is provided, comprising: introducing an editing system into a plant cell, wherein the editing system is targeted to a region of an endogenous IPA1 gene that encodes the IPA1 polypeptide, and contacting the region of the endogenous IPA1 gene with the editing system, thereby introducing a mutation into the endogenous IPA1 gene and generating variation in the IPA1 polypeptide of the plant cell, optionally wherein the endogenous IPA1 gene comprises: (a) a nucleotide sequence having at least 80% sequence identity to a nucleotide sequence of any one of SEQ ID NOs:72, 73, 75, 76, 78, 79, 81, 82, 143, 144, 182, 183, 222, 223, 255 or 256; and/or encodes an amino acid sequence having at least 80% sequence identity to any one of SEQ ID NOs:74, 77, 80, 83, 145, 184, 224, and/or 257, (b) a nucleotide sequence having at least 80% sequence identity to a nucleotide sequence of SEQ ID NO:84 or SEQ ID NO:85 and/or encodes an amino acid sequence having at least 80% sequence identity to SEQ ID NOs:86; and/or (c) a nucleotide sequence having at least 80% sequence identity to a nucleotide sequence of SEQ ID NO:87 or SEQ ID NO:88 and/or encodes an amino acid sequence having at least 80% sequence identity to SEQ ID NOs:39. In some embodiments, the region of the endogenous IPA1 gene that is targeted comprises (a) at least 80% sequence identity to any one of SEQ ID NOs:146-181, 185-221, 225-254 and/or 258-288; (b) at least 80% sequence identity to any one of SEQ ID NOs:90-96 or 332-393; and/or (c) at least 80% sequence identity to any one of SEQ ID NOs:90, 97-103 or 394-445. In some embodiments, contacting the region of the endogenous IPA1 gene in the plant cell with the editing system produces a plant cell comprising in its genome an edited IPA1 gene, optionally wherein the method further comprises (a) regenerating a plant from the plant cell; (b) selfing the plant to produce progeny plants (E1); (c) assaying the progeny plants of (b) for an improved yield trait; and (d) selecting the progeny plants exhibiting an improved yield trait as compared to a control plant. In some embodiments, method further comprises (e) selfing the selected progeny plants of (d) to produce progeny plants (E2); (f) assaying the progeny plants of (e) for an improved yield trait; and (g) selecting the progeny plants exhibiting an improved yield trait as compared to a control plant, optionally repeating (e) through (g) one or more additional times.

In some embodiments, a method of detecting a mutant IPA1 gene (a mutation in an endogenous IPA1 gene) in a plant is provided, the method comprising detecting in the genome of the plant a IPA1 gene having at least one mutation within a region having: (a) at least 80% sequence identity to any one of SEQ ID NOs:146-181, 185-221, 225-254 and/or 258-288; (b) at least 80% sequence identity to any one of SEQ ID NOs:90-96 or 332-393; and/or (c) at least 80% sequence identity to any one of SEQ ID NOs:90, 97-103 or 394-445, optionally, wherein the mutant IPA1 gene that is detected comprises a nucleic acid sequence having: (a) at least 90% identity to any one of the nucleotide sequences of SEQ ID NOs:289-300, (b) at least 90% identity to any one of the nucleotide sequences of SEQ ID NOs:320, 322 or 324, or (c) having at least 90% identity to any one of the nucleotide sequences of SEQ ID NOs:310, 312, 314, 316, or 318.

In some embodiments, a method for editing a specific site in the genome of a plant cell is provided, the method comprising: cleaving, in a site-specific manner, a target site within an endogenous IPA1 gene in the plant cell, wherein the endogenous IPA1 gene may be: (a) an SPL9 gene having at least 80% sequence identity to a nucleotide sequence of any one of SEQ ID NOs:72, 73, 75, 76, 78, 79, 81, 82, 143, 144, 182, 183, 222, 223, 255 or 256 and/or comprising a region having at least 80% sequence identity to a nucleotide sequence of any one of SEQ ID NOs:146-181, 185-221, 225-254 and/or 258-288, (b) a UB2 gene having at least 80% sequence identity to the nucleotide sequence of SEQ ID NO:84 or SEQ ID NO:85 and/or comprising a region having at least 80% sequence identity to a nucleotide sequence of any one of SEQ ID NOs:90-96 or 332-393; and/or (c) a UB3 gene having at least 80% sequence identity to the nucleotide sequence of SEQ ID NO:87 or SEQ ID NO:88 and/or comprising a region having at least 80% sequence identity to a nucleotide sequence of any one of SEQ ID NOs:90, 97-103 or 394-445, thereby generating an edit in the endogenous IPA1 gene of the plant cell. In some embodiments, the edit in the endogenous IPA1 gene results in a mutation (e.g., nonnatural mutation) that is a null allele. In some embodiments, a plant may be regenerated from the plant cell comprising the edit in the endogenous IPA1 gene to produce a plant comprising the edit in its endogenous IPA1 gene. In some embodiments, the regenerated plant comprising the edit in its endogenous IPA1 gene may exhibit a phenotype of one or more of altered plant architecture, improved yield traits and/or increased tolerance/resistance to abiotic and biotic stress as compared to a control plant that does not comprise the edit.

In some embodiments, an edit in an endogenous SPL9 gene may results in a mutated SPL9 gene having at least 90% identity (e.g., 90, 91, 92, 93, 94, 95, 96, 97, 98, 99 or 100%) to any one of the nucleotide sequences of SEQ ID NOs:289-300. In some embodiments, an edit in an endogenous UB3 gene may results in a mutated UB3 gene having at least 90% identity (e.g., 90, 91, 92, 93, 94, 95, 96, 97, 98, 99 or 100%) to any one of the nucleotide sequences of SEQ ID NOs:310, 312, 314, 316, or 318. In some embodiments, an edit in an endogenous UB2 gene may results in a mutated UB2 gene having at least 90% identity (e.g., 90, 91, 92, 93, 94, 95, 96, 97, 98, 99 or 100%) to any one of the nucleotide sequences of SEQ ID NOs:320, 322 or 324.

In some embodiments, a method for making a plant is provided, comprising: (a) contacting a population of plant cells that comprise an endogenous IPA1 gene with a nuclease targeted to the endogenous gene, wherein the nuclease is linked to a nucleic acid binding domain that binds to a target site in the endogenous gene, the endogenous IPA1 gene: (i) is an SPL9 gene comprising a nucleotide sequence having at least 80% sequence identity to the nucleotide sequence of any one of SEQ ID NOs:72, 73, 75, 76, 78, 79, 81, 82, 143, 144, 182, 183, 222, 223, 255 or 256, and/or comprising a region having at least 80% sequence identity to a nucleotide sequence of any one of SEQ ID NOs:146-181, 185-221, 225-254 and/or 258-288; (b) a UB2 gene having at least 80% sequence identity to the nucleotide sequence of SEQ ID NO:84 or SEQ ID NO:85 and/or comprising a region having at least 80% sequence identity to a nucleotide sequence of any one of SEQ ID NOs:90-96 or 332-393; and/or a UB3 gene having at least 80% sequence identity to the nucleotide sequence of SEQ ID NO:87 or SEQ ID NO:88 and/or comprising a region having at least 80% sequence identity to a nucleotide sequence of any one of SEQ ID NOs:90, 97-103 or 394-445; (b) selecting a plant cell from the population comprising a mutation in the endogenous IPA1 gene, wherein the mutation is a substitution and/or a deletion; and (c) growing the selected plant cell into a plant comprising the mutation in the endogenous IPA1 gene.

In some embodiments, a method for altering plant architecture, improving yield traits and/or increasing tolerance/resistance of a plant is provided, the method comprising (a) contacting a plant cell comprising an endogenous IPA1 gene with a nuclease targeted to the endogenous IPA1 gene, wherein the nuclease is linked to a nucleic acid binding domain that binds to a target site in the endogenous IPA1 gene, wherein the endogenous IPA1 gene is: (i) an SPL9 gene comprising a nucleotide sequence having at least 80% sequence identity to the nucleotide sequence of any one of SEQ ID NOs:72, 73, 75, 76, 78, 79, 81, 82, 143, 144, 182, 183, 222, 223, 255 or 256, and/or comprising a region having at least 80% sequence identity to a nucleotide sequence of any one of SEQ ID NOs:146-181, 185-221, 225-254 and/or 258-288; (ii) a UB2 gene having at least 80% sequence identity to the nucleotide sequence of SEQ ID NO:84 or SEQ ID NO:85 and/or comprising a region having at least 80% sequence identity to a nucleotide sequence of any one of SEQ ID NOs:90-96 or 332-393; and/or (iii) a UB3 gene having at least 80% sequence identity to the nucleotide sequence of SEQ ID NO:87 or SEQ ID NO:88 and/or comprising a region having at least 80% sequence identity to a nucleotide sequence of any one of SEQ ID NOs:90, 97-103 or 394-445; and (b) growing the plant cell into a plant, thereby altering plant architecture, improving yield traits and/or increasing tolerance/resistance of the plant.

In some embodiments, a method is provided for producing a plant or part thereof comprising at least one cell having mutation in an endogenous IDEAL PLANT ARCHITECTURE 1 (IPA1) gene, or orthologue thereof, encoding a SQUAMOSA PROMOTER BINDING PROTEIN-LIKE (SPL) transcription factor, the method comprising contacting a target site in the endogenous IPA1 gene in the plant or plant part with a nuclease comprising a cleavage domain and a DNA-binding domain, wherein the DNA binding domain of the nuclease binds to a target site in the endogenous IPA1 gene, wherein the endogenous IPA1 gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NO:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393; and/or (iv) encoding a polypeptide sequence having at least 80% identity to the amino acid sequence of SEQ ID NO:86; or (c) is an endogenous UB3 gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445; and/or (iv) encoding a polypeptide sequence having at least 80% identity to the amino acid sequence of SEQ ID NO:89, thereby producing a plant or part thereof comprising at least one cell having a mutation in the endogenous IPA1 gene.

In some embodiments, a method of producing a plant or part thereof comprising a mutation in an endogenous IPA1 gene and having a phenotype of altered plant architecture, improved yield traits and/or increased tolerance/resistance to abiotic and biotic stress is provided, the method comprising contacting a target site in an endogenous IPA1 gene in the plant or plant part with a nuclease comprising a cleavage domain and a nucleic acid binding domain, wherein the nucleic acid binding domain of the nuclease binds to a target site in the endogenous IPA1 gene, wherein the endogenous IPA gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NO:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393; and/or (iv) encoding a polypeptide sequence having at least 80% identity to the amino acid sequence of SEQ ID NO:86; or (c) is an endogenous UB3 gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445; and/or (iv) encoding a polypeptide sequence having at least 80% identity to the amino acid sequence of SEQ ID NO:89, thereby producing a plant or part thereof having a mutated endogenous IPA1 gene and altered plant architecture, improved yield traits and/or increased tolerance/resistance to abiotic and biotic stress.

In some embodiments, a mutation useful for this invention may be at least one of a base pair deletion, a base pair substitution, and/or a base pair insertion. In some embodiments, the mutation may be a dominant negative mutation, a semi-dominant mutation, a hypermorphic mutation, a hypomorphic mutation, a weak loss-of-function mutation, and/or a null allele. In some embodiments, a mutation may be a non-natural mutation.

In some embodiments, a mutation of a plant or part thereof as described herein, may result in the plant having altered plant architecture, improved yield traits and/or increased tolerance/resistance to abiotic and biotic stress as compared to a plant or plant part not comprising the same mutation. In some embodiments, improved yield traits can include, but is not limited to, one or more of the phenotype(s) of increased kernel row number (about 5% to about 30%, e.g., (e.g., about 5, 6, 7, 8, 9, 10, 11, 12, 13, 14, 15, 16, 17, 18, 19, 20, 21, 22, 23, 24, 25, 26, 27, 28, 29, or 30%, or any range or value therein) without a substantial decrease in ear length, increased kernel size (about 1% to about 25%; e.g., about 1, 2, 3, 4, 5, 6, 7, 8, 9, 10, 11, 12, 13, 14, 15, 16, 17, 18, 19, 20, 21, 22, 23, 24, or 25%, or any range or value therein), increased ear length (about 2% to about 30%; e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30%, or any range or value therein), decreased tiller number (about 2% to about 100%; e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 75, 80, 81, 82, 83, 84, 85, 90, 91, 92, 93, 94, 95, 96, 97, 98, 99, or 100%, or any range or value therein), decreased tassel branch number (about 2% to about 100%; e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 75, 80, 81, 82, 83, 84, 85, 90, 91, 92, 93, 94, 95, 96, 97, 98, 99, or 100%, or any range or value therein), reduced time to flowering (about 5% to about 50%; e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, or 50%, or any range or value therein), increased seed number per plant (about 10% to about 100%; e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 75, 80, 81, 82, 83, 84, 85, 90, 91, 92, 93, 94, 95, 96, 97, 98, 99, or 100%, or any range or value therein), increased pods per node (about 3% to about 50%; e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, or 50%, or any range or value therein) and/or per plant (about 3% to about 50%%; e.g., about 3, 4, 5, 6, 7, 8, 9, 10, 11, 12, 13, 14, 15, 16, 17, 18, 19, 20, 21, 22, 23, 24, 25, 26, 27, 28, 29, or 30%, or any range or value therein), and/or increased seed weight (about 1% to about 20%; e.g., about 1, 2, 3, 4, 5, 6, 7, 8, 9, 10, 11, 12, 13, 14, 15, 16, 17, 18, 19, or 20%, or any range or value therein), in any combination.

In some embodiments, an endogenous SPL9 gene may be present in the plant or part thereof as two paralogous pairs (a) an SPL9a gene and a SPL9b gene and/or (b) a SPL9c gene and a SPL9d gene, optionally wherein at least one of the SPL9a gene, the SPL9b gene, the SPL9c gene and the SPL9d gene comprise a mutation, in any combination, or wherein each of the SPL9a gene, the SPL9b gene, the SPL9c gene and the SPL9d gene comprise a mutation.

In some embodiments, a mutation (optionally a non-natural mutation) in an IPA1 gene may be generated in the first exon of the endogenous SPL9 gene optionally resulting in a premature stop codon and a null allele. In some embodiments, the first exon of the SPL9a gene may be located from about nucleotide 2001 to about nucleotide 2364 with reference to the nucleotide numbering of SEQ ID NO:72, from about nucleotide 1 to about nucleotide 364 with reference to the nucleotide numbering of SEQ ID NO:73, and/or from about nucleotide 2160 to about nucleotide 2523 with reference to the nucleotide numbering of SEQ ID NO:143, the first exon of the SPL9b gene may be located from about nucleotide 2001 to about nucleotide 2370 with reference to the nucleotide numbering of SEQ ID NO:75, from about nucleotide 1 to about nucleotide 370 with reference to the nucleotide numbering of SEQ ID NO:76, and/or from about nucleotide 2098 to about nucleotide 2467 with reference to the nucleotide numbering of SEQ ID NO:182, the first exon of the SPL9c gene may be located from about nucleotide 2001 to about nucleotide 2347 with reference to the nucleotide numbering of SEQ ID NO:78, from about nucleotide 1 to about nucleotide 347 with reference to the nucleotide numbering of SEQ ID NO:79, and/or from about nucleotide 2378 to about nucleotide 2724 with reference to the nucleotide numbering of SEQ ID NO:222, and/or the first exon of the SPL9d gene may be located from about nucleotide 2001 to about nucleotide 2349 with reference to the nucleotide numbering of SEQ ID NO:81, from about nucleotide 1 to about nucleotide 349 with reference to the nucleotide numbering of SEQ ID NO:82 and/or from about nucleotide 2410 to about nucleotide 2758 with reference to the nucleotide numbering of SEQ ID NO:255.

In some embodiments, a mutation (optionally a non-natural mutation) in an SPL9 gene may be in a region of the first exon of the SPL9a gene from about nucleotide 2053 to about nucleotide 2115 with reference to the nucleotide numbering of SEQ ID NO:72 or SEQ ID NO:75, a region of the first exon of the SPL9b gene from about nucleotide 2015 to about nucleotide 2077 with reference to the nucleotide numbering of SEQ ID NO:78 or SEQ ID NO:81, a region of the first exon of the SPL9c gene from about nucleotide 1 to about nucleotide 115 with reference to the nucleotide numbering of SEQ ID NO:73 or SEQ ID NO:76, and/or a region of the first exon of the SPL9d gene from about nucleotide 1 to about nucleotide 77 with reference to the nucleotide numbering of SEQ ID NO:79 or SEQ ID NO:82, optionally in a region of the SPL9a gene having least 80% sequence identity to the nucleotide sequence of any one of SEQ ID NOs:161-177, in a region of the SPL9b gene having least 80% sequence identity to the nucleotide sequence of any one of 201-217, in a region of the SPL9c gene having least 80% sequence identity to the nucleotide sequence of any one of 240-250, or in a region of the SPL9d gene having least 80% sequence identity to the nucleotide sequence of any one of 274-284.

In some embodiments, a mutation in a UB2 gene may be in a region of the third exon of the endogenous UB2 gene (see e.g., third exon of SEQ ID NO:84; e.g., SEQ ID NOs: 358-376, optionally SEQ ID NOs:373-376), optionally resulting in a premature stop codon and a null allele, optionally a hypomorphic or knockout mutation. In some embodiments, a mutation in a UB3 gene may be in a region of the third exon of the endogenous UB3 gene (see e.g., third exon of SEQ ID NO:87; e.g., SEQ ID NOs:408-426, optionally SEQ ID NOs: 415-416, optionally resulting in a premature stop codon and a null allele, optionally a hypomorphic or knockout mutation. In some embodiments, the mutation may be a non-natural mutation. In some embodiments, a mutation in an IPA1 gene may be generated in a miR156 binding site, e.g., an miRNA binding site of an endogenous SPL9 gene, UB2 gene and/or UB3 gene. In some embodiments, (a) the endogenous gene is an SPL9a gene and the miR156 binding site may be located from about nucleotide 6569 to about nucleotide 6588 with reference to the nucleotide numbering of SEQ ID NO:72, from about nucleotide 758 to about nucleotide 777 with reference to the nucleotide numbering of SEQ ID NO:73, and/or from about nucleotide 6624 to about nucleotide 6847 with reference to the nucleotide numbering of SEQ ID NO:143; (b) the endogenous gene is an SPL9b gene and the miR156 binding site may be located from about nucleotide 6269 to about nucleotide 6288 with reference to the nucleotide numbering of SEQ ID NO:75, from about nucleotide 760 to about nucleotide 780 with reference to the nucleotide numbering of SEQ ID NO:76, and/or from about nucleotide 6265 to about nucleotide 6488 with reference to the nucleotide numbering of SEQ ID NO:182, (c) the endogenous gene is an SPL9c gene and the miR156 binding site may be located from about nucleotide 5388 to about nucleotide 5407 with reference to the nucleotide numbering of SEQ ID NO:78, from about nucleotide 761 to about nucleotide 780 with reference to the nucleotide numbering of SEQ ID NO:79, and/or from about nucleotide 5665 to about nucleotide 5887 with reference to the nucleotide numbering of SEQ ID NO:222, and/or (d) the endogenous gene is an SPL9d gene and the miR156 binding site may be located from about nucleotide 5798 to about nucleotide 5817 with reference to the nucleotide numbering of SEQ ID NO:81, from about nucleotide 737 to about nucleotide 756 with reference to the nucleotide numbering of SEQ ID NO:82, and/or from about nucleotide 6120 to about nucleotide 6342 with reference to the nucleotide numbering of SEQ ID NO:255. In some embodiments, the mutation may be a non-natural mutation.

In some embodiments, a mutation in an miR156 binding site may be located: (a) in a region of the endogenous SPL9a gene from about nucleotide 6549 to about nucleotide 6608 with reference to the nucleotide numbering of SEQ ID NO:72 and/or from about nucleotide 738 to about nucleotide 797 with reference to the nucleotide numbering of SEQ ID NO:73, optionally in a region having about 80% sequence identity to any one of the nucleotide sequences of SEQ ID NO:178-181, (b) in a region of the endogenous SPL9b gene from about nucleotide 6250 to about nucleotide 6308 with reference to the nucleotide numbering of SEQ ID NO:75 and/or from about nucleotide 741 to about nucleotide 800 with reference to the nucleotide numbering of SEQ ID NO:76, optionally in a region having about 80% sequence identity to any one of the nucleotide sequences of SEQ ID NO:218-221, (c) in a region of the endogenous SPL9c gene from about nucleotide 5368 to about nucleotide 5427 with reference to the nucleotide numbering of SEQ ID NO:78 and/or from about nucleotide 742 to about nucleotide 800 with reference to the nucleotide numbering of SEQ ID NO:79, optionally in a region having about 80% sequence identity to any one of the nucleotide sequences of SEQ ID NO:251-254, and/or (d) in a region of the of the endogenous SPL9d gene from about nucleotide 5778 to about nucleotide 5837 with reference to the nucleotide numbering of SEQ ID NO:81 and/or from about nucleotide 718 to about nucleotide 775 with reference to the nucleotide numbering of SEQ ID NO:82, optionally in a region having about 80% sequence identity to any one of the nucleotide sequences of SEQ ID NO:285-288. In some embodiments, the mutation may be a non-natural mutation.

In some embodiments, a mutation may be in a the miR156 binding site of a UB2 gene located from about nucleotide 4928 to about nucleotide 4947 with reference to the nucleotide numbering of SEQ ID NO:84 and/or from about nucleotide 815 to about nucleotide 834 with reference to the nucleotide numbering of SEQ ID NO:85. In some embodiments, a mutation may be in an miR156 binding site of a UB3 gene and the miR156 binding site located from about nucleotide 5301 to about nucleotide 5320 with reference to the nucleotide numbering of SEQ ID NO:87 and/or from about nucleotide 848 to about nucleotide 866 with reference to the nucleotide numbering of SEQ ID NO:88. In some embodiments, a mutation in a miR156 binding site: (a) of an endogenous UB2 gene may be located from about nucleotide 4894 to about nucleotide 4967 with reference to the nucleotide numbering of SEQ ID NO:84 and/or from about nucleotide 781 to about nucleotide 854 with reference to the nucleotide numbering of SEQ ID NO:85, and/or (b) of an endogenous UB3 gene may be located from about nucleotide 5267 to about nucleotide 5339 with reference to the nucleotide numbering of SEQ ID NO:87 and/or from about nucleotide 814 to about nucleotide 887 with reference to the nucleotide numbering of SEQ ID NO:88. In some embodiments, the mutation may be a non-natural mutation.

In some embodiments, a mutation in an miR156 binding site of an IPA1 gene may be a substitution or deletion, optionally wherein the deletion may be an in-frame deletion or an out-of-frame deletion. In some embodiments, at least one mutation in the miR156 binding site may be a point mutation, optionally a silent point mutation. In some embodiments, the mutation may be a non-natural mutation. In some embodiments, the point mutation may be a substitution, optionally wherein the substitution is a C>A, T or G, optionally a C>A. In some embodiments, the mutation in the miR156 binding site upregulates the expression of the endogenous IPA1 gene, e.g., the endogenous SPL9a gene, the endogenous SPL9b gene, the endogenous SPL9c gene, the endogenous SPL9d gene, the endogenous UNBRANCHED 2 (UB2) gene, and/or the endogenous UNBRANCHED 3 (UB3) gene.

In some embodiments, the at least one mutation (optionally a non-natural mutation) is a base substitution in a region of the endogenous UB2 gene or the endogenous UB3 gene that is associated with increased kernel row number (KRN), optionally without a substantial decrease in ear length, and/or increased tassel branch number (TBN), optionally, wherein the region of the endogenous UB2 gene associated with increased KRN is from about nucleotide 4379 to about nucleotide 4800 with reference to the nucleotide numbering of SEQ ID NO:84 and/or from about nucleotide 626 to about nucleotide 688 with reference to the nucleotide numbering of SEQ ID NO:85 and/or the region of the endogenous UB3 gene associated with increased KRN is from about nucleotide 5094 to about nucleotide 5157 with reference to the nucleotide numbering of SEQ ID NO:87 and/or from about nucleotide 641 to about nucleotide 703 with reference to the nucleotide numbering of SEQ ID NO:88, and/or wherein the region of the endogenous UB2 gene associated with increased TBN is from about nucleotide 4834 to about nucleotide 4896 with reference to the nucleotide numbering of SEQ ID NO:84 and/or from about nucleotide 721 to about nucleotide 783 with reference to the nucleotide numbering of SEQ ID NO:85 and/or the region of the endogenous UB3 gene associated with increased TBN is from about nucleotide 5204 to about nucleotide 5266 with reference to the nucleotide numbering of SEQ ID NO:87 and/or from about nucleotide 751 to about nucleotide 813 with reference to the nucleotide numbering of SEQ ID NO:88.

In some embodiments, a mutation useful for generating a plant having an altered plant architecture, improved yield traits and/or increased tolerance/resistance to abiotic and biotic stress may be in a 5′ untranslated region (UTR) and/or 3′ UTR of the endogenous SPL9 gene, endogenous UB2 gene or endogenous UB3 gene, optionally, wherein the endogenous SPL9 gene is: (a) an endogenous SPL9a gene and the mutation is in a region of the 5′ UTR located from about nucleotide 1826 to about nucleotide 1981 and/or from about nucleotide 1846 to about nucleotide 1961 with reference to the nucleotide numbering of SEQ ID NO:72, optionally a region having at least 80% sequence identity to a nucleotide sequence of any one of SEQ ID NOs:146-160; (b) an endogenous SPL9b gene and the mutation is in a region of the 5′ UTR located from about nucleotide 1804 to about nucleotide 1973 and/or from about nucleotide 1824 to about nucleotide 1953 with reference to the nucleotide numbering of SEQ ID NO:75, optionally a region having at least 80% sequence identity to a nucleotide sequence of any one of SEQ ID NOs:185-200; (c) an endogenous SPL9c gene and the mutation is in a region of the 5′ UTR located from about nucleotide 1593 to about nucleotide 1783 and/or from about nucleotide 1613 to about nucleotide 1764 with reference to the nucleotide numbering of SEQ ID NO:78, optionally a region having at least 80% sequence identity to a nucleotide sequence of any one of SEQ ID NOs:225-239; and/or (d) an endogenous SPL9d gene and the mutation is in a region of the 5′ UTR located from about nucleotide 1555 to about nucleotide 1740 and/or from about nucleotide 1574 to about nucleotide 1720 with reference to the nucleotide numbering of SEQ ID NO:81, optionally a region having at least 80% sequence identity to a nucleotide sequence of any one of SEQ ID NOs:258-273. In some embodiments, the mutation may be a non-natural mutation.

In some embodiments, a mutation useful for generating a plant having an altered plant architecture, improved yield traits and/or increased tolerance/resistance to abiotic and biotic stress may be in a 5′ untranslated region (UTR) of (a) an endogenous UB2 gene, the 5′ UTR located from about nucleotide 1414 to about nucleotide 1860, from about nucleotide 1414 to about nucleotide 1522, from about nucleotide 1454 to about nucleotide 1481, from about nucleotide 1553 to about nucleotide 1582, from about nucleotide 1597 to about nucleotide 1633, and/or from about nucleotide 1767 to about nucleotide 1819 with reference to the nucleotide numbering of SEQ ID NO:84, optionally a region having at least 80% sequence identity to a nucleotide sequence of any one of SEQ ID NOs:133-136; and/or (b) of an endogenous UB3 gene, the 5′ UTR located from about nucleotide 1327 to about nucleotide 1646, from about nucleotide 1439 to about nucleotide 1467, from about nucleotide 1368 to about nucleotide 1394, from about nucleotide 1549 to about nucleotide 1606, from about nucleotide 1787 to about nucleotide 1855, and/or from about nucleotide 1747 to about nucleotide 1920 with reference to the nucleotide numbering of SEQ ID NO:87, optionally a region having at least 80% sequence identity to a nucleotide sequence of any one of SEQ ID NOs:129-132. In some embodiments, the mutation may be a non-natural mutation.

In some embodiments, a mutation useful for generating a plant having an altered plant architecture, improved yield traits and/or increased tolerance/resistance to abiotic and biotic stress may be in a 3′ untranslated region (UTR) of (a) an endogenous UB2 gene, the 3′ UTR located from about nucleotide 5701 to about nucleotide 5882, and/or from about nucleotide 5742 to about nucleotide 5842 with reference to the nucleotide numbering of SEQ ID NO:84, optionally a region having at least 80% sequence identity to a nucleotide sequence of any one of SEQ ID NOs:140-142; and/or (b) the endogenous UB3, the 3′ UTR gene located from about nucleotide 5940 to about nucleotide 6109, from about nucleotide 5980 to about nucleotide 6069, from about nucleotide 6516 to about nucleotide 6643, and/or from about nucleotide 6556 to about nucleotide 6603 with reference to the nucleotide numbering of SEQ ID NO:87, optionally a region having at least 80% sequence identity to a nucleotide sequence of any one of SEQ ID NOs:137-139. In some embodiments, the mutation may be a non-natural mutation.

In some embodiments, a mutation useful for generating a plant having an altered plant architecture, improved yield traits and/or increased tolerance/resistance to abiotic and biotic stress may be in an intron of an endogenous UB2 gene or an endogenous UB3 gene, optionally wherein the region of the intron that is targeted for a mutation in: (a) the endogenous UB2 gene is located from about nucleotide 2856 to about nucleotide 2971, from about nucleotide 2896 to about nucleotide 2931, from about nucleotide 3753 to about nucleotide 3893, and/or from about nucleotide 3793 to about nucleotide 3853 with reference to the nucleotide numbering of SEQ ID NO:84; and/or (b) the endogenous UB3 gene is located from about nucleotide 2666 to about nucleotide 2784, from about nucleotide 2706 to about nucleotide 2744, from about nucleotide 4017 to about nucleotide 4147, and/or from about nucleotide 4057 to about nucleotide 4107 with reference to the nucleotide numbering of SEQ ID NO:87. In some embodiments, the mutation may be a non-natural mutation.

In some embodiments, the at least one mutation is a dominant negative mutation, a semi-dominant mutation, a hypermorphic mutation, a hypermorphic mutation, a hypomorphic mutation, a weak loss-of-function mutation, or a null allele, optionally wherein the mutation is a non-natural mutation.

In some embodiments, a plant produced by the methods of the invention can exhibit, for example, altered plant architecture, improved yield traits and/or increased tolerance/resistance to abiotic and biotic stress as compared to a plant or plant part devoid of the same mutation. In some embodiments, improved yield traits includes, but is not limited to, one or more of the phenotype(s) of increased kernel row number, optionally without a substantial decrease in ear length, increased kernel size, increased ear length, decreased tiller number, decreased tassel branch number, reduced time to flowering, increased seed number per plant, increased pods per node and/or per plant, and/or increased seed weight.

Any plant or part thereof comprising an endogenous IPA1 gene encoding a SPL transcription factor can be used with the methods and compositions of the invention to provide a plant or part thereof comprising an endogenous IPA1 gene modified as described herein and a plant that exhibits, for example, altered plant architecture, improved yield traits and/or increased tolerance/resistance to abiotic and biotic stress as compared to a plant devoid of the same mutation. A plant useful with the invention can be, for example, a monocot or a dicot.

Non-limiting examples of plants that may be modified as described herein may include, but are not limited to, turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, the nucleic acid constructs of the invention and/or expression cassettes and/or vectors encoding the same may be used to modify maize, soybean, wheat, canola, rice, tomato, pepper, or sunflower. In some embodiments, a plant useful with the invention includes, but is not limited to, corn, soy, canola, wheat, rice, cotton, sugarcane, sugar beet, barley, oats, alfalfa, sunflower, safflower, oil palm, sesame, coconut, tobacco, potato, sweet potato, cassava, coffee, apple, plum, apricot, peach, cherry, pear, fig, banana, citrus, cocoa, avocado, olive, almond, walnut, strawberry, watermelon, pepper, grape, tomato, cucumber, or a Brassica spp (e.g., B. napus, B. oleracea, B. rapa, B. juncea, and/or B. nigra). In some embodiments, the plant is corn. In some embodiments, the plant is soybean.

An endogenous IPA1 gene encoding an SPL transcription factor useful with this invention may comprise, for example, an endogenous SPL9 gene, an endogenous UB2 gene or an endogenous UB3 gene. In some embodiments, an endogenous SPL9 gene useful with this invention: (a) comprises a nucleotide sequence having at least 80% sequence identity to the nucleotide sequence of any one of SEQ ID NOs:72, 75, 78, 81, 143, 182, 222, or 255; (b) comprises a coding sequence having at least 80% sequence identity to the nucleotide sequence of any one of SEQ ID NOs:73, 76, 79, 82, 144, 183, 223, or 256; (c) comprises a region having least 80% sequence identity to the nucleotide sequence of any one of SEQ ID NOs:146-181, 185-221, 225-254 or 258-288, and/or (d) encodes a polypeptide sequence having at least 80% identity to the amino acid sequence of any one of SEQ ID NOs:74, 77, 80, 83, 145, 184, 224, or 257. In some embodiments, an endogenous UB2 gene useful with this invention: (a) comprises a nucleotide sequence having at least 80% sequence identity to the nucleotide sequence of SEQ ID NO:84; (b) comprises a coding sequence having at least 80% sequence identity to the nucleotide sequence of SEQ ID NO:85; (c) comprises a region having least 80% sequence identity to the nucleotide sequence of any one of SEQ ID NOs:90-96 or 332-339, and/or (d) encodes a polypeptide sequence having at least 80% identity to the amino acid sequence of SEQ ID NO:86. In some embodiments, an endogenous UB3 gene useful with this invention: (a) comprises a nucleotide sequence having at least 80% sequence identity to the nucleotide sequence of SEQ ID NO:87; (b) comprises a coding sequence having at least 80% sequence identity to the nucleotide sequence of SEQ ID NO:88; (c) comprises a region having least 80% sequence identity to the nucleotide sequence of any one of SEQ ID NOs:90, 97-103 or 394-445, and/or (d) encodes a polypeptide sequence having at least 80% identity to the amino acid sequence of SEQ ID NO:89.

In some embodiments, a nuclease may cleave an endogenous IPA1 gene encoding an

SPL transcription factor, thereby introducing a mutation into the endogenous IPA1 gene. A nuclease useful with the invention may be any nuclease that can be utilized to edit/modify a target nucleic acid. Such nucleases include, but are not limited to a zinc finger nuclease, transcription activator-like effector nucleases (TALEN), endonuclease (e.g., Fok1) and/or a CRISPR-Cas effector protein. Likewise, any nucleic acid binding domain (e.g., DNA binding domain, RNA binding domain) useful with the invention may be any nucleic acid binding domain that can be utilized to edit/modify a target nucleic acid. Such nucleic acid binding domains include, but are not limited to, a zinc finger, transcription activator-like DNA binding domain (TAL), an argonaute and/or a CRISPR-Cas effector DNA binding domain.

In some embodiments, a method of editing an endogenous IPA1 gene in a plant or plant part is provided, the method comprising contacting a target site in an IPA1 gene in the plant or plant part with a cytosine base editing system comprising a cytosine deaminase and a nucleic acid binding domain that binds to a target site in the IPA gene, the IPA gene is (a) an SPL9 gene encoding a sequence having at least 80% sequence identity to the amino acid sequence of SEQ ID NOs:74, 77, 80, 83, 86, 89, 145, 184, 224, or 257 and/or comprising a sequence having at least 80% sequence identity to the nucleotide sequence of SEQ ID NOs:72, 73, 75, 76, 78, 79, 81, 82, 84, 85, 87, 88, 143, 144, 182, 183, 222, 223, 255 or 256; (b) an UB2 gene encoding a sequence having at least 80% sequence identity to the amino acid sequence of SEQ ID NO:86 and/or comprising a sequence having at least 80% sequence identity to the nucleotide sequence of SEQ ID NO:84; and/or (c) an UB3 gene encoding a sequence having at least 80% sequence identity to the amino acid sequence of SEQ ID NO:89 and/or comprising a sequence having at least 80% sequence identity to the nucleotide sequence of SEQ ID NO:87; thereby editing the endogenous IPA1 gene in the plant or part thereof and producing a plant or part thereof comprising at least one cell having a mutation in the endogenous IPA1 gene.

In some embodiments, a method of editing an endogenous IPA1 gene in a plant or plant part is provided, the method comprising contacting a target site in an IPA1 gene in the plant or plant part with an adenosine base editing system comprising an adenosine deaminase and a nucleic acid binding domain that binds to a target site in the IPA gene is (a) an SPL9 gene encoding a sequence having at least 80% sequence identity to the amino acid sequence of SEQ ID NOs:74, 77, 80, 83, 86, 89, 145, 184, 224, or 257 and/or comprising a sequence having at least 80% sequence identity to the nucleotide sequence of SEQ ID NOs:72, 73, 75, 76, 78, 79, 81, 82, 84, 85, 87, 88, 143, 144, 182, 183, 222, 223, 255 or 256; (b) an UB2 gene encoding a sequence having at least 80% sequence identity to the amino acid sequence of SEQ ID NO:86 and/or comprising a sequence having at least 80% sequence identity to the nucleotide sequence of SEQ ID NO:84; and/or (c) an UB3 gene encoding a sequence having at least 80% sequence identity to the amino acid sequence of SEQ ID NO:89 and/or comprising a sequence having at least 80% sequence identity to the nucleotide sequence of SEQ ID NO:87; thereby editing the endogenous IPA1 gene in the plant or part thereof and producing a plant or part thereof comprising at least one cell having a mutation in the endogenous IPA1 gene.

In some embodiments, a method of detecting a mutant IPA1 gene (a mutation in an endogenous IPA1 gene) is provided, the method comprising detecting in the genome of a plant a deletion in (a) a nucleic acid encoding any one of the amino acid sequences of SEQ ID NOs:74, 77, 80, 83, 86, 89, 145, 184, 224, or 257 and/or comprising a sequence having at least 80% sequence identity to the nucleotide sequence of SEQ ID NOs:72, 73, 75, 76, 78, 79, 81, 82, 84, 85, 87, 88, 143, 144, 182, 183, 222, 223, 255 or 256; (b) a nucleic acid encoding the amino acid sequences of SEQ ID NO:86 and/or comprising a sequence having at least 80% sequence identity to the nucleotide sequence of SEQ ID NO:84; and/or (c) a nucleic acid encoding the amino acid sequences of SEQ ID NO:89 and/or comprising a sequence having at least 80% sequence identity to the nucleotide sequence of SEQ ID NO:87.

In some embodiments, a method of detecting a mutant IPA1 gene (a mutation in an endogenous IPA1 gene) is provided, the method comprising detecting in the genome of a plant a mutated IPA1 gene of any one of SEQ ID NOs:289-300 (e.g., a mutated SPL9a gene having the nucleic acid sequence of any one of SEQ ID NO:295 or SEQ ID NO:301; a mutated SPL9b gene having the nucleic acid sequence of SEQ ID NOs:299; a mutated SPL9c gene having the nucleic acid sequence of any one of SEQ ID NOs:289, 291, 292, 296, or 298; a mutated SPL9d gene having the nucleic acid sequence of any one of SEQ ID NOs:290, 293, 294, or 297); a mutated IPA1 gene of any one of SEQ ID NOs:310, 312, 314, 316, or 318 (e.g., a mutated UB3 gene); or a mutated IPA1 gene of any one of SEQ ID NOs:320, 322, or 324 (e.g., a mutated UB2 gene).

In some embodiments, a mutation in an endogenous IPA1 gene encoding an SPL transcription factor in a plant may be a substitution, a deletion and/or an insertion. In some embodiments, the mutation may be a non-natural mutation. In some embodiments, the mutation in an endogenous IPA1 gene in a plant may be a substitution, a deletion and/or an insertion that results in a dominant negative mutation, a semi-dominant mutation, a weak loss-of-function mutation, hypermorphic mutation, hypomorphic mutation, or a null mutation and a plant exhibiting altered plant architecture, improved yield traits and/or increased tolerance/resistance to abiotic and biotic stress as compared to a plant or plant part devoid of the same mutation. For example, the mutation may be a substitution, a deletion and/or an insertion of one or more amino acid residues (e.g., 1, 2, 3, 4, 5, 6, 7, 8, 9, 10 or more amino acids of the SPL transcription factor) or the mutation may be a substitution, a deletion and/or an insertion of at least 1 nucleotide to about 150 consecutive nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 110, 111, 12, 113, 114, 115, 116, 117, 118, 119, 120, 121, 122, 123, 124, 125, 126, 127, 128, 129, 130, 131, 132, 133, 134, 135, 140, 141, 142, 143, 144, 145, 146, 147, 148, 149, or 150 or more consecutive nucleotides, or any range or value therein) (e.g., a base substitution, deletion and/or insertion) from the endogenous IPA1 gene encoding the SPL transcription factor, optionally wherein the mutation in an IPA1 gene is a deletion and the deletion may be about 4, 5, 7, 8, 9, 10, 11, 12, 13, 29, 54, 68, or 127 consecutive nucleotides and any combination of deletions thereof. In some embodiments, a deletion can be an in-frame deletion or an out-of-frame deletion. In some embodiments, the at least one mutation may be a base substitution to an A, a T, a G, or a C. In some embodiments, the at least one mutation may be a point mutation, optionally a silent point mutation. A point mutation can be a substitution, optionally wherein the substitution is a C>A, T or G, optionally a C>A.

In some embodiments, a mutation in an endogenous IPA1 gene encoding a SPL transcription factor may be made following cleavage by an editing system that comprises a nuclease and a nucleic acid binding domain that binds to a target site within a target nucleic acid (e.g., the IPA1 gene) comprising a nucleotide sequence having at least 80% identity to any one of the nucleotide sequences of SEQ ID NOs:72, 73, 75, 76, 78, 79, 81, 82, 85, 87, 88, 143, 144, 182, 183, 222, 223, 255 or 256, or a encoding a polypeptide comprising a sequence having at least 80% sequence identity to any one of the amino acid sequences of SEQ ID NOs:74, 77, 80, 83, 86, 89, 145, 184, 224, or 257. In some embodiments, the nuclease cleaves the endogenous IPA1 gene and a mutation is introduced into the endogenous IPA1 gene.

Further provided herein are guide nucleic acids (e.g., gRNA, gDNA, crRNA, crDNA) that bind to a target nucleic acid in a SPL9 gene having a gene identification number (gene ID) of Glyma_02G177500 (SPL9a), Glyma_09G113800 (SPL9b), Glyma_03g143100 (SPL9c), and/or Glyma_19g146000 (SPL9d).

In some embodiments, a guide nucleic acid of the invention binds to a target site in an endogenous IPA1 gene, wherein the endogenous IPA1 gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NOs:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UNBRANCHED 3 (UB3) gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89.

In some embodiments, the target site may be in a region of a SPL9 gene having at least about 80% sequence identity to any one of the nucleotide sequences of SEQ ID NOs:146-181, 185-221, 225-254 and/or 258-288.

In some embodiments, the target site may be in a region of a UB2 gene having at least about 80% sequence identity to any one of the nucleotide sequences of SEQ ID NOs:90-96 or 332-393.

In some embodiments, the target site may be in a region of a UB3 gene having at least about 80% sequence identity to any one of the nucleotide sequences of SEQ ID NOs:90, 97-103 or 394-445.

In some embodiments, a guide nucleic acid may comprise a spacer sequence having the nucleotide sequence of any one of SEQ ID NOs:104-142, 301, 326 and/or 327. In some embodiments, a guide nucleic acid may be directed to an SPL9 gene and may comprise the nucleotide sequence of SEQ ID NOs:104-124 and 301. In some embodiments, a guide nucleic acid may be directed to a UB3/UB2 gene and may comprise the nucleotide sequence of SEQ ID NOs:125-142, 326 and/or 327.

In some embodiments, a system is provided comprising a guide nucleic acid of the invention and a CRISPR-Cas effector protein that associates with the guide nucleic acid. In some embodiments, the system further comprises comprising a tracr nucleic acid that associates with the guide nucleic acid and a CRISPR-Cas effector protein, optionally wherein the tracr nucleic acid and the guide nucleic acid are covalently linked, optionally wherein the guide nucleic acid comprises a spacer having the nucleotide sequence of any one of SEQ ID

NOs:104-124 or 301 or SEQ ID NOs:125-142, 326 and/or 327.

The invention further provides a gene editing system comprising a CRISPR-Cas effector protein in association with a guide nucleic acid and the guide nucleic acid comprises a spacer sequence that binds to an endogenous IPA1 gene encoding an SPL transcription factor, wherein the IPA1 gene (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NOs:74, 77, 80, 83, 145, 184, 224, or 257; (b) is an endogenous UNBRANCHED 2 (UB2) gene: (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UNBRANCHED 3 (UB3) gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89. In some embodiments, a spacer sequence of the guide nucleic acid may comprise the nucleotide sequence of any one of SEQ ID NOs:104-124 or 301, or SEQ ID NOs:125-142, 326 or 327. In some embodiments, the gene editing system may further comprise a tracr nucleic acid that associates with the guide nucleic acid and a CRISPR-Cas effector protein, optionally wherein the tracr nucleic acid and the guide nucleic acid are covalently linked, optionally wherein the guide nucleic acid comprises a spacer sequence having a nucleotide sequence of any one of SEQ ID NOs:104-124 or 301 or a combination thereof, or SEQ ID NOs:125-142, 326 or 327, or a combination thereof.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site in a gene.

The present invention further provides a complex comprising a CRISPR-Cas effector protein comprising a cleavage domain and a guide nucleic acid, wherein the guide nucleic acid binds to a target site in a IPA1 gene, wherein the IPA1 gene (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NOs:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UNBRANCHED 3 (UB3) gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89, wherein the cleavage domain cleaves a target strand in the IPA1 gene.

In some embodiments, expression cassettes are provided that comprise (a) a polynucleotide encoding CRISPR-Cas effector protein comprising a cleavage domain and (b) a guide nucleic acid that binds to a target site in an IPA1 gene, wherein the guide nucleic acid comprises a spacer sequence that is complementary to and binds to the target site in the IPA1 gene, wherein the IPA1 gene: (a) is an endogenous SQUAMOSA PROMOTER BINDING PROTEIN-LIKE 9 (SPL9) gene (i) comprising a nucleotide sequence having at least 80% sequence identity to the nucleotide sequence of any one of SEQ ID NOs:72, 75, 78, 81, 143, 182, 222, or 255; (ii) comprising a coding sequence having at least 80% sequence identity to the nucleotide sequence of any one of SEQ ID NOs:73, 76, 79, 82, 144, 183, 223, or 256; (iii) comprising a region having least 80% sequence identity to the nucleotide sequence of any one of SEQ ID NOs:146-181, 185-221, 225-254 and/or 258-288, and/or (iv) encoding a polypeptide sequence having at least 80% identity to the amino acid sequence of any one of SEQ ID NOs:74, 77, 80, 83, 145, 184, 224, or 257; (b) is an endogenous UNBRANCHED 2 (UB2) gene (i) comprising a nucleotide sequence having at least 80% sequence identity to the nucleotide sequence of SEQ ID NO:84; (ii) comprising a coding sequence having at least 80% sequence identity to the nucleotide sequence of SEQ ID NO:85; (iii) comprising a region having least 80% sequence identity to the nucleotide sequence of any one of SEQ ID NOs:90-96 or 332-393, and/or (iv) encoding a polypeptide sequence having at least 80% identity to the amino acid sequence of SEQ ID NO:86; or (c) is an endogenous UNBRANCHED 3 (UB3) gene: (i) comprising a nucleotide sequence having at least 80% sequence identity to the nucleotide sequence of SEQ ID NO:87; (ii) comprising a coding sequence having at least 80% sequence identity to the nucleotide sequence of SEQ ID NO:88; (iii) comprising a region having least 80% sequence identity to the nucleotide sequence of any one of SEQ ID NOs:90, 97-103 or 394-445, and/or (iv) encoding a polypeptide sequence having at least 80% identity to the amino acid sequence of SEQ ID NO:89.

In some embodiments, the target site for a system or an expression cassette may be (a) in a region of the endogenous SPL9 gene having at least 80% sequence identity to the nucleotide sequence of any one of SEQ ID NOs:146-181, 185-221, 225-254 and/or 258-288; (b) in a region of the endogenous UB2 gene located from about nucleotide 2000 to about nucleotide 2391 or from about nucleotide 2225 to about nucleotide 2537 with reference to the nucleotide numbering of SEQ ID NO:84, from about nucleotide 1 to about nucleotide 391 or from about nucleotide 255 to about nucleotide 357 with reference to the nucleotide numbering of SEQ ID NO:85, optionally in a region of the endogenous UB2 gene having at least 80% sequence identity to the nucleotide sequence of any one of SEQ ID NOs:90-96 or 332-393, or (c) in a region of the endogenous UB3 gene located from about nucleotide 2001 to about nucleotide 2403 or from about nucleotide 2268 to about nucleotide 2370 with reference to the nucleotide numbering of SEQ ID NO:87, or from about nucleotide 1 to about nucleotide 403 or from about nucleotide 268 to about nucleotide 371 with reference to the nucleotide numbering of SEQ ID NO:88, optionally in a region of the endogenous UB3 gene having at least 80% sequence identity to the nucleotide sequence of any one of SEQ ID NOs:90, 97-103 or 394-445.

Also provided herein are nucleic acids encoding a mutated IPA1 gene that when present in a plant or plant part results in the plant having altered plant architecture, improved yield traits and/or increased tolerance/resistance to abiotic and biotic stress as compared to a plant or plant part devoid of the same mutation. In some embodiments, a mutation in an SPL9 gene may be a nucleotide sequence having at least 90% (e.g., 90, 91, 92, 93, 94, 95, 96, 97, 98, 99 or 100%) sequence identity to any one of SEQ ID NOs:289-300. In some embodiments, a plant may comprise two or more mutated SPL9 genes having at least 90% sequence identity to any one of SEQ ID NOs:289-300, in any combination. In some embodiments, a mutation in a UB3 gene may be a nucleotide sequence having at least 90% (e.g., 90, 91, 92, 93, 94, 95, 96, 97, 98, 99 or 100%) sequence identity to any one of SEQ ID NOs:310, 312, 314, 316, or 318, or may encode an amino acid sequence having at least 90% identity to any one of SEQ ID NOs:311, 313, 315, 317, or 319. In some embodiments, a mutation in a UB2 gene may be a nucleotide sequence having at least 90% (e.g., 90, 91, 92, 93, 94, 95, 96, 97, 98, 99 or 100%) sequence identity to any one of SEQ ID NOs:320, 322, or 324 or may encode an amino acid sequence having at least 90% identity to any one of SEQ ID NOs:321, 323, or 325. Also provided are soybean plants or parts thereof (e.g., a cell) comprising a mutated nucleic acid of any one of SEQ ID NOs: 289-300, optionally wherein the soybean plants or parts thereof may comprise two or more mutated SPL9 genes having at least 90% sequence identity to any one of SEQ ID NOs:289-300, in any combination.

In some embodiments, a soybean plant or plant part thereof is provided that comprises at least one mutation in at least one endogenous SLP9 gene having a gene identification number (gene ID) of Glyma_02G177500 (SPL9a), Glyma_09G113800 (SPL9b), Glyma_03g143100 (SPL9c), and/or Glyma_19g146000 (SPL9d), optionally the soybean plant or part thereof comprising at least one mutation in an SPL9 gene exhibits a phenotype of one or more of altered plant architecture, improved yield traits and/or increased tolerance/resistance to abiotic and biotic stress as compared to a plant or plant part not comprising the same mutation.

In some embodiments, a mutated endogenous SPL9 gene is provided that comprises a nucleic acid sequence having at least 90% identity to any one of SEQ ID NOs:289-300.

Also provided are corn plants or parts thereof (e.g., a cell) comprising a mutated nucleic acid of any one of SEQ ID NOs:310, 312, 314, 316, or 318, and/or SEQ ID NOs:320, 322, or 324, optionally wherein the corn plants or parts thereof may comprise two or more mutated UB2/UB3 genes having at least 90% sequence identity to any one of SEQ ID NOs: 310, 312, 314, 316, or 318, and/or SEQ ID NOs:320, 322, or 324, in any combination, optionally wherein the corn plant or part thereof exhibits a phenotype of one or more of altered plant architecture, improved yield traits and/or increased tolerance/resistance to abiotic and biotic stress as compared to a plant or plant part not comprising the same mutation.

In some embodiments, a mutated endogenous UB2 gene is provided that comprises a nucleic acid sequence having at least 90% identity to any one of SEQ ID NOs:310, 312, 314, 316, or 318. In some embodiments, a mutated endogenous UB3 gene is provided that comprises a nucleic acid sequence having at least 90% identity to any one of SEQ ID NOs:320, 322, or 324.

In some embodiments, the present invention provides a method of producing a plant comprising a mutation in an endogenous IPA1 gene encoding an SPL transcription factor and at least one polynucleotide of interest, the method comprising crossing a plant of the invention comprising at least one mutation in an endogenous IPA1 gene (a first plant) with a second plant that comprises the at least one polynucleotide of interest to produce progeny plants; and selecting progeny plants comprising at least one mutation in the IPA1 gene and the at least one polynucleotide of interest, thereby producing the plant comprising a mutation in an endogenous IPA1 gene and at least one polynucleotide of interest.

The present invention further provides a method of producing a plant comprising a mutation in an endogenous IPA1 gene encoding an SPL transcription factor and at least one polynucleotide of interest, the method comprising introducing at least one polynucleotide of interest into a plant of the present invention comprising at least one mutation in an endogenous IPA1 gene encoding an SPL transcription factor, thereby producing a plant comprising at least one mutation in an IPA1 gene and at least one polynucleotide of interest.

In some embodiments, a method of producing a plant is provided comprising a mutation in an endogenous IPA1 gene and exhibiting a phenotype of improved root architecture (optionally, exhibiting improved yield traits, increased root biomass, steeper root angle and/or longer roots), comprising crossing a first plant, which is the plant of the present invention, with a second plant that exhibits a phenotype of improved root architecture; and selecting progeny plants comprising the mutation in the IPA1 gene and a phenotype of improved root architecture, thereby producing the plant comprising a mutation in an endogenous IPA1 gene and exhibiting a phenotype of improved root architecture as compared to a control plant.

In some embodiments, a method of controlling weeds in a container (e.g., pot, or seed tray and the like), a growth chamber, a greenhouse, a field, a recreational area, a lawn, or on a roadside, comprising applying an herbicide to one or more (a plurality) plants the present invention growing in a container, a growth chamber, a greenhouse, a field, a recreational area, a lawn, or on a roadside, thereby controlling the weeds in the container, the growth chamber, the greenhouse, the field, the recreational area, the lawn, or on the roadside in which the one or more plants are growing.

In some embodiments, a method of reducing insect predation on a plant is provided, comprising applying an insecticide to one or more plants of the invention, thereby reducing insect predation on the one or more plants, optionally wherein the one or more plants are growing in a container, a growth chamber, a greenhouse, a field, a recreational area, a lawn, or on a roadside.

In some embodiments, a method of reducing fungal disease on a plant is provided, comprising applying a fungicide to one or more plants of the invention, thereby reducing fungal disease on the one or more plants, optionally wherein the one or more plants are growing in a container, a growth chamber, a greenhouse, a field, a recreational area, a lawn, or on a roadside.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, disease resistance, increased yield, increased nutrient use efficiency or abiotic stress resistance.

Thus, plants or plant cultivars which are to be treated with preference in accordance with the invention include all plants which, through genetic modification, received genetic material which imparts particular advantageous useful properties (“traits”) to these plants. Examples of such properties are better plant growth, vigor, stress tolerance, standability, lodging resistance, nutrient uptake, plant nutrition, and/or yield, in particular improved growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processability of the harvested products.

Further examples of such properties are an increased resistance against animal and microbial pests, such as against insects, arachnids, nematodes, mites, slugs and snails owing, for example, to toxins formed in the plants. Among DNA sequences encoding proteins which confer properties of tolerance to such animal and microbial pests, in particular insects, mention will particularly be made of the genetic material from Bacillus thuringiensis encoding the Bt proteins widely described in the literature and well known to those skilled in the art. Mention will also be made of proteins extracted from bacteria such as Photorhabdus (WO97/17432 and WO98/08932). In particular, mention will be made of the Bt Cry or VIP proteins which include the CryIA, CryIAb, CryIAc, CryIIA, CryIIIA, CryIIIB2, Cry9c Cry2Ab, Cry3Bb and CryIF proteins or toxic fragments thereof and also hybrids or combinations thereof, especially the CryIF protein or hybrids derived from a CryIF protein (e.g. hybrid CryIA-CryIF proteins or toxic fragments thereof), the CryIA-type proteins or toxic fragments thereof, preferably the CryIAc protein or hybrids derived from the CryIAc protein (e.g. hybrid CryIAb-CryIAc proteins) or the CryIAb or Bt2 protein or toxic fragments thereof, the Cry2Ae, Cry2Af or Cry2Ag proteins or toxic fragments thereof, the CryIA.105 protein or a toxic fragment thereof, the VIP3Aa19 protein, the VIP3Aa20 protein, the VIP3A proteins produced in the COT202 or COT203 cotton events, the VIP3Aa protein or a toxic fragment thereof as described in Estruch et al. (1996), Proc Natl Acad Sci USA. 28;93(11):5389-94, the Cry proteins as described in WO2001/47952, the insecticidal proteins from Xenorhabdus (as described in WO98/50427), Serratia (particularly from S. entomophila) or Photorhabdus species strains, such as Tc-proteins from Photorhabdus as described in WO98/08932. Also any variants or mutants of any one of these proteins differing in some amino acids (1-10, preferably 1-5) from any of the above named sequences, particularly the sequence of their toxic fragment, or which are fused to a transit peptide, such as a plastid transit peptide, or another protein or peptide, is included herein.

Another and particularly emphasized example of such properties is conferred tolerance to one or more herbicides, for example imidazolinones, sulphonylureas, glyphosate or phosphinothricin. Among DNA sequences encoding proteins (i.e., polynucleotides of interest) which confer properties of tolerance to certain herbicides on the transformed plant cells and plants, mention will be particularly be made to the bar or PAT gene or the Streptomyces coelicolor gene described in WO2009/152359 which confers tolerance to glufosinate herbicides, a gene encoding a suitable EPSPS (5-Enolpyruvylshikimat-3-phosphat-Synthase) which confers tolerance to herbicides having EPSPS as a target, especially herbicides such as glyphosate and its salts, a gene encoding glyphosate-n-acetyltransferase, or a gene encoding glyphosate oxidoreductase. Further suitable herbicide tolerance traits include at least one ALS (acetolactate synthase) inhibitor (e.g. WO2007/024782), a mutated Arabidopsis ALS/AHAS gene (e.g. U.S. Pat. No. 6,855,533), genes encoding 2,4-D-monooxygenases conferring tolerance to 2,4-D (2,4-dichlorophenoxyacetic acid) and genes encoding Dicamba monooxygenases conferring tolerance to dicamba (3,6-dichloro-2-methoxybenzoic acid).

Further examples of such properties are increased resistance against phytopathogenic fungi, bacteria and/or viruses owing, for example, to systemic acquired resistance (SAR), systemin, phytoalexins, elicitors and also resistance genes and correspondingly expressed proteins and toxins.

Particularly useful transgenic events in transgenic plants or plant cultivars which can be treated with preference in accordance with the invention include Event 531/PV-GHBK04 (cotton, insect control, described in WO2002/040677), Event 1143-14A (cotton, insect control, not deposited, described in WO2006/128569); Event 1143-51B (cotton, insect control, not deposited, described in WO2006/128570); Event 1445 (cotton, herbicide tolerance, not deposited, described in US-A 2002-120964 or WO2002/034946); Event 17053 (rice, herbicide tolerance, deposited as PTA-9843, described in WO2010/117737); Event 17314 (rice, herbicide tolerance, deposited as PTA-9844, described in WO2010/117735); Event 281-24-236 (cotton, insect control-herbicide tolerance, deposited as PTA-6233, described in WO2005/103266 or US-A 2005-216969); Event 3006-210-23 (cotton, insect control-herbicide tolerance, deposited as PTA-6233, described in US-A 2007-143876 or WO2005/103266); Event 3272 (corn, quality trait, deposited as PTA-9972, described in WO2006/098952 or US-A 2006-230473); Event 33391 (wheat, herbicide tolerance, deposited as PTA-2347, described in WO2002/027004), Event 40416 (corn, insect control-herbicide tolerance, deposited as ATCC PTA-11508, described in WO 11/075593); Event 43A47 (corn, insect control-herbicide tolerance, deposited as ATCC PTA-11509, described in WO2011/075595); Event 5307 (corn, insect control, deposited as ATCC PTA-9561, described in WO2010/077816); Event ASR-368 (bent grass, herbicide tolerance, deposited as ATCC PTA-4816, described in US-A 2006-162007 or WO2004/053062); Event B16 (corn, herbicide tolerance, not deposited, described in US-A 2003-126634); Event BPS-CV127-9 (soybean, herbicide tolerance, deposited as NCIMB No. 41603, described in WO2010/080829); Event BLRI (oilseed rape, restoration of male sterility, deposited as NCIMB 41193, described in WO2005/074671), Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO2006/128571); Event CE46-02A (cotton, insect control, not deposited, described in WO2006/128572); Event COT102 (cotton, insect control, not deposited, described in US-A 2006-130175 or WO2004/039986); Event COT202 (cotton, insect control, not deposited, described in US-A 2007-067868 or WO2005/054479); Event COT203 (cotton, insect control, not deposited, described in WO2005/054480);); Event DAS21606-3/1606 (soybean, herbicide tolerance, deposited as PTA-11028, described in WO2012/033794), Event DAS40278 (corn, herbicide tolerance, deposited as ATCC PTA-10244, described in WO2011/022469); Event DAS-44406-6/pDAB8264.44.06.l (soybean, herbicide tolerance, deposited as PTA-11336, described in WO2012/075426), Event DAS-14536-7/pDAB8291.45.36.2 (soybean, herbicide tolerance, deposited as PTA-11335, described in WO2012/075429), Event DAS-59122-7 (corn, insect control-herbicide tolerance, deposited as ATCC PTA 11384, described in US-A 2006-070139); Event DAS-59132 (corn, insect control-herbicide tolerance, not deposited, described in WO2009/100188); Event DAS68416 (soybean, herbicide tolerance, deposited as ATCC PTA-10442, described in WO2011/066384 or WO2011/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO2008/054747); Event DP-32138-1 (corn, hybridization system, deposited as ATCC PTA-9158, described in US-A 2009-0210970 or WO2009/103049); Event DP-356043-5 (soybean, herbicide tolerance, deposited as ATCC PTA-8287, described in US-A 2010-0184079 or WO2008/002872); Event EE-I (brinjal, insect control, not deposited, described in WO 07/091277); Event Fil 17 (corn, herbicide tolerance, deposited as ATCC 209031, described in US-A 2006-059581 or WO 98/044140); Event FG72 (soybean, herbicide tolerance, deposited as PTA-11041, described in WO2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO98/044140); Event GHB119 (cotton, insect control-herbicide tolerance, deposited as ATCC PTA-8398, described in WO2008/151780); Event GHB614 (cotton, herbicide tolerance, deposited as ATCC PTA-6878, described in US-A 2010-050282 or WO2007/017186); Event GJ11 (corn, herbicide tolerance, deposited as ATCC 209030, described in US-A 2005-188434 or WO98/044140); Event GM RZ13 (sugar beet, virus resistance, deposited as NCIMB-41601, described in WO2010/076212); Event H7-I (sugar beet, herbicide tolerance, deposited as NCIMB 41158 or NCIMB 41159, described in US-A 2004-172669 or WO 2004/074492); Event JOPLINI (wheat, disease tolerance, not deposited, described in US-A 2008-064032); Event LL27 (soybean, herbicide tolerance, deposited as NCIMB41658, described in WO2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO2003/013224 or US-A 2003-097687); Event LLRICE06 (rice, herbicide tolerance, deposited as ATCC 203353, described in U.S. Pat. No. 6,468,747 or WO2000/26345); Event LLRice62 (rice, herbicide tolerance, deposited as ATCC 203352, described in WO2000/26345), Event LLRICE601 (rice, herbicide tolerance, deposited as ATCC PTA-2600, described in US-A 2008-2289060 or WO2000/26356); Event LY038 (corn, quality trait, deposited as ATCC PTA-5623, described in US-A 2007-028322 or WO2005/061720); Event MIR162 (corn, insect control, deposited as PTA-8166, described in US-A 2009-300784 or WO2007/142840); Event MIR604 (corn, insect control, not deposited, described in US-A 2008-167456 or WO2005/103301); Event MON15985 (cotton, insect control, deposited as ATCC PTA-2516, described in US-A 2004-250317 or WO2002/100163); Event MON810 (corn, insect control, not deposited, described in US-A 2002-102582); Event MON863 (corn, insect control, deposited as ATCC PTA-2605, described in WO2004/011601 or US-A 2006-095986); Event MON87427 (corn, pollination control, deposited as ATCC PTA-7899, described in WO2011/062904); Event MON87460 (corn, stress tolerance, deposited as ATCC PTA-8910, described in WO2009/111263 or US-A 2011-0138504); Event MON87701 (soybean, insect control, deposited as ATCC PTA-8194, described in US-A 2009-130071 or WO2009/064652); Event MON87705 (soybean, quality trait—herbicide tolerance, deposited as ATCC PTA-9241, described in US-A 2010-0080887 or WO2010/037016); Event MON87708 (soybean, herbicide tolerance, deposited as ATCC PTA-9670, described in WO2011/034704); Event MON87712 (soybean, yield, deposited as PTA-10296, described in WO2012/051199), Event MON87754 (soybean, quality trait, deposited as ATCC PTA-9385, described in WO2010/024976); Event MON87769 (soybean, quality trait, deposited as ATCC PTA-8911, described in US-A 2011-0067141 or WO2009/102873); Event MON88017 (corn, insect control —herbicide tolerance, deposited as ATCC PTA-5582, described in US-A 2008-028482 or WO2005/059103); Event MON88913 (cotton, herbicide tolerance, deposited as ATCC PTA-4854, described in WO2004/072235 or US-A 2006-059590); Event MON88302 (oilseed rape, herbicide tolerance, deposited as PTA-10955, described in WO2011/153186), Event MON88701 (cotton, herbicide tolerance, deposited as PTA-11754, described in WO2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO2006/130436); Event MSI 1 (oilseed rape, pollination control-herbicide tolerance, deposited as ATCC PTA-850 or PTA-2485, described in WO2001/031042); Event MS8 (oilseed rape, pollination control-herbicide tolerance, deposited as ATCC PTA-730, described in WO2001/041558 or US-A 2003-188347); Event NK603 (corn, herbicide tolerance, deposited as ATCC PTA-2478, described in US-A 2007-292854); Event PE-7 (rice, insect control, not deposited, described in WO2008/114282); Event RF3 (oilseed rape, pollination control —herbicide tolerance, deposited as ATCC PTA-730, described in WO2001/041558 or US-A 2003-188347); Event RT73 (oilseed rape, herbicide tolerance, not deposited, described in WO2002/036831 or US-A 2008-070260); Event SYHT0H2/SYN-000H2-5 (soybean, herbicide tolerance, deposited as PTA-11226, described in WO2012/082548), Event T227-1 (sugar beet, herbicide tolerance, not deposited, described in WO2002/44407 or US-A 2009-265817); Event T25 (corn, herbicide tolerance, not deposited, described in US-A 2001-029014 or WO2001/051654); Event T304-40 (cotton, insect control-herbicide tolerance, deposited as ATCC PTA-8171, described in US-A 2010-077501 or WO2008/122406); Event T342-142 (cotton, insect control, not deposited, described in WO2006/128568); Event TC1507 (corn, insect control-herbicide tolerance, not deposited, described in US-A 2005-039226 or WO2004/099447); Event VIP1034 (corn, insect control-herbicide tolerance, deposited as ATCC PTA-3925, described in WO2003/052073), Event 32316 (corn, insect control-herbicide tolerance, deposited as PTA-11507, described in WO2011/084632), Event 4114 (corn, insect control-herbicide tolerance, deposited as PTA-11506, described in WO2011/084621), event EE-GM3/FG72 (soybean, herbicide tolerance, ATCC Accession No PTA-11041) optionally stacked with event EE-GM1/LL27 or event EE-GM2/LL55 (WO2011/063413A2), event DAS-68416-4 (soybean, herbicide tolerance, ATCC Accession No PTA-10442, WO2011/066360A1), event DAS-68416-4 (soybean, herbicide tolerance, ATCC Accession No PTA-10442, WO2011/066384A1), event DP-040416-8 (corn, insect control, ATCC Accession No PTA-11508, WO2011/075593A1), event DP-043A47-3 (corn, insect control, ATCC Accession No PTA-11509, WO2011/075595A1), event DP-004114-3 (corn, insect control, ATCC Accession No PTA-11506, WO2011/084621A1), event DP-032316-8 (corn, insect control, ATCC Accession No PTA-11507, WO2011/084632A1), event MON-88302-9 (oilseed rape, herbicide tolerance, ATCC Accession No PTA-10955, WO2011/153186A1), event DAS-21606-3 (soybean, herbicide tolerance, ATCC Accession No. PTA-11028, WO2012/033794A2), event MON-87712-4 (soybean, quality trait, ATCC Accession No. PTA-10296, WO2012/051199A2), event DAS-44406-6 (soybean, stacked herbicide tolerance, ATCC Accession No. PTA-11336, WO2012/075426A1), event DAS-14536-7 (soybean, stacked herbicide tolerance, ATCC Accession No. PTA-11335, WO2012/075429A1), event SYN-000H2-5 (soybean, herbicide tolerance, ATCC Accession No. PTA-11226, WO2012/082548A2), event DP-061061-7 (oilseed rape, herbicide tolerance, no deposit No available, WO2012071039A1), event DP-073496-4 (oilseed rape, herbicide tolerance, no deposit No available, US2012131692), event 8264.44.06.1 (soybean, stacked herbicide tolerance, Accession No PTA-11336, WO2012075426A2), event 8291.45.36.2 (soybean, stacked herbicide tolerance, Accession No. PTA-11335, WO2012075429A2), event SYHT0H2 (soybean, ATCC Accession No. PTA-11226, WO2012/082548A2), event MON88701 (cotton, ATCC Accession No PTA-11754, WO2012/134808A1), event KK179-2 (alfalfa, ATCC Accession No PTA-11833, WO2013/003558A1), event pDAB8264.42.32.1 (soybean, stacked herbicide tolerance, ATCC Accession No PTA-11993, WO2013/010094A1), event MZDT09Y (corn, ATCC Accession No PTA-13025, WO2013/012775A1).

The genes/events (e.g., polynucleotides of interest), which impart the desired traits in question, may also be present in combinations with one another in the transgenic plants. Examples of transgenic plants which may be mentioned are the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (with the fruits apples, pears, citrus fruits and grapes), with particular emphasis being given to maize, soya beans, wheat, rice, potatoes, cotton, sugar cane, tobacco and oilseed rape. Traits which are particularly emphasized are the increased resistance of the plants to insects, arachnids, nematodes and slugs and snails, as well as the increased resistance of the plants to one or more herbicides.

Commercially available examples of such plants, plant parts or plant seeds that may be treated with preference in accordance with the invention include commercial products, such as plant seeds, sold or distributed under the GENUITY®, DROUGHTGARD®, SMARTSTAX®, RIB COMPLETE®, ROUNDUP READY®, VT DOUBLE PRO®, VT TRIPLE PRO®, BOLLGARD II®, ROUNDUP READY 2 YIELD®, YIELDGARD®, ROUNDUP READY® 2 XTEND™, INTACTA RR2 PRO®, VISTIVE GOLD®, and/or XTENDFLEX™ trade names.

Nucleic acid constructs of the invention (e.g., a construct comprising a sequence specific nucleic acid binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids (e.g., endogenous IPA1 genes encoding SPL transcription factors) and/or their expression.

Any plant comprising an endogenous IPA1 gene encoding a SPL transcription factor that is capable of conferring altered plant architecture, improved yield traits and/or increased tolerance/resistance to abiotic and biotic stress when modified as described herein (e.g., mutated, e.g., base edited, cleaved, nicked, etc.) using the polypeptides, polynucleotides, RNPs, nucleic acid constructs, expression cassettes, and/or vectors of the invention.

An “increased kernel row number” or (e.g., a corn plant) as used herein refers to an increase in kernel row number by about 5% to about 30% (e.g., about 5, 6, 7, 8, 9, 10, 11, 12, 13, 14, 15, 16, 17, 18, 19, 20, 21, 22, 23, 24, 25, 26, 27, 28, 29, or 30% or any range or value therein; e.g., about 5% to about 10%, about 5% to about 15%, about 5% to about 20%, about 5% to about 25%, about 5% to about 30%, about 10% to about 15%, about 10% to about 20%, about 10% to about 25%, about 10% to about 30%, about 20% to about 30%, about 25% to about 30%, and any range or value therein) (e.g., about 1, 2, 3, or 4 more rows) as compared to a plant or part thereof that does not comprise the mutated endogenous IPA1 gene. In some embodiments, plants exhibiting increased kernel row number as described herein (e.g., a plant that produces ears having increased kernel row number) produce ears that are also not substantially decreased in length. An ear of a plant comprising a mutation as described herein that is “not substantially decreased in length” refers to an ear of a plant has a length that is reduced by less than 30% (e.g., reduced by 0% or reduced by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30%) as compared to a plant that does not comprise the same IPA1 mutation.

As used herein, “altered plant architecture” refers to a structure of the plant that has been altered through a modification of the plant's genome as described herein. Such structure can include, but is not limited to, the number of branches, number of nodes, number of pods (e.g., number of pods on the mainstem and branches), the number of flowers, plant biomass, increased root biomass, steeper root angle and/or longer roots.

The term “abiotic stress” as used herein refers to outside, nonliving, factors which can cause harmful effects to plants. Accordingly, abiotic stresses can include, but are not limited to, cold temperature that results in freezing, chilling, heat or high temperatures, drought, high light intensity, low light intensity, salinity, osmotic stress, ozone, high plant density, nutrient deficiency/toxicity, and/or combinations thereof. Parameters for the abiotic stress factors are species specific and even variety specific and therefore vary widely according to the species/variety exposed to the abiotic stress. Thus, while one species may be severely impacted by a high temperature of 23° C., another species may not be impacted until at least 30° C., and the like. Temperatures above 30° C. result in dramatic reductions in the yields of most important crops. This is due to reductions in photosynthesis that begin at approximately 20-25° C., and the increased carbohydrate demands of crops growing at higher temperatures. The critical temperatures are not absolute but vary depending upon such factors as the acclimatization of the crop to prevailing environmental conditions. In addition, because most crops are exposed to multiple abiotic stresses at one time, the interaction between the stresses affects the response of the plant. For example, damage from excess light occurs at lower light intensities as temperatures increase beyond the photosynthetic optimum. Water stressed plants are less able to cool overheated tissues due to reduced transpiration, further exacerbating the impact of excess (high) heat and/or excess (high) light intensity. Thus, the particular parameters for high/low temperature, light intensity, drought and the like, which impact crop productivity will vary with species, variety, degree of acclimatization and the exposure to a combination of environmental conditions. “Increased abiotic stress resistance/tolerance” refers to an increase in resistance or tolerance to a stress of about 5% to about 100% (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 75, 80, 81, 82, 83, 84, 85, 90, 91, 92, 93, 94, 95, 96, 97, 98, 99, or 100%, or any range or value therein), as compared to a control plant.

The term “biotic stress” as used herein refers to living or biotic factors that have harmful effects on plants. Such biotic factors include, but are not limited to, disease causing organisms (bacteria, fungi, fungi-like organisms, nematodes, viruses, phytoplasmas, insects, parasitic plants, and the like). “Increased biotic stress resistance/tolerance” refers to an increase in resistance or tolerance to a stress of about 15% to about 200% (about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 110, 111, 12, 113, 114, 115, 116, 117, 118, 119, 120, 121, 122, 123, 124, 125, 126, 127, 128, 129, 130, 131, 132, 133, 134, 135, 140, 141, 142, 143, 144, 145, 146, 147, 148, 149, 150, 151, 152, 153, 154, 155, 156, 157,158, 159, 160, 161, 162, 163, 164, 165, 170, 171, 172, 173, 174, 175, 180, 185, 186, 187, 188, 189, 190, 191, 192, 193, 194, 195, 196, 197, 198, 199, or 200%, or any range or value therein) when compared to a control plant..

The term “plant part,” as used herein, includes but is not limited to reproductive tissues (e.g., petals, sepals, stamens, pistils, receptacles, anthers, pollen, flowers, fruits, flower bud, ovules, seeds, and embryos); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit. An editing system useful with this invention can be any site-specific (sequence-specific) genome editing system now known or later developed, which system can introduce mutations in target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide. In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing an IPA1 gene encoding an SPL transcription factor may comprise contacting a target nucleic acid (e.g., a nucleic acid encoding an SPL transcription factor) with a base-editing fusion protein (e.g., a sequence specific nucleic acid binding protein, a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific nucleic acid binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing an IPA1 gene encoding an SPL transcription factor may comprise contacting a target nucleic acid (e.g., an IPA1 nucleic acid encoding an SPL transcription factor) with a sequence-specific nucleic acid binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific nucleic acid binding fusion protein to the target nucleic acid and the sequence-specific nucleic acid binding fusion protein is capable of recruiting the deaminase fusion protein to the target nucleic acid via the peptide tag-affinity polypeptide interaction, thereby editing a locus within the target nucleic acid. In some embodiments, the sequence-specific nucleic acid binding fusion protein may be fused to the affinity polypeptide that binds the peptide tag and the deaminase may be fuse to the peptide tag, thereby recruiting the deaminase to the sequence-specific nucleic acid binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific nucleic acid binding fusion proteins, deaminase fusion proteins and guides may be provided as ribonucleoproteins (RNPs).

In some embodiments, methods such as prime editing may be used to generate a mutation in an endogenous IPA gene encoding an SPL transcription factor. In prime editing, RNA-dependent DNA polymerase (reverse transcriptase, RT) and reverse transcriptase templates (RT template) are used in combination with sequence specific nucleic acid binding domains that confer the ability to recognize and bind the target in a sequence-specific manner, and which can also cause a nick of the PAM-containing strand within the target. The nucleic acid binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantageous of the various methods of recruiting proteins for use in the editing to the target site, such methods including both non-covalent and covalent interactions between the proteins and nucleic acids used in the selected process of genome editing.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a sequence-specific nucleic acid binding domain (e.g., a sequence-specific DNA binding domain) may be a CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, CasI, CasIB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, CsbI, Csb2, Csb3, CsxI7, CsxI4, Csx10, Csx16, CsaX, Csx3, Csx1, CsxI5, CsfI, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain, e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g, Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO:59 and SEQ ID NO:60 or the nucleotide sequences of SEQ ID NOs:61-71.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease see, e.g., SEQ ID NOs:1-20). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat or a mouse. Thus, in some embodiments, a cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27, SEQ ID NO:28 or SEQ ID NO:29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25,

SEQ ID NO:26, SEQ ID NO:27, SEQ ID NO:28 or SEQ ID NO:29). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82)/0, 83%, 84%, 85%, 86%, 87%, 88%, 89%, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coll. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs:31-40 (e.g., SEQ ID NOs:31, 32, 33, 34, 35, 36, 37, 38, 39 or 40). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific nucleic acid binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt function; and/or generation of point mutations in genomic DNA to disrupt splice junctions.

The nucleic acid constructs of the invention encoding a base editor comprising a sequence-specific nucleic acid binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, CasI, CasIB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, CscI, Csc2, Csa5, Csn2, Csm2, Csm3, Csm4, Csm5, Csm6, Cmr1, Cmr3, Cmr4, Cmr5, Cmr6, CsbI, Csb2, Csb3, CsxI7, CsxI4, Csx10, Csx16, CsaX, Csx3, CsxI, CsxI5, CsfI, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, CasI, CasIB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, CscI, Csc2, Csa5, Csn2, Csm2, Csm3, Csm4, Csm5, Csm6, Cmr1, Cmr3, Cmr4, Cmr5, Cmr6, CsbI, Csb2, Csb3, CsxI7, CsxI4, Csx10, Csx16, CsaX, Csx3, CsxI, CsxI5, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, Ill, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., consecutive nucleotides of portion/region of a sequence (a) having at least 80% sequence identity to the nucleotide sequence of any one of SEQ ID NOs:72, 73, 75, 76, 78, 79, 81, 82, 143, 144, 182, 183, 222, 223, 255 or 256 and/or encoding a sequence having at least 80% sequence identity to the amino acid sequences of any one of SEQ ID NOs:74, 77, 80, 83, 145, 184, 224, or 257 (e.g., a region having least 80% sequence identity to the nucleotide sequence of any one of SEQ ID NOs:146-181, 185-221, 225-254 and/or 258-288); (b) having at least 80% sequence identity to the nucleotide sequence of SEQ ID NO:84 or 85 and/or encoding a sequence having at least 80% sequence identity to the amino acid sequence of SEQ ID NO:86 (e.g., a region having least 80% sequence identity to the nucleotide sequence of any one of SEQ ID NOs:90-96 or 332-393); and/or (c) having at least 80% sequence identity to the nucleotide sequence of SEQ ID NO:87 or 88 and/or encoding a sequence having at least 80% sequence identity to the amino acid sequence of SEQ ID NO:89 (e.g., a region having least 80% sequence identity to the nucleotide sequence of any one of SEQ ID NOs:90, 97-103 or 394-445). In some embodiments, a spacer sequence may include, but is not limited to, the nucleotide sequence of any one of SEQ ID NOs:104-142 or 301, or SEQ ID NOs:125-142, 326 and/or 327, or a reverse complement thereof. The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (such as for a Type V CRISPR-Cas), or the 3′ region of a spacer sequence of a guide nucleic acid may be identical to a target DNA, while the 5′ region of the spacer may be substantially complementary to the target DNA (such as for a Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
5′-NNNNNNNNNNNNNNNNNNN-3′ RNA Spacer (SEQ ID NO: 42)
        ||||||||||||||||||||
3′AAANNNNNNNNNNNNNNNNNNN-5′ Target strand (SEQ ID NO: 43)
    ||||
5′TTTNNNNNNNNNNNNNNNNNNN-3′ Non-target strand (SEQ ID NO: 44)
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5c-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g, one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains, CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG® octapeptide, a strep tag or strep tag H, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs:45-47.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs:48-58.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together; e.g. dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided herein are cells comprising one or more polynucleotides, guide nucleic acids, nucleic acid constructs, expression cassettes or vectors of the invention.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. Editing the miR156 Binding Site in Soybean SPL9 Genes

A strategy was developed for altering regulation of the SPL9 gene by generating edits in the miR156 binding site in the promoter of the soybean SPL9 genes. An editing construct was designed with spacer PWsp693 (ATTTGACAGAAGAGAGAGAGCAC) (SEQ ID NO:301) to edit the SPL9c (SEQ ID NO:222) and SPL9d (SEQ ID NO:255) genes. Another editing construct was designed with additional spacers, PWsp1071 (ACTTGACAGAAGAGAGAGAGCAC) (SEQ ID NO:114) and PWsp1072 (TCTTGACAGAAGAGAGAGAGCAC) (SEQ ID NO:115), to edit the SPL9a (SEQ ID NO:143) and SPL9b (SEQ ID NO:182) genes.

Lines carrying edits in the SPL9 genes were screened and those that showed about 10% of the sequencing reads having edits in the targeted gene were advanced to the next generation. The edited alleles generated by these two constructs are summarized in Table 1.

TABLE 1
Edited alleles

| E0 plant | | |
|---|---|---|
| name | Edited sequence | Notes |
| CE44978 | SPL9c contains an 8 bp deletion | The 8 bp deletion in SPL9c |
| | beginning at position 5766 bp in | results in a frame shift |
| | SEQ ID NO: 289; deleted sequence | mutation giving rise to an |
| | is TGCTCTCT | early stop codon. |
| CE44978 | SPL9d contains a 7 bp deletion | The 7 bp deletion in SPL9d |
| | corresponding to position 6220 | results in a frame shift |
| | bp in SEQ ID NO: 290; deleted | mutation giving rise to an |
| | sequence is GTGCTCT. | early stop codon |
| CE44907 | SPL9c contains a 13 bp deletion | The 13 bp deletion in SPL9c |
| | corresponding to position 5757 | results in a frame shift |
| | bp of SEQ ID NO:292, deleted | mutation giving rise to an |
| | sequence is TCGAGCTGTGCTC | early stop codon. |
| | SEQ ID NO: 307 | |
| CE44907 | SPL9c contains a 29 bp deletion | The 29 bp deletion in SPL9c |
| | corresponding to position 5757 | overlaps entirely with the |
| | bp of SEQ ID NO: 291, deleted | 13 bp deletion described |
| | sequence is | above. The 29 bp deletion |
| | TCGAGCTGTGCTCTCTCTCTTCTGTCAAA | results in a frame shift |
| | SEQ ID NO: 308 | mutation giving rise to |
| | | an early stop codon. |
| CE44907 | SPL9d contains an 11 bp deletion | The 11 bp deletion in SPL9d |
| | corresponding to position 6218 | overlaps entirely with the |
| | bp of SEQ ID NO: 294; deleted | 5 bp deletion described |
| | sequence is CTGTGCTCTCT | below. The 11 bp deletion |
| | SEQ ID NO:309 | results in a frame shift |
| | | mutation giving rise to an |
| | | early stop codon. |
| CE44907 | SPL9d contains a 5 bp deletion | The 5 bp deletion in SPL9d |
| | corresponding to position 6224 | results in a frame shift |
| | bp of SEQ ID NO: 293; deleted | mutation giving rise to an |
| | sequence is TCTCT | early stop codon. |
| CE56385 | SPL9a contains a 9 bp deletion | The 9 bp deletion in SPL9a |
| | corresponding to position 6720 | results in an in-frame |
| | of SEQ ID NO:295; deleted | deletion in the region |
| | sequence is CTGTGCTCT | of miR156. |

### Example 2: Knocking Down SPL9 Genes in Soybean

A strategy to generate hypomorphic edits/mutations of soybean SPL9 genes was developed to reduce expression of the SPL9 gene(s). The editing construct pWISE2878 was designed with the of spacers PWsp1128 (SEQ ID NO:104), PWsp1129 (SEQ ID NO:105) and

PWsp1130 (SEQ ID NO:106) to edit the SPL9a (SEQ ID NO:143) and SPL9b (SEQ ID NO:182) genes. The editing construct pWISE2879 was designed with the spacers PWsp1134 (SEQ ID NO:110), PWsp1135 (SEQ ID NO:111), PWsp1136 (SEQ ID NO:112) and PWsp1137 (SEQ ID NO:113) to edit the SPL9c (SEQ ID NO:222) and SPL9d (SEQ ID NO:255) genes.

Lines carrying edits in the SPL9 genes were screened and those that showed about 10% of the sequencing reads having edits in the targeted gene were advanced to the next generation. The edited alleles generated by these two constructs are summarized in Table 2.

TABLE 2
Edited alleles

| E0 name | Edited sequence | Deleted sequence |
|---|---|---|
| CE52366 | SPL9b contains a | CTCTCAAGTCTCAA |
| | 54 bp deletion | CGAAATGAATTAAT |
| | corresponding to | GTATATACACTCAC |
| | position 1982 of | TCACTCTCGTTA |
| | SEQ ID NO: 299 | SEQ ID NO: 302 |
| CE52366 | SPL9a contains a | CACTCTAGTTAG |
| | 12 bp deletion | SEQ ID NO:303 |
| | corresponding to | |
| | position 2097 of | |
| | SEQ ID NO: 300 | |
| CE56666 | SPL9c contains | 2001 10 bp |
| | three deletions | deletion |
| | of 10 bp | (AAATTGTCTG) |
| | corresponding to | SEQ ID NO: 304 |
| | position 2001; 4 | 2113 4 bp |
| | bp corresponding | deletion |
| | to position 2113; | (TGTT) |
| | and 4 bp | 2131 4 bp |
| | corresponding | deletion |
| | to position 2131; | (CACT) |
| | all with respect | |
| | to SEQ ID NO: 296 | |
| CE56666 | SPL9c contains a | ATTCTTGGTTTTT |
| | 68 bp deletion | GGACAGACAAGGG |
| | corresponding to | TGTGTTTGGTTCT |
| | position 2084 of | CTGACTACACTCA |
| | SEQ ID NO: 298 | CTCACTCCTTCTC |
| | | TCT |
| | | SEQ ID NO:305 |
| CE56666 | SPL9d contains a | TTGAAGCAATGAG |
| | 127 bp deletion | ATGAGAGGAAATT |
| | corresponding to | GTTGAGTTGAGAA |
| | position 1981 of | GAAAAAAAAAGAA |
| | SEQ ID NO: 297 | GAGAGGGTACAAT |
| | | CTGTCTCTCGTAG |
| | | CTGGCTGGCTCCA |
| | | TTATTTTTGGTTT |
| | | TGGACAGACAAGG |
| | | GTGTGTTTGG |
| | | SEQ ID NO: 306 |

### Example 3: Analysis of Phenotype

The E0 plants identified in Example 1 and Example 2 were transferred to the greenhouse to set seed. The greenhouse grown plants were also evaluated for yield traits including total pod count, total seed number, average seeds per pod, seed dry weight and hundred seed weight.

In addition to the yield traits identified above, the greenhouse grown plants were evaluated at the R6 growth stage for plant architectural features including plant height, stem thickness, number of nodes on the mainstem, number of branches, pods on branches, pods on mainstem, and pod per node on the mainstem.

TABLE 3
Phenotype data for E1 generation

| | | Plant | | | | | Pods per | | | |
|---|---|---|---|---|---|---|---|---|---|---|
| | Genotype/ | height | Nodes on | Number of | Pods on | Pods on | node on | Pods | Seeds | Seeds |
| Family | Edit | (cm) | Main-stem | branches | branches | Main-stem | main-stem | per plant | per plant | per pod |
| E1- | Hom | 105 | 33.5 | 19 | 50 | 34.5 | 1 | 84.5 | 215 | 2.6 |
| CE44978 | SPL9c | | | | | | | | | |
| | 8D; Hom | | | | | | | | | |
| | SPL9d 7D | | | | | | | | | |
| E1- | Cmpd-het | 109 | 38 | 17 | 66 | 47 | 1.2 | 113 | 240 | 2.1 |
| CE44907 | SPL9c | | | | | | | | | |
| | 29D/13D; | | | | | | | | | |
| | Cmpd-het | | | | | | | | | |
| | SPL9d | | | | | | | | | |
| | 5D/11D | | | | | | | | | |
| | Hom | 105 | 36 | 21 | 62 | 56 | 1.6 | 118 | 260 | 2.2 |
| | SPL9c | | | | | | | | | |
| | 29D; | | | | | | | | | |
| | Cmpd-het | | | | | | | | | |
| | SPL9d | | | | | | | | | |
| | 5D/11D | | | | | | | | | |
| | Cmpd-het | 102 | 33 | 22 | 52 | 57 | 1.7 | 109 | 203 | 1.9 |
| | SPL9c | | | | | | | | | |
| | 29D/13D; | | | | | | | | | |
| | Hom | | | | | | | | | |
| | SPL9d 5D | | | | | | | | | |
| | Het | 107 | 39 | 17 | 58 | 40 | 1 | 98 | 237 | 2.4 |
| | SPL9c | | | | | | | | | |
| | 29D; Hom | | | | | | | | | |
| | SPL9d 5D | | | | | | | | | |
| E1- | Hom 9D | 109.67 | 24.2 | 14.8 | 72 | 61.2 | 2.5 | 133.2 | 178 | 1.35 |
| CE56385 | IF SPL9a | | | | | | | | | |
| E1- | Hom for | 102 | 26 | 18 | 49 | 50 | 1.9 | 99 | 208 | 2.10 |
| CE56666 | 2 × 4D and | | | | | | | | | |
| | 10D in | | | | | | | | | |
| | SPL9c; | | | | | | | | | |
| | Hom for | | | | | | | | | |
| | 127D in | | | | | | | | | |
| | SPL9d | | | | | | | | | |
| | Hom for | 104.5 | 25.5 | 18.5 | 49.5 | 56 | 2.2 | 105.5 | 119.5 | 1.12 |
| | 68D | | | | | | | | | |
| | SPL9c; | | | | | | | | | |
| | Hom for | | | | | | | | | |
| | 127D in | | | | | | | | | |
| | SPL9d | | | | | | | | | |
| E1- | Hom 54D | 106 | 24 | 13 | 19 | 44 | 1.83 | 63 | 120 | 1.9 |
| CE52366 | SPL9b; | | | | | | | | | |
| | hom 12D | | | | | | | | | |
| | SPL9a | | | | | | | | | |
| WT | | 107.5 | 26 | 15.7 | 44.7 | 58.7 | 2.3 | 103.4 | 256.9 | 2.5 |

The phenotypic analysis of the E1 generation suggests that some combinations of edits in the SPL9 genes result in architectural changes that may lead to an increase in plant yield.

### Example 4: Edited Alleles Corn UB2/3

An editing strategy was developed to generate modified alleles in the corn UB2 gene Zm00001d031451 (SEQ ID NO:84) and/or UB3 gene Zm100001d052890 (SEQ ID NO:87) to alter kernel row number. To generate a range of alleles, multiple CRISPR guide nucleic acids comprising spacers described in Table 4 and having complementarity (reverse) to targets within the UB2 and/or UB3 gene were designed and placed into transformation constructs.

TABLE 4
Spacer (SEQ ID NO)

PWsp719
(CAGTGGCAAGCGGGCTTGGATCC)
SEQ ID NO: 326)
PWsp1073
(CAGTGGCAAGCAGCGAGCCTAGA)
SEQ ID NO: 327)
PWsp715 (SEQ ID NO: 125)
PWsp716 (SEQ ID NO: 126)
PWsp717 (SEQ ID NO: 127)
PWsp718(SEQ ID NO: 128)

Dried excised maize embryos were transformed using Agrobacterium to deliver the editing constructs. Healthy non-chimeric plants (E0) were selected and transferred from media to growth media and ultimately transferred to a greenhouse to complete the plants' life cycle. Tissue was collected from regenerating plants (E0 generation) for DNA extraction and subsequent molecular screening was employed to assess transgene copy and editing efficacy. Plants identified to be (1) healthy, non-chimeric and fertile, with (2) no transgenes or low transgene copy number and (3) alterations in either UB2 or UB3 gene were selfed to produce the E1 generation.

### Example 5. Edited Alleles

Edited alleles of UB3 gene Zm00001 d052890 were generated and are further described Table 5.

TABLE 5
Edited alleles of UB3 gene Zm00001d052890

| Allele of UB3 | Edit description | Protein description |
|---|---|---|
| Allele 1 | 12 bp deletion | In frame deletion of amino |
| (SEQ ID NO: 310) | (GCGAGCCTAGAT) (SEQ | acids “ASLD” at position 225- |
| CE96238 | ID NO: 328) starting at | 228 of SEQ ID NO: 89 giving |
| | position 5126 of SEQ ID | rise to the amino acid |
| | NO: 87 | sequence of SEQ ID NO: 311 |
| Allele 2 | 7 bp deletion (AGCCTAG) | Out of frame mutation |
| (SEQ ID NO: 312) | starting at position 5129 of | resulting in the amino acid |
| CE96317 | SEQ ID NO: 87 | sequence of SEQ ID |
| | | NO: 313) |
| Allele 3 | 5 bp deletion (CGAGC) | Out of frame mutation |
| (SEQ ID NO: 314) | starting at position 5127 of | leading to an early stop |
| CE96442 | SEQ ID NO: 87 | codon and giving rise to the |
| | | amino acid sequence of SEQ |
| | | ID NO: 315 |
| Allele 4 | 9 bp deletion (GCTCTCTCT) | In frame deletion of amino |
| (SEQ ID NO: 316) | starting at position 5303 of | acids “ALS” at position 284- |
| CE145128 | SEQ ID NO: 87 | 286 of SEQ ID NO: 86 giving |
| | | rise to the amino acid |
| | | sequence of SEQ ID NO: 317 |
| Allele 5 | Compound deletion of 13 bp | Out of frame mutation giving |
| (SEQ ID NO: 318) | (TGTGCTCTCTCTC) (SEQ | rise to the amino acid |
| CE145064 | ID NO: 329) starting at | sequence of SEQ ID |
| | position 5300 of SEQ ID | NO: 319) |
| | NO: 87; and 6 bp (GTAAAG) | |
| | deletion starting at position | |
| | 6019 of SEQ ID NO: 87 | |

Edited alleles of UB2 gene Zm00001d031451 were generated and are further described Table 6.

TABLE 6

| Allele of UB2 | Edit description | Protein description |
|---|---|---|
| Allele 6 | Compound deletion of 13 bp | Out of frame mutation |
| (SEQ ID NO: 320) | (GTGCTCTCTOTCT) (SEQ | resulting in the amino acid |
| CE145064 | ID NO: 330) starting at | sequence of SEQ ID |
| | position 4928 of SEQ ID | NO: 321 |
| | NO: 84; and 18 bp | |
| | (GTTGTTTAAGTAAAGCGA) | |
| | (SEQ ID NO: 331) deletion | |
| | starting at 5776 position of | |
| | SEQ ID NO: 84 | |
| Allele 7 | 5 bp (GCTGT) deletion | Out of frame mutation |
| (SEQ ID NO: 322) | starting at position 4925 of | leading to an early stop |
| CE145128 | SEQ ID NO: 84 | codon resulting in the amino |
| | | acid sequence of SEQ ID |
| | | NO: 323 |
| Allele 8 | 9 bp (GTGCTCTCT) deletion | In frame deletion of amino |
| (SEQ ID NO: 324) | starting at position 4928 of | acids “CAL” at position 272- |
| CE145138 | SEQ ID NO: 84 | 274 of SEQ ID NO: 86 giving |
| | | rise to the amino acid |
| | | sequence of SEQ ID |
| | | NO: 325 |

### Example 6. Phenotypic Assessment of Trait Activity

Seeds were sown in flats and later transferred to pots after seedlings were established. All materials were cultivated under standard greenhouse conditions and grown to reproductive maturity. Following standard practices, emerging ears were covered with small paper bags prior to the emergence of silk, and tassels were covered during anthesis for the capture of pollen on a plant-by-plant basis. In some cases, anthesis and silking was not synchronized, and ears were not pollinated. These were designated as ‘unpollinated’ ears and were evaluated separately for kernel row number determination (as described below) once all ears were removed from the plants after dry-down.

After ear harvest and dry-down, kernel row number was manually counted for all ears. Data represent the average of three row counts per ear taken from the mid-section of the ear where row lineages were most defined. In order to prevent double counting of rows, a marker (e.g., paper clip) was inserted in between the rows where the counts initiated and to designate where row counting should cease.

All ears were photo-documented with a Canon digital camera and EOS application. Images were subsequently imported into ImageJ and all ears were measured using the line trace function. Ear length was determined in centimeters by a setting scale in the image analysis program to output distance in centimeters after ears were traced with lines along the length of ear from its tip to the base of ear. Un-edited germplasm, and lines transformed with a Gus plasmid were used as wild-type controls for phenotyping.

### Example 7. Phenotype Analysis of Edited Alleles from Example 5

E0 plants generated as described in Example 4 were allowed to self pollinate in the greenhouse and to set E1 seed. E1 seed was planted and allowed to self pollinate in the greenhouse to set E2 seed. E2 seed was planted and grown in the greenhouse and allowed to self pollinate and the resulting ears were analyzed as described in Example 6 for kernel row number. Table 7 summarizes the results generated for alleles of UB3 and demonstrates that altered alleles of the UB3 gene Zm00001d052890 alter kernel row number and may increase plant yield. Table 8 summarizes the results generated for plants with edited alleles of both UB2 and UB3 and demonstrates that the combination of altered alleles in UB2 and UB3 affect kernel row number and may affect plant yield.

TABLE 7
Kernel row number edited alleles UB3 gene Zm00001d052890

| | | Standard | |
|---|---|---|---|
| Genotype | Mean | Deviation | number |
| Null segregant | 16.3 | 1.3 | 10 |
| control | | | |
| Heterozygous | 16.9 | 1.1 | 10 |
| Allele 1 | | | |
| Homozygous | 16.5 | 1.2 | 10 |
| Allele 1 | | | |
| Homozygous | 16.8 | 1.4 | 10 |
| Allele 2 | | | |
| Homozygous | 16.1 | 1.5 | 10 |
| Allele 3 | | | |
| Transformation | 15.7 | 1.1 | 8 |
| control | | | |
| Wild type control | 15.5 | 2.5 | 8 |

TABLE 8
Kernel row number edited alleles of UB2 and UB3

| UB3 | UB2 | KRN | range | number |
|---|---|---|---|---|
| Homozygous | Homozygous | 14.7 | 14-15.3 | 2 |
| Allele 5 | Allele 6 | | | |
| Homozygous | Homozygous | 15.2 | 12-18 | 13 |
| Allele 4 | Allele 7 | | | |
| Homozygous | Homozygous | 16.2 | 15.7-17.3 | 6 |
| Allele 4 | Allele 8 | | | |

| | | | |
|---|---|---|---|
| Wild type control | 15.6 | 12-18 | 5 |

### Example 8. Phenotypic Characterization Soybean

Plants CE44978 and CE56385 described in Example 1 and Example 3 were self pollinated and the resulting E2 seed collected. The E2 populations were planted in the greenhouse and evaluated for yield traits as described in Example 3. Yield trait phenotype data is summarized in Tables 9 and 10 and demonstrates that edited alleles of SPL9 alters plant architecture and may lead to increased plant yield.

TABLE 9
Phenotype data soybean

| | | | Pods on | |
|---|---|---|---|---|
| | Nodes on main | Branches per | branches | Pods on main |
| | stem (standard | plant (standard | (standard | stem (standard |
| | deviation) | deviation) | deviation) | deviation) |
| | (number of | (number of | (number of | (number of |
| genotype | plants) | plants) | plants) | plants) |
| CE44978 E2 | 25.3 (1.6) (16) | 18.1 (1.6) (16) | 103.3 (21.4) (16) | 42.3 (7.3) (15) |
| (Homozygous | | | | |
| SPL9c 8D; | | | | |
| Homozygous | | | | |
| SPL9d 7D) | | | | |
| CE56385 E2 | 18.4 (0.8) (19) | 14.4 (2.0) (20) | 113.8 (21.0) (21) | 56.4 (10.5) (21) |
| (Homozygous | | | | |
| 9D in frame | | | | |
| SPL9a) | | | | |
| Wild type control | 18.7 (0.6) (19) | 15.2 (2.0) (20) | 115.4 (25.8) (20) | 38.3 (8.8) (20) |

TABLE 10
Phenotype data soybean

| | Pods per node | | | |
|---|---|---|---|---|
| | main stem | Pods per plant | Seeds per plant | Seeds per pod |
| | (standard | (standard | (standard | (standard |
| | deviation) | deviation) | deviation) | deviation) |
| | (number of | (number of | (number of | (number of |
| genotype | plants) | plants) | plants) | plants) |
| CE44978 E2 | 1.7 (0.2) (15) | 146.8 (22.9) (16) | 217.7 (40.5) (14) | 1.6 (0.4) (16) |
| (Homozygous | | | | |
| SPL9c 8D; | | | | |
| Homozygous | | | | |
| SPL9d 7D) | | | | |
| CE56385 E2 | 3.0 (0.5) (21) | 170.2 (25.5) (21) | 307.0 (70.3) (22) | 1.9 (0.3) (21) |
| (Homozygous | | | | |
| 9D in frame | | | | |
| SPL9a) | | | | |
| Wild type control | 2.0 (0.5) (20) | 153.7 (24.8) (20) | 264.4 (51.9) (20) | 1.7 (0.3) (20) |

The foregoing is illustrative of the present invention and is not to be construed as limiting thereof. The invention is defined by the following claims, with equivalents of the claims to be included therein.

1. A plant or plant part thereof comprising at least one mutation in an endogenous IDEAL PLANT ARCHITECTURE 1 (IPA1) gene, or orthologue thereof, encoding a SQUAMOSA PROMOTER BINDING PROTEIN-LIKE (SPL) transcription factor, optionally wherein the endogenous IPA1 gene encoding a SPL transcription factor is an endogenous SQUAMOSA PROMOTER BINDING PROTEIN-LIKE 9 (SPL9) gene, an endogenous UNBRANCHED 2 (UB2) gene, or an endogenous UNBRANCHED 3 (UB3) gene,
wherein the endogenous SPL9 gene:
(a) comprises a nucleotide sequence having at least 80% sequence identity to the nucleotide sequence of any one of SEQ ID NOs:72, 75, 78, 81, 143, 182, 222, or 255;
(b) comprises a coding sequence having at least 80% sequence identity to the nucleotide sequence of any one of SEQ ID NOs:73, 76, 79, 82, 144, 183, 223, or 256;
(c) comprises a region having least 80% sequence identity to the nucleotide sequence of any one of SEQ ID NOs:146-181, 185-221, 225-254 and/or 258-288; and/or
(d) encodes a polypeptide sequence having at least 80% identity to the amino acid sequence of any one of SEQ ID NOs:74, 77, 80, 83, 145, 184, 224, or 257;
wherein the endogenous UB2 gene:
(a) comprises a nucleotide sequence having at least 80% sequence identity to the nucleotide sequence of SEQ ID NO:84;
(b) comprises a coding sequence having at least 80% sequence identity to the nucleotide sequence of SEQ ID NO:85;
(c) comprises a region having least 80% sequence identity to the nucleotide sequence of any one of SEQ ID NOs:90-96 or 332-393; and/or
(d) encodes a polypeptide sequence having at least 80% identity to the amino acid sequence of SEQ ID NO:86; and
the endogenous UB3 gene:
a) comprises a nucleotide sequence having at least 80% sequence identity to the nucleotide sequence of SEQ ID NO:87;
(b) comprises a coding sequence having at least 80% sequence identity to the nucleotide sequence of SEQ ID NO:88;
(c) comprises a region having least 80% sequence identity to the nucleotide sequence of any one of SEQ ID NOs:90, 97-103 or 394-445; and/or
(d) encodes a polypeptide sequence having at least 80% identity to the amino acid sequence of SEQ ID NO:89.

2-3. (canceled)

4. The plant or plant part thereof of claim 1, wherein the at least one mutation is a base deletion, a base substitution, and/or a base insertion; optionally wherein the base deletion is an out-of-frame deletion and/or the base insertion is an out-of-frame insertion.

5-12. (canceled)

13. The plant or plant part thereof of claim 1, wherein the IPA1 gene is an SPL9 gene, which is an SPL9a gene, an SPL9b gene, an SPL9c gene, and/or an SPL9d gene and the at least one mutation is present in at least one of the SPL9a gene, the SPL9b gene, the SPL9c gene, and/or the SPL9d gene, wherein the at least one mutation is in the first exon, optionally wherein the first exon of the SPL9a gene is located from about nucleotide 2001 to about nucleotide 2364 with reference to the nucleotide numbering of SEQ ID NO:72, from about nucleotide 1 to about nucleotide 364 with reference to the nucleotide numbering of SEQ ID NO:73, and/or from about nucleotide 2160 to about nucleotide 2523 with reference to the nucleotide numbering of SEQ ID NO:143, the first exon of the SPL9b gene is located from about nucleotide 2001 to about nucleotide 2370 with reference to the nucleotide numbering of SEQ ID NO:75, from about nucleotide 1 to about nucleotide 370 with reference to the nucleotide numbering of SEQ ID NO:76, and/or from about nucleotide 2098 to about nucleotide 2467 with reference to the nucleotide numbering of SEQ ID NO:182, the first exon of the SPL9c gene is located from about nucleotide 2001 to about nucleotide 2347 with reference to the nucleotide numbering of SEQ ID NO:78, from about nucleotide 1 to about nucleotide 347 with reference to the nucleotide numbering of SEQ ID NO:79, and/or from about nucleotide 2378 to about nucleotide 2724 with reference to the nucleotide numbering of SEQ ID NO:222, and/or the first exon of the SPL9d gene is located from about nucleotide 2001 to about nucleotide 2349 with reference to the nucleotide numbering of SEQ ID NO:81, from about nucleotide 1 to about nucleotide 349 with reference to the nucleotide numbering of SEQ ID NO:82 and/or from about nucleotide 2410 to about nucleotide 2758 with reference to the nucleotide numbering of SEQ ID NO:255; and/or the at least one mutation is in a region of the first exon of the SPL9a gene from about nucleotide 2053 to about nucleotide 2115 with reference to the nucleotide numbering of SEQ ID NO:72 or SEQ ID NO:75, a region of the first exon of the SPL9b gene from about nucleotide 2015 to about nucleotide 2077 with reference to the nucleotide numbering of SEQ ID NO:78 or SEQ ID NO:81, a region of the first exon of the SPL9c gene from about nucleotide 1 to about nucleotide 115 with reference to the nucleotide numbering of SEQ ID NO:73 or SEQ ID NO:76, and/or a region of the first exon of the SPL9d gene from about nucleotide 1 to about nucleotide 77 with reference to the nucleotide numbering of SEQ ID NO:79 or SEQ ID NO:82, optionally in a region of the SPL9a gene having least 80% sequence identity to the nucleotide sequence of any one of SEQ ID NOs:161-177, in a region of the SPL9b gene having least 80% sequence identity to the nucleotide sequence of any one of SEQ ID NOs:201-217, in a region of the SPL9c gene having least 80% sequence identity to the nucleotide sequence of any one of SEQ ID NOs:240-250, or in a region of the SPL9d gene having least 80% sequence identity to the nucleotide sequence of any one of SEQ ID NOs:274-284.

14-16. (canceled)

17. The plant or plant part thereof of claim 1, wherein the IPA1 gene is a UB2 gene or UB3 gene and the at least one mutation is located in the third exon of the endogenous UB2 gene and/or UB3 gene, optionally the at least one mutation in the endogenous UB2 gene is in a region having at least 80% sequence identity to any one of SEQ ID NOs:358-376, optionally SEQ ID NOs:373-376, and optionally the at least one mutation in the endogenous UB3 gene is in a region having at least 80% sequence identity to any one of SEQ ID NOs:408-426, optionally SEQ ID NOs:415-416.

18. (canceled)

19. The plant or plant part thereof of claim 1, wherein the at least one mutation is in a miR156 binding site of the endogenous IPA1 gene, or orthologue thereof, wherein
(a) (i) the endogenous IPA1 gene is an SPL9a gene and the miR156 binding site is located from about nucleotide 6569 to about nucleotide 6588 with reference to the nucleotide numbering of SEQ ID NO:72, from about nucleotide 758 to about nucleotide 777 with reference to the nucleotide numbering of SEQ ID NO:73, and/or from about nucleotide 6624 to about nucleotide 6847 with reference to the nucleotide numbering of SEQ ID NO:143,
(ii) the endogenous IPA1 gene is an SPL9b gene and the miR156 binding site is located from about nucleotide 6269 to about nucleotide 6288 with reference to the nucleotide numbering of SEQ ID NO:75, from about nucleotide 760 to about nucleotide 780 with reference to the nucleotide numbering of SEQ ID NO:76, and/or from about nucleotide 6265 to about nucleotide 6488 with reference to the nucleotide numbering of SEQ ID NO:182,
(iii) the endogenous IPA1 gene is an SPL9c gene and the miR156 binding site is located from about nucleotide 5388 to about nucleotide 5407 with reference to the nucleotide numbering of SEQ ID NO:78, from about nucleotide 761 to about nucleotide 780 with reference to the nucleotide numbering of SEQ ID NO:79, and/or from about nucleotide 5665 to about nucleotide 5887 with reference to the nucleotide numbering of SEQ ID NO:222, and/or
(iv) the endogenous IPA1 gene is an SPL9d gene and the miR156 binding site is located from about nucleotide 5798 to about nucleotide 5817 with reference to the nucleotide numbering of SEQ ID NO:81, from about nucleotide 737 to about nucleotide 756 with reference to the nucleotide numbering of SEQ ID NO:82, and/or from about nucleotide 6120 to about nucleotide 6342 with reference to the nucleotide numbering of SEQ ID NO:255; and/or
the mutation in the miR156 binding site is located:
(i) in a region of the endogenous SPL9a gene from about nucleotide 6549 to about nucleotide 6608 with reference to the nucleotide numbering of SEQ ID NO:72 and/or from about nucleotide 738 to about nucleotide 797 with reference to the nucleotide numbering of SEQ ID NO:73, optionally in a region having about 80% sequence identity to any one of the nucleotide sequences of SEQ ID NO:178-181,
(ii) in a region of the endogenous SPL9b gene from about nucleotide 6250 to about nucleotide 6308 with reference to the nucleotide numbering of SEQ ID NO:75 and/or from about nucleotide 741 to about nucleotide 800 with reference to the nucleotide numbering of SEQ ID NO:76, optionally in a region having about 80% sequence identity to any one of the nucleotide sequences of SEQ ID NO:218-221,
(iii) in a region of the endogenous SPL9c gene from about nucleotide 5368 to about nucleotide 5427 with reference to the nucleotide numbering of SEQ ID NO:78 and/or from about nucleotide 742 to about nucleotide 800 with reference to the nucleotide numbering of SEQ ID NO:79, optionally in a region having about 80% sequence identity to any one of the nucleotide sequences of SEQ ID NO:251-254, and/or
(iv) in a region of the of the endogenous SPL9d gene from about nucleotide 5778 to about nucleotide 5837 with reference to the nucleotide numbering of SEQ ID NO:81 and/or from about nucleotide 718 to about nucleotide 775 with reference to the nucleotide numbering of SEQ ID NO:82, optionally in a region having about 80% sequence identity to any one of the nucleotide sequences of SEQ ID NO:285-288; and/or
(b) the endogenous IPA1 gene is:
(i) a UB2 gene and the miR156 binding site is located from about nucleotide 4928 to about nucleotide 4947 with reference to the nucleotide numbering of SEQ ID NO:84 and/or from about nucleotide 815 to about nucleotide 834 with reference to the nucleotide numbering of SEQ ID NO:85, and/or
(ii) a UB3 gene and the miR156 binding site is located from about nucleotide 5301 to about nucleotide 5320 with reference to the nucleotide numbering of SEQ ID NO:87 and/or from about nucleotide 848 to about nucleotide 866 with reference to the nucleotide numbering of SEQ ID NO:88; and/or
the mutation in the miR156 binding site:
(ii) of the endogenous UB2 gene is located from about nucleotide 4894 to about nucleotide 4967 with reference to the nucleotide numbering of SEQ ID NO:84 or from about nucleotide 781 to about nucleotide 854 with reference to the nucleotide numbering of SEQ ID NO:85, and/or
(ii) of the endogenous UB3 gene is located from about nucleotide 5267 to about nucleotide 5339 with reference to the nucleotide numbering of SEQ ID NO:87 or from about nucleotide 814 to about nucleotide 887 with reference to the nucleotide numbering of SEQ ID NO:88.

20-30. (canceled)

31. The plant or plant part thereof of claim 1, wherein the at least one mutation is in a 5′ untranslated region (UTR) of the endogenous IPA1 gene, wherein the endogenous IPA1 gene is:
(a) an SPL9a gene and the at least one mutation is in a region of the 5′ UTR located from about nucleotide 1826 to about nucleotide 1981 and/or from about nucleotide 1846 to about nucleotide 1961 with reference to the nucleotide numbering of SEQ ID NO:72, optionally in a region having at least 80% sequence identity to a nucleotide sequence of any one of SEQ ID NOs:146-160;
(b) an SPL9b gene and the at least one mutation is in a region of the 5′ UTR located from about nucleotide 1804 to about nucleotide 1973 and/or from about nucleotide 1824 to about nucleotide 1953 with reference to the nucleotide numbering of SEQ ID NO:75, optionally a region having at least 80% sequence identity to a nucleotide sequence of any one of SEQ ID NOs:185-200;
(c) an SPL9c gene and the at least one mutation is in a region of the 5′ UTR located from about nucleotide 1593 to about nucleotide 1783 and/or from about nucleotide 1613 to about nucleotide 1764 with reference to the nucleotide numbering of SEQ ID NO:78, optionally a region having at least 80% sequence identity to a nucleotide sequence of any one of SEQ ID NOs:225-239;
(d) an SPL9d gene and the at least one mutation is in a region of the 5′ UTR located from about nucleotide 1555 to about nucleotide 1740 and/or from about nucleotide 1574 to about nucleotide 1720 with reference to the nucleotide numbering of SEQ ID NO:81, optionally a region having at least 80% sequence identity to a nucleotide sequence of any one of SEQ ID NOs:258-273;
(e) a UB2 gene and the at least one mutation is in a region of the 5′ UTR located from about nucleotide 1414 to about nucleotide 1860, from about nucleotide 1414 to about nucleotide 1522, from about nucleotide 1454 to about nucleotide 1481, from about nucleotide 1553 to about nucleotide 1582, from about nucleotide 1597 to about nucleotide 1633, and/or from about nucleotide 1767 to about nucleotide 1819 with reference to the nucleotide numbering of SEQ ID NO:84, optionally wherein the region of the 5′ UTR is a promoter or is in a promoter, optionally a region having at least 80% sequence identity to a nucleotide sequence of any one of SEQ ID NOs:90-96 or 332-357; and/or
(f) a UB3 gene and the at least one mutation is in a region of the 5′ UTR located from about nucleotide 1327 to about nucleotide 1646, from about nucleotide 1439 to about nucleotide 1467, from about nucleotide 1368 to about nucleotide 1394, from about nucleotide 1549 to about nucleotide 1606, from about nucleotide 1787 to about nucleotide 1855, and/or from about nucleotide 1747 to about nucleotide 1920 with reference to the nucleotide numbering of SEQ ID NO:87, optionally wherein the region of the 5′ UTR is a promoter or is in a promoter, optionally a region having at least 80% sequence identity to a nucleotide sequence of any one of SEQ ID NOs:394-407.

32-33. (canceled)

34. The plant or plant part thereof of claim 1, wherein the at least one mutation is in a 3′ UTR of the endogenous IPA1 gene and the endogenous IPA1 gene is
(a) a UB2 gene and the at least one mutation is in a region of the 3′ UTR located from about nucleotide 5701 to about nucleotide 5882, and/or from about nucleotide 5742 to about nucleotide 5842 with reference to the nucleotide numbering of SEQ ID NO:84, optionally a region having at least 80% sequence identity to a nucleotide sequence of any one of SEQ ID NOs:377-393; and/or
(b) a UB3 gene and the at least one mutation is in a region of the 3′ UTR located from about nucleotide 5940 to about nucleotide 6109, from about nucleotide 5980 to about nucleotide 6069, from about nucleotide 6516 to about nucleotide 6643, and/or from about nucleotide 6556 to about nucleotide 6603 with reference to the nucleotide numbering of SEQ ID NO:87, optionally a region having at least 80% sequence identity to a nucleotide sequence of any one of SEQ ID NOs:427-445.

35. The plant or plant part thereof of claim 1, wherein the at least one mutation is in an intron of the endogenous IPA1 gene, wherein the endogenous gene
(a) is a UB2 gene and the at least one mutation is in a region of the intron located from about nucleotide 2856 to about nucleotide 2971, from about nucleotide 2896 to about nucleotide 2931, from about nucleotide 3753 to about nucleotide 3893, and/or from about nucleotide 3793 to about nucleotide 3853 with reference to the nucleotide numbering of SEQ ID NO:84; and/or
(b) is a UB3 gene and the at least one mutation is in a region of the intron located from about nucleotide 2666 to about nucleotide 2784, from about nucleotide 2706 to about nucleotide 2744, from about nucleotide 4017 to about nucleotide 4147, and/or from about nucleotide 4057 to about nucleotide 4107 with reference to the nucleotide numbering of SEQ ID NO:87.

36-44. (canceled)

45. The plant or part thereof of claim 1, wherein the IPA1 gene is a SPL9 gene and the at least one mutation results in a mutated SPL9 gene having at least 90% identity to any one of the nucleotide sequences of SEQ ID NOs:289-300, wherein the IPA1 gene is a UB2 gene and the at least one mutation results in a mutated UB2 gene having at least 90% identity to any one of the nucleotide sequences of SEQ ID NOs:320, 322 or 324, or is a UB3 gene and the at least one mutation results in a mutated UB2 gene having at least 90% identity to any one of the nucleotide sequences of SEQ ID NOs:310, 312, 314, 316, or 318; or wherein the IPA1 gene is a UB2 gene and the at least one mutation results in a mutated UB2 gene encoding a mutated polypeptide having at least 90% identity to any one of SEQ ID NOs:321, 323 or 325, or is a UB3 gene and the at least one mutation results in a mutated UB2 gene encoding a mutated polypeptide having at least 90% identity to any one of SEQ ID NOs:311, 313, 315, 317, or 319.

46-53. (canceled)

54. A plant cell comprising at least one mutation in one or more endogenous IDEAL PLANT ARCHITECTURE 1 (IPA1) genes, or orthologue thereof, encoding a SQUAMOSA PROMOTER BINDING PROTEIN-LIKE (SPL) transcription factor, wherein the at least one mutations is a substitution, insertion and/or a deletion that is introduced using an editing system that comprises a nucleic acid binding domain that binds to a target site in the one or more endogenous IPA1 genes, wherein the one or more endogenous IPA1 genes is a SQUAMOSA PROMOTER BINDING PROTEIN-LIKE 9 (SPL9) gene, endogenous UNBRANCHED 2 (UB2) gene, or an endogenous UNBRANCHED 3 (UB3) gene, optionally wherein the target site is within a region of one or more:
(a) SPL9 genes, the region having at least 80% sequence identity to any one of SEQ ID NOs:146-181, 185-221, 225-254 and/or 258-288;
(b) UB2 genes, the region having at least 80% sequence identity to any one of SEQ ID NOs:90-96 or 332-393; and/or
(c) UB3 genes, the region having at least 80% sequence identity to any one of SEQ ID NOs:90, 97-103 or 394-445.

55-59. (canceled)

60. The plant cell of claim 54, wherein the editing system further comprises a nuclease, and the nucleic acid binding domain binds to a target site in:
(a) an SPL9 gene having at least 80% sequence identity to a nucleotide sequence of any one of SEQ ID NOs:72, 73, 75, 76, 78, 79, 81, 82, 143, 144, 182, 183, 222, 223, 255 or 256; and/or comprising a region having at least 80% sequence identity to a nucleotide sequence of any one of SEQ ID NOs:146-181, 185-221, 225-254 and/or 258-288,
(b) a UB2 gene having at least 80% sequence identity to the nucleotide sequence of SEQ ID NO:84 or SEQ ID NO:85 and/or comprising a region having at least 80% sequence identity to a nucleotide sequence of any one of SEQ ID NOs:90-96 or 332-393; and/or
(c) a UB3 gene having at least 80% sequence identity to the nucleotide sequence of SEQ ID NO:87 or SEQ ID NO:88 and/or comprising a region having at least 80% sequence identity to a nucleotide sequence of any one of SEQ ID NOs:90, 97-103 or 394-445; and the at least one mutation within the endogenous IPA1 gene is made following cleavage by the nuclease.

61-62. (canceled)

63. The plant cell of claim 54, wherein the at least one mutation within the one or more endogenous IPA1 gene is an insertion and/or a deletion, optionally the at least one mutation is an out-of-frame insertion or an out-of-frame deletion that results in a premature stop codon.

64-65. (canceled)

66. The plant cell of claim 54, wherein the endogenous IPA1 gene is a SPL9 gene and the at least one mutation results in a mutated SPL9 gene having at least 90% identity to any one of the nucleotide sequences of SEQ ID NOs:289-300, wherein the IPA1 gene is a UB2 gene and the at least one mutation results in a mutated UB2 gene having at least 90% identity to any one of the nucleotide sequences of SEQ ID NOs:320, 322 or 324, or is a UB3 gene and the at least one mutation results in a mutated UB2 gene having at least 90% identity to any one of the nucleotide sequences of SEQ ID NOs:310, 312, 314, 316, or 318, and/or the IPA1 gene is a UB2 gene and the at least one mutation results in a mutated UB2 gene encoding a mutated polypeptide having at least 90% identity to any one of SEQ ID NOs:321, 323 or 325, or is a UB3 gene and the at least one mutation results in a mutated UB2 gene encoding a mutated polypeptide having at least 90% identity to any one of SEQ ID NOs:311, 313, 315, 317, or 319.

67-89. (canceled)

90. A method for producing a plant or part thereof comprising at least one cell having a mutation in an endogenous IDEAL PLANT ARCHITECTURE 1 (IPA1) gene, or orthologue thereof, encoding a SQUAMOSA PROMOTER BINDING PROTEIN-LIKE (SPL) transcription factor, the method comprising contacting a target site in the endogenous IPA1 gene in the plant or plant part with a nuclease comprising a cleavage domain and a DNA-binding domain, wherein the DNA binding domain of the nuclease binds to a target site in the endogenous IPA1 gene, wherein the endogenous IPA1 gene:
(a) is an endogenous SQUAMOSA PROMOTER BINDING PROTEIN-LIKE 9 (SPL9) gene
(i) comprising a nucleotide sequence having at least 80% sequence identity to the nucleotide sequence of any one of SEQ ID NOs:72, 75, 78, 81, 143, 182, 222, or 255;
(ii) comprising a coding sequence having at least 80% sequence identity to the nucleotide sequence of any one of SEQ ID NOs:73, 76, 79, 82, 144, 183, 223, or 256;
(iii) comprising a region having least 80% sequence identity to the nucleotide sequence of any one of SEQ ID NOs:146-181, 185-221, 225-254 or 258-288, and/or
(iv) encoding a polypeptide sequence having at least 80% identity to the amino acid sequence of any one of SEQ ID NOs:74, 77, 80, 83, 145, 184, 224, or 257;
(b) is an endogenous UNBRANCHED 2 (UB2) gene
(i) comprising a nucleotide sequence having at least 80% sequence identity to the nucleotide sequence of SEQ ID NO:84;
(ii) comprising a coding sequence having at least 80% sequence identity to the nucleotide sequence of SEQ ID NO:85;
(iii) comprising a region having least 80% sequence identity to the nucleotide sequence of any one of SEQ ID NOs:90-96 or 332-393, and/or
(iv) encoding a polypeptide sequence having at least 80% identity to the amino acid sequence of SEQ ID NO:86; or
(c) is an endogenous UB3 gene:
(i) comprising a nucleotide sequence having at least 80% sequence identity to the nucleotide sequence of SEQ ID NO:87;
(ii) comprising a coding sequence having at least 80% sequence identity to the nucleotide sequence of SEQ ID NO:88;
(iii) comprising a region having least 80% sequence identity to the nucleotide sequence of any one of SEQ ID NOs:90, 97-103 or 394-445, and/or
(iv) encoding a polypeptide sequence having at least 80% identity to the amino acid sequence of SEQ ID NO:89, thereby producing a plant or part thereof comprising at least one cell having a mutation in the endogenous IPA1 gene, optionally wherein the mutation is at least one of a base pair deletion, a base pair substitution, and/or a base pair insertion.

91-96. (canceled)

97. The method of claim 90, wherein the mutation is in the first exon of the endogenous SPL9 gene, optionally wherein the endogenous SPL9 gene is a SPL9a gene and the first exon of the SPL9a gene is located from about nucleotide 2001 to about nucleotide 2364 with reference to the nucleotide numbering of SEQ ID NO:72, from about nucleotide 1 to about nucleotide 364 with reference to the nucleotide numbering of SEQ ID NO:73, and/or from about nucleotide 2160 to about nucleotide 2523 with reference to the nucleotide numbering of SEQ ID NO:143, the endogenous SPL9 gene is a SPL9b gene and the first exon of the SPL9b gene is located from about nucleotide 2001 to about nucleotide 2370 with reference to the nucleotide numbering of SEQ ID NO:75, from about nucleotide 1 to about nucleotide 370 with reference to the nucleotide numbering of SEQ ID NO:76, and/or from about nucleotide 2098 to about nucleotide 2467 with reference to the nucleotide numbering of SEQ ID NO:182, the endogenous SPL9 gene is a SPL9c gene and the first exon of the SPL9c gene is located from about nucleotide 2001 to about nucleotide 2347 with reference to the nucleotide numbering of SEQ ID NO:78, from about nucleotide 1 to about nucleotide 347 with reference to the nucleotide numbering of SEQ ID NO:79, and/or from about nucleotide 2378 to about nucleotide 2724 with reference to the nucleotide numbering of SEQ ID NO:222, and/or the endogenous SPL9 gene is a SPL9d gene and the first exon of the SPL9d gene is located from about nucleotide 2001 to about nucleotide 2349 with reference to the nucleotide numbering of SEQ ID NO:81, from about nucleotide 1 to about nucleotide 349 with reference to the nucleotide numbering of SEQ ID NO:82 and/or from about nucleotide 2410 to about nucleotide 2758 with reference to the nucleotide numbering of SEQ ID NO:255, and/or the mutation is in a region of the first exon of the SPL9a gene from about nucleotide 2053 to about nucleotide 2115 with reference to the nucleotide numbering of SEQ ID NO:72 or SEQ ID NO:75, a region of the first exon of the SPL9b gene from about nucleotide 2015 to about nucleotide 2077 with reference to the nucleotide numbering of SEQ ID NO:78 or SEQ ID NO:81, a region of the first exon of the SPL9c gene from about nucleotide 1 to about nucleotide 115 with reference to the nucleotide numbering of SEQ ID NO:73 or SEQ ID NO:76, and/or a region of the first exon of the SPL9d gene from about nucleotide 1 to about nucleotide 77 with reference to the nucleotide numbering of SEQ ID NO:79 or SEQ ID NO:82, optionally in a region of the SPL9a gene having least 80% sequence identity to the nucleotide sequence of any one of SEQ ID NOs:161-177, in a region of the SPL9b gene having least 80% sequence identity to the nucleotide sequence of any one of 201-217, in a region of the SPL9c gene having least 80% sequence identity to the nucleotide sequence of any one of 240-250, or in a region of the SPL9d gene having least 80% sequence identity to the nucleotide sequence of any one of 274-284.

98-99. (canceled)

100. The method of claim 90, wherein the mutation is in the third exon of the endogenous UB2 gene or the endogenous UB3 gene, optionally wherein the at least one mutation located in the third exon of the endogenous UB2 gene is located in a region having at least 80% sequence identity to any one of SEQ ID NOs:358-376, optionally SEQ ID NOs:373-376 and/or the at least one mutation located in the third exon of the endogenous UB3 gene is located in a region having at least 80% sequence identity to any one of SEQ ID NOs:408-426, optionally SEQ ID NOs:415-416.

101. (canceled)

102. The method of claim 90, wherein the mutation is in a miR156 binding site of the endogenous SPL9 gene, UB2 gene and/or UB3 gene, optionally wherein
(a) the endogenous SPL9 gene is an SPL9a gene and the miR156 binding site is from about nucleotide 6569 to about nucleotide 6588 with reference to the nucleotide numbering of SEQ ID NO:72, from about nucleotide 758 to about nucleotide 777 with reference to the nucleotide numbering of SEQ ID NO:73, and/or from about nucleotide 6624 to about nucleotide 6847 with reference to the nucleotide numbering of SEQ ID NO:143,
(b) the endogenous SPL9 gene is an SPL9b gene and the miR156 binding site is from about nucleotide 6269 to about nucleotide 6288 with reference to the nucleotide numbering of SEQ ID NO:75, from about nucleotide 760 to about nucleotide 780 with reference to the nucleotide numbering of SEQ ID NO:76, and/or from about nucleotide 6265 to about nucleotide 6488 with reference to the nucleotide numbering of SEQ ID NO:182,
(c) the endogenous SPL9 gene is an SPL9c gene and the miR156 binding site is from about nucleotide 5388 to about nucleotide 5407 with reference to the nucleotide numbering of SEQ ID NO:78, from about nucleotide 761 to about nucleotide 780 with reference to the nucleotide numbering of SEQ ID NO:79, and/or from about nucleotide 5665 to about nucleotide 5887 with reference to the nucleotide numbering of SEQ ID NO:222,
(d) the endogenous SPL9 gene is an SPL9d gene and the miR156 binding site is from about nucleotide 5798 to about nucleotide 5817 with reference to the nucleotide numbering of SEQ ID NO:81, from about nucleotide 737 to about nucleotide 756 with reference to the nucleotide numbering of SEQ ID NO:82, and/or from about nucleotide 6120 to about nucleotide 6342 with reference to the nucleotide numbering of SEQ ID NO:255,
(e) the endogenous gene is a UB2 gene and the miR156 binding site is from about nucleotide 4928 to about nucleotide 4947 with reference to the nucleotide numbering of SEQ ID NO:84 and/or from about nucleotide 815 to about nucleotide 834 with reference to the nucleotide numbering of SEQ ID NO:85, and/or
(f) the endogenous gene is a UB3 gene and the miR156 binding site is from about nucleotide 5301 to about nucleotide 5320 with reference to the nucleotide numbering of SEQ ID NO:87 and/or from about nucleotide 848 to about nucleotide 866 with reference to the nucleotide numbering of SEQ ID NO:88, and/or,
wherein the mutation in the miR156 binding site is located:
(a) in a region of the endogenous SPL9a gene from about nucleotide 6549 to about nucleotide 6608 with reference to the nucleotide numbering of SEQ ID NO:72 and/or from about nucleotide 738 to about nucleotide 797 with reference to the nucleotide numbering of SEQ ID NO:73, optionally in a region having about 80% sequence identity to any one of the nucleotide sequences of SEQ ID NO:178-181,
(b) in a region of the endogenous SPL9b gene from about nucleotide 6250 to about nucleotide 6308 with reference to the nucleotide numbering of SEQ ID NO:75 and/or from about nucleotide 741 to about nucleotide 800 with reference to the nucleotide numbering of SEQ ID NO:76, optionally in a region having about 80% sequence identity to any one of the nucleotide sequences of SEQ ID NO:218-221,
(c) in a region of the endogenous SPL9c gene from about nucleotide 5368 to about nucleotide 5427 with reference to the nucleotide numbering of SEQ ID NO:78 and/or from about nucleotide 742 to about nucleotide 800 with reference to the nucleotide numbering of SEQ ID NO:79, optionally in a region having about 80% sequence identity to any one of the nucleotide sequences of SEQ ID NO:251-254,
(d) in a region of the of the endogenous SPL9d gene from about nucleotide 5778 to about nucleotide 5837 with reference to the nucleotide numbering of SEQ ID NO:81 and/or from about nucleotide 718 to about nucleotide 775 with reference to the nucleotide numbering of SEQ ID NO:82, optionally in a region having about 80% sequence identity to any one of the nucleotide sequences of SEQ ID NO:285-288,
(e) in a region of the endogenous UB2 gene is located from about nucleotide 4894 to about nucleotide 4967 with reference to the nucleotide numbering of SEQ ID NO:84 and/or from about nucleotide 781 to about nucleotide 854 with reference to the nucleotide numbering of SEQ ID NO:85, and/or
(f) in a region of the endogenous UB3 gene is located from about nucleotide 5267 to about nucleotide 5339 with reference to the nucleotide numbering of SEQ ID NO:87 and/or from about nucleotide 814 to about nucleotide 887 with reference to the nucleotide numbering of SEQ ID NO:88.

103-106. (canceled)

107. The method of claim 102, wherein the mutation in the miR156 binding site is a substitution or deletion, optionally wherein the deletion is an in-frame deletion or an out-of-frame deletion.

108-113. (canceled)

114. The method of claim 90, wherein the mutation is in a 5′ untranslated region (UTR) of the endogenous SPL9 gene, endogenous UB2 gene or endogenous UB3 gene, optionally wherein
(i) the endogenous SPL9 gene is:
(a) an endogenous SPL9a gene and the mutation is in a region of the 5′ UTR located from about nucleotide 1826 to about nucleotide 1981 and/or from about nucleotide 1846 to about nucleotide 1961 with reference to the nucleotide numbering of SEQ ID NO:72, optionally a region having at least 80% sequence identity to a nucleotide sequence of any one of SEQ ID NOs:146-160;
(b) an endogenous SPL9b gene and the mutation is in a region of the 5′ UTR located from about nucleotide 1804 to about nucleotide 1973 and/or from about nucleotide 1824 to about nucleotide 1953 with reference to the nucleotide numbering of SEQ ID NO:75, optionally a region having at least 80% sequence identity to a nucleotide sequence of any one of SEQ ID NOs:185-200;
(c) an endogenous SPL9c gene and the mutation is in a region of the 5′ UTR located from about nucleotide 1593 to about nucleotide 1783 and/or from about nucleotide 1613 to about nucleotide 1764 with reference to the nucleotide numbering of SEQ ID NO:78, optionally a region having at least 80% sequence identity to a nucleotide sequence of any one of SEQ ID NOs:225-239; and/or
(d) an endogenous SPL9d gene and the mutation is in a region of the 5′ UTR located from about nucleotide 1555 to about nucleotide 1740 and/or from about nucleotide 1574 to about nucleotide 1720 with reference to the nucleotide numbering of SEQ ID NO:81, optionally a region having at least 80% sequence identity to a nucleotide sequence of any one of SEQ ID NOs:258-273;
(ii) the mutation is in a region of the 5′ UTR of:
(a) the endogenous UB2 gene located from about nucleotide 1414 to about nucleotide 1860, from about nucleotide 1414 to about nucleotide 1522, from about nucleotide 1454 to about nucleotide 1481, from about nucleotide 1553 to about nucleotide 1582, from about nucleotide 1597 to about nucleotide 1633, and/or from about nucleotide 1767 to about nucleotide 1819 with reference to the nucleotide numbering of SEQ ID NO:84, optionally a region having at least 80% sequence identity to a nucleotide sequence of any one of SEQ ID NOs:90-96 or 332-357; and/or
(b) of the endogenous UB3 gene located from about nucleotide 1327 to about nucleotide 1646, from about nucleotide 1439 to about nucleotide 1467, from about nucleotide 1368 to about nucleotide 1394, from about nucleotide 1549 to about nucleotide 1606, from about nucleotide 1787 to about nucleotide 1855, and/or from about nucleotide 1747 to about nucleotide 1920 with reference to the nucleotide numbering of SEQ ID NO:87, optionally a region having at least 80% sequence identity to a nucleotide sequence of any one of SEQ ID NOs:394-407.

115-116. (canceled)

117. The method of claim 90, wherein the mutation is in a region of the 3′ untranslated region (UTR) of:
(a) the endogenous UB2 gene that is located from about nucleotide 5701 to about nucleotide 5882, and/or from about nucleotide 5742 to about nucleotide 5842 with reference to the nucleotide numbering of SEQ ID NO:84, optionally a region having at least 80% sequence identity to a nucleotide sequence of any one of SEQ ID NOs:377-393; and/or
(b) the endogenous UB3 gene that is located from about nucleotide 5940 to about nucleotide 6109, from about nucleotide 5980 to about nucleotide 6069, from about nucleotide 6516 to about nucleotide 6643, and/or from about nucleotide 6556 to about nucleotide 6603 with reference to the nucleotide numbering of SEQ ID NO:87, optionally a region having at least 80% sequence identity to a nucleotide sequence of any one of SEQ ID NOs:427-445.

118. The method of claim 90, wherein the mutation is in an intron of the endogenous UB2 gene or the endogenous UB3 gene, optionally wherein the mutation is in a region of the intron of:
(a) the endogenous UB2 gene located from about nucleotide 2856 to about nucleotide 2971, from about nucleotide 2896 to about nucleotide 2931, from about nucleotide 3753 to about nucleotide 3893, and/or from about nucleotide 3793 to about nucleotide 3853 with reference to the nucleotide numbering of SEQ ID NO:84; and/or
(b) of the endogenous UB3 gene located from about nucleotide 2666 to about nucleotide 2784, from about nucleotide 2706 to about nucleotide 2744, from about nucleotide 4017 to about nucleotide 4147, and/or from about nucleotide 4057 to about nucleotide 4107 with reference to the nucleotide numbering of SEQ ID NO:87.

119-122. (canceled)

123. The method of claim 90, wherein the endogenous IPA1 gene is (a) a SPL9 gene and the mutation results in a mutated SPL9 gene having at least 90% identity to any one of the nucleotide sequences of SEQ ID NOs:289-300, (b) a UB2 gene and the at least one mutation results in a mutated UB2 gene having at least 90% identity to any one of the nucleotide sequences of SEQ ID NOs:320, 322 or 324, (c) a UB3 gene and the at least one mutation results in a mutated UB2 gene having at least 90% identity to any one of the nucleotide sequences of SEQ ID NOs:310, 312, 314, 316, or 318, (d) a UB2 gene and the at least one mutation results in a mutated UB2 gene encoding a mutated polypeptide having at least 90% identity to any one of SEQ ID NOs:321, 323 or 325, or (e) a UB3 gene and the at least one mutation results in a mutated UB2 gene encoding a mutated polypeptide having at least 90% identity to any one of SEQ ID NOs:311, 313, 315, 317, or 319.

124-141. (canceled)

142. A mutated nucleic acid encoding:
(a) a SPL9 polypeptide, the mutated nucleic acid comprising a sequence having at least 90% sequence identity to any one of SEQ ID NOs:289-300;
(b) a UB2 polypeptide, the mutated nucleic acid comprising a sequence having at least 90% sequence identity to any one of SEQ ID NOs:320, 322, or 324; or
(c) a UB3 polypeptide, the mutated nucleic acid comprising a sequence having at least 90% sequence identity to any one of SEQ ID NOs:310, 312, 314, 316, or 318.

143-159. (canceled)


SEQUENCE LISTING (445 sequences)

SEQ ID NO: 1 - protein, 1228 aa, unidentified
  source 1..1228: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  REGION 1..1206: note = Lachnospiraceae sp.
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  source 1..1233: note = Lachnospiraceae sp.
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  source 1..1227: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  VARIANT 1073: note = Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, synthetic construct
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, synthetic construct
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, synthetic construct
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
  source 1..1592: mol_type = other DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
  source 1..2000: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
  source 1..621: mol_type = other DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 160 aa, synthetic construct
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 27 - protein, 207 aa, synthetic construct
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 28 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 29 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 30 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 33 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 34 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 35 - protein, 1763 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - protein, 1565 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 37 - protein, 1565 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 39 - protein, 167 aa, synthetic construct
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 40 - protein, 167 aa, synthetic construct
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 41 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 42-44 - skipped (3 entries)

SEQ ID NO: 45 - protein, 24 aa, synthetic construct
EELLSKNYHLENEVARLKKGSGSG

SEQ ID NO: 46 - protein, 241 aa, synthetic construct
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 47 - protein, 277 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 48 - DNA, 66 bp, Saccharomyces bayanus
  source 1..66: mol_type = other DNA
ttcttgtcgtacttatagatcgctacgttatttcaattttgaaaatctgagtcctgggagtgcgga

SEQ ID NO: 49 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 50 - protein, 482 aa, synthetic construct
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 51 - DNA, 10 bp, Methanobacterium thermoautotrophicum
  source 1..10: mol_type = other DNA
aatttttgga

SEQ ID NO: 52 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 53 - DNA, 25 bp, Emesvirus zinderi
  source 1..25: isolate = Bacteriophage MS2; mol_type = other DNA
gcgcacatgaggatcacccatgtgc

SEQ ID NO: 54 - protein, 116 aa, Emesvirus zinderi
  source 1..116: note = Bacteriophage MS2
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 55 - DNA, 26 bp, Pepevirus rubrum
  source 1..26: isolate = Bacteriophage PP7; mol_type = other DNA
ataaggagtttatatggaaaccctta

SEQ ID NO: 56 - protein, 127 aa, Pepevirus rubrum
  source 1..127: note = Bacteriophage PP7
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 57 - DNA, 19 bp, unidentified
  misc_feature 1..19: note = Shigella phage
  source 1..19: mol_type = other DNA
ctgaatgcctgcgagcatc

SEQ ID NO: 58 - protein, 62 aa, unidentified
  REGION 1..62: note = Shigella phage
MKSIRCKNCNKLLFKADSFDHIEIRCPRCKRHIIMLNACEHPTEKHCGKREKITHSDETVRY

SEQ ID NO: 59 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 60 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 61 - DNA, 4101 bp, synthetic construct
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 62 - DNA, 4101 bp, synthetic construct
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 63 - DNA, 4092 bp, synthetic construct
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 64 - DNA, 4101 bp, synthetic construct
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 65 - DNA, 4101 bp, synthetic construct
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 66 - DNA, 4092 bp, synthetic construct
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 67 - DNA, 4101 bp, synthetic construct
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 68 - DNA, 4101 bp, synthetic construct
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 69 - DNA, 4101 bp, synthetic construct
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 70 - DNA, 3307 bp, synthetic construct
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 71 - DNA, 4101 bp, synthetic construct
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 72 - DNA, 7296 bp, Glycine max
  source 1..7296: mol_type = genomic DNA
gttgtctaagcaagtgcgtgagatgttgtaattgtatttcaattcttataataaataaaaagtttgaatagctttttagttcttgtaatttagcatttttttgcttttcattcatgcaatttttttttgttttcaatccttgcaaattatgcttgttttattttcagtccttaaaatgatttagatagttttttttctatttaaagtattatctaaactgctttaagaaaaaaaaaaacaaatacaatttgttagctatgacgaacacacatgtaaggttttggggacaattgccccacattttttttatttgtatgtgtaataaatttttttcacccccataaaaaataggtattgccccctttaagacaactttcttttaaatgaacgtataaaatgaatcaaaacaaagaataaattcatactaatttcactactttatccttttaatttttgataatattgataataaaaaaattatagaatatttttgaagtatgaaaaatactagatgaccattgtaatttgtataagtttaaacattttgaatttcttttgtcataacattatgtattttgaaaattttgtgatatttttattttgcaaaaatattataaagtattggttgggttaattatgtttttggttcctaaactttttgtaaaattcccttgtagtctctaaacaatttttataatatttttggtccctataatttctttcattaatagttctagtttctattatcaagcaacaatgttaatttatgacataaccaatcacattaacttgtcacaccatcaagccgtgatgacaattaggtgtaattctatgaggaatttaaaatacaatttataaaaaaaattacacatgattgttccatcaaacccttatgatgtgataagttattatgatgctaacatgaatattacattatcaattaatattgttactcaataacaagtattaaaaccatcaaaggggaagagtaaaagtataaggaccacacatactaaaaaattgtttaaagactaaaagtaaaatttgtaaaaagttttggaaaaaaatataattaatcctaaactatttcttggtcctatgaagatttttttttggattcacagttattttcaaggactaaaacaatttttttctttaaaaaattaaaaataaaaaatactaaatcacaacaactaaaaagtatttaaatctaaataaaaaaaaggaaaaaaaaattcttttgacaccaacaaactgacaaattgagagacatagaaagaaaaatagataaaaataagagttactaataagatgtttaaaagaaaaagatgttaaaatatcaccactcaaaaaataaacattgacatcagtactccatttactaaattgataaaatttcagcattaaattgataacattgacattattaattattactcacaactaaatgaattaatgtataactagctagtactagagggagcatcagcatcagcactgattagccagcagcagcaataggttctggttcagtcaccaaagcaccaccctcttcaaggactaaataattatattctttaaaaaaacaaaataaaaaaaatactaaattacaacaactaaaaagtatttaaatttaaaaaaaataaggaaaaaaaaatcttttgacactaacaaattgacaaattgagagacatagaaagaaaaagaaaaaaaaatattaatataataaataacgtgatcagttaaaaaaaatagataaaaataaaaattactaataagatgtttaaatgaaaaagatgttaaaacatcaccacttaaaaaataaacattgacatcagtactccatttactacattgataaaatttcagcattaaattgataacattgacattattaactattagtctcaacgaaatgaattaatgtatacacccactcactctagttagctagtactaaagggagcatcagcatcagcactcagtagccagcagcagcaatgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctcttatctctttctctctctctcactttctacttttttatttattattgacttcaaaaagaaataaaaaaatcttcttttgttttttttttttttttagttggcttgttatattgggtagttctgcttctgctttggtttattttcctcatcttactccttggtttctgctatttttagtgtttttgctattttgggtgcttttggaatactgctatgctattcacactctctttttagaaaaaaaaataatggtttttgtttttctttccaatttttacatcctattcttttgtggttttggctgtatcttttacattgcttgtttttcatttcagggctttaatcgtgctgattttgttctctgtagattttttttattttattttttatgttgattgcgtgtaatgagtcatgctcttctcttacttcgtaatgaagtgtgtgaaggtttgtttttctatggtttctaatgctgcttaatgattggagaatggttcacgtttctggtttgatgcatctgttggtgctgtgagttgtagatgatagcataaccatggggttgtgttgtttatcttgtagttaacttatgggtagtgatttccgaaggaatatgctttagaaaacaatggtgctgttgtgagcttgtgttgtttcccccttcccctttcatggtatcatgaagtgctcattaaaggaaaacggagctactagggtaggaaataggtgtagaaatgagtgcaaaacaacaactaagttgcataacatttaaagcaatgtagagtggactcagataaatgaaaagtttaggattctgttttgacataccatgccttcaagagtgtcaagatttggaacaataagtccataatatacaattttttcccttttaatttgttatgtgaagttgatattgtccttttgttcatgagatgatacctctctcatgtaagttcattccataagaaaagtgctttttccccaacattatcttatgcagatcatgcaatgcccccataaggtcatagggtctgatgttgtatctttgtcttgctggacaatgcaatccatggtaacttgctattgtgactgttagataactcaatgggacttgaatcagttaattacagaaataacagttagaaactgttatatgtttaactcaagcttgattacaactgaaattactttactccaatttcattgttgctagcaaatgctacagggcattttagggatttgcttaatggcaaaatgtccctaacaagtgtgtaattttggaggtggatggttatcattttttgggtggttttctgactgtaagatgcaaaatctggcctatttgtttgcagtattttttgtgacaataagaatgaggtgaaacaaaagagccatttgagttctatgatctgttggacattgtgtaaaatgttgcagatctcttccgacaaatgtccaaaccaattacgcttattgggtttgagatattttagagaatgattctcgtcctgcagagctaaaattttcaaattaagatgcaacctccctttgccaacttgttctctaagttcctattgatggtctggtcttgcgggatccatatcgtagatcattttagaaaagaatctcttctagatgacttgatattgaacattatatgttgtagacacatgtgaccttcaatccattcaagatgcaacatgttctcaggggaaagatagatctcattgactgacaattatgggatgttaattacttatgttgaataatgatgtttagcaactaaaaggtgtaggaaatttctggatgctctcccagtatggatatagtacttgttgagaactatggcataattcttgtagctaacccctttgaatgtgaaaaaaattgtttggttgttgattgctgctcttctgttttatggcatggttggaacatttttttttttatcatctgatgtgtggtttattggtaattaattgaagttgaactttctttcactggtgtaaagatgtagatggaatggtgtgtaaggaaaaatttgatggctacttagtatgaaaaatttgatgaaaagtaccacaaaaatagacatggctgatttccatttgatttttttataagtaatctaatttgttaattagcagaaagcctcttctttgattttgtgcaaattcatttgtaatgttttcatatcacatagcaccggtcggtgtgtgaatgctatttacaattattagacttgcaggttcctttccacagaagctacattggtgtatatatatatatatatatatatatatatctgtcattaacatattctgtgtttctctactgtgcttaactcaagttcaaatgttttcttactttacaatgagtttaaacatgccttgaggtaacgtgctaattaagctttatctgggcttgtaataggatgttatgatattctatagttgttgtgaccttaaattggaatagtattaacatatggtgttaaaaaatttcaaatgttgctctcttttctccaaatctcatttgtcaattctctgaggctatgatgatctggaaaacttgtgttacaatgggagtttcaagtttcaactgctttggggtttatcggcagttcagcacaagtctatgaatgtggtaagcaaccactgttgttgtggagcttggatagctctctgatggctgtgaacaatgaagtgtaacttttatccctttttgggctttttgtcccgtcaagggttgaaaaggattttattgcacatatttcataccaatttaaggtgcatactattttccataatgaaattaatcagaaagagggatcaacaaatgcatagtgctgttatgaatgttcaagacagtggattaaaaaggttttggagagtacaaatgggcatttttcagaactactaatttccgcattgcagatgagcatatggtatttggtgaaacaaaaggtcatctattcttattttggtaacagagtgaatctatattttaaggtttgtatcttgtattcaagctcaacgacttctctttcaattatgctagtatactgtataaattgtactaaacataatgttatccctcttagcaagaagcgagcgagaaatttttggtatctatgttttagtactgatatccatattgaagttgagttcgaagtatcttggtaccatgggatgttccctgattgcatgttgagaggattttttgctgatgctatcacaacccttgtatccaatgcattaagatttctgtaagatatgggaattgcatcttatggaaatatacgtatgttcttggctttatacatattagctttgttctgtacttttgtcacagttaggaagtatgagtgataaattctctcatttgtaatgtgatcatttaatttaatgaaagtataaaatacttcaaatgcttccaattctgtggtaataacaaatcaacagcattgcatgtaatgattagaagcaattttgtcatgttagaaattctcaaatcttgtgtcatttcgtgttttcttttgtagtatacttttacttaaacttggtagcaattatatttcagacatcttgtgtctgatcatactctctttgtatataaatctggaagcatttatacttcagaaaagctaatatcctctttcatggcccttgcccaggtttcatctgctttctgaatttgatcaaggaaaacgtagctgccgcaagcgacttgctggccataatgagcgccgacgaaagcccccaccaagctccgtattaacctctcgctatggcagactctctctgcctatttttggttagaaattatcagtaaaacatcaattccaaaccactctctctgctttatgtatccctaggaagcatcaataatttcagtttttgctattgcatataaaacattcaactgtttcccttatgtttaacagataacaatggtcgagacgatggctttctgatggaatttgcttcataccccaagcttactttgagaaatacattgccaactcaaagatcaactgagccggctccagggaaccatgctgcaacactcacctggcaaggaggttcagacactccatctgaatttttcctgcaaggatcagggagtagtggtggaacaagcttccttagttcaaaacatcccttagtggaaagttacactggagtcactgactcaaactgtgctctctctcttctgtcaagtcaaacctggggttccagaaacacagccccagcaacaagttttgaactgaacaacaacttgttcaatttcaatgggacatccatgacacaatttgctgtggcgtctttgcaagctgcagccatccatcaacttccaaatgctacctcatggttgtacctaaagggagttgattctggtaattgttcgcctgagcgtgtccccaatcacggtcttgctcaaatttctccccctcttcatagccatgttcatagtgagcttgatgtgtcacagcagggcaggaggcattacatgatctaggggagtccagggcctgtgagtcttctcactggtctctttgaagccccttctttgctatcagaaagtatgaggggaatctgtgcctatttgatcacctaactactagcattttggtttctttgaacttagaagtaaggtgggtatatccttcccttgtttgtggaacattgaaaattgaaacatccttattttggcttgtttgaacttataagcaaggtaggtagccttgtttctttgtggtacttttcagacattactagtttggtgttggaacttagaagcaagatgggtagtcttgttttattttgtggaaatttcagacctgtatcatttgaatgtgggtatgattgatgttttatagcatctacgtctgtgattccactgaatgcatattccttatattgctat

SEQ ID NO: 73 - DNA, 1077 bp, Glycine max
  source 1..1077: mol_type = other DNA
atgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtttcatctgctttctgaatttgatcaaggaaaacgtagctgccgcaagcgacttgctggccataatgagcgccgacgaaagcccccaccaagctccgtattaacctctcgctatggcagactctctctgcctatttttgataacaatggtcgagacgatggctttctgatggaatttgcttcataccccaagcttactttgagaaatacattgccaactcaaagatcaactgagccggctccagggaaccatgctgcaacactcacctggcaaggaggttcagacactccatctgaatttttcctgcaaggatcagggagtagtggtggaacaagcttccttagttcaaaacatcccttagtggaaagttacactggagtcactgactcaaactgtgctctctctcttctgtcaagtcaaacctggggttccagaaacacagccccagcaacaagttttgaactgaacaacaacttgttcaatttcaatgggacatccatgacacaatttgctgtggcgtctttgcaagctgcagccatccatcaacttccaaatgctacctcatggttgtacctaaagggagttgattctggtaattgttcgcctgagcgtgtccccaatcacggtcttgctcaaatttctccccctcttcatagccatgttcatagtgagcttgatgtgtcacagcagggcaggaggcattacatgatctag

SEQ ID NO: 74 - protein, 358 aa, Glycine max
MGSASVTKAPPSSSSSPNSSSTESLDGLKFGQKIYFEEVSVAAAAATTTQGGNKTRGGVSASKKGRGGSVQAPRCQVAGCKVDLSGAKAYYSRHKVCAMHSKSPTVTVAGLEQRFCQQCSRFHLLSEFDQGKRSCRKRLAGHNERRRKPPPSSVLTSRYGRLSLPIFDNNGRDDGFLMEFASYPKLTLRNTLPTQRSTEPAPGNHAATLTWQGGSDTPSEFFLQGSGSSGGTSFLSSKHPLVESYTGVTDSNCALSLLSSQTWGSRNTAPATSFELNNNLFNFNGTSMTQFAVASLQAAAIHQLPNATSWLYLKGVDSGNCSPERVPNHGLAQISPPLHSHVHSELDVSQQGRRHYMI

SEQ ID NO: 75 - DNA, 6953 bp, Glycine max
  source 1..6953: mol_type = genomic DNA
ggatgttgagtgaagtgtcaccgcatgaagtgtcatagtgtgagagccgcaaagaagtaaaaagttagggatgttacgccactcgttaacccctgtgatttttcactctttttctcttgtcacgtcattggaaatgaatgtcatcatgctaaattttaacataaatttatgctattcagttggataaaatgaaaataaagaaatatttttagtaagaaattatatttaacaagaaaaaatatcactatttcatatcatgctagagaacacatgttaaaaaatatgaagacaaaaagaatcaagagaccttgacaaattctaacatgtgtgactaaagtatgatagtgaggtatgacatctatttatattgtgtatcttttaacgatttcgataactttttgttttgttaacattgtctaggagtgatgtgtattatgttgttcctacttgtgtgtgtagtgatttcattgcattctactcacattaagtatctagaatcccagtattagacttaggtgtggtttgtattggtttctctcacttacgttgttagaatggttttatcgcatcttacttatgcattcaagtatctagaagcctagtattgagcctagatgtagttttatagcaccctactcaggtgttaagtatctagaatcctagtattgactttgttaacctttattgtcatttgtcaatgaattgcgtattatattgctcctatccgtgtgtagagtgattttatcacgctactcacgtgtttggtattggatctaggttcttcaaacttatgagctttgttagcttacttaggtgatccattgaataatgaatttggttaggtcatcctctcaaatttatcttttattttgaacattttggtcaacctattatgattggaggttcacaacttcttaatcgttgagcttaaaagatgtaaatcctggggtgattagtcactaactagacaatatacaacccatcaaacactgaacctaaaaataaatatttgttgtgattgatttctctaactagccagtccataaataattatttaatttctaaaaaaataaaacattttagttataactcaagaatgaaaagaataatgtatttaaatttatagttctagataaaatcattttcttattttactttaaattaaattaagtgattttttatataaaagaatattttatttgtattaaattaaaattaattttttggtatgaaattattaactatttttaaattattttaaatatatttttaaaaataattattataaaaccaaaatattattatacaaaacactttgtgatttaataaccataagtgatagcaacacattttataacactctatttaatatatttttattatagattaaaatttattaaaaattacaaatgacacttattaagaaaaattaaaaactaaatcattaagtatatgaataaactaaaataagattattttaatttaataaaaaatctcaagttcaaatcttatgttaaaactgtttaaaatacttagaaaaagaattttatcacctataatgatattttttaactcaaatataattattatttttggataatacttgtgatctaaataaaaaattaacttataatattttataatttgtaataaatttcaatcaaataattaaaaaatattttaaaagcaaataacgtttctcaaatatataaaaataaacattaaccatcaaaacgtctaactaattaattgatcagtttctatgaattattctaatattcttttaattcttataaataaataaacattaaatcattactccatttaattaaattgataaaatttcagcattaaattgatgacattgacattattattaactactactctcaagtctcaacgaaatgaattaatgtatatacactcactcactctcgttagctagtagctagagggagcatcagcatcagcatcagcactgagtagcagcagcaggagcaatgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtcaccatcctctctctctctctctctttaccttttgtttatttattattgacttcaaaaagggaaaaaatcttcttttgtttttgttactcttttgtttttgctatttttagctggcttgctatattgggtaattctacttctgctttggtttcctttcctcatcttacttccattgtttttgctatttttagtgcatttgctatactgctatgctattcacatgctcttgttttttagaagaaaaatggttttgtttttcattccaatttttacatccctttgttgtgtggtttttggctgtatcttttacatttgttgcttttcatttcaggaatttaatcatgctgattttgttccttgtgattgttctctgtagattttttttaagttgattgcgtgtaatgagtcttgcacttctcttacttcataatgaagtgtgtgcaaggtttgttttactttggtttctaatgtttctttatgattggataattgttaatgtgtctggatttgatgcatctattggtgctgtgagttgtagatgataacataaccatggtgttgtgttgtttatcttgtagttatggttagtgattggcgagggaatatgctttagaaaccaattgcttttgtgagcttgtgttgtttcttctttcctccttcatggtatcatggagtgctcatgaaagggaaaggggagttctcagcgaaagaaaaaactgtacaaatgagtgtgaaacagctgcataacatgtgaatcaatgtaaattggactctgataagtgaaaaatttagggtagtttagacataccaagccttcaagagggtcaatatataaatcaagatttggaataataagaccatgcaatactattttcccatttttaacttgttatttgctcatgaaatgatacctctctcatgtaatgtaagttcattccacaagaaaagtgcttttttccccacattattgtcatatacaaatcatgtaaggcctccttaaggtaggtagggtctaatgtcctatctttgtcttgttaacaactcaatggaaatgactaaaattaatgttactggataattcaattaacttgcttgtgactgttagatacctcaatggggcttgaatcaaagatgtaagattgtaagtgaaaagggaatagggttagaagggagaggaagagagggatatcttttggaagttagatggttaacagaagagggactgggctctcaaattccgagtgcagagagaagagtggtctgtgttctcattgttcaatatagaattatcattcttgttaccgaattctgatctagatcttaagacagtatccattttctgttcagtagtgaaagcttgattacatctgaatttactttcctcttcaatttgactcatcattgctagcaaatgttacagggcattttagggatgtgtttaatggcaaaatgttactaacaagtgggtagttttggagatggatggttatcataatttggatagttttctgaccataagatttggaatacttggcctacttgtttgccagtatttttggacaatagttatgaggtgaaataaaagagccacttaagtgacctgattggttgggcattgcataaaatgttgcagatttgtcctccactgacaaaccaattatatttttccagattaagatattttagggaattattctcttcatgcagggctaaaattttcaaaaggaattgccaacatgtgctctaagttcctattgatggttgatttcatacaccttcgttcatttgtcttgtgtattgtggtattcatatgaagatcatattagaaaataatctcttctaaaactacttaaaattgaacattcaagccattcaagatgctgtatgttctctggtgaaagataaaccttgtcgattgacaattgtgggatgttaattacttatgtggaataatggtgtttagcaactaaaaggtatagggatttttttggatgttctcctggcatggatatagtacatgttaagcactatagaataatggtgtttagcaactaaaaggtatagggattcaaatgtttttttaaacttaataacaagagtttaaacataccttgaggtagtgtgctaatgaagctttgtctgggctcttaatatgatgttatgttattatattctatggttgttgtgacttgtgaccttaaattggaatggtattatctcatggtgttaaaaatctcaaatgtcattctttttcctccaaatctcatttttcaattctttgatgctatgattatctggagaacttgtgttacaatgagagtttcaactgttttggggcttatcagcacaaatctattaatgtggtaagcttccactattgttagggagcttagatagctctctggtggctgtttcctgtgaagtattagtctattaatgtcccttttgggcttttgttccgtccaaggtcgaataggattttattgcacattttccataccctctttaaggtgcttcctattttccataatgaaattaaccagaaagagggaagggatcaataaatcgacagtgctgttatgaatcaagacagtgtttaaaaaggttttggagacatagcacagatggatgttttcaaatctactattttcagcattgtagatgagcaaacacgttaatgggcgaaacaaaatgtcatgtattcttattttggtaagaaagtgaatgtatattttaaggtctgtattctgtattcaagctcaatgcttctctttcaattatgcaagtatatattgtaccaaacatgaaatttaagttcgaagtatcttggatgggatgttccttgattgtttgttgagaggattttttgctgatgccatcacaatccttagtttaaaacatttgtgacttaggctgtatttccattatgatatactgtatccaatgcattaagatttctgataagatatgggaattgcttcttatggaaatatacctatggtattggctctatacatattagctttgttctgtaatgttgtcacaatgactaagttttactgataaattctctcatttgtaatgtgaatttaatataatgaaaggagaaatagttcaaatgcttccaattctgtggtaacaacaggtcaacagcattgcatgtaatgctaagaagcgattttgtcatgttagaattctcaaatcttgtgtcatctcatgttctcttttatagtaatgttttacttgctagaaattatatttcagaaatcttgtgtctgatcatactctttggtatcatgcttttatataaacatggaagcatttatatttcagaaaagttaatatcctctctgatggcccttgcccaggtttcatctgctttctgaatttgatgaaggaaaacgaagctgccgcaggcgacttgctggtcataatgagcgccgacgaaagcccccgccaagctccacactagcctctcactatggcagactctctacgcctatttttggttagaaattatcagtaaaacatcaattcgaaaccactctctctgcttaatgtatccctaggaagtaggaagcatcaataatttcagtttttgctattccttataaaacattcaactgtttcacccatgtttaacagataacagtggccgagccggtggctttctgatggaatttgcttcatacccaaaggttacattgcgaaatacactgccaacacaaagatcagccgagccggctctcggtaaccatgctgcaacacttacctggcaaggaagctcagacacaccatctgaatttttcctgcaagaatcagggagtggtggaacaagcttccttggttccaaacatcccttactggaaagttacactggagtgactgactcaaactgtgctctctctcttctgtcaagacaaacctggggttccagaaacacagccccagcaacaagtgttgagctgaacaacaacttgttgaatttcaatgggacatccatgacacaattttccgcggcttcttcacaaactgcagccatccatcaacttccaaatgccacctcatggttgtacctaaagggagttggttctggtgattgttcgcctgagggtgtccccgatcttggtctaggtcaaatttcgccgcctcttgatagccatcttcatggtgagcttaatgtgtcacagcagggcaggaggcattacatggatctaggggaatccagggcatgtgagtcttctcactggtctctttgacgcacttctatgctatctgttactatttgatcagttaccagactggcactaactactagcattttggtttctttgaacttagaagcaaggtgggtatatccttcttttggttgtggaactttgaaaattgaaacatccttattttggtttgtttgaacatatcagcaaggtaggtagccttgttttttttttttttttttgtggtacctttcagacattattagtttggtattggaacttagaagcaagatggggttgtcctgttttattttgtggaaatttcagacgtgtaaatcattttattgtgggtatgatcgatgttt

SEQ ID NO: 76 - DNA, 1122 bp, Glycine max
  source 1..1122: mol_type = other DNA
atgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtttcatctgctttctgaatttgatgaaggaaaacgaagctgccgcaggcgacttgctggtcataatgagcgccgacgaaagcccccgccaagctccacactagcctctcactatggcagactctctacgcctatttttgataacagtggccgagccggtggctttctgatggaatttgcttcatacccaaaggttacattgcgaaatacactgccaacacaaagatcagccgagccggctctcggtaaccatgctgcaacacttacctggcaaggaagctcagacacaccatctgaatttttcctgcaagaatcagggagtggtggaacaagcttccttggttccaaacatcccttactggaaagttacactggagtgactgactcaaactgtgctctctctcttctgtcaagacaaacctggggttccagaaacacagccccagcaacaagtgttgagctgaacaacaacttgttgaatttcaatgggacatccatgacacaattttccgcggcttcttcacaaactgcagccatccatcaacttccaaatgccacctcatggttgtacctaaagggagttggttctggtgattgttcgcctgagggtgtccccgatcttggtctaggtcaaatttcgccgcctcttgatagccatcttcatggtgagcttaatgtgtcacagcagggcaggaggcattacatggatctaggggaatccagggcatgtgagtcttctcactggtctctttga

SEQ ID NO: 77 - protein, 373 aa, Glycine max
MSSGSVTKAPPPSSSSSPNSSSESLDGLKFGQKIYFEDVSVAATTTTQGAYKTSRVVSSPSSASKKGRGGSLQPPRCQVEGCKVDLSGAKAYYSRHKVCTMHSKFPTVIVAGLEQRFCQQCSRFHLLSEFDEGKRSCRRRLAGHNERRRKPPPSSTLASHYGRLSTPIFDNSGRAGGFLMEFASYPKVTLRNTLPTQRSAEPALGNHAATLTWQGSSDTPSEFFLQESGSGGTSFLGSKHPLLESYTGVTDSNCALSLLSRQTWGSRNTAPATSVELNNNLLNFNGTSMTQFSAASSQTAAIHQLPNATSWLYLKGVGSGDCSPEGVPDLGLGQISPPLDSHLHGELNVSQQGRRHYMDLGESRACESSHWSL

SEQ ID NO: 78 - DNA, 6100 bp, Glycine max
  source 1..6100: mol_type = genomic DNA
gtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcgagctgtgctctctctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggag

SEQ ID NO: 79 - DNA, 1104 bp, Glycine max
  source 1..1104: mol_type = other DNA
atggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcagcgaattgattcttgcagcccccaggtttcatcagcttcctgagtttgatcaaggaaaaagaagttgccgcaggcgactagctggccataatgaacgtcggagaaagcccccacccagctccctcttgacctctcgctatgctagactttcttcatctgcttttgatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcgagctgtgctctctctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaa

SEQ ID NO: 80 - protein, 367 aa, Glycine max
MASDAKLSSSESLNGLKFGQKIYFEDVGFATPATSLTSSATTATTVTSSSSSSSRKGRGGSVQPAQPPRCQVEGCEVDLSNAKTYYSRHKVCGMHSKSPTVIVAGLQQRFCQQCSSELILAAPRFHQLPEFDQGKRSCRRRLAGHNERRRKPPPSSLLTSRYARLSSSAFDHSGRGGNFLMELASYPKLSLRNSLPTPRSSELAPGNQTSTLSWHGNSETSSDLFLQGSVGGTSFAGPGHPPGESYTGVTTSSCALSLLSNQTWGSRNPAPSLGLNNMVNFHGTPMTQPAASSHGASIHQLPNTPWCFKGIDSGNCSPEGVPDLGLGQISQPLSSQLHGELDLSQQGRRHYMDLEQSRAYESAHWSL

SEQ ID NO: 81 - DNA, 6430 bp, Glycine max
  source 1..6430: mol_type = genomic DNA
aaaactatcattgttgtacacggtcaaaaataatacatacacatatgtacatagatacatgcacacacacacaaacatatatgacatatcatatgagatgaatatgattattatgagagtgaaatgattaaatttgaaccgtatatccatatatgcaaggttaagatttacattctagatttaatctgtttagtctcatataatatgctttctctaataagatatgtcatctctctccctccctctctctctctctctctctatatatatatatatatatatgacatgtcatatgagaggaaaatattttatataagagtgaaatgattaaatttgaaccatataatcaaacatgaacaatcaagatttaatcttaaaaagtcatgcgaccttttaagtagttacatcgtattaaatcttaactttatacagttcaaatttaatcttctcactcccatataagatatttcctctcataagattttgtagcggcgtcaatcatattggtggcgatgatggtgatcatggtgatgatcacattggtggtagtagtggtggtgagggtgatgacaacgacaacgaccacggtcatgtgatggtgacagtagagattaacgatcatggtagtggttgcggtggagtttatgatagttatggtggaaacagtcataacatgataatggtcatggttatgataggtttaagatatcttaggacatcacatattaaatctttaagatatatatttttttgttttgtaaatcttatggagatttataattgaataaaattttaatttgaattttaaatgttttaatcttgatctctttcattttaatctaatcgttgaaattaattattttcattttcacataagaacataagatgattattctcatatgatagatacgtcttttaaacacatacacacttgtaaattagctatgaaaaaattgaaaggataataactttattgtcttagattataaaatgtggttatgccgtgatataaaaaagtatgtaaatctataaacaacatgtgttgatttaattgttaaaaagaaattaaaaaatacttaccttttgagtcaatcaaatcgttgattcaagtcgaatcaaatcgattcatgtcgaatcatatgcatattgaatccaactaatagttgagattcaataggtgtctaattctagactaatgggttgagccgagtctaataataacatgtttggataaatggtgcttaatttcaattcaattattttttcacgtttatagtaaagaactctttgtagctttgagattttcacggtgaaaaattgatttttgtggtgccaccgcagaaccaaagataccataagtcgccaatgaattgcaattgcaaggtcaagattttttttcgtttgattctgaaatgaatatataatgatgatatgttgtaaaatttgggtccatggatacatccctttcgtatgtagcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttccactttactctcccactaccactcctttcatgacttcaacttcaacaaccacctctttctctctcacttcttctcttgctcttatcaaaacaaaccactatgaataatactaacgataataatagttacaacaatacactctagcttctagcctgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtcaaaccccttctttcattattattattattattacctgaaacaaaactgaaaattctctttttcttcttcttgtgctattttaatattgaggtaagataaagtagggtagggccttgtttattttattcagtttttttgctcgttattctgcggtttggggtgtaggtttgtaggtgagatatgttatcctgttttgtcggtgcagttataggtgtgtgtgttacttgtttggagttggtttagttccttaatcgtggctttgcttttgttcaaggctttggtgtggtgtgattctttgtgtctgttcttgagatgtttgttgttcagttatgatttggctgattgtactcggtatactttacttttttttcccctcctagcgtagctttaaagtgcttatctgaacggctgtcttataacgcatgtgaaatattattgttttccttttcagcgaattgattcttgcacccccgggtgtgtttggtattaaaaataataacttcgtttgtacggaaaaaaggcttgaagtgggtgtgcaatttgtagtcttaaaataacacaaacactactatgctgttttccctctagcctttgagggggccctcaattgtgtagtgaactattgatatgctgttacagttttctcattactgtttttgtgtttggattatcttttttggagaaataatattttttttttccttttatagattactagagagcctttgaaaaaaaataaaataatgataattttccaaatataatctgttaccaaacatgaccaaactctttaggtatgtgatctgtgtaaagatacatccatgtattttactgtcatgaatcaagttgttttatagtgtgaaggcatgttatttgtactacaagagcaattccttgttgatgtgaacctaggattcttactcaaggcttgcattattatcaaaatggtagttttctgctaattggagtgagagtgactttagccttcccttgtctggtcatccataacgtgggtcctgattgttgaatttgatttatctatcttaaacagataggttgtctgattgtttatgtaatttccttgcatgaagacactagtcatgtattgcctccttgactaatgttagttttcatactgtttagtttgacttgttgatacattattgagaatgagtgcaattggtgttgctggatcatgaaatcctaaccacatgctttgcttatgtgactattcatctctattctgttttatggtgctagctatgtcagactgtcagttgtcacatgtactatccatttcaatttgactcattagtgctagaggatgctactgtgcattctaggatagtattcaatggtgtaataagcttgtattttggagacagatggccatcataattggaaagtttccaaccccaggatatggagaatatggtctatctgtcaatgaatgtggttttatatgcagtttttttttggagctgggataactttctgttggctgttctaagagtaatttgtgtctttttttttgtctcatggttcttccacagttgaaaagggttatctaagtcaaaagttacacttttccttaataatgaaaaacaatactcttctgaatagggtgacaatgaattgaaccaactcgaaaatagttcggaactcgattcaataatcagctcgttgaatttggttcatgaaccaaatgagctgaatttgagctcgttaaataaatgacccaaacttgagctgctaatagttcaactcatttgattcatgaattggctcaatatatatatatatataattgattgggttttctattattttatatatatctagggcagggtagtttttatttatgcagactttggttataattatgttttttttgtcataccttgtggccctacaaagctaatagaataagttcagttttgtcttttcacaaaaattaaacaatcattttatctaaaatctaccaataaattgaaaatttgaattgtataggatttaaaaaatttgacttttggttcatgtgatccaagctaacaagctgaattgaactgtttgtgagcttgaactgagttgagttcgagctgaaaaataaaggttcatgaaccaaaagtcaaatttttcagcaaaagatattaatgagccgaactgaactcgactcgtttccggacctacttctgaatattctaagttaaaatacatatcatgagaaagatcgtgaaaagatatttaagatggactttttagcatccaatctcccagcatttcagatgtactatcggtggatatacattgtggtttatcccctgtatttaagcttgatgacttctcttttgaattattctggtgtctttcactaaagagtaaatacaatgccatcccttttggtaatatagaagataaacaacttcattggtattgttttggtcttattttactaatacgttaaagctgaattccaagtatcatggagccgcagaatgttacttgagcaatgtttgaggattgcttcagtaaaacagggctcaaggagcatcttatactgctaatatgccatctcattcaatgccaaacaagctcacatgatgtgcatcatatggaatacccttgttcttgggtttatacatattgcttatgttctgcaatctttgcaatgatgactaattcaaataagttagaaattccatctcatttgcatctgcatttaatgaaacaacaaatattttaagtgcttccaatttgtggcaacaacagcatgtcatgctaaggtaacagtagacttgtcatgttagcatttcttaagtcttggcacttgggtatgcttgtactcattcacctctctttgacataaattggaatcatttatatttgtaaaagataatatcatgtttcatagtcttgcacaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttggttagaaactatcagtaaaatatcagtttcatgtctcttccaattttttcatagggaagcatatttagtttcagcttgtgtcattcctggcatacaaaagtaaaacaaactctaatgtctggcacttttattgacagataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctgtgctctctctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcatggtgagctggacctgtcccaacagggcaggaggcattatatggatctagaacagtccagggcatatgaatctgctcattggtcactttaatgcacttgtttttttttgtcagctttgtaataacatgtagcagaaacctgtgcctacttggttacttaccagattgatgataactattgcttgttgttttgttttgttttaactgataagcaaggtgggtagccttgcctcatttgtggaactttcaggcaccgaatgaatctcacctgagagtgtgttttgttagtagtgtcttaaacacaggtatggtgttttctgccattgccgtcacctctggaattatttgtgattgggaggacagcctctttgatgtttgcta

SEQ ID NO: 82 - DNA, 1080 bp, Glycine max
  source 1..1080: mol_type = other DNA
atggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttgataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctgtgctctctctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcatggtgagctggacctgtcccaacagggcaggaggcattatatggatctagaacagtccagggcatatgaatctgctcattggtcactttaa

SEQ ID NO: 83 - protein, 359 aa, Glycine max
MASDAKLSSSESLNGLKFGQKIYFEDVGLATPATSLTSSSSSSSAATVTSSSSSRKGRGGSVQPAQPPRCQVEGCKVDLSDAKAYYSRHKVCGMHSKSPSVIVAGLQQRFCQQCSRFHQLPEFDQGKRSCRRRLAGHNERRRKPPTSSLLTSRYARLSSSAFDNSGRGSNFLMELTSYPKLSLRNSLPTPRSSELAPGNQTSTLSWNGNSETSSDLFLQGSVGGTSFASPGHPPGESYIGVTDTSCALSLLSNQTWGSRNTAPSLGLSNMINFNGTPLTQLAASSHGASIHQLPNTSWFFKGIDSGNCSPEVVPDLGLGQISQPLNSQLHGELDLSQQGRRHYMDLEQSRAYESAHWSL

SEQ ID NO: 84 - DNA, 7325 bp, Zea mays
  source 1..7325: mol_type = genomic DNA
cctgatacatagaattgaaaagagattgtcctattcctctaaatgttcatattgttttataagtggctatggaaaaacaaatgccaaccaaagcacaaagttttctactgattgtggttaaaaacagacttaacacaaggaatatgttgagacgaaagaacatggcacttgagtcatattcgtgtgaaaactgcatttgacaaaaggaggaaactctatatcacctgtttcttagatgtaactttgcaaaggcctgctggaattccattgctatgacccctcctagaattgctcatccggaagaagcctcggctaacctcaggcaataacttaatattcctttctctatggaaataatcatcctcatgacatggagtatctggaaatgtcgaaatgcatggatatttaaaaacaaagacccaacagtgcaacattgcaaaaataaattctcaaaggaattactcctggtcatctagagagcaagaggaaaatatgacaattcaatccagactgtcttaatcagtggcagtcttaaccctatagctcattcggtttaccttctcaactattcaattaggattctcctgtacatacactacgctgtaatttaccattattaatacaaaatttacagtaggagtctctccctcctgatctttaaaaaaccacttgatcaccttctccttacgttcaaaggacaacactattacttcgtttggatgttggaatttggtagcaaggaatcgaatagatgttgaataccaaatcgctaaagatatttagcgctcccctatcgatgcaacgctgctcgcttgagcccctctatgctctacagtacaagggctatacacaggaatccgcgaaattcggcctgaggctatccgcagcggttcttcctaatttttccctctatatcacttttttgcgtcacatcatcaacatttcaccccctatttttttcatctcccgcagcggttccccctatattatcccctatacccaactaaaaatataaaatatcattatctaaccatatttatcttttattactatttttatcaattattaaataggggagcactgtgcaaggggcgctacagtgctccccttgatctgggggacgtgcgcgccctctccttacgctgcagcgcgtaggggctctttagtgtcagtcgctgcgggcctagagatcccgtacgtgagagacagaggaggggtcacggcgtcgcaagcgctgcggccagtctgagcaaaccaaccctttgtctgaaatgtcgtgcgcgcgcattatttgttagttaggtaaaataatattttatggctgaggtagggagcggaagaatttaggagaaaacccgttgcagaaaagagaaatgtaggagataaaatctgatgatgttgcgcgcggtgccgccacaagcccacaagcttgcgcagcctccctccgcccgtccctggcgtctacaaaaagccctcggcaggtggcgagtggagacgctccctcatacctaccagccaaaccaagcaagcagggaatccccacaaggcagcagatggaagcgaaaggtggcggtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggaggagagagaagagagggagccagtccagctccagccagccggacgcccggaccaacaccaccccgccccccccccccgccccaccggcccgccctctcgcgctgccgtacccacggccccacgcacgttcacaagtctcgtcgccgccgcaatggagtccggcggtggcggggacgaccagctgcacggcctcaagttcggcaagaagatctacttcgaggacgccgccggctccagcagcggcagcagcagcggcggtggcagcgcgcccgcgcctccagcgacgcagcagccgtcgccgccggccgcttcgcctagggccccggccggcggcggcaggaggggcagggccgcggccggcggcgcgggcccctcgacggcgcccgcgcccgcgcgctgccaggtcgacggctgcaacgttgacctcaccgacgtcaagcccgcctactactgccgccacaaggtgtgcaaaatgcactccaaggagccccgcgtcctcgtcaacggcctcgagcagcgcttctgccagcagtgcagcaggtcaccacccagccccgccccgccattgctgcacagttgcactccccccctcttccacctccacctctcattcaattgattgtttccagttgtcacttgttggttgcattcgctctgcagtctgctcccccgtgttttttttttccagtcccactcccttcattcacagtctagggttttagggaggctacctagacgaagaccagacggtttcaggtccatcataccacggacacgctacaaaactatagatgcgctaaacagtgggagctagggaaagaaacttggctcggtttgacggagcttccatcccatttcatctatggcgacggcttctcctcctgaagctgaacaagaacgaaggaggaaaaaaacaaaagctgctttggcattactgctacgctttaatgtttcgcttgaatggtgctcatgagcacaggtaggtctgatggatgctcctcctaaaaatatgatagagtgatgtgactcagcctgcccctgccctgcctgcctgtctgtcagttcgctttgcattagattccaccgctaatactactcgggtatcccagtgaagcctgtcagctaacaagccattacaataagcgattagtagccttgagcctggttatacgtgtggtccccaaaaaaggaacagcaacagtgaggaattttcaaaggagtctttgctttgccgatgcagtagtattttaatttagaggagtcccaactttgcacaggttgacactatccattgttttagctagctagcctgcctcaaatatagcattatacattgtttgtccctgtacttaaccatactacatcgggcatccttcgcccccgccccccccccccccccccctttttttttttgcattatgcaaatggtcccccgaggaagaacaagttgtagcctttgcaatgtgttgttcttggcgctgcaactttacaggttgtacgatcctgttcatccggtgtaagatctggaggatttctagtcccttcaggtgctacaccaaactagactgttcctggaacagctttgtttcctttggatacaattagcattttcattcataaatcttaattcagtcatctatctaatttgacatacaatagagagagagagagaaactaaacatgatggtggcgagctgcatctaatttgatatgggtagggcctggacacattaccctagcggcgaccgcaggctttgtgctttaggactaggctacaggagtgtcctcatgtccccagcaaaagagaagggcgccacagttctggatttggggctctctactaaaccacactgccacttatagcaacaaggcatatcattagagaagagctaagttggaccagccaccagtaccacttggcgttattagatggaaacctcattattggtgggtgtgtactgttgtgttaagcccggcatcacacatctctatgaaatgattacacacacactttctctttgcttttcttttctttttagtatggatgtgcaggtgtcattatgagcggcataagacagaggttaatgttaatctttaatgggggatgtgcagtagtacaaccctaacctagggagaagagaaatgttactctctttgatgattgtactggcattcccactggctttgacctaaagtgtggtttcctatatccatgtcgcgctaagcgctatgcatgttgctagctactactgagataaactagtgccttcaacttgctacgtactgtcccaccattcagcttgcactttattctactgtaagagttactactatggtaaatggtttatgcttggtccatggaactttttactgcctgatttgtttgcattctacacaaaagctatgaatctacagcaatgtatatgtggatttatagaatgcaacttccattacttataaaaattctgttgctgtgccaccaacttccattacctgtaataaactgggatcaagggcgcgtgaatgaacacacctctgtttgtcgcatcaatcgcaggttccaccagctgcctgaattcgaccagctaaagaagagctgccgcaaacgcctcgcaggccacaacgagcgccggaggaggccgccgcctggaccccttgcgtcacgatacggccgtcacgctgcgtcgctcggcggtaaccaaaagctggtttttaccctgtgatgtcgtctcggcgaccaaagttcccgtcttttttttgtaaccgctaatgttcatgtgcttggtccagaagagcccggcaggctcagaagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagccacagccacagccacgctgcgccaacagcgggtttcgacggcggcagccctgtggcgccctccctcatggcggcgagtagctacatcgcgccgagcccctggaccgagaccgactcctggggccacgaaggcgggcggagcgtgcctcagctgccacctgacgacgtccccctcggcgaggtgcactccggctcgagcagccaccacggccagttctcaggtgagctcgagctcgccctgcagggaaacaggccagcgccagggtcggcggcaccgccagcgccgcgcaataatcagggctccgcgggcacgttcgaccaggctggcaacacgatggactggtcgctctaggccgggggccgaaataaagcgcagcagatcctagccgcgcagggagatggaggaggctgaaagtctgatgctgcacgctgcttgcttagcttagagcatccagcatgataagagtgatgaactgatgcggtgtcaggagactagtttattctgtgcttcaccatgaactcgtctgtcgcggcgtctatgtagacagacggcacacgtccgtggttcgaagttaccagtatgcttgtcggggactttgctcgcgatgcattaatctcattgttcaccatccagaaatctcatgttctctactcgtttgctggcccgtccatgctgccgctgctgtggtctttacatcaccatggctccgcctccatgcatcatcatttctactgaataataatgtgcttgtaccctccaacgttgccgcctggcattgccaagaatcggttccaagggggtgttgtttaagtaaagcgagaccacaggcacagcgcggctttgcagcatgctcttggcaccagtggtgtggtgtggtgttgctgctgatagactgaccgacggcagaatgagatccacgcagcagcttctttcaaatccacaaaattttctcagtggagaacaccagcacctttcctgcgaaagattttttccccttcccttgcgaattcatttcccttttgcccaaccacacagtctgctgcgcaggtgtttcaatttccttcgctcttttgtccagtctccactgttggttgagcagccggcgttctcgatttcttcactgttctggatctgttgtacaggtcgggactgtagctgccgctgcgctgcgtgattgatggtcggaacagttctgaggaacccacgctcgaaagaaaaccaatagaactgcatcgaccgcaaatgctgtcgtctttcgccatatgcagctcctgcgacgatgaacagtgatcgttccaggcttaccatcggtgctcagtgatccttagacgacacacagtgcagggattctatcgtcacactgtagatgctcgagagagagtagggttgggcattcacgagtcacgactacaagctcgagcagattgtcagtgttaaaagacagatttcaatatggtcgcagattctttgtcaatatatatatatatatatatatatatatatatatatatatatatatatatatattgttgggggccttcgtcctccgaaggtcctcaaaaacatgatttgacaatgttttccaagtggattatgtgaacaggtaccttcggactcagaattacgaacatgaagtgcggtcaggacgaagcctgaaccagacgaaggacgggctgattaccaagctccacgaaggatggtgcacagccgaagctgtgcgcagggaagcttcgtcgcgatagcagagaggggaaccgacttaaagatgaaaagactacgtggaccttgtcaggtttccataagtcattgacaaatgtaatggacataaatgtaattttacgtgggctgcgtcccgcgtctataaatagatgaacagtactcccgtactgttcacgttgacttggcatttgcttttgcgtcacgcttgtactttttatcttctctcaggaccgaaggtacatttgtaatttgaaatcatttctatttttccatgttaataaaatagaaatgattctatgatgatgcttatattatatttcatgctttatatgaatccttcgtcattacttgttatgtttacgaaggtatgtctcttatgaccttcgtccgaaactcattatttcccgaagggacataatgcttcgaaggacgaaggactttaacacttaacactttttatgttgccttgttcttaattcttagcatttgagaacaagtccccaacattggcgcccacctccggtgaactcacttccacttcgagttttttgaacaccttcggaagctatagaccctcgctatggcaccgaagaaagcttcagcgactggggctgcagcactgcaaccgctgg

SEQ ID NO: 85 - DNA, 1212 bp, Zea mays
  source 1..1212: mol_type = other DNA
atggagtccggcggtggcggggacgaccagctgcacggcctcaagttcggcaagaagatctacttcgaggacgccgccggctccagcagcggcagcagcagcggcggtggcagcgcgcccgcgcctccagcgacgcagcagccgtcgccgccggccgcttcgcctagggccccggccggcggcggcaggaggggcagggccgcggccggcggcgcgggcccctcgacggcgcccgcgcccgcgcgctgccaggtcgacggctgcaacgttgacctcaccgacgtcaagcccgcctactactgccgccacaaggtgtgcaaaatgcactccaaggagccccgcgtcctcgtcaacggcctcgagcagcgcttctgccagcagtgcagcaggttccaccagctgcctgaattcgaccagctaaagaagagctgccgcaaacgcctcgcaggccacaacgagcgccggaggaggccgccgcctggaccccttgcgtcacgatacggccgtcacgctgcgtcgctcggcgaagagcccggcaggctcagaagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagccacagccacagccacgctgcgccaacagcgggtttcgacggcggcagccctgtggcgccctccctcatggcggcgagtagctacatcgcgccgagcccctggaccgagaccgactcctggggccacgaaggcgggcggagcgtgcctcagctgccacctgacgacgtccccctcggcgaggtgcactccggctcgagcagccaccacggccagttctcaggtgagctcgagctcgccctgcagggaaacaggccagcgccagggtcggcggcaccgccagcgccgcgcaataatcagggctccgcgggcacgttcgaccaggctggcaacacgatggactggtcgctctag

SEQ ID NO: 86 - protein, 403 aa, Zea mays
MESGGGGDDQLHGLKFGKKIYFEDAAGSSSGSSSGGGSAPAPPATQQPSPPAASPRAPAGGGRRGRAAAGGAGPSTAPAPARCQVDGCNVDLTDVKPAYYCRHKVCKMHSKEPRVLVNGLEQRFCQQCSRFHQLPEFDQLKKSCRKRLAGHNERRRRPPPGPLASRYGRHAASLGEEPGRLRSFMLDFSYPRVSSAMRGGFPAVRPGGERVPGGIQWQAGLDPRHHQGAVAGYGAHYGSEGGSSSSARPPVFPGPELPPGGCLAGVPADSSCALSLLSTQPWDAAHSHSHSHAAPTAGFDGGSPVAPSLMAASSYIAPSPWTETDSWGHEGGRSVPQLPPDDVPLGEVHSGSSSHHGQFSGELELALQGNRPAPGSAAPPAPRNNQGSAGTFDQAGNTMDWSL

SEQ ID NO: 87 - DNA, 7686 bp, Zea mays
  source 1..7686: mol_type = genomic DNA
ttctatcctcaagcataaataaaaatcgtagcaacgtacgagcaccttgctagtagctctagaaataagtattttttgcgagaagacatgtttacaacatacaaatacagaacagacaaaccacataggaataaatcagacaccggtttgctaaattgaaacccgatgaagagcctcgccgaccccgcatcgatgaggagccccactccgccctaacgaggagcctcgctctgcatccgccagctcgaccaccacctccatggacaagtagcgaacattcgggatccttttagcccgatatagagaacgacactctccatacttgaggtcgttgccctccatacctgagccgacgatctcatcgatgtcgccttccgccctactcctggcatggtgggcggtgtggatggtggaggaggcatgaggggtagagggagggggcgtgacaggagaagaaggcgtgttggaggaaccatagatcgagaaggtgcgggtaacggggaggagaagagaaaaggtgcagggctgagaaagaacgaaccattttttgtgtgacaagcggtagtgatggataaattcttgtaaactctacgtttatgttcgttttagctgttttggggtagggaacgatgtactccattttttactataactctataaacccatggatttttttctctccagaataattgaggtatttgagtagaaagcttttactctactttaaagttgtgctctatagctttgtcaaacaggccctaatttagcagtacagtggattaaattttttaaacttgatctgaatttgataggctaccaccctccgttttaaaatagtagttgttttaactcttggtttttaatttgggacggagggagtatcaatttgttgaatttaaaggtcgattaaccgttttgtacttctgaattcgtaagtgtttggttgataaaaatataagaccatgtttggttcctttagtcactagactaactaaagtttagtcatgtataccgtttggttctagtgactaaaccggactaaagaacattaattactgtaaataatgattgtattacccctattaattagtagatgtttgttgcaaaaaagtgaagatgacagataaggtaaaaaacatattttagtcccttttagtcaccgttttggtgactaaagaattaaagtttagtcactctactttagtcagcatgtttgcttatttaggaactaaaagtgactaaactttagccacctcaaataaactaggcctaaatctgggattattttcgtgtattctcgagacagaacgatttgatatgtgggtacaggcggcctgccagccgggcttctgccactgtaccgcggcgcggtgcgccaaagatggcatgcggctgagcggcctcggcctcgctcggctcgccgccacaagcgcaagtacctggcgtctacaaaaagccctcggcaggacgccagttgttgggacgggggacggggctataccaaccagccaaaaccaagcagggaatcacaccattcaccaagcagcagatggaagcgaaaggtggcgccgcggcgtggctagctaggccggctcgtgcgcacgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctccctctctttccttcctattagaggagagagaagagggggagagccagtcagccagccagcccaagaacagcgtacgtacgttgtcaagtcccgtcgccgccgcagcagcttcgctcctcccgccccgcccaccgtcgcgctcggcggcggggagcgagcaatggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggtcgccaccccgcccccgccattgctttctcattcagttacttccttttcagtttgacagctagttgcatccgctctgcttcctgtgttttatttccgcttcttgtctagggtttatggaaacttggctaaaattaaacagttttaggttcactattatcgatgtacatcacatctatgggctatggcgatgacaattgttttttttcctgacaccttctctttctggagaatgaaggattttttttaaaaaaaaacaaagatgagaacttgctttggtatcactactatactttaatgcttggtttgaatggtgctcatgagaactttaaagttggtaggtttgatgggtgctcctaaatatgatagagtgatgtgactcagccttgcctgcctgtctgtcagtttgttctgcattagattccagtgttaacttgcctgtcagttaacaagccattactatatggaatttgccttgagcttgatcggattgtgggtggtctggtccccaaaggaacaccactgaggaatttcaaaggagcctttgccgatgtggttgagtattaatctagagttccaactttacaagttgataatagtgctcattgcttagtctgcctcaaaaatagcactagacattatttgtcccctgtacttaaccaaactacatttggggtccttcacaaaaaaaaaacctacattaggcatgatcagttggccctcagtggtccccaaggaagaacaggcttgtctttgccactgtattattcttggtgttacaactttacaggttgtatgatactattcatattcaatgtttagtgcctaaaaaaacatgctcctctattttggctactaatacttaacaaactaaattaaattcattcggttctagatctggagaaaaaagtctagtcccttcaggtggtacaccgaacttaggttgtttctggaatagcttgtttcagtaggatacacttagcatattcattcagaaatttgctactgatgcctaatgatagcaagtgtatggaacgatcgagctgtctggtgcctaatgatagtaagcgtatgggaagatcgatctgtattgtgtcttagtaccagaacaatgcagcagaggggaaggaaagaaaaaagtagaatgtagttacgatcctacagacagctggttgtttcggtaatttggttctctgtaatttgacaatggtagcccgtaggattaaggcagtgtttagttaacttcaccgtatcatattctgtaatatatagtttcaatttttgttaggttcgatccaagcttttggttttctttgtcgataaattagttacaacagaaaaacaaaacatgatatataatggaagtgtggaactacatgtcttaaacatcgatatgcgtaggggtaggacctgggcacaccaccctatcgaccacaccttttgtgctttgggactaggctaaaggggtgtcctaacgtccccagcaaagaaaaggtgccacacttctggatttgggctgtactaaaccacagcaccacatagccacaagccacttatagcaacaacacatcattagagaagctaagttggaccatccacctgctttgctgttaaggacacctggcttgacatttgcatggaaatctcattggtggatgtgcattgttgtgttaggcccggcatctcacatctctgtgaatgattacatgcactttctctttgctttcttttagtatggatgtgcaggtgtaattatgagtgacataagacagaggttaatcttttaatggatgttcagtactggcctagcccagtaggaagagaaatgttactcttgagggcttgtacgttttgctctcaatccatatagattgggtgagatcgagtggatttaaatcctaaacaagtgaaaatctttcataattttttccaatcccattcaatccacatgggataggaataaccgaacaagcccgaatgtatagcattcccactggctttgacctaaagtatggttttttatatctatgtcgcactatgcatgttgctactaagataagttgcgctattaaggtaaactagcactttgaacttactattatcccatcattcaacttgcatttcattctattgtaagtgttattatggttgatgcttgatccatgaatttaatacttcctgcttcgtttgcattatacacaaaaattatcaatctacatcaataatatatgtggatttggagaatgcaactagcttgtattcctcataaaaccctgctactgtgcctcctcgcctgtaagttgaatcaaggacgcttgagtgaccacatctctctgtccacactgatcacaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtggtacaaaaaagctgcttcatttctttctacttggtgacttgggtgaccaaagtttcagttttttcgtatctgttgctaatgttcctacgcttgatccagaagagcccggcggcaggttcagaagcttcatgctggatttctcatacccaagggttccaagcgccatgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctaggaggccaaataaaagcgcggcacatcgtcgtagccgcacagcgagatggagctggaagcctggctccgctagctagctacatgcgctgtttgcttgcctagaggctagagcatccagtatgataataagcgaggaactagtgcagtgccggccaggagactgatttcttttgcgtttgtccatgaactcgtctgttgcgtctatgtacacctccatggtttgaactcgtcggggactttgctcgatgtgttgttattaatctcatgctcatccttccgctaatgttgcctggcggcatcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcggcacctttcctgtgaaaaaagagattttattgtttccttcctcctgtctcgaatcacagcttgtcgtgcagctagctgttccgatctctctcgctcgttttcgtccagtccctccctgttcgctgatcgagcaggcgtttgcggttttctccccagtgagtagagacagcgagtgttctggatctgtcgtacaggtcggggactgtagctgccggtgcacagcgtgcgtgattcagggccccgcccaaacaggccgcaggaacccacacactagaacaccaatgaaagctgcatcagcctcagatgcggcagcctgtagactacgtagatcctgcgacgatgaacagcgaccgtggatcctcagacgacgacgcacggtacagagatcctatcaccgcactgtagatgctcgaggaaatagctagggctggggcgttcacgagtgccagctgatacagacccgaggagtgtaagatgtttccgttggagaatccactcactcagtttttttttcttagtagcatgtaaatgcaacagctgaagttgaagaaaagtagccgattttacagactgaagctttgagagtttgcacacccctctcgagcattgcacacctccattggaaaatcccatcctgaagccaccgtcgtcttccctcggagagcggcggcgcttgttgctgggcctaaatgtatgttaaggccggcccatgtggtggtttatggggaggatccacttccgactgggggattcggccttgctggtcgcatcgggatgggtgggtccagctacgagtaggggttaggctatctgcaagcagtatccctaaatttttcctctatatcactttttccctctattttcccctattttttcatctcccgcagcggttcctccctaaatactccccatataccccactataactactattttctatatcaactatcaattttttatctactaacaattactcgtggatccacaacacagtgtttggggatgaacagtgacacgctatatctgggggagagagaagggggccggcgggtaggggacgctgtagggggcagcgctgcggccgtagagtatccctacagacgccatacaagggagggggctgccgagggggcagcgttgcagttagccttacaacgtttgatagacaccatttttatgttaccagttggagtcttttgagaagtctatagcagacaagtaaacaaaaataacgagacagactcttcctgaactatctctacacgactttatatatataatctcttagctatatttatattgtttttaactatatttataatataggagttctagttgtgttgtcggcaaatagtcgctgcaaactaagcctcagtccaaaaaggtcgccacgagataagatactttcagagggcgagtgtacacgtataccggtgactagtcttgccttttttttaaaaaaaacgtggaagttcttcctcttattatttcaaaaaaaacaaag

SEQ ID NO: 88 - DNA, 1233 bp, Zea mays
  source 1..1233: mol_type = other DNA
atggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtgagcccggcggcaggttcagaagcttcatgctggatttctcatacccaagggttccaagcgccatgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctag

SEQ ID NO: 89 - protein, 410 aa, Zea mays
MEAGGASGGDDGDGQLHGLKFGKKIYFEDAGGSSSSGGSGSASATAPPATQQPSPPAASPRAAPGGGRRGRAAAGGAGPSLPPAPPRCLVDGCNADLTDAKTYYCRHKVCEMHSKEPRVVVNGLELRFCQQCSRFHNLAEFDQQKKSCRKRLAGHNERRRRPPPGPLASRYGRLAAPLGEPGGRFRSFMLDFSYPRVPSAMRDGFPAVRPGGERVPGSSIQWQAASLDPPPPHHQSAAAGYGAHSYGSPGSSSSSSRPPVFPAGPELPPGGGCLAGVPTDSSCALSLLSTQPWDAAGHSAGHGHGHGHAASLPATAGFDGNPVAMASSYIAPPSPWTDSRAHEGGRRNVPQLPPDVPLGDVHSGPGTHHGQFSGELELALQGNRPAPAPRNGQGSAGAFDQAGSTTEWSL

SEQ ID NO: 90 - DNA, 29 bp, Zea mays
  source 1..29: mol_type = genomic DNA
cctggcgtctacaaaaagccctcggcagg

SEQ ID NO: 91 - DNA, 30 bp, Zea mays
  source 1..30: mol_type = genomic DNA
gatggaagcgaaaggtggcggtggcgcggc

SEQ ID NO: 92 - DNA, 38 bp, Zea mays
  source 1..38: mol_type = genomic DNA
ggcgcggctagctaggccggctcgtgtgcgtgcgcgag

SEQ ID NO: 93 - DNA, 54 bp, Zea mays
  source 1..54: mol_type = genomic DNA
ttttaaaggcaacagcgagtcccgctgctgtccctctccttcgccacctccctc

SEQ ID NO: 94 - DNA, 36 bp, Zea mays
  source 1..36: mol_type = genomic DNA
cctgcctgtctgtcagttcgctttgcattagattcc

SEQ ID NO: 95 - DNA, 61 bp, Zea mays
  source 1..61: mol_type = genomic DNA
attggtgggtgtgtactgttgtgttaagcccggcatcacacatctctatgaaatgattaca

SEQ ID NO: 96 - DNA, 101 bp, Zea mays
  source 1..101: mol_type = genomic DNA
gcctggcattgccaagaatcggttccaagggggtgttgtttaagtaaagcgagaccacaggcacagcgcggctttgcagcatgctcttggcaccagtggtg

SEQ ID NO: 97 - DNA, 27 bp, Zea mays
  source 1..27: mol_type = genomic DNA
gcggtgcgccaaagatggcatgcggct

SEQ ID NO: 98 - DNA, 58 bp, Zea mays
  source 1..58: mol_type = genomic DNA
gatggaagcgaaaggtggcgccgcggcgtggctagctaggccggctcgtgcgcacgcg

SEQ ID NO: 99 - DNA, 94 bp, Zea mays
  source 1..94: mol_type = genomic DNA
ttttgaaggcaacagcgagccctgctgtccctctgcttcgccacctcctccctccctctctttccttcctattagaggagagagaagaggggga

SEQ ID NO: 100 - DNA, 39 bp, Zea mays
  source 1..39: mol_type = genomic DNA
tttgaatggtgctcatgagaactttaaagttggtaggtt

SEQ ID NO: 101 - DNA, 51 bp, Zea mays
  source 1..51: mol_type = genomic DNA
tgtgcattgttgtgttaggcccggcatctcacatctctgtgaatgattaca

SEQ ID NO: 102 - DNA, 90 bp, Zea mays
  source 1..90: mol_type = genomic DNA
ggcatcgccaggaatcggttccaaggcagcgttgttcatgtaaagcgagaccgcaggctttgcagcgtgctctcagcaccagtggtgtgg

SEQ ID NO: 103 - DNA, 48 bp, Zea mays
  source 1..48: mol_type = genomic DNA
cacggtacagagatcctatcaccgcactgtagatgctcgaggaaatag

SEQ ID NO: 104 - DNA, 23 bp, synthetic construct
  misc_feature 1..23: note = Synthetic oligonucleotide
  source 1..23: mol_type = other DNA
tttactacattgataaaatttca

SEQ ID NO: 105 - DNA, 23 bp, synthetic construct
  misc_feature 1..23: note = Synthetic oligonucleotide
  source 1..23: mol_type = other DNA
tttcgttgagactaatagttaat

SEQ ID NO: 106 - DNA, 23 bp, synthetic construct
  misc_feature 1..23: note = Synthetic oligonucleotide
  source 1..23: mol_type = other DNA
tttagtactagctaactagagtg

SEQ ID NO: 107 - DNA, 23 bp, synthetic construct
  misc_feature 1..23: note = Synthetic oligonucleotide
  source 1..23: mol_type = other DNA
tttaattaaattgataaaatttc

SEQ ID NO: 108 - DNA, 23 bp, synthetic construct
  misc_feature 1..23: note = Synthetic oligonucleotide
  source 1..23: mol_type = other DNA
tttcgttgagacttgagagtagt

SEQ ID NO: 109 - DNA, 23 bp, synthetic construct
  misc_feature 1..23: note = Synthetic oligonucleotide
  source 1..23: mol_type = other DNA
tctagctactagctaacgagagt

SEQ ID NO: 110 - DNA, 23 bp, synthetic construct
  misc_feature 1..23: note = Synthetic oligonucleotide
  source 1..23: mol_type = other DNA
aaccaatgagagaaattgtctgt

SEQ ID NO: 111 - DNA, 23 bp, synthetic construct
  misc_feature 1..23: note = Synthetic oligonucleotide
  source 1..23: mol_type = other DNA
gttctctgactacactcactcac

SEQ ID NO: 112 - DNA, 23 bp, synthetic construct
  misc_feature 1..23: note = Synthetic oligonucleotide
  source 1..23: mol_type = other DNA
gttctctgactacactcactcac

SEQ ID NO: 113 - DNA, 23 bp, synthetic construct
  misc_feature 1..23: note = Synthetic oligonucleotide
  source 1..23: mol_type = other DNA
aagcaatgagatgagaggaaatt

SEQ ID NO: 114 - DNA, 23 bp, synthetic construct
  misc_feature 1..23: note = Synthetic oligonucleotide
  source 1..23: mol_type = other DNA
acttgacagaagagagagagcac

SEQ ID NO: 115 - DNA, 23 bp, synthetic construct
  misc_feature 1..23: note = Synthetic oligonucleotide
  source 1..23: mol_type = other DNA
tcttgacagaagagagagagcac

SEQ ID NO: 116 - DNA, 23 bp, synthetic construct
  misc_feature 1..23: note = Synthetic oligonucleotide
  source 1..23: mol_type = other DNA
tggccaaacttcaagccatcaag

SEQ ID NO: 117 - DNA, 23 bp, synthetic construct
  misc_feature 1..23: note = Synthetic oligonucleotide
  source 1..23: mol_type = other DNA
ctactcccgctacctctcttact

SEQ ID NO: 118 - DNA, 23 bp, synthetic construct
  misc_feature 1..23: note = Synthetic oligonucleotide
  source 1..23: mol_type = other DNA
gtgcgaagtagatctgagtaatg

SEQ ID NO: 119 - DNA, 23 bp, synthetic construct
  misc_feature 1..23: note = Synthetic oligonucleotide
  source 1..23: mol_type = other DNA
aggatgttggtcttgctactcca

SEQ ID NO: 120 - DNA, 23 bp, synthetic construct
  misc_feature 1..23: note = Synthetic oligonucleotide
  source 1..23: mol_type = other DNA
gtgcaaagtagatctgagtgatg

SEQ ID NO: 121 - DNA, 23 bp, synthetic construct
  misc_feature 1..23: note = Synthetic oligonucleotide
  source 1..23: mol_type = other DNA
aacaagaggtggggtttctgctt

SEQ ID NO: 122 - DNA, 23 bp, synthetic construct
  misc_feature 1..23: note = Synthetic oligonucleotide
  source 1..23: mol_type = other DNA
tgccatgcactccaagtccccta

SEQ ID NO: 123 - DNA, 23 bp, synthetic construct
  misc_feature 1..23: note = Synthetic oligonucleotide
  source 1..23: mol_type = other DNA
acaacaacaactcaaggggccta

SEQ ID NO: 124 - DNA, 23 bp, synthetic construct
  misc_feature 1..23: note = Synthetic oligonucleotide
  source 1..23: mol_type = other DNA
taccatgcactccaagttcccta

SEQ ID NO: 125 - DNA, 23 bp, synthetic construct
  misc_feature 1..23: note = Synthetic oligonucleotide
  source 1..23: mol_type = other DNA
ggccagggaacaccggcggcctc

SEQ ID NO: 126 - DNA, 23 bp, synthetic construct
  misc_feature 1..23: note = Synthetic oligonucleotide
  source 1..23: mol_type = other DNA
ggccagcagggaacaccggtggc

SEQ ID NO: 127 - DNA, 23 bp, synthetic construct
  misc_feature 1..23: note = Synthetic oligonucleotide
  source 1..23: mol_type = other DNA
cgcggactccagctgtgctctct

SEQ ID NO: 128 - DNA, 23 bp, synthetic construct
  misc_feature 1..23: note = Synthetic oligonucleotide
  source 1..23: mol_type = other DNA
cacggactctagctgtgctctct

SEQ ID NO: 129 - DNA, 23 bp, synthetic construct
  misc_feature 1..23: note = Synthetic oligonucleotide
  source 1..23: mol_type = other DNA
gtgtctgtctccgcgcgctcgca

SEQ ID NO: 130 - DNA, 23 bp, synthetic construct
  misc_feature 1..23: note = Synthetic oligonucleotide
  source 1..23: mol_type = other DNA
tctctctctcagcaccagccacc

SEQ ID NO: 131 - DNA, 23 bp, synthetic construct
  misc_feature 1..23: note = Synthetic oligonucleotide
  source 1..23: mol_type = other DNA
aaacaactctctctctctctctc

SEQ ID NO: 132 - DNA, 23 bp, synthetic construct
  misc_feature 1..23: note = Synthetic oligonucleotide
  source 1..23: mol_type = other DNA
tctgcttcgccacctcctccctc

SEQ ID NO: 133 - DNA, 23 bp, synthetic construct
  misc_feature 1..23: note = Synthetic oligonucleotide
  source 1..23: mol_type = other DNA
tgtatggctgtggctgtctcctc

SEQ ID NO: 134 - DNA, 23 bp, synthetic construct
  misc_feature 1..23: note = Synthetic oligonucleotide
  source 1..23: mol_type = other DNA
tgaccgtgctggtggtggtgggc

SEQ ID NO: 135 - DNA, 23 bp, synthetic construct
  misc_feature 1..23: note = Synthetic oligonucleotide
  source 1..23: mol_type = other DNA
tctctcttcttccactccctcct

SEQ ID NO: 136 - DNA, 23 bp, synthetic construct
  misc_feature 1..23: note = Synthetic oligonucleotide
  source 1..23: mol_type = other DNA
gctgctgtccctctccttcgcca

SEQ ID NO: 137 - DNA, 23 bp, synthetic construct
  misc_feature 1..23: note = Synthetic oligonucleotide
  source 1..23: mol_type = other DNA
aaggcagcgttgttcatgtaaag

SEQ ID NO: 138 - DNA, 23 bp, synthetic construct
  misc_feature 1..23: note = Synthetic oligonucleotide
  source 1..23: mol_type = other DNA
tcagtcagcctgtctatcggcac

SEQ ID NO: 139 - DNA, 23 bp, synthetic construct
  misc_feature 1..23: note = Synthetic oligonucleotide
  source 1..23: mol_type = other DNA
ctgagaaaatttctgtagatttg

SEQ ID NO: 140 - DNA, 23 bp, synthetic construct
  misc_feature 1..23: note = Synthetic oligonucleotide
  source 1..23: mol_type = other DNA
aagggggtgttgtttaagtaaag

SEQ ID NO: 141 - DNA, 23 bp, synthetic construct
  misc_feature 1..23: note = Synthetic oligonucleotide
  source 1..23: mol_type = other DNA
cgcagcagcttctttcaaatcca

SEQ ID NO: 142 - DNA, 23 bp, synthetic construct
  misc_feature 1..23: note = Synthetic oligonucleotide
  source 1..23: mol_type = other DNA
ctgagaaaattttgtggatttga

SEQ ID NO: 143 - DNA, 9440 bp, Glycine max
  source 1..9440: mol_type = genomic DNA
caaaaaatacaaatcataaaagacactcattaaataaaaagtaagacttcaaaattttataatttgtaattatttcaaccaaacaattttaaaaaatttcagaaaaaattattaaagaagtacatataacgtttctcaaacatataaaaataaacattcacaacgttgtctaagcaagtgcatgagatgttgtaattgtatttcaattcttataataaataaaaagtttgaatagctttttagttcttgtaatttagcattttttgcttttcattcatgcaatttttttttgttttcaatccttgcaaattatgcttgttttattttcagtccttaaaatgatttagatagtttttttttctatttaaagtattatctaaactgctttaagaaaaaaaaaacaaatacaatttgttagctgtgacgaacacacatgtaaggttttggggacaattgccccacattttttttatttgtatgtgtaataaatttttttcacccccataaaaaataggtattgccccctttaagacaactttcttttaaatgaacgtataaaatgaatcaaaacaaagaataaattcatactaatttcactactttatccttttaatttttataatattgataataaaaaaaattatagaatatttttgaagtatgaaaaatactagatgaccattgtaatttgtataagtttaaacattttgaatttttttgtcataacattatgtattttgtaaaattttgtgatatttttattttgcgaaaatattataaactattggttgggttaattatgtttttggttcctaaactttttgtaaaattcccttgtagtctctaaacaatttttgtaatgtttttggtccctataatttctttcattaatagttctagtttctattatcaagcaacaatgttaatttatgacataaccaatcacattaacttgtcacaccatcaagccgtgatgacaattaagtgtaattctatgaggaatttaaaatacaatttataaaaaaaattacatatgattgttccatcaaacccttatgatgtgataagtatgctgctaacatgaatattacattatcaattaatgttgttactcaataacaagtattaaaaccatcaaaggggaagagtaaaagtataaggaccacacatactaaaaaattgtttaaagactaaaagtaaaatttgtaaaaagttttggaaaaaaatataattaatcctaaactatttcttggtcttatgaagatttttttttggattcacagttattttcaaggactaaaacaattttttttctttaaaaaattaaaaataaaaaatactaaatcacaacaactaaaaagtatttaaatctaaataaaaaaaaaggaaaaaaaagtcttttgacaccaacaaactgacaaattgagagacatagaaagaaaaatagataaaaataagagttactaataagatgtttaaaagaaaaagatgttaaaatatcaccactcaaaaaataaacattgacatcagtactccatttactaaattgataaaatttcagcattaaattgataacattgacattattaattattactcacaactaaatgaattaatgtataactagctagtactagagggagcatcagcatcagcactgattagccagcagcagcaataggttctggttcagtcaccaaagcaccaccctcttcaaggactaaataattatattctttaaaaaacaaaataaaaaaaatactaaattacaacaactaaaaagtatttaaatttaaaaaaaataaggaaaaaaaatcttttgacactaacaaattgacaaattgagagacatagaaagaaaaagaaaaaaaaaatattaatataataaataacgtgatcagttaaaaaaaatagataaaaataaaaatgactaataagatgtttaaatgaaaaagatgttaaaacatcaccacttaaaaaataaacattgacatcagtactccatttactaaattgataaaatttcagcattaaattgataacattgacattattaactattagtctcaacgaaatgaattaatgtatacacccactcactctagttagctagtactaaagggagcatcagcatcagcactcagtagccagcagcagcaatgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctcttatctctttctctctctctcactttctacttttttatttattattgacttcaaaaagaaataaaaaaatcttcttttgtttttttttttttttttagttggcttgttatattgggtagttctgcttctgctttggtttattttcctcatcttactccttggtttctgctatttttagtgtttttgctattttgggtgcttttggaatactgctatgctattcacactctctttttagaaaaaaaataatggtttttgtttttctttccaatttttacatcctattcttttgtggttttggctgtatcttttacattgcttgtttttcatttcagggctttaatcgtgctgattttgttctctgtagattttttttattttattttttatgttgattgcgtgtaatgagtcatgctcttctcttacttcgtaatgaagtgtgtgaaggtttgtttttctatggtttctaatgctgcttaatgattggagaatggttcacgtttctggtttgatgcatctgttggtgctgtgagttgtagatgatagcataaccatggggttgtgttgtttatcttgtagttaacttatgggtagtgatttgcgaaggaatatgctttagaaaacaatggtgctgttgtgagcttgtgttgtttcccccttcccctttcatggtatcatgaagtgctcattaaaggaaaacggagctactagggtaggaaataggtgtagaaatgagtgcaaaacaacaactaagttgcataacatttaaagcaatgtagagtggactcagataaatgaaaagtttaggattctgttttgacataccatgccttcaagagtgtcaagatttggaacaataagtccataatatacaattttttcccttttaatttgttatgtgaagttgatattgtccttttgttcatgagatgatacctctctcatgcaagttcattccataagaaaagtgctttttccccaacattatcttatgcagatcatgcaatgcccccataaggtcatagggtctgatgttgtatctttgtcttgctggacaatgcaatccatggtaacttgctattgtgactgttagataactcaatgggacttgaatcagttaattacagaaataacagttagaaactgttatatgtttaactcaagcttgattacaactgaaattactttactccaatttcattgttgctagcaaatgctacagggcattttagggatttgcttaatggcaaaatgtccctaacaagtgtgtaattttggaggtggatggttatcattttttgggtggttttctgactgtaagatgcaaaatctggcctatttgtttgcagtattttttgtgacaataagaatgaggtgaaacaaaagagccatttgagttctatgatctgttggacattgtgtaaaatgttgcagatctcttccgacaaatgtccaaaccaattactcttattgggtttgagatattttagagaatgattctcgtcctgcagagctaaaattttcaaattaagatgcaacctccctttgccaacttgttctctaagttcctattgatggtctggtcttgtgggatccatatcgtagatcattttagaaaagaatctcttctagatgacttgatattgaacattatatgttgtagacacatgtgaccttcaatccattcaagatgcaacatgttctcaggggaaagatagatctcattgactgacaattatgggatgttaattacttatgttgaataatgatgtttagcaactaaaaggtgtaggaaatttctggatgctctcccagtatggatatagtacttgttgagaactatggcataattcttgtagctaacccctttgaatgtgaaaaaaattgtttggttgttgattgctgctcttctgttttatggcatggttggaacattttttttttatcatctgatgtgtggtttattggtaattaattgaagttgaactttctttcactggtgtaaagatgtagatggaatggtgtgtaaggaaaaatttgatggctacttagtatgaaaaatttgatgaaaagtaccacaaaaatagacatggctgatttccatttgatttttttataagtaatctaatttgttaattagcagaaagcctcttctttgattttgtgcaaattcatttgtaatgttttcatatcacatagcaccggtcggtgtgtgaatgctatttacaattattagacttgcaggttcctttccacagaagctacattggtgtgtatatatatatatatatatatatatatatatataacatattctgtgtttctctactgtgcttaactcaagttcaaatgttttcttactttacaatgagtttaaacatgccttgaggtaacgtgctaattaagctttatctgggctcgtaataggatgttatgatattctatagttgttgtgaccttaaattggaatagtattaacatatggtgttaaaaaatttcaaatgttgctctcttttctccaaatctcatttgtcaattctctgaggctatgatgatctggaaaacttgtgttacaatgggagtttcaagtttcaactgctttggggtttatcggcagttcagcacaagtctatgaatgtggtaagcaaccactgttgttgtggagcttggatagctctctgatggctgtgaacaatgaagtgtaacttttatccctttttgggctttttgtcccgtcaagggttgaaaaggattttattgcacatatttcataccaatttaaggtgcatactattttccataatgaaattaatcagaaagagggatcaacaaatgcatagtgctgttatgaatgttcaagacagtggattaaaaaggttttggagagtacaaatgggcatttttcagaactactaatttccgcattgcagatgagcatatggtatttggtgaaacaaaaggtcatctattcttattttggtaacagagtgaatctatattttaaggtttgtatcttgtattcaagctcaacgacttctctttcaattatgctagtatactgtataaattgtactaaacataatgttatccctcttagcaagaagcgagcgagaaatttttggtatctatgttttagtactgatatccatattgaagttgagttcgaagtatcttggtaccatgggatgttccctgattgcatgttgagaggattttttgctgatgctatcacaacccttgtatccaatgcattaagatttctgtaagatatgggaattgcatcttatggaaatatacgtatgttcttggctttatacatattagctttgttctgtacttttgtcacagttaggaagtatgagtgataaattctctcatttgtaatgtgatcatttaatttaatgaaagtataaaatacttcaaatgcttccaattctgtggtaataacaaatcaacagcattgcatgtaatgattagaagcaattttgtcatgttagaaattctcaaatcttgtgtcatttcgtgttttcttttgtagtatacttttacttaaacttggtagcaattatatttcagacatcttgtgtctgatcatactctctttgtatataaatctggaagcatttatacttcagaaaagctaatatcctctttcatggccctttcccaggtttcatctgctttctgaatttgatcaaggaaaacgtagctgccgcaagcgacttgctggccataatgagcgccgacgaaagcccccaccaagctccatattaacctctcgctatggcagactctctgtgcctatttttggttagaaattatcagtaaaacatcaattccaaaccactctctctgctttatgtatccctaggaagcatcaataatttcagtttttgctattgcatataaaacattcaactgtttcccttatgtttaacagataacaatggtcgagacgatggctttctgatggaatttgcttcataccccaagcttactttgagaaatacattgccaactcaaagatcaactgagccggctcctgggaaccatgctgcaacactcacctggcaaggaggttcagacactccatctgaatttttcctgcaaggatcagggagtagtggtggaacaagcttccttagttcaaaacatcccttagtggaaagttacactggagtcactgactcaaactgtgctctctctcttctgtcaagtcaaacctggggttccagaaacacagccccagcaacaagttttgaactgaacaacaacttgttcaatttcaatgggacatccatgacacaatttgctgtggcgtctttgcaagctgcagccatccatcaacttccaaatgctacctcatggttgtacctaaagggagttgattctggtaattgttcgcctgagcgtgtccccaatcacggtcttgctcaaatttctccccctcttcatagccatgttcatagtgagcttgatgtgtcacagcagggcaggaggcattacatgatctaggggagtccagggcctgtgagtcttctcactggtctctttgaagccccttctttgctatcagaaagtatgaggggaatctgtgcctatttgatcacctaactactagcattttggtttctttgaacttagaagtaaggtgggtatatccttcccttgtttgtggaacattgaaaattgaaacatccttattttggcttgtttgaacttataagcaaggtaggtagccttgtttctttgtggtacttttcagacattactagtttggtgttggaacttagaagcaagatgggtagtcttgttttattttgtggaaatttcagacctgtatcatttgaatgtgggtatgattgatgttttatagcatctacgtctgtgattccactgactgcatattccttatattgctatagctgtgctagaactactttcgagtttttttgttagaatctatagattaaatgataataataagtcttttggtattaggattaggattatgatttttaaaagactttttttttcaataaaaaatatatagattaaatgataacgataagtcttttggtatttaattaagttttttaaaatgaatttccatgaatttcatgcgatcttttaatatgatatacaaatatcgcacctattaaacaatcttatccaatttagatttttttttctcttttttgttggttgtcacacctttctctatcccatcacacatgttccattctctctctctctctctctctctctctctctctctatatatatatatatatatatatatatatatatatatatatatatatatatatatatatatatatatatattgcatcttaagtttaccaaacaaataatgaagacaataattttgaatcaagagcgaggacaagaagattctaaagcatggaggattagtttagaagaagatagtactagaattaattgacttaaaaaaaaataaagaaaaaaagtgtgatggaaaaatgttcttgaaaagcttgtccataaagagaagacattaattaaaaaagtgtgatgtaaagtaattttatatttttattcaattgcaaatcatcatttgtatgatttttaaagtgattatcataaaattcagtggatttatcatacatgatagattgtggttggatgacaataaatttattttacactattggtgcattgcctagtttctctgaatcatatcgtatctttatcatttttcatggactcttgtaattttgaatgttttctaagaatcttgaatccttacaaacttttaaattcatacaatcctttcaaatattttaatttcttatgttataaattcattaaaattcaaactataaaatcttaattacaaaaatcttttaaaaagttttgataagtcataacattctcacatagttttttaaaatgcatacaattttttatgtcaaaatagtcttttaaaatttcaatccaatatatatccctaaaagaaaataaaagataaagatgaagagtaatttttttcaaacttacttttggagtaacatgatttttctctctctctctctctctctctctctctctatatatatatatatatatatatatatatatatatatatatgttttatatagtgggttttgccacccattacatcaactcccttgtggatcctcctgagcccaaaagagaaaaccacaagagagttgctaggtgcacccagcagaattgttggtgcacccaacaattatgtgaatgggcaaaattgtccttgtttaaaagtttaaaaaataaacgcgcccattcagtgttgcttcttccgtgagtgcatccatctaccatgtctgctcgtcttcttcgcgcttctccttgttctccgtgcctcggctgccatttccgttcgttttgggaggttttttggtgccatttctgtgtttccaccgtcgaggttagtacctcctatctcccctcccctttaatgtccaatggtgatttagtttaggattttttggctgtagggtagatgacttcgtttctctgcaagtttgtctgcgaaaatggtgaagttggagcgttttccgtttacggaagaacttattccgtatgaaggaaattcttctaacagaagaagtttttccgtaatttcctacggaagaaggttcttccgtatgaagtaaatctttgtatggaagaaccttcttccgtattaacttagggaagaacttctttcgtagactcttacagaagaacttctttcgtatgactcttacggaagaacttcttccgtaagaaggtttccaatttttttattttttaattaattttttttataaattaagttttatttctttgttttgatttataataggtatatttagttttttaataataaaaaattgta

SEQ ID NO: 144 - DNA, 1023 bp, Glycine max
  source 1..1023: mol_type = other DNA
atgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtttcatctgctttctgaatttgatcaaggaaaacgtagctgccgcaagcgacttgctggccataatgagcgccgacgaaagcccccaccaagctccatattaacctctcgctatggcagactctctgtgcctatttttgataacaatggtcgagacgatggctttctgatggaatttgcttcataccccaagcttactttgagaaatacattgccaactcaaagatcaactgagccggctcctgggaaccatgctgcaacactcacctggcaaggaggttcagacactccatctgaatttttcctgcaaggatcagggagtagtggtggaacaagcttccttagttcaaaacatcccttagtggaaagttacactggagtcactgactcaaactgtgctctctctcttctgtcaagtcaaacctggggttccagaaacacagccccagcaacaagttttgaactgaacaacaacttgttcaatttcaatgggacatccatgacacaatttgctgtggcgtctttgcaagctgcagccatccatcaacttccaaatgctacctcatggttgtacctaaagggagttgattctgcagggcaggaggcattacatgatctaggggagtccagggcctgtgagtcttctcactggtctctttga

SEQ ID NO: 145 - protein, 340 aa, Glycine max
MGSASVTKAPPSSSSSPNSSSTESLDGLKFGQKIYFEEVSVAAAAATTTQGGNKTRGGVSASKKGRGGSVQAPRCQVAGCKVDLSGAKAYYSRHKVCAMHSKSPTVTVAGLEQRFCQQCSRFHLLSEFDQGKRSCRKRLAGHNERRRKPPPSSILTSRYGRLSVPIFDNNGRDDGFLMEFASYPKLTLRNTLPTQRSTEPAPGNHAATLTWQGGSDTPSEFFLQGSGSSGGTSFLSSKHPLVESYTGVTDSNCALSLLSSQTWGSRNTAPATSFELNNNLFNFNGTSMTQFAVASLQAAAIHQLPNATSWLYLKGVDSAGQEALHDLGESRACESSHWSL

SEQ ID NO: 146 - DNA, 254 bp, Glycine max
  source 1..254: mol_type = genomic DNA
aaaaaaatagataaaaataaaaatgactaataagatgtttaaatgaaaaagatgttaaaacatcaccacttaaaaaataaacattgacatcagtactccatttactaaattgataaaatttcagcattaaattgataacattgacattattaactattagtctcaacgaaatgaattaatgtatacacccactcactctagttagctagtactaaagggagcatcagcatcagcactcagtagccagcagcagc

SEQ ID NO: 147 - DNA, 156 bp, Glycine max
  source 1..156: mol_type = genomic DNA
acattgacatcagtactccatttactaaattgataaaatttcagcattaaattgataacattgacattattaactattagtctcaacgaaatgaattaatgtatacacccactcactctagttagctagtactaaagggagcatcagcatcagcac

SEQ ID NO: 148 - DNA, 116 bp, Glycine max
  source 1..116: mol_type = genomic DNA
tttactaaattgataaaatttcagcattaaattgataacattgacattattaactattagtctcaacgaaatgaattaatgtatacacccactcactctagttagctagtactaaa

SEQ ID NO: 149 - DNA, 223 bp, Glycine max
  source 1..223: mol_type = genomic DNA
aaaaaaatagataaaaataaaaatgactaataagatgtttaaatgaaaaagatgttaaaacatcaccacttaaaaaataaacattgacatcagtactccatttactaaattgataaaatttcagcattaaattgataacattgacattattaactattagtctcaacgaaatgaattaatgtatacacccactcactctagttagctagtactaaagggagca

SEQ ID NO: 150 - DNA, 143 bp, Glycine max
  source 1..143: mol_type = genomic DNA
aaatgaaaaagatgttaaaacatcaccacttaaaaaataaacattgacatcagtactccatttactaaattgataaaatttcagcattaaattgataacattgacattattaactattagtctcaacgaaatgaattaatgta

SEQ ID NO: 151 - DNA, 103 bp, Glycine max
  source 1..103: mol_type = genomic DNA
catcaccacttaaaaaataaacattgacatcagtactccatttactaaattgataaaatttcagcattaaattgataacattgacattattaactattagtct

SEQ ID NO: 152 - DNA, 63 bp, Glycine max
  source 1..63: mol_type = genomic DNA
acattgacatcagtactccatttactaaattgataaaatttcagcattaaattgataacattg

SEQ ID NO: 153 - DNA, 206 bp, Glycine max
  source 1..206: mol_type = genomic DNA
aagatgttaaaacatcaccacttaaaaaataaacattgacatcagtactccatttactaaattgataaaatttcagcattaaattgataacattgacattattaactattagtctcaacgaaatgaattaatgtatacacccactcactctagttagctagtactaaagggagcatcagcatcagcactcagtagccagcagcagc

SEQ ID NO: 154 - DNA, 142 bp, Glycine max
  source 1..142: mol_type = genomic DNA
atcagtactccatttactaaattgataaaatttcagcattaaattgataacattgacattattaactattagtctcaacgaaatgaattaatgtatacacccactcactctagttagctagtactaaagggagcatcagcat

SEQ ID NO: 155 - DNA, 103 bp, Glycine max
  source 1..103: mol_type = genomic DNA
attgataaaatttcagcattaaattgataacattgacattattaactattagtctcaacgaaatgaattaatgtatacacccactcactctagttagctagta

SEQ ID NO: 156 - DNA, 63 bp, Glycine max
  source 1..63: mol_type = genomic DNA
aaattgataacattgacattattaactattagtctcaacgaaatgaattaatgtatacaccca

SEQ ID NO: 157 - DNA, 161 bp, Glycine max
  source 1..161: mol_type = genomic DNA
tactccatttactaaattgataaaatttcagcattaaattgataacattgacattattaactattagtctcaacgaaatgaattaatgtatacacccactcactctagttagctagtactaaagggagcatcagcatcagcactcagtagccagcagcagc

SEQ ID NO: 158 - DNA, 121 bp, Glycine max
  source 1..121: mol_type = genomic DNA
gataacattgacattattaactattagtctcaacgaaatgaattaatgtatacacccactcactctagttagctagtactaaagggagcatcagcatcagcactcagtagccagcagcagc

SEQ ID NO: 159 - DNA, 101 bp, Glycine max
  source 1..101: mol_type = genomic DNA
ctattagtctcaacgaaatgaattaatgtatacacccactcactctagttagctagtactaaagggagcatcagcatcagcactcagtagccagcagcagc

SEQ ID NO: 160 - DNA, 63 bp, Glycine max
  source 1..63: mol_type = genomic DNA
aattaatgtatacacccactcactctagttagctagtactaaagggagcatcagcatcagcac

SEQ ID NO: 161 - DNA, 441 bp, Glycine max
  source 1..441: mol_type = genomic DNA
catcagcactcagtagccagcagcagcaatgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctcttatctctttctctctctctcactttctacttttttatttatt

SEQ ID NO: 162 - DNA, 281 bp, Glycine max
  source 1..281: mol_type = genomic DNA
actcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttg

SEQ ID NO: 163 - DNA, 241 bp, Glycine max
  source 1..241: mol_type = genomic DNA
cttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtccccta

SEQ ID NO: 164 - DNA, 112 bp, Glycine max
  source 1..112: mol_type = genomic DNA
cttgatggcttgaagtttggccagaaaatctactttgaggaagtgagtgttgctgcagcagcagcaacaacaactcaagggggcaacaaaacaagaggtggggtttctgctt

SEQ ID NO: 165 - DNA, 152 bp, Glycine max
  source 1..152: mol_type = genomic DNA
aacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtccccta

SEQ ID NO: 166 - DNA, 223 bp, Glycine max
  source 1..223: mol_type = genomic DNA
catcagcactcagtagccagcagcagcaatgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaaggga

SEQ ID NO: 167 - DNA, 143 bp, Glycine max
  source 1..143: mol_type = genomic DNA
tcagtcaccaaagcaccaccctcttcctcctcatcacccaactcttcttctactgagtcccttgatggcttgaagtttggccagaaaatctactttgaggaagtgagtgttgctgcagcagcagcaacaacaactcaaggggg

SEQ ID NO: 168 - DNA, 103 bp, Glycine max
  source 1..103: mol_type = genomic DNA
ctcttcctcctcatcacccaactcttcttctactgagtcccttgatggcttgaagtttggccagaaaatctactttgaggaagtgagtgttgctgcagcagca

SEQ ID NO: 169 - DNA, 63 bp, Glycine max
  source 1..63: mol_type = genomic DNA
actcttcttctactgagtcccttgatggcttgaagtttggccagaaaatctactttgaggaag

SEQ ID NO: 170 - DNA, 223 bp, Glycine max
  source 1..223: mol_type = genomic DNA
ctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaa

SEQ ID NO: 171 - DNA, 143 bp, Glycine max
  source 1..143: mol_type = genomic DNA
ctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaag

SEQ ID NO: 172 - DNA, 103 bp, Glycine max
  source 1..103: mol_type = genomic DNA
ttgctgcagcagcagcaacaacaactcaagggggcaacaaaacaagaggtggggtttctgcttcaaagaagggaaggggtggctcagtgcaagctcctaggtg

SEQ ID NO: 173 - DNA, 63 bp, Glycine max
  source 1..63: mol_type = genomic DNA
acaactcaagggggcaacaaaacaagaggtggggtttctgcttcaaagaagggaaggggtggc

SEQ ID NO: 174 - DNA, 223 bp, Glycine max
  source 1..223: mol_type = genomic DNA
agggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctcttatctctttctctctctctcactttctacttttttatttatt

SEQ ID NO: 175 - DNA, 143 bp, Glycine max
  source 1..143: mol_type = genomic DNA
tgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctctta

SEQ ID NO: 176 - DNA, 103 bp, Glycine max
  source 1..103: mol_type = genomic DNA
tgagtggtgctaaggcttactattctaggcacaaagtttgtgccatgcactccaagtcccctactgtcactgttgctggtttggagcaaaggttttgccaaca

SEQ ID NO: 177 - DNA, 63 bp, Glycine max
  source 1..63: mol_type = genomic DNA
tattctaggcacaaagtttgtgccatgcactccaagtcccctactgtcactgttgctggtttg

SEQ ID NO: 178 - DNA, 223 bp, Glycine max
  source 1..223: mol_type = genomic DNA
gaatttttcctgcaaggatcagggagtagtggtggaacaagcttccttagttcaaaacatcccttagtggaaagttacactggagtcactgactcaaactgtgctctctctcttctgtcaagtcaaacctggggttccagaaacacagccccagcaacaagttttgaactgaacaacaacttgttcaatttcaatgggacatccatgacacaatttgctgtgg

SEQ ID NO: 179 - DNA, 143 bp, Glycine max
  source 1..143: mol_type = genomic DNA
gcttccttagttcaaaacatcccttagtggaaagttacactggagtcactgactcaaactgtgctctctctcttctgtcaagtcaaacctggggttccagaaacacagccccagcaacaagttttgaactgaacaacaacttg

SEQ ID NO: 180 - DNA, 103 bp, Glycine max
  source 1..103: mol_type = genomic DNA
cccttagtggaaagttacactggagtcactgactcaaactgtgctctctctcttctgtcaagtcaaacctggggttccagaaacacagccccagcaacaagtt

SEQ ID NO: 181 - DNA, 63 bp, Glycine max
  source 1..63: mol_type = genomic DNA
tggagtcactgactcaaactgtgctctctctcttctgtcaagtcaaacctggggttccagaaa

SEQ ID NO: 182 - DNA, 9050 bp, Glycine max
  source 1..9050: mol_type = genomic DNA
aaattgattgttgttgcttcttctgcattctaagcctatccctcttaagattattgaggccacaaggtctaacacagtgaaggattctgaccagcaaggatgttgagtgaagtgtcaccgcatgaagtgtcatagtgtgagagccgcaaagaagtaaaaagttagggatgttacgccactcgttaacccctgtgatttttcactctttttctcttgtcacgtcattggaaatgaatgtcatcatgctaaattttaacataaatttatgctattcagttggataaaatgaaaataaagaaatatttttagtaagaaattatatttaacaagaaaaaatatcactatttcatatcatgctagagaacacatgttaaaaaatatgaagacaaaaagaatcaagagaccttgacaaattctaacatgtgtgactaaagtatgatagtgaggtatgacatctatttatattgtgtatcttttaacgatttcgataactttttgttttgttaacattgtctaggagtgatgtgtattatgttgttcctacttgtgtgtgtagtgatttcattgcattctactcacattaagtatctagaatcccagtattagacttaggtgtggtttgtattggtttctctcacttacgttgttagaatggttttatcgcatcttacttatgcattcaagtatctagaagcctagtattgagcctagatgtagttttatagcaccctactcaggtgttaagtatctagaatcctagtattgactttgttaacctttattgtcatttgtcaatgaattgcgtattatattgctcctatccgtgtgtagagtgattttatcacgctactcacgtgtttggtattggatctaggttcttcaaacttatgagctttgttagcttacttaggtgatccattgaataatgaatttggttaggtcatcctctcaaatttatcttttattttgaacattttggtcaacctattatgattggaggttcacaacttcttaatcgttgagcttaaaagatgtaaatcctggggtgattagtcactaactagacaatatacaacccatcaaacactgaacctaaaaataaatatttgttgtgattgatttctctaactagccagtccataaataattatttaatttctaaaaaaataaaacattttagttataactcaagaatgaaaagaataatgtatttaaatttatagttctagataaaatcattttcttattttactttaaattaaattaagtgattttttatataaaagaatattttatttgtattaaattaaaattaattttttggtatgaaattattaactatttttaaattattttaaatatatttttaaaaataattattataaaaccaaaatattattatacaaaacactttgtgatttaataaccataagtgatagcaacacattttataacactctatttaatatatttttattatagattaaaatttattaaaaattacaaatgacacttattaagaaaaattaaaaactaaatcattaagtatatgaataaactaaaataagattattttaatttaataaaaaatctcaagttcaaatcttatgttaaaactgtttaaaatacttagaaaaagaattttatcacctataatgatattttttaactcaaatataattattatttttggataatacttgtgatctaaataaaaaattaacttataatattttataatttgtaataaatttcaatcaaataattaaaaaatattttaaaagcaaataacgtttctcaaatatataaaaataaacattaaccatcaaaacgtctaactaattaattgatcagtttctatgaattattctaatattcttttaattcttataaataaataaacattaaatcattactccatttaattaaattgataaaatttcagcattaaattgatgacattgacattattattaactactactctcaagtctcaacgaaatgaattaatgtatatacactcactcactctcgttagctagtagctagagggagcatcagcatcagcatcagcactgagtagcagcagcaggagcaatgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtcaccatcctctctctctctctctctttaccttttgtttatttattattgacttcaaaaagggaaaaaatcttcttttgtttttgttactcttttgtttttgctatttttagctggcttgctatattgggtaattctacttctgctttggtttcctttcctcatcttacttccattgtttttgctatttttagtgcatttgctatactgctatgctattcacatgctcttgttttttagaagaaaaatggttttgtttttcattccaatttttacatccctttgttgtgtggtttttggctgtatcttttacatttgttgcttttcatttcaggaatttaatcatgctgattttgttccttgtgattgttctctgtagattttttttaagttgattgcgtgtaatgagtcttgcacttctcttacttcataatgaagtgtgtgcaaggtttgttttactttggtttctaatgtttctttatgattggataattgttaatgtgtctggatttgatgcatctattggtgctgtgagttgtagatgataacataaccatggtgttgtgttgtttatcttgtagttatggttagtgattggcgagggaatatgctttagaaaccaattgcttttgtgagcttgtgttgtttcttctttcctccttcatggtatcatggagtgctcatgaaagggaaaggggagttctcagcgaaagaaaaaactgtacaaatgagtgtgaaacagctgcataacatgtgaatcaatgtaaattggactctgataagtgaaaaatttagggtagtttagacataccaagccttcaagagggtcaatatataaatcaagatttggaataataagaccatgcaatactattttcccatttttaacttgttatttgctcatgaaatgatacctctctcatgtaatgtaagttcattccacaagaaaagtgcttttttccccacattattgtcatatacaaatcatgtaaggcctccttaaggtaggtagggtctaatgtcctatctttgtcttgttaacaactcaatggaaatgactaaaattaatgttactggataattcaattaacttgcttgtgactgttagatacctcaatggggcttgaatcaaagatgtaagattgtaagtgaaaagggaatagggttagaagggagaggaagagagggatatcttttggaagttagatggttaacagaagagggactgggctctcaaattccgagtgcagagagaagagtggtctgtgttctcattgttcaatatagaattatcattcttgttaccgaattctgatctagatcttaagacagtatccattttctgttcagtagtgaaagcttgattacatctgaatttactttcctcttcaatttgactcatcattgctagcaaatgttacagggcattttagggatgtgtttaatggcaaaatgttactaacaagtgggtagttttggagatggatggttatcataatttggatagttttctgaccataagatttggaatacttggcctacttgtttgccagtatttttggacaatagttatgaggtgaaataaaagagccacttaagtgacctgattggttgggcattgcataaaatgttgcagatttgtcctccactgacaaaccaattatatttttccagattaagatattttagggaattattctcttcatgcagggctaaaattttcaaaaggaattgccaacatgtgctctaagttcctattgatggttgatttcatacaccttcgttcatttgtcttgtgtattgtggtattcatatgaagatcatattagaaaataatctcttctaaaactacttaaaattgaacattcaagccattcaagatgctgtatgttctctggtgaaagataaaccttgtcgattgacaattgtgggatgttaattacttatgtggaataatggtgtttagcaactaaaaggtatagggatttttttggatgttctcctggcatggatatagtacatgttaagcactatagaataatggtgtttagcaactaaaaggtatagggattcaaatgtttttttaaacttaataacaagagtttaaacataccttgaggtagtgtgctaatgaagctttgtctgggctcttaatatgatgttatgttattatattctatggttgttgtgacttgtgaccttaaattggaatggtattatctcatggtgttaaaaatctcaaatgtcattctttttcctccaaatctcatttttcaattctttgatgctatgattatctggagaacttgtgttacaatgagagtttcaactgttttggggcttatcagcacaaatctattaatgtggtaagcttccactattgttagggagcttagatagctctctggtggctgtttcctgtgaagtattagtctattaatgtcccttttgggcttttgttccgtccaaggtcgaataggattttattgcacattttccataccctctttaaggtgcttcctattttccataatgaaattaaccagaaagagggaagggatcaataaatcgacagtgctgttatgaatcaagacagtgtttaaaaaggttttggagacatagcacagatggatgttttcaaatctactattttcagcattgtagatgagcaaacacgttaatgggcgaaacaaaatgtcatgtattcttattttggtaagaaagtgaatgtatattttaaggtctgtattctgtattcaagctcaatgcttctctttcaattatgcaagtatatattgtaccaaacatgaaatttaagttcgaagtatcttggatgggatgttccttgattgtttgttgagaggattttttgctgatgccatcacaatccttagtttaaaacatttgtgacttaggctgtatttccattatgatatactgtatccaatgcattaagatttctgataagatatgggaattgcttcttatggaaatatacctatggtattggctctatacatattagctttgttctgtaatgttgtcacaatgactaagttttactgataaattctctcatttgtaatgtgaatttaatataatgaaaggagaaatagttcaaatgcttccaattctgtggtaacaacaggtcaacagcattgcatgtaatgctaagaagcgattttgtcatgttagaattctcaaatcttgtgtcatctcatgttctcttttatagtaatgttttacttgctagaaattatatttcagaaatcttgtgtctgatcatactctttggtatcatgcttttatataaacatggaagcatttatatttcagaaaagttaatatcctctctgatggcccttgcccaggtttcatctgctttctgaatttgatgaaggaaaacgaagctgccgcaggcgacttgctggtcataatgagcgccgacgaaagcccccgccaagctccacactagcctctcactatggcagactctctacgcctatttttggttagaaattatcagtaaaacatcaattcgaaaccactctctctgcttaatgtatccctaggaagtaggaagcatcaataatttcagtttttgctattccttataaaacattcaactgtttcacccatgtttaacagataacagtggccgagccggtggctttctgatggaatttgcttcatacccaaaggttacattgcgaaatacactgccaacacaaagatcagccgagccggctctcggtaaccatgctgcaacacttacctggcaaggaagctcagacacaccatctgaatttttcctgcaagaatcagggagtggtggaacaagcttccttggttccaaacatcccttactggaaagttacactggagtgactgactcaaactgtgctctctctcttctgtcaagacaaacctggggttccagaaacacagccccagcaacaagtgttgagctgaacaacaacttgttgaatttcaatgggacatccatgacacaattttccgcggcttcttcacaagctgcagccatccatcaacttccaaatgccacctcatggttgtacctaaagggagttggttctggtgattgttcgcctgagggtgtccccgatcttggtctaggtcaaatttcgccgcctcttgatagccatcttcatggtgagcttaatgtgtcacagcagggcaggaggcattacatggatctaggggaatccagggcatgtgagtcttctcactggtctctttgacgcacttctatgctatctgttactatttgatcagttaccagactggcactaactactagcattttggtttctttgaacttagaagcaaggtgggtatatccttcttttggttgtggaactttgaaaattgaaacatccttattttggtttgtttgaacatatcagcaaggtaggtagccttgttttttttttttttttttgtggtacctttcagacattattagtttggtattggaacttagaagcaagatggggttgtcctgttttattttgtggaaatttcagacgtgtaaatcattttattgtgggtatgatcgatgttttctagcatatatgtctgtgattccactgaatgcatattccttatattgttatagctgtacttggagttatttgttaaaatcattagattaaggggatgtattggattagtattttaaagaattttaaaagattttttttataaaaaaatagaaatttaaatgttaaaaataagtcttttagtatacaattaagattttttttaccttcaaaaaagtcttatgatatttaaaagtatataatttttcattttgatgaattattttttaaaataaatttcattggattttatgtgattttttagtaggatatctaaatattttacctattaaacaatcttattcaaatcctccagatttttctggactttcttgattgattgtaacacctttctatttctcatcacacatatttccttctacttagtagaagatgttatatatacattcataactttttttcacaaagaatgttgttcaaacaaatttccaattgaacaaataggttaataccaaataatgaagacaatagttttgaaccaagagtgagaatgagaagattttaatgcatggagggttaatatagaaagatatgaaactagaatcaatagactaaaaaaaaattgttgtggaatttttttgaaaacttgtccataagcagaagccataaattaattggcttaattgtgcttttaattctctgacttttcaatttttgatgaattttatcctttaagatacttgctaattttattctatgccaatttattcctaacataactttactttttacttcaattttttatttatttttttggcaaattttacactcgacttttttttttaatcctatcattttatatttttggtaaattttaccccaacattttatatttactaatggataaatgataaggggtaaaagttgcaagtttttcaaaaattgggaataaaatgtgtcaaattaatttggaggataaaattcataaaaaaagaaaaagttaagataaaaaatgtaattaaatataattaattaaataacaattttggtgtatgaaagtagctcaaggtcaaagacattcttagagtaatatactgatattttgaataattacattaccatatatttatctaattgaaatttaatttatttgaaaaaattgaattgattttacttttttaaacattagagtttaatggttatgtccggataatgtatactaattttagacggtgattcaatcataaataattgttataactttgaaagtaattatcataaaaatctacaaacatgatgtgttttaattggattaaaaatattacattaacatatatttatctaatttaaatttaatttatttgaaattgtactaattttactttttttatttaaaccttagagtttaatattatgtactgataatgtaaattaattttagactattattcaatcataaatagttgttgatataacttttaaagtgattatcacaaaaatctacaaacatgatgtgttgtaattggattgacgataaaattattttacactattagtgcattgcctaatttctctaaatctcatattatctttatcattttcctttgacttttgtaattttgaatgttttctagaacttcgtgatctttacaaatcttttaaatttatacaatccttgttggataagtggcctcaataacttaaggagggggggggggggtgaattaagtttcaaaattttcccgttaacaaatgttaaccccattttaaatgatatatgataggctcagaatgcaaaaagaagaagaagcaatcaatttaataatgttttttttaaatgcacaagataaagtaaactataataaaataactgagataagggaagagagaattacaaacttgttttatcctcgttcggccacttcccgtgcctacgtccagttcttaagtaactcacttgatatttttcactatctctgtaaatcctttatagattttgaac

SEQ ID NO: 183 - DNA, 1110 bp, Glycine max
  source 1..1110: mol_type = other DNA
atgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtttcatctgctttctgaatttgatgaaggaaaacgaagctgccgcaggcgacttgctggtcataatgagcgccgacgaaagcccccgccaagctccacactagcctctcactatggcagactctctacgcctatttttgtttttgctattccttataaaacattcaactgtttcacccatgttacattgcgaaatacactgccaacacaaagatcagccgagccggctctcggtaaccatgctgcaacacttacctggcaaggaagctcagacacaccatctgaatttttcctgcaagaatcagggagtggtggaacaagcttccttggttccaaacatcccttactggaaagttacactggagtgactgactcaaactgtgctctctctcttctgtcaagacaaacctggggttccagaaacacagccccagcaacaagtgttgagctgaacaacaacttgttgaatttcaatgggacatccatgacacaattttccgcggcttcttcacaagctgcagccatccatcaacttccaaatgccacctcatggttgtacctaaagggagttggttctggtgattgttcgcctgagggtgtccccgatcttggtctaggtcaaatttcgccgcctcttgatagccatcttcatggtgagcttaatgtgtcacagcagggcaggaggcattacatggatctaggggaatccagggcatgtgagtcttctcactggtctctttga

SEQ ID NO: 184 - protein, 369 aa, Glycine max
MSSGSVTKAPPPSSSSSPNSSSESLDGLKFGQKIYFEDVSVAATTTTQGAYKTSRVVSSPSSASKKGRGGSLQPPRCQVEGCKVDLSGAKAYYSRHKVCTMHSKFPTVIVAGLEQRFCQQCSRFHLLSEFDEGKRSCRRRLAGHNERRRKPPPSSTLASHYGRLSTPIFVFAIPYKTFNCFTHVTLRNTLPTQRSAEPALGNHAATLTWQGSSDTPSEFFLQESGSGGTSFLGSKHPLLESYTGVTDSNCALSLLSRQTWGSRNTAPATSVELNNNLLNFNGTSMTQFSAASSQAAAIHQLPNATSWLYLKGVGSGDCSPEGVPDLGLGQISPPLDSHLHGELNVSQQGRRHYMDLGESRACESSHWSL

SEQ ID NO: 185 - DNA, 277 bp, Glycine max
  source 1..277: mol_type = genomic DNA
aaccatcaaaacgtctaactaattaattgatcagtttctatgaattattctaatattcttttaattcttataaataaataaacattaaatcattactccatttaattaaattgataaaatttcagcattaaattgatgacattgacattattattaactactactctcaagtctcaacgaaatgaattaatgtatatacactcactcactctcgttagctagtagctagagggagcatcagcatcagcatcagcactgagtagcagcagcaggagca

SEQ ID NO: 186 - DNA, 130 bp, Glycine max
  source 1..130: mol_type = genomic DNA
tttaattaaattgataaaatttcagcattaaattgatgacattgacattattattaactactactctcaagtctcaacgaaatgaattaatgtatatacactcactcactctcgttagctagtagctaga

SEQ ID NO: 187 - DNA, 82 bp, Glycine max
  source 1..82: mol_type = genomic DNA
tttaattaaattgataaaatttcagcattaaattgatgacattgacattattattaactactactctcaagtctcaacgaaa

SEQ ID NO: 188 - DNA, 71 bp, Glycine max
  source 1..71: mol_type = genomic DNA
actactctcaagtctcaacgaaatgaattaatgtatatacactcactcactctcgttagctagtagctaga

SEQ ID NO: 189 - DNA, 223 bp, Glycine max
  source 1..223: mol_type = genomic DNA
aaccatcaaaacgtctaactaattaattgatcagtttctatgaattattctaatattcttttaattcttataaataaataaacattaaatcattactccatttaattaaattgataaaatttcagcattaaattgatgacattgacattattattaactactactctcaagtctcaacgaaatgaattaatgtatatacactcactcactctcgttagctagt

SEQ ID NO: 190 - DNA, 143 bp, Glycine max
  source 1..143: mol_type = genomic DNA
tgaattattctaatattcttttaattcttataaataaataaacattaaatcattactccatttaattaaattgataaaatttcagcattaaattgatgacattgacattattattaactactactctcaagtctcaacgaaat

SEQ ID NO: 191 - DNA, 103 bp, Glycine max
  source 1..103: mol_type = genomic DNA
ttaattcttataaataaataaacattaaatcattactccatttaattaaattgataaaatttcagcattaaattgatgacattgacattattattaactacta

SEQ ID NO: 192 - DNA, 63 bp, Glycine max
  source 1..63: mol_type = genomic DNA
aacattaaatcattactccatttaattaaattgataaaatttcagcattaaattgatgacatt

SEQ ID NO: 193 - DNA, 218 bp, Glycine max
  source 1..218: mol_type = genomic DNA
tttaattcttataaataaataaacattaaatcattactccatttaattaaattgataaaatttcagcattaaattgatgacattgacattattattaactactactctcaagtctcaacgaaatgaattaatgtatatacactcactcactctcgttagctagtagctagagggagcatcagcatcagcatcagcactgagtagcagcagcaggagca

SEQ ID NO: 194 - DNA, 143 bp, Glycine max
  source 1..143: mol_type = genomic DNA
atttaattaaattgataaaatttcagcattaaattgatgacattgacattattattaactactactctcaagtctcaacgaaatgaattaatgtatatacactcactcactctcgttagctagtagctagagggagcatcagc

SEQ ID NO: 195 - DNA, 103 bp, Glycine max
  source 1..103: mol_type = genomic DNA
tttcagcattaaattgatgacattgacattattattaactactactctcaagtctcaacgaaatgaattaatgtatatacactcactcactctcgttagctag

SEQ ID NO: 196 - DNA, 63 bp, Glycine max
  source 1..63: mol_type = genomic DNA
cattgacattattattaactactactctcaagtctcaacgaaatgaattaatgtatatacact

SEQ ID NO: 197 - DNA, 170 bp, Glycine max
  source 1..170: mol_type = genomic DNA
aaattgataaaatttcagcattaaattgatgacattgacattattattaactactactctcaagtctcaacgaaatgaattaatgtatatacactcactcactctcgttagctagtagctagagggagcatcagcatcagcatcagcactgagtagcagcagcaggagca

SEQ ID NO: 198 - DNA, 130 bp, Glycine max
  source 1..130: mol_type = genomic DNA
ttattattaactactactctcaagtctcaacgaaatgaattaatgtatatacactcactcactctcgttagctagtagctagagggagcatcagcatcagcatcagcactgagtagcagcagcaggagca

SEQ ID NO: 199 - DNA, 103 bp, Glycine max
  source 1..103: mol_type = genomic DNA
caagtctcaacgaaatgaattaatgtatatacactcactcactctcgttagctagtagctagagggagcatcagcatcagcatcagcactgagtagcagcagc

SEQ ID NO: 200 - DNA, 63 bp, Glycine max
  source 1..63: mol_type = genomic DNA
taatgtatatacactcactcactctcgttagctagtagctagagggagcatcagcatcagcat

SEQ ID NO: 201 - DNA, 447 bp, Glycine max
  source 1..447: mol_type = genomic DNA
tcagcactgagtagcagcagcaggagcaatgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtcaccatcctctctctctctctctctttaccttttgtttatttattattg

SEQ ID NO: 202 - DNA, 287 bp, Glycine max
  source 1..287: mol_type = genomic DNA
ccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttg

SEQ ID NO: 203 - DNA, 247 bp, Glycine max
  source 1..247: mol_type = genomic DNA
cttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttcccta

SEQ ID NO: 204 - DNA, 80 bp, Glycine max
  source 1..80: mol_type = genomic DNA
cttgatggcttgaagtttggccagaaaatctactttgaggatgtgagtgttgcagcaacaacaacaactcaaggggccta

SEQ ID NO: 205 - DNA, 190 bp, Glycine max
  source 1..190: mol_type = genomic DNA
acaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttcccta

SEQ ID NO: 206 - DNA, 223 bp, Glycine max
  source 1..223: mol_type = genomic DNA
tcagcactgagtagcagcagcaggagcaatgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaag

SEQ ID NO: 207 - DNA, 143 bp, Glycine max
  source 1..143: mol_type = genomic DNA
tcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaa

SEQ ID NO: 208 - DNA, 103 bp, Glycine max
  source 1..103: mol_type = genomic DNA
accctcttcttcttcatcacccaactcttcctctgagtcccttgatggcttgaagtttggccagaaaatctactttgaggatgtgagtgttgcagcaacaaca

SEQ ID NO: 209 - DNA, 63 bp, Glycine max
  source 1..63: mol_type = genomic DNA
ccaactcttcctctgagtcccttgatggcttgaagtttggccagaaaatctactttgaggatg

SEQ ID NO: 210 - DNA, 223 bp, Glycine max
  source 1..223: mol_type = genomic DNA
accaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagta

SEQ ID NO: 211 - DNA, 143 bp, Glycine max
  source 1..143: mol_type = genomic DNA
tcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttc

SEQ ID NO: 212 - DNA, 103 bp, Glycine max
  source 1..103: mol_type = genomic DNA
tggccagaaaatctactttgaggatgtgagtgttgcagcaacaacaacaactcaaggggcctacaaaacaagtcgtgtggtttcttccccttcttctgcttca

SEQ ID NO: 213 - DNA, 63 bp, Glycine max
  source 1..63: mol_type = genomic DNA
aggatgtgagtgttgcagcaacaacaacaactcaaggggcctacaaaacaagtcgtgtggttt

SEQ ID NO: 214 - DNA, 223 bp, Glycine max
  source 1..223: mol_type = genomic DNA
agggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtcaccatcctctctctctctctctctttaccttttgtttatttattattg

SEQ ID NO: 215 - DNA, 143 bp, Glycine max
  source 1..143: mol_type = genomic DNA
tgagggctgcaaagtagatctgagtggtgctaaggcttactattctaggcacaaagtttgtaccatgcactccaagttccctactgttattgttgctggtttggagcaaaggttttgccaacagtgtagcaggtcaccatcct

SEQ ID NO: 216 - DNA, 103 bp, Glycine max
  source 1..103: mol_type = genomic DNA
tgagtggtgctaaggcttactattctaggcacaaagtttgtaccatgcactccaagttccctactgttattgttgctggtttggagcaaaggttttgccaaca

SEQ ID NO: 217 - DNA, 63 bp, Glycine max
  source 1..63: mol_type = genomic DNA
tattctaggcacaaagtttgtaccatgcactccaagttccctactgttattgttgctggtttg

SEQ ID NO: 218 - DNA, 223 bp, Glycine max
  source 1..223: mol_type = genomic DNA
tctgaatttttcctgcaagaatcagggagtggtggaacaagcttccttggttccaaacatcccttactggaaagttacactggagtgactgactcaaactgtgctctctctcttctgtcaagacaaacctggggttccagaaacacagccccagcaacaagtgttgagctgaacaacaacttgttgaatttcaatgggacatccatgacacaattttccgcgg

SEQ ID NO: 219 - DNA, 141 bp, Glycine max
  source 1..141: mol_type = genomic DNA
agcttccttggttccaaacatcccttactggaaagttacactggagtgactgactcaaactgtgctctctctcttctgtcaagacaaacctggggttccagaaacacagccccagcaacaagtgttgagctgaacaacaac

SEQ ID NO: 220 - DNA, 101 bp, Glycine max
  source 1..101: mol_type = genomic DNA
tcccttactggaaagttacactggagtgactgactcaaactgtgctctctctcttctgtcaagacaaacctggggttccagaaacacagccccagcaacaa

SEQ ID NO: 221 - DNA, 60 bp, Glycine max
  source 1..60: mol_type = genomic DNA
tggagtgactgactcaaactgtgctctctctcttctgtcaagacaaacctggggttccag

SEQ ID NO: 222 - DNA, 8477 bp, Glycine max
  source 1..8477: mol_type = genomic DNA
attgccgaattaagctgacaaaaaataatcaaacttaaaagtctcaaaataattacggaactactaaaccaaaggagctccttctgtgcgatgtaaacatttttttcttatggtttaaaaatataaatatataaattgaaaaaagtaattaaaattgacaatttcttaatatgcgttttctcttatttattattttcatatgtatatattattattttaaaacgtatattaattaatgttattttaaccttttaaaataaaaaatttccttatgacacgtaataaaaagaaacaaaagtaacatgttaagttctaggttttcattagtaattaataagtaaataaataataaataaagtgtttcattttcatattttgtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcgagctgtgctctctctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggagaacaaggcatgtttggttttcagtaaggaagactcctatgcaggttttaataattaaaaccaaaaggggtgttcggttttcagctgaatctgattgtaaactgagttttgtctcaattttgttttgaagtttagtttgaaatgagagaaaaggtgggcttgtttttgcaaaactaaattcgtttaaatttgagtttgtaaactttaatccaaacaagcaaatttgttcaaaagtcagttaatttcgtaacacaatgttgagtttagtgaatgtttagattaagatttgtaaatgcaagttctaactcaaaatttcatttagcaaattgagtttgcaaaactagtcttttcttcttcaagttttcattttaaattgagtttcaaggaaaaatttaggtaaatctcagttcacaatcgactttaatttaaaccaaaggtttttcagttttactttttgaatagattttgggtcctcctaactgaaaactgaacaggcctttaaatgatagaagagatttatttcatttgttaatgaatgttcatatgcaacttgtttagagcttttggaagtgatatttctattttcatttttcttttccatcactctcgttctcaattttttccatcatcttcgttcaaaatttattttcctcttgttctgctgccaacctgcttgaggaaccaactgaaataaaatcattacccgtggaccaaaaataaaattgaaattccaaatccaaccttagttatgcaattaaatcccaagtcatcgtgattcatgagcactgtcctgacctttcacccttctctacaacacaattttatgcctacaaacccgccaatggaaccagtgtctactgcattgcccctgtaccattattaaagtactaccatatactattccctgcaatctacataaagatagtaaggaatgaaaccaaaattatggaaatttgaaaaatgttaccctgcaatttttatatgaataccatgataggaaagggggaatgaacttaaggagcctagtatataatgaaacaaaagggtgggggatgcaggtaaccaaaattatggaaattccaaaaatgtcatcatgatgagtgaagacatggttaaatgaccttttgttgttttcagaatttttgaaaacttggagttaatattttttcgttgcaagtgtatgcactaaggatgttccctagataattaatgttgagctggggtggatggagtgttgggagcacgtgttgaggttggttgttaggtgtggtagctaacagtgtattgtgtgacaatagtaattatttgcactttaacactcaagttaatagaaatttttggtagagtaacaacaatattgaatgatgcgtatctagagaagggagggttctgctatttataggcgttgggtggttttaattaaatgagattgaattagttaagctgtcaatcccatactgcttctgtaaacacttgtatctatgtgattcgtatgacttataaggtagttacttgaaatagtgataagaatgacactcatatctaagtgagaaggtgtcccaccttgtaaggtatatgatgtgttggattcttattgggttgaattgttggggtgtagaaacttcaaatctatttagaatggatatggatcaaatctttttcatataggtatcctaaaattcaacctttattaacatgattaaaagacttaattatttgtcaattatgttaggctttgttggcaagtttaatttttaaaattgaaaataaaaatgctctaacatattttcgttttcactttttgtttcattgaaaacataaagaaaaacagagaaatcataaagccaataaaataggccttggctgttggttttctggatggatttttgcttggcttctggattagaagcctacctttttcatttgttgaatagaagcctaccttttaataccttataagcaatatttgaataatctatttttattatcatttttctggtt

SEQ ID NO: 223 - DNA, 1104 bp, Glycine max; identical to SEQ ID NO: 79
  source 1..1104: mol_type = other DNA

SEQ ID NO: 224 - protein, 367 aa, Glycine max; identical to SEQ ID NO: 80

SEQ ID NO: 225 - DNA, 352 bp, Glycine max
  source 1..352: mol_type = genomic DNA
tagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactccttt

SEQ ID NO: 226 - DNA, 193 bp, Glycine max
  source 1..193: mol_type = genomic DNA
gttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctca

SEQ ID NO: 227 - DNA, 152 bp, Glycine max
  source 1..152: mol_type = genomic DNA
aaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcac

SEQ ID NO: 228 - DNA, 132 bp, Glycine max
  source 1..132: mol_type = genomic DNA
aaccaatgagagaaattgtctgtgttgagaagaaaaaagaaaaaaagaaaaaagagagggtacaatctgtctctcgtagctggcttggctccattattcttggtttttggacagacaagggtgtgtttggtt

SEQ ID NO: 229 - DNA, 223 bp, Glycine max
  source 1..223: mol_type = genomic DNA
tagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtg

SEQ ID NO: 230 - DNA, 143 bp, Glycine max
  source 1..143: mol_type = genomic DNA
ttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctgg

SEQ ID NO: 231 - DNA, 103 bp, Glycine max
  source 1..103: mol_type = genomic DNA
acttgttaggcctggaatggttaaaaacttggaacttttgaaccaatgagagaaattgtctgtgttgagaagaaaaaagaaaaaaagaaaaaagagagggtac

SEQ ID NO: 232 - DNA, 64 bp, Glycine max
  source 1..64: mol_type = genomic DNA
gttaaaaacttggaacttttgaaccaatgagagaaattgtctgtgttgagaagaaaaaagaaaa

SEQ ID NO: 233 - DNA, 223 bp, Glycine max
  source 1..223: mol_type = genomic DNA
gagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttac

SEQ ID NO: 234 - DNA, 143 bp, Glycine max
  source 1..143: mol_type = genomic DNA
aaaagagagggtacaatctgtctctcgtagctggcttggctccattattcttggtttttggacagacaagggtgtgtttggttctctgactacactcactcactccttctctcttctctctcagtgtacctgactctgccacc

SEQ ID NO: 235 - DNA, 103 bp, Glycine max
  source 1..103: mol_type = genomic DNA
tctctcgtagctggcttggctccattattcttggtttttggacagacaagggtgtgtttggttctctgactacactcactcactccttctctcttctctctca

SEQ ID NO: 236 - DNA, 63 bp, Glycine max
  source 1..63: mol_type = genomic DNA
tccattattcttggtttttggacagacaagggtgtgtttggttctctgactacactcactcac

SEQ ID NO: 237 - DNA, 223 bp, Glycine max
  source 1..223: mol_type = genomic DNA
aagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactccttt

SEQ ID NO: 238 - DNA, 144 bp, Glycine max
  source 1..144: mol_type = genomic DNA
tctctcgtagctggcttggctccattattcttggtttttggacagacaagggtgtgtttggttctctgactacactcactcactccttctctcttctctctcagtgtacctgactctgccaccttccctacccttccaccccct

SEQ ID NO: 239 - DNA, 103 bp, Glycine max
  source 1..103: mol_type = genomic DNA
tccattattcttggtttttggacagacaagggtgtgtttggttctctgactacactcactcactccttctctcttctctctcagtgtacctgactctgccacc

SEQ ID NO: 240 - DNA, 356 bp, Glycine max
  source 1..356: mol_type = genomic DNA
actccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtag

SEQ ID NO: 241 - DNA, 196 bp, Glycine max
  source 1..196: mol_type = genomic DNA
ttttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctaga

SEQ ID NO: 242 - DNA, 156 bp, Glycine max
  source 1..156: mol_type = genomic DNA
ctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatg

SEQ ID NO: 243 - DNA, 223 bp, Glycine max
  source 1..223: mol_type = genomic DNA
actccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtc

SEQ ID NO: 244 - DNA, 143 bp, Glycine max
  source 1..143: mol_type = genomic DNA
ctgagtccctcaacggtttgaaattcggccaaaaaatctattttgaggatgtgggttttgctactcccgctacctctcttacttcttctgctactactgctaccactgttacttcttcttcttcttcctcttcaagaaaagga

SEQ ID NO: 245 - DNA, 103 bp, Glycine max
  source 1..103: mol_type = genomic DNA
aaattcggccaaaaaatctattttgaggatgtgggttttgctactcccgctacctctcttacttcttctgctactactgctaccactgttacttcttcttctt

SEQ ID NO: 246 - DNA, 63 bp, Glycine max
  source 1..63: mol_type = genomic DNA
ttttgaggatgtgggttttgctactcccgctacctctcttacttcttctgctactactgctac

SEQ ID NO: 247 - DNA, 223 bp, Glycine max
  source 1..223: mol_type = genomic DNA
ctactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtag

SEQ ID NO: 248 - DNA, 143 bp, Glycine max
  source 1..143: mol_type = genomic DNA
aagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtca

SEQ ID NO: 249 - DNA, 103 bp, Glycine max
  source 1..103: mol_type = genomic DNA
ctgttcaaccagctcaacctccaaggtgtcaagttgaagggtgcgaagtagatctgagtaatgcaaaaacttactattctagacacaaagtttgtggcatgca

SEQ ID NO: 250 - DNA, 63 bp, Glycine max
  source 1..63: mol_type = genomic DNA
ccaaggtgtcaagttgaagggtgcgaagtagatctgagtaatgcaaaaacttactattctaga

SEQ ID NO: 251 - DNA, 223 bp, Glycine max
  source 1..223: mol_type = genomic DNA
tcatctgaccttttcttgcaaggttcggtgggtgggacaagcttcgccggcccgggacatcctccaggggaaagttacactggggtcaccacctcgagctgtgctctctctcttctgtcaaatcaaacatggggttctagaaacccagcaccaagtcttgggttgaataacatggtaaatttccacgggacgcccatgacacagcctgctgcatcatctcatg

SEQ ID NO: 252 - DNA, 140 bp, Glycine max
  source 1..140: mol_type = genomic DNA
gcttcgccggcccgggacatcctccaggggaaagttacactggggtcaccacctcgagctgtgctctctctcttctgtcaaatcaaacatggggttctagaaacccagcaccaagtcttgggttgaataacatggtaaat

SEQ ID NO: 253 - DNA, 100 bp, Glycine max
  source 1..100: mol_type = genomic DNA
cctccaggggaaagttacactggggtcaccacctcgagctgtgctctctctcttctgtcaaatcaaacatggggttctagaaacccagcaccaagtcttg

SEQ ID NO: 254 - DNA, 60 bp, Glycine max
  source 1..60: mol_type = genomic DNA
tggggtcaccacctcgagctgtgctctctctcttctgtcaaatcaaacatggggttctag

SEQ ID NO: 255 - DNA, 8896 bp, Glycine max
  source 1..8896: mol_type = genomic DNA
aaattactacttaatttcatggattgattcatttattcatgcgcttttgtaattcggatactaactgtgaaaaagttcatttattttctagattaagtttatttgataagtctaaaacacacgtttcacgggtcggaccttaaatgagacaaactagtccatatacttaccccctaaaattaatgttatcaaatttggatcagtcatcgagtcgatttagatactgggtcacaagttgacccaatgaatcactagtcgatcacatgatcccgtctatattttaaaaatcataacatgtaaacttacacttaatatataagttttaacaaaatccaacatatctagaagcataattatgaacttgtttaaaattttaagcacacaatatattattatcatctatttattaaaactatcattgttgtacacggtcaaaaataatacatacacatatgtacatagatacatgcacacacacacaaacatatatgacatatcatatgagatgaatatgattattatgagagtgaaatgattaaatttgaaccgtatatccatatatgcaaggttaagatttacattctatatttaatctgtttagtctcatataatatgctttctctaataagatatgtcatctctctctccctctctctctctctctctctctctatatatatatatatatatgacatgtcatatgagagaaaaatattttatataagagtgaaatgattaaatttgaaccatataatcaaacatgaataatcaagatttaatcttaaaaagtcatgcgaccttttaagtagttacatcaaattaaatcttaactttatacagttcaaatttaatcttctcactcccatataagatatttcctctcataagatttggtagcggcgtcaatcatattggtggcgatgatggtgatcatggtgatgatcacattggtggtagtagtggtggtaagggtgatgacaacgacaacgaccacggtcatgtgatggtgacagtagagattaacgatcatggtagtggttgcggtggagtttatgatagttatggtggaaacagtcataacatgataatggtcatggttgtgataggtttaagatatcttaggacatcacatattaaatctttaagatatatatttttttgttttgtaaatcttatggagattttataattgaataaaattttaatttgaattttaaatgttttaatcttgatctctttcattttaatctaatcgttgaaattaattattttcattttcatataagaacataagatgattattctcatatgatagatacatcttttaaacacatacacacttgtaaattagctatgaaaaaattgaaaggataataactttattgtcttagattataaaatgtggttatgccgtgatataaaaaagtatgcaaatctataaataacatgtgttgatttaattgttaaaaagaaattaaaaaatacttaccttttgagtcaatcaaatcgttgattcaagtcgaatcaaatcgattcatgtcgaatcatatgcatatcgaatccaactaatagttgagattcaataggtgtctaattctagactaatgggttgagccgagtctaataataacatgtttggataaatggcgcttaatttcaattcaattattttttcacgtttatagtaaagaactctttgtagctttgagattttcacggtgaaaaattgatttttgtggtgccaccgcagaaccaaagataccataagtcgccaatgaattgcaattgcaaggtcaagattttttttcgtttgattctgaaatgaatatataatgatgatatgttgtaaaatttgggtccatggatacatccctttcgtatgtagcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttccactttactctcccactaccactcctttcatgacttcaacttcaacaaccacctctttctctctcacttcttctcttgctcttatcaaaacaaaccactatgaataatactaacgataataatagttacaacaatacactctagcttctagcctgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtcaaaccccttctttcattattattattattattacctgaaacaaaactgaaaattctctttttcttcttcttgtgctattttaatattgaggtaagataaagtagggtagggccttgtttattttattcagtttttttgctcgttattctgcggtttggggtgtaggtttgtaggtgagatatgttatcctgttttgtcggtgcagttataggtgtgtgtgttacttgtttggagttggtttagttccttaatcgtggctttgcttttgttcaaggctttggtgtggtgtgattctttgtgtctgttcttgagatgtttgttgttcagttatgatttggctgattgtactcggtatactttacttttttttcccctcctagcgtagctttaaagtgcttatctgaacggctgtcttataacgcatgtgaaatattattgttttccttttcagcgaattgattcttgcacccccgggtgtgtttggtattaaaaataataacttcgtttgtacggaaaaaggcttgaagtgggtgtgcaatttgtagtcttaaaataacacaaacactactatgctgttttccctctagcctttgagggggccctcaattgtgtagtgaactattgatatgctgttacagttttctcattactgtttttgtgtttggattatcttttttggagaaataatatttttttttccttttatagattactagagagcctttgaaaaaaaaaaaataatgataattttccaaatataatctgttaccaaacatgaccaaactctttaggtatgtgatctgtgtaaagatacatccatgtattttactgtcatgaatcaagttgttttatagtgtgaaggcatgttatttgtactacaagagcaattccttgttgatgtgaacctaggattcttactcaaggcttgcattattatcaaaatggtagttttctgctaattggagtgagagtgactttagccttcccttgtctggtcatccataacgtgggtcctgattgttgaatttgatttatctatcttaaacagataggttgtctgattgtttatgtaatttccttgcatgaagacactagtcatgtattgcctccttgactaatgttagttttcatactgtttagtttgacttgttgatacattattgagaatgagtgcaattggtgctgctggatcatgaaatcctaaccacatgctttgcttatgtgactattcatctctattctgttttatggtgctagctatgtcagactgtcagttgtcacatgtactatccatttcaatttgactcattagtgctagaggatgctactgtgcattctaggatagtattcaatggtgtaataagcttgtattttggagacagatggccatcataattggaaagtttccaaccccaggatatggagaatatggtctatctgtcaatgaatgtggttttatatgcagtttttttttggagctgggataactttctgttggctgttctaagagtaatttgtgtctttttttttgtctcatggttcttccacagttgaaaagggttatctaagtcaaaagttacacttttccttaataatgaaaaacaatactcttctgaatagggtgacaatgaattgaaccaactcgaaaatagttcggaactcgattcaataatcagctcgttgaatttggttcatgaaccaaatgagctgaatttgagctcgttaaataaatgacccaaacttgagctgctaatagttcaactcatttgattcatgaattggctcaatatatatatatatatatatatatatatatataattgattgggttttctattattttatatatatctagggcagggtagtttttatttatgcagactttggttataattatgttttttttgtcataccttgtggccctacaaagctaatagaataagttcagttttgtcttttcacaaaaattaaacaatcattttatctaaaatctaccaataaattgaaaatttgaattgtataggatttaaaaaatttgacttttggttcatgtgatccaagctaacaagctgaattgaactgtttgtgagcttgaactgagttgagttcgagctgaaaaataaaggttcatgaaccaaaagtcaaatttttcagcaaaagatattaatgagccgaactgaactcgactcgtttccggacctacttctgaatattctaagttaaaatacatatcatgagaaagatcgtgaaaagatatttaagatggactttttagcatccaatctcccagcatttcagatgtactatcggtggatatacattgtggtttatcccctgtatttaagcttgatgacttctcttttgaattattctggtgtctttcactaaagagtaaatacaatgccatcccttttggtaatatagaagataaacaacttcattggtattgttttggtcttattttactaatacgttaaagctgaattccaagtatcatggagccgcagaatgttacttgagcaatgtttgaggattacttcagtaaaacagggctcaaggagcatcttatactgctaatatgccatctcattcaatgccaaacaagctcacatgatgtgcatcatatggaatacccttgttcttgggtttatacatattgcttatgttctgcaatctttgcaatgatgactaattcaaataagttagaaattccatctcatttgcatctgcatttaatgaaacaacaaatattttaagtgcttccaatttgtggcaacaacagcatgtcatgctaaggtaacagtagacttgtcatgttagcatttcttaagtcttggcacttgggtatgcttgtactcattcacctctctttgacataaattggaatcatttatatttgtaaaagataatatcatgtttcatagtcttgcacaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttggttagaaactatcagtaaaatatcagtttcatgtctcttccaattttttcatagggaagcatatttagtttcagcttgtgtcattcctggcatacaaaagtaaaacaaactctaatgtctggcacttttattgacagataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctgtgctctctctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcacggtgagctggacctgtcccaacagggcaggaggcattatatggatctagaacagtccagggcatatgaatctgctcattggtcactttaatgcacttgttttttttttgtcagctttgtaataacatgtagcagaaacctgtgcctacttggttacttaccagattgatgataactattgcttgttgttttgttttgttttaactgataagcaaggtgggtagccttgcctcatttgtggaactttcaggcaccgaatgaatctcacctgagagtgtgttttgttagtagtgtcttaagcacaggtatggtgttttctgccattgccgtcacctctggaattatttgtgattgggaggacagcctctttgatgtttgctaccaccgctccattttatttgatattatcttttgactatctgtcaactaagtgttgcacacattgcttgcctaagaacattttgcacagcattaagttgaatgacttggataacaaggcatgcttggttttcagttgggaggtgctcaacgtacatttcagaaggtaaaactaaaaaagctctttgtatggttttcaattgaatccaattgtaaattgggttttgtctcaaaacttagtttcaaatgaaagaaaaggtagggttgtttttgcaaaatgaagttcatataaattgaggtttgtaaactttaattaaaacatgcaacctgttcaaaaatcacttcattttttcaacacaatattagggtcacaaaacaattcagtgcatgtttacattagagtttgtaaatctaagtttgaactcagaatttcattttgctaattgagttagcaaaaagaatcttttcttctttcatttcaaattgagtatcaagacaaaattgaggtagctctcagtttataatatgatttttggtataaatcataagcatcctctagtgtaggtaatttagtttgagttagataagattactataagaagcaaagttttctctccgtagttgcatatctaggactcatttggaatctttggttaagtttgaatgatctggtgtagttgatttacgtgttggacagtagtttacaataaaactaaaactagtttttcagttttactttttgaatacatgttggggcctcctagatcaaaaagagattaattttatttattaatgaatttcggtatgcaaatcgtttagaagttacaactattggaagtgatatttctatgaataccatgataggaaagggaaaaaaacttaaaggagtctagaatagaatgaaacaaaaggggtggggatgcaggtaatcaaaattatggaaattccaaaaatgttatcatgatgagtgaagaagacatgttaaatgactttttgttgttttcagaatttttgaagatttggagtgaatattttttcgttgcaagtgtgtgcactaaggatattccctagataatttatgttgaactgaggtggacggggtgttgggagcacgtgttgaggttggttgttggatgttgagatcctctggtggttgtgacaatagtaattatttgcactttaacactcaagttaataagaattttggtagagtaataacaatattaactgatgcgtatctagagaagggagggttctgctatttataggtgttgggtggtttgaattaaatgagattggattagttaagctgtcaatctactgtcttagaatcccatacttcatctgtaaacacacatggtgtttatgcaattggtatgacttataaggtagttgcttaaaacagtagtgagaatgacactcctatcaaagtaagtgtcccaccttacaaggtatatgatgtgtagggcttggaattcgtttagaatggataatggtcgaatctttttcatgtaagcatctgaaatcgaacccttaaaagacttatttatttaccaattatgttgagttttgttggtgagtttaatttttaaaattgaaaataaaaatactcaaacatattttcttttacattttctgtttcactgaaaatataaagaaaaacagagatgatgaagcaaataaaataggcgtcggttgatggcttatttttgcttggctttcgggattataagcttgccattaataccttacaagaagtatttgaataatctatttttattatcatttttatggttggttcacatgcttccaaggaacccaacagttagtgcggaaattattgtttggtatttcaaataaaagaacccaataattagtgtgaaattattgcttggcatttcaaataaaagaggttgacttgtttagcttataaatgattttgttatcaactttgttcgaaattatttttgataaatattataggataagcaataatttttattcatattattatcattttataaattggtttggcatagcgcagca

SEQ ID NO: 256 - DNA, 1080 bp, Glycine max
  source 1..1080: mol_type = other DNA
atggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttgataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctgtgctctctctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcacggtgagctggacctgtcccaacagggcaggaggcattatatggatctagaacagtccagggcatatgaatctgctcattggtcactttaa

SEQ ID NO: 257 - protein, 359 aa, Glycine max; identical to SEQ ID NO: 83

SEQ ID NO: 258 - DNA, 346 bp, Glycine max
  source 1..346: mol_type = genomic DNA
agcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttc

SEQ ID NO: 259 - DNA, 186 bp, Glycine max
  source 1..186: mol_type = genomic DNA
ttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctca

SEQ ID NO: 260 - DNA, 146 bp, Glycine max
  source 1..146: mol_type = genomic DNA
aagcaatgagatgagaggaaattgttgagttgagaagaaaaaaaaagaagagagggtacaatctgtctctcgtagctggctggctccattatttttggttttggacagacaagggtgtgtttggttctctgactacactcactcac

SEQ ID NO: 261 - DNA, 126 bp, Glycine max
  source 1..126: mol_type = genomic DNA
aagcaatgagatgagaggaaattgttgagttgagaagaaaaaaaaagaagagagggtacaatctgtctctcgtagctggctggctccattatttttggttttggacagacaagggtgtgtttggtt

SEQ ID NO: 262 - DNA, 223 bp, Glycine max
  source 1..223: mol_type = genomic DNA
agcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttg

SEQ ID NO: 263 - DNA, 143 bp, Glycine max
  source 1..143: mol_type = genomic DNA
gcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctgg

SEQ ID NO: 264 - DNA, 103 bp, Glycine max
  source 1..103: mol_type = genomic DNA
ttgtaagggcctggaatggtttaaaacttggaactttttgaagcaatgagatgagaggaaattgttgagttgagaagaaaaaaaaagaagagagggtacaatc

SEQ ID NO: 265 - DNA, 63 bp, Glycine max
  source 1..63: mol_type = genomic DNA
ttaaaacttggaactttttgaagcaatgagatgagaggaaattgttgagttgagaagaaaaaa

SEQ ID NO: 266 - DNA, 223 bp, Glycine max
  source 1..223: mol_type = genomic DNA
caatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctaccctt

SEQ ID NO: 267 - DNA, 144 bp, Glycine max
  source 1..144: mol_type = genomic DNA
aaagaagagagggtacaatctgtctctcgtagctggctggctccattatttttggttttggacagacaagggtgtgtttggttctctgactacactcactcactccttctctcttctctctcagtgtaccacactctctgccac

SEQ ID NO: 268 - DNA, 103 bp, Glycine max
  source 1..103: mol_type = genomic DNA
tgtctctcgtagctggctggctccattatttttggttttggacagacaagggtgtgtttggttctctgactacactcactcactccttctctcttctctctca

SEQ ID NO: 269 - DNA, 63 bp, Glycine max
  source 1..63: mol_type = genomic DNA
ctccattatttttggttttggacagacaagggtgtgtttggttctctgactacactcactcac

SEQ ID NO: 270 - DNA, 223 bp, Glycine max
  source 1..223: mol_type = genomic DNA
gttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttc

SEQ ID NO: 271 - DNA, 143 bp, Glycine max
  source 1..143: mol_type = genomic DNA
tgtctctcgtagctggctggctccattatttttggttttggacagacaagggtgtgtttggttctctgactacactcactcactccttctctcttctctctcagtgtaccacactctctgccacctatggcccagcgtcaact

SEQ ID NO: 272 - DNA, 103 bp, Glycine max
  source 1..103: mol_type = genomic DNA
ctccattatttttggttttggacagacaagggtgtgtttggttctctgactacactcactcactccttctctcttctctctcagtgtaccacactctctgcca

SEQ ID NO: 273 - DNA, 63 bp, Glycine max
  source 1..63: mol_type = genomic DNA
gacagacaagggtgtgtttggttctctgactacactcactcactccttctctcttctctctca

SEQ ID NO: 274 - DNA, 371 bp, Glycine max
  source 1..371: mol_type = genomic DNA
tgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtag

SEQ ID NO: 275 - DNA, 211 bp, Glycine max
  source 1..211: mol_type = genomic DNA
cggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctaga

SEQ ID NO: 276 - DNA, 171 bp, Glycine max
  source 1..171: mol_type = genomic DNA
aggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatg

SEQ ID NO: 277 - DNA, 223 bp, Glycine max
  source 1..223: mol_type = genomic DNA
tgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctc

SEQ ID NO: 278 - DNA, 143 bp, Glycine max
  source 1..143: mol_type = genomic DNA
ccaaactctcctcttctgagtccctcaacggtttgaaattcggccaaaaaatctattttgaggatgttggtcttgctactccagccacctctcttacttcttcttcttcttcttcttctgctgctactgttacttcttcttct

SEQ ID NO: 279 - DNA, 103 bp, Glycine max
  source 1..103: mol_type = genomic DNA
tccctcaacggtttgaaattcggccaaaaaatctattttgaggatgttggtcttgctactccagccacctctcttacttcttcttcttcttcttcttctgctg

SEQ ID NO: 280 - DNA, 63 bp, Glycine max
  source 1..63: mol_type = genomic DNA
cggccaaaaaatctattttgaggatgttggtcttgctactccagccacctctcttacttcttc

SEQ ID NO: 281 - DNA, 223 bp, Glycine max
  source 1..223: mol_type = genomic DNA
cttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtag

SEQ ID NO: 282 - DNA, 143 bp, Glycine max
  source 1..143: mol_type = genomic DNA
aaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtca

SEQ ID NO: 283 - DNA, 103 bp, Glycine max
  source 1..103: mol_type = genomic DNA
ctgttcaaccagctcaacctcccaggtgtcaagttgaagggtgcaaagtagatctgagtgatgcaaaagcttactattctagacacaaggtctgtggcatgca

SEQ ID NO: 284 - DNA, 63 bp, Glycine max
  source 1..63: mol_type = genomic DNA
cccaggtgtcaagttgaagggtgcaaagtagatctgagtgatgcaaaagcttactattctaga

SEQ ID NO: 285 - DNA, 223 bp, Glycine max
  source 1..223: mol_type = genomic DNA
tcatctgaccttttcttgcaaggttcggtgggtgggacaagcttcgccagcccgggacatcctccaggggaaagttacattggggtcaccgacacgagctgtgctctctctcttctgtcaaatcaaacatggggttctagaaacacagcaccaagtcttgggttgagtaacatgataaatttcaacgggacacccttgacacaacttgctgcatcatctcatg

SEQ ID NO: 286 - DNA, 139 bp, Glycine max
  source 1..139: mol_type = genomic DNA
gcttcgccagcccgggacatcctccaggggaaagttacattggggtcaccgacacgagctgtgctctctctcttctgtcaaatcaaacatggggttctagaaacacagcaccaagtcttgggttgagtaacatgataaa

SEQ ID NO: 287 - DNA, 100 bp, Glycine max
  source 1..100: mol_type = genomic DNA
cctccaggggaaagttacattggggtcaccgacacgagctgtgctctctctcttctgtcaaatcaaacatggggttctagaaacacagcaccaagtcttg

SEQ ID NO: 288 - DNA, 60 bp, Glycine max
  source 1..60: mol_type = genomic DNA
tggggtcaccgacacgagctgtgctctctctcttctgtcaaatcaaacatggggttctag

SEQ ID NO: 289 - DNA, 8469 bp, synthetic construct
  misc_feature 1..8469: note = Synthetic polynucleotide
  source 1..8469: mol_type = other DNA
attgccgaattaagctgacaaaaaataatcaaacttaaaagtctcaaaataattacggaactactaaaccaaaggagctccttctgtgcgatgtaaacatttttttcttatggtttaaaaatataaatatataaattgaaaaaagtaattaaaattgacaatttcttaatatgcgttttctcttatttattattttcatatgtatatattattattttaaaacgtatattaattaatgttattttaaccttttaaaataaaaaatttccttatgacacgtaataaaaagaaacaaaagtaacatgttaagttctaggttttcattagtaattaataagtaaataaataataaataaagtgtttcattttcatattttgtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcgagctgctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggagaacaaggcatgtttggttttcagtaaggaagactcctatgcaggttttaataattaaaaccaaaaggggtgttcggttttcagctgaatctgattgtaaactgagttttgtctcaattttgttttgaagtttagtttgaaatgagagaaaaggtgggcttgtttttgcaaaactaaattcgtttaaatttgagtttgtaaactttaatccaaacaagcaaatttgttcaaaagtcagttaatttcgtaacacaatgttgagtttagtgaatgtttagattaagatttgtaaatgcaagttctaactcaaaatttcatttagcaaattgagtttgcaaaactagtcttttcttcttcaagttttcattttaaattgagtttcaaggaaaaatttaggtaaatctcagttcacaatcgactttaatttaaaccaaaggtttttcagttttactttttgaatagattttgggtcctcctaactgaaaactgaacaggcctttaaatgatagaagagatttatttcatttgttaatgaatgttcatatgcaacttgtttagagcttttggaagtgatatttctattttcatttttcttttccatcactctcgttctcaattttttccatcatcttcgttcaaaatttattttcctcttgttctgctgccaacctgcttgaggaaccaactgaaataaaatcattacccgtggaccaaaaataaaattgaaattccaaatccaaccttagttatgcaattaaatcccaagtcatcgtgattcatgagcactgtcctgacctttcacccttctctacaacacaattttatgcctacaaacccgccaatggaaccagtgtctactgcattgcccctgtaccattattaaagtactaccatatactattccctgcaatctacataaagatagtaaggaatgaaaccaaaattatggaaatttgaaaaatgttaccctgcaatttttatatgaataccatgataggaaagggggaatgaacttaaggagcctagtatataatgaaacaaaagggtgggggatgcaggtaaccaaaattatggaaattccaaaaatgtcatcatgatgagtgaagacatggttaaatgaccttttgttgttttcagaatttttgaaaacttggagttaatattttttcgttgcaagtgtatgcactaaggatgttccctagataattaatgttgagctggggtggatggagtgttgggagcacgtgttgaggttggttgttaggtgtggtagctaacagtgtattgtgtgacaatagtaattatttgcactttaacactcaagttaatagaaatttttggtagagtaacaacaatattgaatgatgcgtatctagagaagggagggttctgctatttataggcgttgggtggttttaattaaatgagattgaattagttaagctgtcaatcccatactgcttctgtaaacacttgtatctatgtgattcgtatgacttataaggtagttacttgaaatagtgataagaatgacactcatatctaagtgagaaggtgtcccaccttgtaaggtatatgatgtgttggattcttattgggttgaattgttggggtgtagaaacttcaaatctatttagaatggatatggatcaaatctttttcatataggtatcctaaaattcaacctttattaacatgattaaaagacttaattatttgtcaattatgttaggctttgttggcaagtttaatttttaaaattgaaaataaaaatgctctaacatattttcgttttcactttttgtttcattgaaaacataaagaaaaacagagaaatcataaagccaataaaataggccttggctgttggttttctggatggatttttgcttggcttctggattagaagcctacctttttcatttgttgaatagaagcctaccttttaataccttataagcaatatttgaataatctatttttattatcatttttctggtt

SEQ ID NO: 290 - DNA, 8889 bp, synthetic construct
  misc_feature 1..8889: note = Synthetic polynucleotide
  source 1..8889: mol_type = other DNA
aaattactacttaatttcatggattgattcatttattcatgcgcttttgtaattcggatactaactgtgaaaaagttcatttattttctagattaagtttatttgataagtctaaaacacacgtttcacgggtcggaccttaaatgagacaaactagtccatatacttaccccctaaaattaatgttatcaaatttggatcagtcatcgagtcgatttagatactgggtcacaagttgacccaatgaatcactagtcgatcacatgatcccgtctatattttaaaaatcataacatgtaaacttacacttaatatataagttttaacaaaatccaacatatctagaagcataattatgaacttgtttaaaattttaagcacacaatatattattatcatctatttattaaaactatcattgttgtacacggtcaaaaataatacatacacatatgtacatagatacatgcacacacacacaaacatatatgacatatcatatgagatgaatatgattattatgagagtgaaatgattaaatttgaaccgtatatccatatatgcaaggttaagatttacattctatatttaatctgtttagtctcatataatatgctttctctaataagatatgtcatctctctctccctctctctctctctctctctctctatatatatatatatatatgacatgtcatatgagagaaaaatattttatataagagtgaaatgattaaatttgaaccatataatcaaacatgaataatcaagatttaatcttaaaaagtcatgcgaccttttaagtagttacatcaaattaaatcttaactttatacagttcaaatttaatcttctcactcccatataagatatttcctctcataagatttggtagcggcgtcaatcatattggtggcgatgatggtgatcatggtgatgatcacattggtggtagtagtggtggtaagggtgatgacaacgacaacgaccacggtcatgtgatggtgacagtagagattaacgatcatggtagtggttgcggtggagtttatgatagttatggtggaaacagtcataacatgataatggtcatggttgtgataggtttaagatatcttaggacatcacatattaaatctttaagatatatatttttttgttttgtaaatcttatggagattttataattgaataaaattttaatttgaattttaaatgttttaatcttgatctctttcattttaatctaatcgttgaaattaattattttcattttcatataagaacataagatgattattctcatatgatagatacatcttttaaacacatacacacttgtaaattagctatgaaaaaattgaaaggataataactttattgtcttagattataaaatgtggttatgccgtgatataaaaaagtatgcaaatctataaataacatgtgttgatttaattgttaaaaagaaattaaaaaatacttaccttttgagtcaatcaaatcgttgattcaagtcgaatcaaatcgattcatgtcgaatcatatgcatatcgaatccaactaatagttgagattcaataggtgtctaattctagactaatgggttgagccgagtctaataataacatgtttggataaatggcgcttaatttcaattcaattattttttcacgtttatagtaaagaactctttgtagctttgagattttcacggtgaaaaattgatttttgtggtgccaccgcagaaccaaagataccataagtcgccaatgaattgcaattgcaaggtcaagattttttttcgtttgattctgaaatgaatatataatgatgatatgttgtaaaatttgggtccatggatacatccctttcgtatgtagcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttccactttactctcccactaccactcctttcatgacttcaacttcaacaaccacctctttctctctcacttcttctcttgctcttatcaaaacaaaccactatgaataatactaacgataataatagttacaacaatacactctagcttctagcctgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtcaaaccccttctttcattattattattattattacctgaaacaaaactgaaaattctctttttcttcttcttgtgctattttaatattgaggtaagataaagtagggtagggccttgtttattttattcagtttttttgctcgttattctgcggtttggggtgtaggtttgtaggtgagatatgttatcctgttttgtcggtgcagttataggtgtgtgtgttacttgtttggagttggtttagttccttaatcgtggctttgcttttgttcaaggctttggtgtggtgtgattctttgtgtctgttcttgagatgtttgttgttcagttatgatttggctgattgtactcggtatactttacttttttttcccctcctagcgtagctttaaagtgcttatctgaacggctgtcttataacgcatgtgaaatattattgttttccttttcagcgaattgattcttgcacccccgggtgtgtttggtattaaaaataataacttcgtttgtacggaaaaaggcttgaagtgggtgtgcaatttgtagtcttaaaataacacaaacactactatgctgttttccctctagcctttgagggggccctcaattgtgtagtgaactattgatatgctgttacagttttctcattactgtttttgtgtttggattatcttttttggagaaataatatttttttttccttttatagattactagagagcctttgaaaaaaaaaaaataatgataattttccaaatataatctgttaccaaacatgaccaaactctttaggtatgtgatctgtgtaaagatacatccatgtattttactgtcatgaatcaagttgttttatagtgtgaaggcatgttatttgtactacaagagcaattccttgttgatgtgaacctaggattcttactcaaggcttgcattattatcaaaatggtagttttctgctaattggagtgagagtgactttagccttcccttgtctggtcatccataacgtgggtcctgattgttgaatttgatttatctatcttaaacagataggttgtctgattgtttatgtaatttccttgcatgaagacactagtcatgtattgcctccttgactaatgttagttttcatactgtttagtttgacttgttgatacattattgagaatgagtgcaattggtgctgctggatcatgaaatcctaaccacatgctttgcttatgtgactattcatctctattctgttttatggtgctagctatgtcagactgtcagttgtcacatgtactatccatttcaatttgactcattagtgctagaggatgctactgtgcattctaggatagtattcaatggtgtaataagcttgtattttggagacagatggccatcataattggaaagtttccaaccccaggatatggagaatatggtctatctgtcaatgaatgtggttttatatgcagtttttttttggagctgggataactttctgttggctgttctaagagtaatttgtgtctttttttttgtctcatggttcttccacagttgaaaagggttatctaagtcaaaagttacacttttccttaataatgaaaaacaatactcttctgaatagggtgacaatgaattgaaccaactcgaaaatagttcggaactcgattcaataatcagctcgttgaatttggttcatgaaccaaatgagctgaatttgagctcgttaaataaatgacccaaacttgagctgctaatagttcaactcatttgattcatgaattggctcaatatatatatatatatatatatatatatatataattgattgggttttctattattttatatatatctagggcagggtagtttttatttatgcagactttggttataattatgttttttttgtcataccttgtggccctacaaagctaatagaataagttcagttttgtcttttcacaaaaattaaacaatcattttatctaaaatctaccaataaattgaaaatttgaattgtataggatttaaaaaatttgacttttggttcatgtgatccaagctaacaagctgaattgaactgtttgtgagcttgaactgagttgagttcgagctgaaaaataaaggttcatgaaccaaaagtcaaatttttcagcaaaagatattaatgagccgaactgaactcgactcgtttccggacctacttctgaatattctaagttaaaatacatatcatgagaaagatcgtgaaaagatatttaagatggactttttagcatccaatctcccagcatttcagatgtactatcggtggatatacattgtggtttatcccctgtatttaagcttgatgacttctcttttgaattattctggtgtctttcactaaagagtaaatacaatgccatcccttttggtaatatagaagataaacaacttcattggtattgttttggtcttattttactaatacgttaaagctgaattccaagtatcatggagccgcagaatgttacttgagcaatgtttgaggattacttcagtaaaacagggctcaaggagcatcttatactgctaatatgccatctcattcaatgccaaacaagctcacatgatgtgcatcatatggaatacccttgttcttgggtttatacatattgcttatgttctgcaatctttgcaatgatgactaattcaaataagttagaaattccatctcatttgcatctgcatttaatgaaacaacaaatattttaagtgcttccaatttgtggcaacaacagcatgtcatgctaaggtaacagtagacttgtcatgttagcatttcttaagtcttggcacttgggtatgcttgtactcattcacctctctttgacataaattggaatcatttatatttgtaaaagataatatcatgtttcatagtcttgcacaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttggttagaaactatcagtaaaatatcagtttcatgtctcttccaattttttcatagggaagcatatttagtttcagcttgtgtcattcctggcatacaaaagtaaaacaaactctaatgtctggcacttttattgacagataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctctctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcacggtgagctggacctgtcccaacagggcaggaggcattatatggatctagaacagtccagggcatatgaatctgctcattggtcactttaatgcacttgttttttttttgtcagctttgtaataacatgtagcagaaacctgtgcctacttggttacttaccagattgatgataactattgcttgttgttttgttttgttttaactgataagcaaggtgggtagccttgcctcatttgtggaactttcaggcaccgaatgaatctcacctgagagtgtgttttgttagtagtgtcttaagcacaggtatggtgttttctgccattgccgtcacctctggaattatttgtgattgggaggacagcctctttgatgtttgctaccaccgctccattttatttgatattatcttttgactatctgtcaactaagtgttgcacacattgcttgcctaagaacattttgcacagcattaagttgaatgacttggataacaaggcatgcttggttttcagttgggaggtgctcaacgtacatttcagaaggtaaaactaaaaaagctctttgtatggttttcaattgaatccaattgtaaattgggttttgtctcaaaacttagtttcaaatgaaagaaaaggtagggttgtttttgcaaaatgaagttcatataaattgaggtttgtaaactttaattaaaacatgcaacctgttcaaaaatcacttcattttttcaacacaatattagggtcacaaaacaattcagtgcatgtttacattagagtttgtaaatctaagtttgaactcagaatttcattttgctaattgagttagcaaaaagaatcttttcttctttcatttcaaattgagtatcaagacaaaattgaggtagctctcagtttataatatgatttttggtataaatcataagcatcctctagtgtaggtaatttagtttgagttagataagattactataagaagcaaagttttctctccgtagttgcatatctaggactcatttggaatctttggttaagtttgaatgatctggtgtagttgatttacgtgttggacagtagtttacaataaaactaaaactagtttttcagttttactttttgaatacatgttggggcctcctagatcaaaaagagattaattttatttattaatgaatttcggtatgcaaatcgtttagaagttacaactattggaagtgatatttctatgaataccatgataggaaagggaaaaaaacttaaaggagtctagaatagaatgaaacaaaaggggtggggatgcaggtaatcaaaattatggaaattccaaaaatgttatcatgatgagtgaagaagacatgttaaatgactttttgttgttttcagaatttttgaagatttggagtgaatattttttcgttgcaagtgtgtgcactaaggatattccctagataatttatgttgaactgaggtggacggggtgttgggagcacgtgttgaggttggttgttggatgttgagatcctctggtggttgtgacaatagtaattatttgcactttaacactcaagttaataagaattttggtagagtaataacaatattaactgatgcgtatctagagaagggagggttctgctatttataggtgttgggtggtttgaattaaatgagattggattagttaagctgtcaatctactgtcttagaatcccatacttcatctgtaaacacacatggtgtttatgcaattggtatgacttataaggtagttgcttaaaacagtagtgagaatgacactcctatcaaagtaagtgtcccaccttacaaggtatatgatgtgtagggcttggaattcgtttagaatggataatggtcgaatctttttcatgtaagcatctgaaatcgaacccttaaaagacttatttatttaccaattatgttgagttttgttggtgagtttaatttttaaaattgaaaataaaaatactcaaacatattttcttttacattttctgtttcactgaaaatataaagaaaaacagagatgatgaagcaaataaaataggcgtcggttgatggcttatttttgcttggctttcgggattataagcttgccattaataccttacaagaagtatttgaataatctatttttattatcatttttatggttggttcacatgcttccaaggaacccaacagttagtgcggaaattattgtttggtatttcaaataaaagaacccaataattagtgtgaaattattgcttggcatttcaaataaaagaggttgacttgtttagcttataaatgattttgttatcaactttgttcgaaattatttttgataaatattataggataagcaataatttttattcatattattatcattttataaattggtttggcatagcgcagca

SEQ ID NO: 291 - DNA, 8448 bp, synthetic construct
  misc_feature 1..8448: note = Synthetic polynucleotide
  source 1..8448: mol_type = other DNA
attgccgaattaagctgacaaaaaataatcaaacttaaaagtctcaaaataattacggaactactaaaccaaaggagctccttctgtgcgatgtaaacatttttttcttatggtttaaaaatataaatatataaattgaaaaaagtaattaaaattgacaatttcttaatatgcgttttctcttatttattattttcatatgtatatattattattttaaaacgtatattaattaatgttattttaaccttttaaaataaaaaatttccttatgacacgtaataaaaagaaacaaaagtaacatgttaagttctaggttttcattagtaattaataagtaaataaataataaataaagtgtttcattttcatattttgtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggagaacaaggcatgtttggttttcagtaaggaagactcctatgcaggttttaataattaaaaccaaaaggggtgttcggttttcagctgaatctgattgtaaactgagttttgtctcaattttgttttgaagtttagtttgaaatgagagaaaaggtgggcttgtttttgcaaaactaaattcgtttaaatttgagtttgtaaactttaatccaaacaagcaaatttgttcaaaagtcagttaatttcgtaacacaatgttgagtttagtgaatgtttagattaagatttgtaaatgcaagttctaactcaaaatttcatttagcaaattgagtttgcaaaactagtcttttcttcttcaagttttcattttaaattgagtttcaaggaaaaatttaggtaaatctcagttcacaatcgactttaatttaaaccaaaggtttttcagttttactttttgaatagattttgggtcctcctaactgaaaactgaacaggcctttaaatgatagaagagatttatttcatttgttaatgaatgttcatatgcaacttgtttagagcttttggaagtgatatttctattttcatttttcttttccatcactctcgttctcaattttttccatcatcttcgttcaaaatttattttcctcttgttctgctgccaacctgcttgaggaaccaactgaaataaaatcattacccgtggaccaaaaataaaattgaaattccaaatccaaccttagttatgcaattaaatcccaagtcatcgtgattcatgagcactgtcctgacctttcacccttctctacaacacaattttatgcctacaaacccgccaatggaaccagtgtctactgcattgcccctgtaccattattaaagtactaccatatactattccctgcaatctacataaagatagtaaggaatgaaaccaaaattatggaaatttgaaaaatgttaccctgcaatttttatatgaataccatgataggaaagggggaatgaacttaaggagcctagtatataatgaaacaaaagggtgggggatgcaggtaaccaaaattatggaaattccaaaaatgtcatcatgatgagtgaagacatggttaaatgaccttttgttgttttcagaatttttgaaaacttggagttaatattttttcgttgcaagtgtatgcactaaggatgttccctagataattaatgttgagctggggtggatggagtgttgggagcacgtgttgaggttggttgttaggtgtggtagctaacagtgtattgtgtgacaatagtaattatttgcactttaacactcaagttaatagaaatttttggtagagtaacaacaatattgaatgatgcgtatctagagaagggagggttctgctatttataggcgttgggtggttttaattaaatgagattgaattagttaagctgtcaatcccatactgcttctgtaaacacttgtatctatgtgattcgtatgacttataaggtagttacttgaaatagtgataagaatgacactcatatctaagtgagaaggtgtcccaccttgtaaggtatatgatgtgttggattcttattgggttgaattgttggggtgtagaaacttcaaatctatttagaatggatatggatcaaatctttttcatataggtatcctaaaattcaacctttattaacatgattaaaagacttaattatttgtcaattatgttaggctttgttggcaagtttaatttttaaaattgaaaataaaaatgctctaacatattttcgttttcactttttgtttcattgaaaacataaagaaaaacagagaaatcataaagccaataaaataggccttggctgttggttttctggatggatttttgcttggcttctggattagaagcctacctttttcatttgttgaatagaagcctaccttttaataccttataagcaatatttgaataatctatttttattatcatttttctggtt

SEQ ID NO: 292 - DNA, 8464 bp, synthetic construct
  misc_feature 1..8464: note = Synthetic polynucleotide
  source 1..8464: mol_type = other DNA
attgccgaattaagctgacaaaaaataatcaaacttaaaagtctcaaaataattacggaactactaaaccaaaggagctccttctgtgcgatgtaaacatttttttcttatggtttaaaaatataaatatataaattgaaaaaagtaattaaaattgacaatttcttaatatgcgttttctcttatttattattttcatatgtatatattattattttaaaacgtatattaattaatgttattttaaccttttaaaataaaaaatttccttatgacacgtaataaaaagaaacaaaagtaacatgttaagttctaggttttcattagtaattaataagtaaataaataataaataaagtgtttcattttcatattttgtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccattattcttggtttttggacagacaagggtgtgtttggttctctgactacactcactcactccttctctcttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctctctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggagaacaaggcatgtttggttttcagtaaggaagactcctatgcaggttttaataattaaaaccaaaaggggtgttcggttttcagctgaatctgattgtaaactgagttttgtctcaattttgttttgaagtttagtttgaaatgagagaaaaggtgggcttgtttttgcaaaactaaattcgtttaaatttgagtttgtaaactttaatccaaacaagcaaatttgttcaaaagtcagttaatttcgtaacacaatgttgagtttagtgaatgtttagattaagatttgtaaatgcaagttctaactcaaaatttcatttagcaaattgagtttgcaaaactagtcttttcttcttcaagttttcattttaaattgagtttcaaggaaaaatttaggtaaatctcagttcacaatcgactttaatttaaaccaaaggtttttcagttttactttttgaatagattttgggtcctcctaactgaaaactgaacaggcctttaaatgatagaagagatttatttcatttgttaatgaatgttcatatgcaacttgtttagagcttttggaagtgatatttctattttcatttttcttttccatcactctcgttctcaattttttccatcatcttcgttcaaaatttattttcctcttgttctgctgccaacctgcttgaggaaccaactgaaataaaatcattacccgtggaccaaaaataaaattgaaattccaaatccaaccttagttatgcaattaaatcccaagtcatcgtgattcatgagcactgtcctgacctttcacccttctctacaacacaattttatgcctacaaacccgccaatggaaccagtgtctactgcattgcccctgtaccattattaaagtactaccatatactattccctgcaatctacataaagatagtaaggaatgaaaccaaaattatggaaatttgaaaaatgttaccctgcaatttttatatgaataccatgataggaaagggggaatgaacttaaggagcctagtatataatgaaacaaaagggtgggggatgcaggtaaccaaaattatggaaattccaaaaatgtcatcatgatgagtgaagacatggttaaatgaccttttgttgttttcagaatttttgaaaacttggagttaatattttttcgttgcaagtgtatgcactaaggatgttccctagataattaatgttgagctggggtggatggagtgttgggagcacgtgttgaggttggttgttaggtgtggtagctaacagtgtattgtgtgacaatagtaattatttgcactttaacactcaagttaatagaaatttttggtagagtaacaacaatattgaatgatgcgtatctagagaagggagggttctgctatttataggcgttgggtggttttaattaaatgagattgaattagttaagctgtcaatcccatactgcttctgtaaacacttgtatctatgtgattcgtatgacttataaggtagttacttgaaatagtgataagaatgacactcatatctaagtgagaaggtgtcccaccttgtaaggtatatgatgtgttggattcttattgggttgaattgttggggtgtagaaacttcaaatctatttagaatggatatggatcaaatctttttcatataggtatcctaaaattcaacctttattaacatgattaaaagacttaattatttgtcaattatgttaggctttgttggcaagtttaatttttaaaattgaaaataaaaatgctctaacatattttcgttttcactttttgtttcattgaaaacataaagaaaaacagagaaatcataaagccaataaaataggccttggctgttggttttctggatggatttttgcttggcttctggattagaagcctacctttttcatttgttgaatagaagcctaccttttaataccttataagcaatatttgaataatctatttttattatcatttttctggtt

SEQ ID NO: 293 - DNA, 8891 bp, synthetic construct
  misc_feature 1..8891: note = Synthetic polynucleotide
  source 1..8891: mol_type = other DNA
aaattactacttaatttcatggattgattcatttattcatgcgcttttgtaattcggatactaactgtgaaaaagttcatttattttctagattaagtttatttgataagtctaaaacacacgtttcacgggtcggaccttaaatgagacaaactagtccatatacttaccccctaaaattaatgttatcaaatttggatcagtcatcgagtcgatttagatactgggtcacaagttgacccaatgaatcactagtcgatcacatgatcccgtctatattttaaaaatcataacatgtaaacttacacttaatatataagttttaacaaaatccaacatatctagaagcataattatgaacttgtttaaaattttaagcacacaatatattattatcatctatttattaaaactatcattgttgtacacggtcaaaaataatacatacacatatgtacatagatacatgcacacacacacaaacatatatgacatatcatatgagatgaatatgattattatgagagtgaaatgattaaatttgaaccgtatatccatatatgcaaggttaagatttacattctatatttaatctgtttagtctcatataatatgctttctctaataagatatgtcatctctctctccctctctctctctctctctctctctatatatatatatatatatgacatgtcatatgagagaaaaatattttatataagagtgaaatgattaaatttgaaccatataatcaaacatgaataatcaagatttaatcttaaaaagtcatgcgaccttttaagtagttacatcaaattaaatcttaactttatacagttcaaatttaatcttctcactcccatataagatatttcctctcataagatttggtagcggcgtcaatcatattggtggcgatgatggtgatcatggtgatgatcacattggtggtagtagtggtggtaagggtgatgacaacgacaacgaccacggtcatgtgatggtgacagtagagattaacgatcatggtagtggttgcggtggagtttatgatagttatggtggaaacagtcataacatgataatggtcatggttgtgataggtttaagatatcttaggacatcacatattaaatctttaagatatatatttttttgttttgtaaatcttatggagattttataattgaataaaattttaatttgaattttaaatgttttaatcttgatctctttcattttaatctaatcgttgaaattaattattttcattttcatataagaacataagatgattattctcatatgatagatacatcttttaaacacatacacacttgtaaattagctatgaaaaaattgaaaggataataactttattgtcttagattataaaatgtggttatgccgtgatataaaaaagtatgcaaatctataaataacatgtgttgatttaattgttaaaaagaaattaaaaaatacttaccttttgagtcaatcaaatcgttgattcaagtcgaatcaaatcgattcatgtcgaatcatatgcatatcgaatccaactaatagttgagattcaataggtgtctaattctagactaatgggttgagccgagtctaataataacatgtttggataaatggcgcttaatttcaattcaattattttttcacgtttatagtaaagaactctttgtagctttgagattttcacggtgaaaaattgatttttgtggtgccaccgcagaaccaaagataccataagtcgccaatgaattgcaattgcaaggtcaagattttttttcgtttgattctgaaatgaatatataatgatgatatgttgtaaaatttgggtccatggatacatccctttcgtatgtagcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttccactttactctcccactaccactcctttcatgacttcaacttcaacaaccacctctttctctctcacttcttctcttgctcttatcaaaacaaaccactatgaataatactaacgataataatagttacaacaatacactctagcttctagcctgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtcaaaccccttctttcattattattattattattacctgaaacaaaactgaaaattctctttttcttcttcttgtgctattttaatattgaggtaagataaagtagggtagggccttgtttattttattcagtttttttgctcgttattctgcggtttggggtgtaggtttgtaggtgagatatgttatcctgttttgtcggtgcagttataggtgtgtgtgttacttgtttggagttggtttagttccttaatcgtggctttgcttttgttcaaggctttggtgtggtgtgattctttgtgtctgttcttgagatgtttgttgttcagttatgatttggctgattgtactcggtatactttacttttttttcccctcctagcgtagctttaaagtgcttatctgaacggctgtcttataacgcatgtgaaatattattgttttccttttcagcgaattgattcttgcacccccgggtgtgtttggtattaaaaataataacttcgtttgtacggaaaaaggcttgaagtgggtgtgcaatttgtagtcttaaaataacacaaacactactatgctgttttccctctagcctttgagggggccctcaattgtgtagtgaactattgatatgctgttacagttttctcattactgtttttgtgtttggattatcttttttggagaaataatatttttttttccttttatagattactagagagcctttgaaaaaaaaaaaataatgataattttccaaatataatctgttaccaaacatgaccaaactctttaggtatgtgatctgtgtaaagatacatccatgtattttactgtcatgaatcaagttgttttatagtgtgaaggcatgttatttgtactacaagagcaattccttgttgatgtgaacctaggattcttactcaaggcttgcattattatcaaaatggtagttttctgctaattggagtgagagtgactttagccttcccttgtctggtcatccataacgtgggtcctgattgttgaatttgatttatctatcttaaacagataggttgtctgattgtttatgtaatttccttgcatgaagacactagtcatgtattgcctccttgactaatgttagttttcatactgtttagtttgacttgttgatacattattgagaatgagtgcaattggtgctgctggatcatgaaatcctaaccacatgctttgcttatgtgactattcatctctattctgttttatggtgctagctatgtcagactgtcagttgtcacatgtactatccatttcaatttgactcattagtgctagaggatgctactgtgcattctaggatagtattcaatggtgtaataagcttgtattttggagacagatggccatcataattggaaagtttccaaccccaggatatggagaatatggtctatctgtcaatgaatgtggttttatatgcagtttttttttggagctgggataactttctgttggctgttctaagagtaatttgtgtctttttttttgtctcatggttcttccacagttgaaaagggttatctaagtcaaaagttacacttttccttaataatgaaaaacaatactcttctgaatagggtgacaatgaattgaaccaactcgaaaatagttcggaactcgattcaataatcagctcgttgaatttggttcatgaaccaaatgagctgaatttgagctcgttaaataaatgacccaaacttgagctgctaatagttcaactcatttgattcatgaattggctcaatatatatatatatatatatatatatatatataattgattgggttttctattattttatatatatctagggcagggtagtttttatttatgcagactttggttataattatgttttttttgtcataccttgtggccctacaaagctaatagaataagttcagttttgtcttttcacaaaaattaaacaatcattttatctaaaatctaccaataaattgaaaatttgaattgtataggatttaaaaaatttgacttttggttcatgtgatccaagctaacaagctgaattgaactgtttgtgagcttgaactgagttgagttcgagctgaaaaataaaggttcatgaaccaaaagtcaaatttttcagcaaaagatattaatgagccgaactgaactcgactcgtttccggacctacttctgaatattctaagttaaaatacatatcatgagaaagatcgtgaaaagatatttaagatggactttttagcatccaatctcccagcatttcagatgtactatcggtggatatacattgtggtttatcccctgtatttaagcttgatgacttctcttttgaattattctggtgtctttcactaaagagtaaatacaatgccatcccttttggtaatatagaagataaacaacttcattggtattgttttggtcttattttactaatacgttaaagctgaattccaagtatcatggagccgcagaatgttacttgagcaatgtttgaggattacttcagtaaaacagggctcaaggagcatcttatactgctaatatgccatctcattcaatgccaaacaagctcacatgatgtgcatcatatggaatacccttgttcttgggtttatacatattgcttatgttctgcaatctttgcaatgatgactaattcaaataagttagaaattccatctcatttgcatctgcatttaatgaaacaacaaatattttaagtgcttccaatttgtggcaacaacagcatgtcatgctaaggtaacagtagacttgtcatgttagcatttcttaagtcttggcacttgggtatgcttgtactcattcacctctctttgacataaattggaatcatttatatttgtaaaagataatatcatgtttcatagtcttgcacaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttggttagaaactatcagtaaaatatcagtttcatgtctcttccaattttttcatagggaagcatatttagtttcagcttgtgtcattcctggcatacaaaagtaaaacaaactctaatgtctggcacttttattgacagataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctgtgcctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcacggtgagctggacctgtcccaacagggcaggaggcattatatggatctagaacagtccagggcatatgaatctgctcattggtcactttaatgcacttgttttttttttgtcagctttgtaataacatgtagcagaaacctgtgcctacttggttacttaccagattgatgataactattgcttgttgttttgttttgttttaactgataagcaaggtgggtagccttgcctcatttgtggaactttcaggcaccgaatgaatctcacctgagagtgtgttttgttagtagtgtcttaagcacaggtatggtgttttctgccattgccgtcacctctggaattatttgtgattgggaggacagcctctttgatgtttgctaccaccgctccattttatttgatattatcttttgactatctgtcaactaagtgttgcacacattgcttgcctaagaacattttgcacagcattaagttgaatgacttggataacaaggcatgcttggttttcagttgggaggtgctcaacgtacatttcagaaggtaaaactaaaaaagctctttgtatggttttcaattgaatccaattgtaaattgggttttgtctcaaaacttagtttcaaatgaaagaaaaggtagggttgtttttgcaaaatgaagttcatataaattgaggtttgtaaactttaattaaaacatgcaacctgttcaaaaatcacttcattttttcaacacaatattagggtcacaaaacaattcagtgcatgtttacattagagtttgtaaatctaagtttgaactcagaatttcattttgctaattgagttagcaaaaagaatcttttcttctttcatttcaaattgagtatcaagacaaaattgaggtagctctcagtttataatatgatttttggtataaatcataagcatcctctagtgtaggtaatttagtttgagttagataagattactataagaagcaaagttttctctccgtagttgcatatctaggactcatttggaatctttggttaagtttgaatgatctggtgtagttgatttacgtgttggacagtagtttacaataaaactaaaactagtttttcagttttactttttgaatacatgttggggcctcctagatcaaaaagagattaattttatttattaatgaatttcggtatgcaaatcgtttagaagttacaactattggaagtgatatttctatgaataccatgataggaaagggaaaaaaacttaaaggagtctagaatagaatgaaacaaaaggggtggggatgcaggtaatcaaaattatggaaattccaaaaatgttatcatgatgagtgaagaagacatgttaaatgactttttgttgttttcagaatttttgaagatttggagtgaatattttttcgttgcaagtgtgtgcactaaggatattccctagataatttatgttgaactgaggtggacggggtgttgggagcacgtgttgaggttggttgttggatgttgagatcctctggtggttgtgacaatagtaattatttgcactttaacactcaagttaataagaattttggtagagtaataacaatattaactgatgcgtatctagagaagggagggttctgctatttataggtgttgggtggtttgaattaaatgagattggattagttaagctgtcaatctactgtcttagaatcccatacttcatctgtaaacacacatggtgtttatgcaattggtatgacttataaggtagttgcttaaaacagtagtgagaatgacactcctatcaaagtaagtgtcccaccttacaaggtatatgatgtgtagggcttggaattcgtttagaatggataatggtcgaatctttttcatgtaagcatctgaaatcgaacccttaaaagacttatttatttaccaattatgttgagttttgttggtgagtttaatttttaaaattgaaaataaaaatactcaaacatattttcttttacattttctgtttcactgaaaatataaagaaaaacagagatgatgaagcaaataaaataggcgtcggttgatggcttatttttgcttggctttcgggattataagcttgccattaataccttacaagaagtatttgaataatctatttttattatcatttttatggttggttcacatgcttccaaggaacccaacagttagtgcggaaattattgtttggtatttcaaataaaagaacccaataattagtgtgaaattattgcttggcatttcaaataaaagaggttgacttgtttagcttataaatgattttgttatcaactttgttcgaaattatttttgataaatattataggataagcaataatttttattcatattattatcattttataaattggtttggcatagcgcagca

SEQ ID NO: 294 - DNA, 8885 bp, synthetic construct
  misc_feature 1..8885: note = Synthetic polynucleotide
  source 1..8885: mol_type = other DNA
aaattactacttaatttcatggattgattcatttattcatgcgcttttgtaattcggatactaactgtgaaaaagttcatttattttctagattaagtttatttgataagtctaaaacacacgtttcacgggtcggaccttaaatgagacaaactagtccatatacttaccccctaaaattaatgttatcaaatttggatcagtcatcgagtcgatttagatactgggtcacaagttgacccaatgaatcactagtcgatcacatgatcccgtctatattttaaaaatcataacatgtaaacttacacttaatatataagttttaacaaaatccaacatatctagaagcataattatgaacttgtttaaaattttaagcacacaatatattattatcatctatttattaaaactatcattgttgtacacggtcaaaaataatacatacacatatgtacatagatacatgcacacacacacaaacatatatgacatatcatatgagatgaatatgattattatgagagtgaaatgattaaatttgaaccgtatatccatatatgcaaggttaagatttacattctatatttaatctgtttagtctcatataatatgctttctctaataagatatgtcatctctctctccctctctctctctctctctctctctatatatatatatatatatgacatgtcatatgagagaaaaatattttatataagagtgaaatgattaaatttgaaccatataatcaaacatgaataatcaagatttaatcttaaaaagtcatgcgaccttttaagtagttacatcaaattaaatcttaactttatacagttcaaatttaatcttctcactcccatataagatatttcctctcataagatttggtagcggcgtcaatcatattggtggcgatgatggtgatcatggtgatgatcacattggtggtagtagtggtggtaagggtgatgacaacgacaacgaccacggtcatgtgatggtgacagtagagattaacgatcatggtagtggttgcggtggagtttatgatagttatggtggaaacagtcataacatgataatggtcatggttgtgataggtttaagatatcttaggacatcacatattaaatctttaagatatatatttttttgttttgtaaatcttatggagattttataattgaataaaattttaatttgaattttaaatgttttaatcttgatctctttcattttaatctaatcgttgaaattaattattttcattttcatataagaacataagatgattattctcatatgatagatacatcttttaaacacatacacacttgtaaattagctatgaaaaaattgaaaggataataactttattgtcttagattataaaatgtggttatgccgtgatataaaaaagtatgcaaatctataaataacatgtgttgatttaattgttaaaaagaaattaaaaaatacttaccttttgagtcaatcaaatcgttgattcaagtcgaatcaaatcgattcatgtcgaatcatatgcatatcgaatccaactaatagttgagattcaataggtgtctaattctagactaatgggttgagccgagtctaataataacatgtttggataaatggcgcttaatttcaattcaattattttttcacgtttatagtaaagaactctttgtagctttgagattttcacggtgaaaaattgatttttgtggtgccaccgcagaaccaaagataccataagtcgccaatgaattgcaattgcaaggtcaagattttttttcgtttgattctgaaatgaatatataatgatgatatgttgtaaaatttgggtccatggatacatccctttcgtatgtagcaccagctagcaagtgtgtgacagggacagaaaaagttgcacaaaacgacagttacacttgtaagggcctggaatggtttaaaacttggaactttttgaagcaatgagatgagaggaaattgttgagttgagaagaaaaaaaaagaagagagggtacaatctgtctctcgtagctggctggctccattatttttggttttggacagacaagggtgtgtttggttctctgactacactcactcactccttctctcttctctctcagtgtaccacactctctgccacctatggcccagcgtcaactctctccttttccctacccttccacctcctcttttgtcttccactttactctcccactaccactcctttcatgacttcaacttcaacaaccacctctttctctctcacttcttctcttgctcttatcaaaacaaaccactatgaataatactaacgataataatagttacaacaatacactctagcttctagcctgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtcaaaccccttctttcattattattattattattacctgaaacaaaactgaaaattctctttttcttcttcttgtgctattttaatattgaggtaagataaagtagggtagggccttgtttattttattcagtttttttgctcgttattctgcggtttggggtgtaggtttgtaggtgagatatgttatcctgttttgtcggtgcagttataggtgtgtgtgttacttgtttggagttggtttagttccttaatcgtggctttgcttttgttcaaggctttggtgtggtgtgattctttgtgtctgttcttgagatgtttgttgttcagttatgatttggctgattgtactcggtatactttacttttttttcccctcctagcgtagctttaaagtgcttatctgaacggctgtcttataacgcatgtgaaatattattgttttccttttcagcgaattgattcttgcacccccgggtgtgtttggtattaaaaataataacttcgtttgtacggaaaaaggcttgaagtgggtgtgcaatttgtagtcttaaaataacacaaacactactatgctgttttccctctagcctttgagggggccctcaattgtgtagtgaactattgatatgctgttacagttttctcattactgtttttgtgtttggattatcttttttggagaaataatatttttttttccttttatagattactagagagcctttgaaaaaaaaaaaataatgataattttccaaatataatctgttaccaaacatgaccaaactctttaggtatgtgatctgtgtaaagatacatccatgtattttactgtcatgaatcaagttgttttatagtgtgaaggcatgttatttgtactacaagagcaattccttgttgatgtgaacctaggattcttactcaaggcttgcattattatcaaaatggtagttttctgctaattggagtgagagtgactttagccttcccttgtctggtcatccataacgtgggtcctgattgttgaatttgatttatctatcttaaacagataggttgtctgattgtttatgtaatttccttgcatgaagacactagtcatgtattgcctccttgactaatgttagttttcatactgtttagtttgacttgttgatacattattgagaatgagtgcaattggtgctgctggatcatgaaatcctaaccacatgctttgcttatgtgactattcatctctattctgttttatggtgctagctatgtcagactgtcagttgtcacatgtactatccatttcaatttgactcattagtgctagaggatgctactgtgcattctaggatagtattcaatggtgtaataagcttgtattttggagacagatggccatcataattggaaagtttccaaccccaggatatggagaatatggtctatctgtcaatgaatgtggttttatatgcagtttttttttggagctgggataactttctgttggctgttctaagagtaatttgtgtctttttttttgtctcatggttcttccacagttgaaaagggttatctaagtcaaaagttacacttttccttaataatgaaaaacaatactcttctgaatagggtgacaatgaattgaaccaactcgaaaatagttcggaactcgattcaataatcagctcgttgaatttggttcatgaaccaaatgagctgaatttgagctcgttaaataaatgacccaaacttgagctgctaatagttcaactcatttgattcatgaattggctcaatatatatatatatatatatatatatatatataattgattgggttttctattattttatatatatctagggcagggtagtttttatttatgcagactttggttataattatgttttttttgtcataccttgtggccctacaaagctaatagaataagttcagttttgtcttttcacaaaaattaaacaatcattttatctaaaatctaccaataaattgaaaatttgaattgtataggatttaaaaaatttgacttttggttcatgtgatccaagctaacaagctgaattgaactgtttgtgagcttgaactgagttgagttcgagctgaaaaataaaggttcatgaaccaaaagtcaaatttttcagcaaaagatattaatgagccgaactgaactcgactcgtttccggacctacttctgaatattctaagttaaaatacatatcatgagaaagatcgtgaaaagatatttaagatggactttttagcatccaatctcccagcatttcagatgtactatcggtggatatacattgtggtttatcccctgtatttaagcttgatgacttctcttttgaattattctggtgtctttcactaaagagtaaatacaatgccatcccttttggtaatatagaagataaacaacttcattggtattgttttggtcttattttactaatacgttaaagctgaattccaagtatcatggagccgcagaatgttacttgagcaatgtttgaggattacttcagtaaaacagggctcaaggagcatcttatactgctaatatgccatctcattcaatgccaaacaagctcacatgatgtgcatcatatggaatacccttgttcttgggtttatacatattgcttatgttctgcaatctttgcaatgatgactaattcaaataagttagaaattccatctcatttgcatctgcatttaatgaaacaacaaatattttaagtgcttccaatttgtggcaacaacagcatgtcatgctaaggtaacagtagacttgtcatgttagcatttcttaagtcttggcacttgggtatgcttgtactcattcacctctctttgacataaattggaatcatttatatttgtaaaagataatatcatgtttcatagtcttgcacaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttggttagaaactatcagtaaaatatcagtttcatgtctcttccaattttttcatagggaagcatatttagtttcagcttgtgtcattcctggcatacaaaagtaaaacaaactctaatgtctggcacttttattgacagataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcacggtgagctggacctgtcccaacagggcaggaggcattatatggatctagaacagtccagggcatatgaatctgctcattggtcactttaatgcacttgttttttttttgtcagctttgtaataacatgtagcagaaacctgtgcctacttggttacttaccagattgatgataactattgcttgttgttttgttttgttttaactgataagcaaggtgggtagccttgcctcatttgtggaactttcaggcaccgaatgaatctcacctgagagtgtgttttgttagtagtgtcttaagcacaggtatggtgttttctgccattgccgtcacctctggaattatttgtgattgggaggacagcctctttgatgtttgctaccaccgctccattttatttgatattatcttttgactatctgtcaactaagtgttgcacacattgcttgcctaagaacattttgcacagcattaagttgaatgacttggataacaaggcatgcttggttttcagttgggaggtgctcaacgtacatttcagaaggtaaaactaaaaaagctctttgtatggttttcaattgaatccaattgtaaattgggttttgtctcaaaacttagtttcaaatgaaagaaaaggtagggttgtttttgcaaaatgaagttcatataaattgaggtttgtaaactttaattaaaacatgcaacctgttcaaaaatcacttcattttttcaacacaatattagggtcacaaaacaattcagtgcatgtttacattagagtttgtaaatctaagtttgaactcagaatttcattttgctaattgagttagcaaaaagaatcttttcttctttcatttcaaattgagtatcaagacaaaattgaggtagctctcagtttataatatgatttttggtataaatcataagcatcctctagtgtaggtaatttagtttgagttagataagattactataagaagcaaagttttctctccgtagttgcatatctaggactcatttggaatctttggttaagtttgaatgatctggtgtagttgatttacgtgttggacagtagtttacaataaaactaaaactagtttttcagttttactttttgaatacatgttggggcctcctagatcaaaaagagattaattttatttattaatgaatttcggtatgcaaatcgtttagaagttacaactattggaagtgatatttctatgaataccatgataggaaagggaaaaaaacttaaaggagtctagaatagaatgaaacaaaaggggtggggatgcaggtaatcaaaattatggaaattccaaaaatgttatcatgatgagtgaagaagacatgttaaatgactttttgttgttttcagaatttttgaagatttggagtgaatattttttcgttgcaagtgtgtgcactaaggatattccctagataatttatgttgaactgaggtggacggggtgttgggagcacgtgttgaggttggttgttggatgttgagatcctctggtggttgtgacaatagtaattatttgcactttaacactcaagttaataagaattttggtagagtaataacaatattaactgatgcgtatctagagaagggagggttctgctatttataggtgttgggtggtttgaattaaatgagattggattagttaagctgtcaatctactgtcttagaatcccatacttcatctgtaaacacacatggtgtttatgcaattggtatgacttataaggtagttgcttaaaacagtagtgagaatgacactcctatcaaagtaagtgtcccaccttacaaggtatatgatgtgtagggcttggaattcgtttagaatggataatggtcgaatctttttcatgtaagcatctgaaatcgaacccttaaaagacttatttatttaccaattatgttgagttttgttggtgagtttaatttttaaaattgaaaataaaaatactcaaacatattttcttttacattttctgtttcactgaaaatataaagaaaaacagagatgatgaagcaaataaaataggcgtcggttgatggcttatttttgcttggctttcgggattataagcttgccattaataccttacaagaagtatttgaataatctatttttattatcatttttatggttggttcacatgcttccaaggaacccaacagttagtgcggaaattattgtttggtatttcaaataaaagaacccaataattagtgtgaaattattgcttggcatttcaaataaaagaggttgacttgtttagcttataaatgattttgttatcaactttgttcgaaattatttttgataaatattataggataagcaataatttttattcatattattatcattttataaattggtttggcatagcgcagca

SEQ ID NO: 295 - DNA, 9444 bp, synthetic construct
  misc_feature 1..9444: note = Synthetic polynucleotide
  source 1..9444: mol_type = other DNA
caaaaaatacaaatcataaaagacactcattaaataaaaagtaagacttcaaaattttataatttgtaattatttcaaccaaacaattttaaaaaatttcagaaaaaattattaaagaagtacatataacgtttctcaaacatataaaaataaacattcacaacgttgtctaagcaagtgcatgagatgttgtaattgtatttcaattcttataataaataaaaagtttgaatagctttttagttcttgtaatttagcattttttgcttttcattcatgcaatttttttttgttttcaatccttgcaaattatgcttgttttattttcagtccttaaaatgatttagatagtttttttttctatttaaagtattatctaaactgctttaagaaaaaaaaaacaaatacaatttgttagctgtgacgaacacacatgtaaggttttggggacaattgccccacattttttttatttgtatgtgtaataaatttttttcacccccataaaaaataggtattgccccctttaagacaactttcttttaaatgaacgtataaaatgaatcaaaacaaagaataaattcatactaatttcactactttatccttttaatttttataatattgataataaaaaaaattatagaatatttttgaagtatgaaaaatactagatgaccattgtaatttgtataagtttaaacattttgaatttttttgtcataacattatgtattttgtaaaattttgtgatatttttattttgcgaaaatattataaactattggttgggttaattatgtttttggttcctaaactttttgtaaaattcccttgtagtctctaaacaatttttgtaatgtttttggtccctataatttctttcattaatagttctagtttctattatcaagcaacaatgttaatttatgacataaccaatcacattaacttgtcacaccatcaagccgtgatgacaattaagtgtaattctatgaggaatttaaaatacaatttataaaaaaaattacatatgattgttccatcaaacccttatgatgtgataagtatgctgctaacatgaatattacattatcaattaatgttgttactcaataacaagtattaaaaccatcaaaggggaagagtaaaagtataaggaccacacatactaaaaaattgtttaaagactaaaagtaaaatttgtaaaaagttttggaaaaaaatataattaatcctaaactatttcttggtcttatgaagatttttttttggattcacagttattttcaaggactaaaacaattttttttctttaaaaaattaaaaataaaaaatactaaatcacaacaactaaaaagtatttaaatctaaataaaaaaaaaggaaaaaaaagtcttttgacaccaacaaactgacaaattgagagacatagaaagaaaaatagataaaaataagagttactaataagatgtttaaaagaaaaagatgttaaaatatcaccactcaaaaaataaacattgacatcagtactccatttactaaattgataaaatttcagcattaaattgataacattgacattattaattattactcacaactaaatgaattaatgtataactagctagtactagagggagcatcagcatcagcactgattagccagcagcagcaataggttctggttcagtcaccaaagcaccaccctcttcaaggactaaataattatattctttaaaaaacaaaataaaaaaaatactaaattacaacaactaaaaagtatttaaatttaaaaaaaataaggaaaaaaaatcttttgacactaacaaattgacaaattgagagacatagaaagaaaaagaaaaaaaaaatattaatataataaataacgtgatcagttaaaaaaaatagataaaaataaaaatgactaataagatgtttaaatgaaaaagatgttaaaacatcaccacttaaaaaataaacattgacatcagtactccatttactaaattgataaaatttcagcattaaattgataacattgacattattaactattagtctcaacgaaatgaattaatgtatacacccactcactctagttagctagtactaaagggagcatcagcatcagcactcagtagccagcagcagcaatgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctcttatctctttctctctctctcactttctacttttttatttattattgacttcaaaaagaaataaaaaaatcttcttttgtttttttttttttttttagttggcttgttatattgggtagttctgcttctgctttggtttattttcctcatcttactccttggtttctgctatttttagtgtttttgctattttgggtgcttttggaatactgctatgctattcacactctctttttagaaaaaaaataatggtttttgtttttctttccaatttttacatcctattcttttgtggttttggctgtatcttttacattgcttgtttttcatttcagggctttaatcgtgctgattttgttctctgtagattttttttattttattttttatgttgattgcgtgtaatgagtcatgctcttctcttacttcgtaatgaagtgtgtgaaggtttgtttttctatggtttctaatgctgcttaatgattggagaatggttcacgtttctggtttgatgcatctgttggtgctgtgagttgtagatgatagcataaccatggggttgtgttgtttatcttgtagttaacttatgggtagtgatttgcgaaggaatatgctttagaaaacaatggtgctgttgtgagcttgtgttgtttcccccttcccctttcatggtatcatgaagtgctcattaaaggaaaacggagctactagggtaggaaataggtgtagaaatgagtgcaaaacaacaactaagttgcataacatttaaagcaatgtagagtggactcagataaatgaaaagtttaggattctgttttgacataccatgccttcaagagtgtcaagatttggaacaataagtccataatatacaattttttcccttttaatttgttatgtgaagttgatattgtccttttgttcatgagatgatacctctctcatgcaagttcattccataagaaaagtgctttttccccaacattatcttatgcagatcatgcaatgcccccataaggtcatagggtctgatgttgtatctttgtcttgctggacaatgcaatccatggtaacttgctattgtgactgttagataactcaatgggacttgaatcagttaattacagaaataacagttagaaactgttatatgtttaactcaagcttgattacaactgaaattactttactccaatttcattgttgctagcaaatgctacagggcattttagggatttgcttaatggcaaaatgtccctaacaagtgtgtaattttggaggtggatggttatcattttttgggtggttttctgactgtaagatgcaaaatctggcctatttgtttgcagtattttttgtgacaataagaatgaggtgaaacaaaagagccatttgagttctatgatctgttggacattgtgtaaaatgttgcagatctcttccgacaaatgtccaaaccaattactcttattgggtttgagatattttagagaatgattctcgtcctgcagagctaaaattttcaaattaagatgcaacctccctttgccaacttgttctctaagttcctattgatggtctggtcttgtgggatccatatcgtagatcattttagaaaagaatctcttctagatgacttgatattgaacattatatgttgtagacacatgtgaccttcaatccattcaagatgcaacatgttctcaggggaaagatagatctcattgactgacaattatgggatgttaattacttatgttgaataatgatgtttagcaactaaaaggtgtaggaaatttctggatgctctcccagtatggatatagtacttgttgagaactatggcataattcttgtagctaacccctttgaatgtgaaaaaaattgtttggttgttgattgctgctcttctgttttatggcatggttggaacattttttttttatcatctgatgtgtggtttattggtaattaattgaagttgaactttctttcactggtgtaaagatgtagatggaatggtgtgtaaggaaaaatttgatggctacttagtatgaaaaatttgatgaaaagtaccacaaaaatagacatggctgatttccatttgatttttttataagtaatctaatttgttaattagcagaaagcctcttctttgattttgtgcaaattcatttgtaatgttttcatatcacatagcaccggtcggtgtgtgaatgctatttacaattattagacttgcaggttcctttccacagaagctacattggtgtgtatatatatatatatatatatatatatatatataacatattctgtgtttctctactgtgcttaactcaagttcaaatgttttcttactttacaatgagtttaaacatgccttgaggtaacgtgctaattaagctttatctgggctcgtaataggatgttatgatattctatagttgttgtgaccttaaattggaatagtattaacatatggtgttaaaaaatttcaaatgttgctctcttttctccaaatctcatttgtcaattctctgaggctatgatgatctggaaaacttgtgttacaatgggagtttcaagtttcaactgctttggggtttatcggcagttcagcacaagtctatgaatgtggtaagcaaccactgttgttgtggagcttggatagctctctgatggctgtgaacaatgaagtgtaacttttatccctttttgggctttttgtcccgtcaagggttgaaaaggattttattgcacatatttcataccaatttaaggtgcatactattttccataatgaaattaatcagaaagagggatcaacaaatgcatagtgctgttatgaatgttcaagacagtggattaaaaaggttttggagagtacaaatgggcatttttcagaactactaatttccgcattgcagatgagcatatggtatttggtgaaacaaaaggtcatctattcttattttggtaacagagtgaatctatattttaaggtttgtatcttgtattcaagctcaacgacttctctttcaattatgctagtatactgtataaattgtactaaacataatgttatccctcttagcaagaagcgagcgagaaatttttggtatctatgttttagtactgatatccatattgaagttgagttcgaagtatcttggtaccatgggatgttccctgattgcatgttgagaggattttttgctgatgctatcacaacccttgtatccaatgcattaagatttctgtaagatatgggaattgcatcttatggaaatatacgtatgttcttggctttatacatattagctttgttctgtacttttgtcacagttaggaagtatgagtgataaattctctcatttgtaatgtgatcatttaatttaatgaaagtataaaatacttcaaatgcttccaattctgtggtaataacaaatcaacagcattgcatgtaatgattagaagcaattttgtcatgttagaaattctcaaatcttgtgtcatttcgtgttttcttttgtagtatacttttacttaaacttggtagcaattatatttcagacatcttgtgtctgatcatactctctttgtatataaatctggaagcatttatacttcagaaaagctaatatcctctttcatggccctttcccaggtttcatctgctttctgaatttgatcaaggaaaacgtagctgccgcaagcgacttgctggccataatgagcgccgacgaaagcccccaccaagctccatattaacctctcgctatggcagactctctgtgcctatttttggttagaaattatcagtaaaacatcaattccaaaccactctctctgctttatgtatccctaggaagcatcaataatttcagtttttgctattgcatataaaacattcaactgtttcccttatgtttaacagataacaatggtcgagacgatggctttctgatggaatttgcttcataccccaagcttactttgagaaatacattgccaactcaaagatcaactgagccggctcctgggaaccatgctgcaacactcacctggcaaggaggttcagacactccatctgaatttttcctgcaaggatcagggagtagtggtggaacaagcttccttagttcaaaacatcccttagtggaaagttacactggagtcactgactctctctctcttctgtcaagtcaaacctggggttccagaaacacagccccagcaacaagttttgaactgaacaacaacttgttcaatttcaatgggacatccatgacacaatttgctgtggcgtctttgcaagctgcagccatccatcaacttccaaatgctacctcatggttgtacctaaagggagttgattctggtaattgttcgcctgagcgtgtccccaatcacggtcttgctcaaatttctccccctcttcatagccatgttcatagtgagcttgatgtgtcacagcagggcaggaggcattacatgatctaggggagtccagggcctgtgagtcttctcactggtctctttgaagccccttctttgctatcagaaagtatgaggggaatctgtgcctatttgatcacctaactactagcattttggtttctttgaacttagaagtaaggtgggtatatccttcccttgtttgtggaacattgaaaattgaaacatccttattttggcttgtttgaacttataagcaaggtaggtagccttgtttctttgtggtacttttcagacattactagtttggtgttggaacttagaagcaagatgggtagtcttgttttattttgtggaaatttcagacctgtatcatttgaatgtgggtatgattgatgttttatagcatctacgtctgtgattccactgactgcatattccttatattgctatagctgtgctagaactactttcgagtttttttgttagaatctatagattaaatgataataataagtcttttggtattaggattaggattatgatttttaaaagactttttttttcaataaaaaatatatagattaaatgataacgataagtcttttggtatttaattaagttttttaaaatgaatttccatgaatttcatgcgatcttttaatatgatatacaaatatcgcacctattaaacaatcttatccaatttagatttttttttctcttttttgttggttgtcacacctttctctatcccatcacacatgttccattctctctctctctctctctctctctctctctctctatatatatatatatatatatatatatatatatatatatatatatatatatatatatatatatatatatattgcatcttaagtttaccaaacaaataatgaagacaataattttgaatcaagagcgaggacaagaagattctaaagcatggaggattagtttagaagaagatagtactagaattaattgacttaaaaaaaaataaagaaaaaaagtgtgatggaaaaatgttcttgaaaagcttgtccataaagagaagacattaattaaaaaagtgtgatgtaaagtaattttatatttttattcaattgcaaatcatcatttgtatgatttttaaagtgattatcataaaattcagtggatttatcatacatgatagattgtggttggatgacaataaatttattttacactattggtgcattgcctagtttctctgaatcatatcgtatctttatcatttttcatggactcttgtaattttgaatgttttctaagaatcttgaatccttacaaacttttaaattcatacaatcctttcaaatattttaatttcttatgttataaattcattaaaattcaaactataaaatcttaattacaaaaatcttttaaaaagttttgataagtcataacattctcacatagttttttaaaatgcatacaattttttatgtcaaaatagtcttttaaaatttcaatccaatatatatccctaaaagaaaataaaagataaagatgaagagtaatttttttcaaacttacttttggagtaacatgatttttctctctctctctctctctctctctctctctatatatatatatatatatatatatatatatatatatatatgttttatatagtgggttttgccacccattacatcaactcccttgtggatcctcctgagcccaaaagagaaaaccacaagagagttgctaggtgcacccagcagaattgttggtgcacccaacaattatgtgaatgggcaaaattgtccttgtttaaaagtttaaaaaataaacgcgcccattcagtgttgcttcttccgtgagtgcatccatctaccatgtctgctcgtcttcttcgcgcttctccttgttctccgtgcctcggctgccatttccgttcgttttgggaggttttttggtgccatttctgtgtttccaccgtcgaggttagtacctcctatctcccctcccctttaatgtccaatggtgatttagtttaggattttttggctgtagggtagatgacttcgtttctctgcaagtttgtctgcgaaaatggtgaagttggagcgttttccgtttacggaagaacttattccgtatgaaggaaattcttctaacagaagaagtttttccgtaatttcctacggaagaaggttcttccgtatgaagtaaatctttgtatggaagaaccttcttccgtattaacttagggaagaacttctttcgtagactcttacagaagaacttctttcgtatgactcttacggaagaacttcttccgtaagaaggtttccaatttttttattttttaattaattttttttataaattaagttttatttctttgttttgatttataataggtatatttagttttttaataataaaaaattgtatgcttgcatggtt

SEQ ID NO: 296 - DNA, 8459 bp, synthetic construct
  misc_feature 1..8459: note = Synthetic polynucleotide
  source 1..8459: mol_type = other DNA
attgccgaattaagctgacaaaaaataatcaaacttaaaagtctcaaaataattacggaactactaaaccaaaggagctccttctgtgcgatgtaaacatttttttcttatggtttaaaaatataaatatataaattgaaaaaagtaattaaaattgacaatttcttaatatgcgttttctcttatttattattttcatatgtatatattattattttaaaacgtatattaattaatgttattttaaccttttaaaataaaaaatttccttatgacacgtaataaaaagaaacaaaagtaacatgttaagttctaggttttcattagtaattaataagtaaataaataataaataaagtgtttcattttcatattttgtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagtgttgagaagaaaaaagaaaaaaagaaaaaagagagggtacaatctgtctctcgtagctggcttggctccattattcttggtttttggacagacaagggtgtggttctctgactacactcactccttctctcttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcgagctgtgctctctctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggagaacaaggcatgtttggttttcagtaaggaagactcctatgcaggttttaataattaaaaccaaaaggggtgttcggttttcagctgaatctgattgtaaactgagttttgtctcaattttgttttgaagtttagtttgaaatgagagaaaaggtgggcttgtttttgcaaaactaaattcgtttaaatttgagtttgtaaactttaatccaaacaagcaaatttgttcaaaagtcagttaatttcgtaacacaatgttgagtttagtgaatgtttagattaagatttgtaaatgcaagttctaactcaaaatttcatttagcaaattgagtttgcaaaactagtcttttcttcttcaagttttcattttaaattgagtttcaaggaaaaatttaggtaaatctcagttcacaatcgactttaatttaaaccaaaggtttttcagttttactttttgaatagattttgggtcctcctaactgaaaactgaacaggcctttaaatgatagaagagatttatttcatttgttaatgaatgttcatatgcaacttgtttagagcttttggaagtgatatttctattttcatttttcttttccatcactctcgttctcaattttttccatcatcttcgttcaaaatttattttcctcttgttctgctgccaacctgcttgaggaaccaactgaaataaaatcattacccgtggaccaaaaataaaattgaaattccaaatccaaccttagttatgcaattaaatcccaagtcatcgtgattcatgagcactgtcctgacctttcacccttctctacaacacaattttatgcctacaaacccgccaatggaaccagtgtctactgcattgcccctgtaccattattaaagtactaccatatactattccctgcaatctacataaagatagtaaggaatgaaaccaaaattatggaaatttgaaaaatgttaccctgcaatttttatatgaataccatgataggaaagggggaatgaacttaaggagcctagtatataatgaaacaaaagggtgggggatgcaggtaaccaaaattatggaaattccaaaaatgtcatcatgatgagtgaagacatggttaaatgaccttttgttgttttcagaatttttgaaaacttggagttaatattttttcgttgcaagtgtatgcactaaggatgttccctagataattaatgttgagctggggtggatggagtgttgggagcacgtgttgaggttggttgttaggtgtggtagctaacagtgtattgtgtgacaatagtaattatttgcactttaacactcaagttaatagaaatttttggtagagtaacaacaatattgaatgatgcgtatctagagaagggagggttctgctatttataggcgttgggtggttttaattaaatgagattgaattagttaagctgtcaatcccatactgcttctgtaaacacttgtatctatgtgattcgtatgacttataaggtagttacttgaaatagtgataagaatgacactcatatctaagtgagaaggtgtcccaccttgtaaggtatatgatgtgttggattcttattgggttgaattgttggggtgtagaaacttcaaatctatttagaatggatatggatcaaatctttttcatataggtatcctaaaattcaacctttattaacatgattaaaagacttaattatttgtcaattatgttaggctttgttggcaagtttaatttttaaaattgaaaataaaaatgctctaacatattttcgttttcactttttgtttcattgaaaacataaagaaaaacagagaaatcataaagccaataaaataggccttggctgttggttttctggatggatttttgcttggcttctggattagaagcctacctttttcatttgttgaatagaagcctaccttttaataccttataagcaatatttgaataatctatttttattatcatttttctggtt

SEQ ID NO: 297 - DNA, 8769 bp, synthetic construct
  misc_feature 1..8769: note = Synthetic polynucleotide
  source 1..8769: mol_type = other DNA
aaattactacttaatttcatggattgattcatttattcatgcgcttttgtaattcggatactaactgtgaaaaagttcatttattttctagattaagtttatttgataagtctaaaacacacgtttcacgggtcggaccttaaatgagacaaactagtccatatacttaccccctaaaattaatgttatcaaatttggatcagtcatcgagtcgatttagatactgggtcacaagttgacccaatgaatcactagtcgatcacatgatcccgtctatattttaaaaatcataacatgtaaacttacacttaatatataagttttaacaaaatccaacatatctagaagcataattatgaacttgtttaaaattttaagcacacaatatattattatcatctatttattaaaactatcattgttgtacacggtcaaaaataatacatacacatatgtacatagatacatgcacacacacacaaacatatatgacatatcatatgagatgaatatgattattatgagagtgaaatgattaaatttgaaccgtatatccatatatgcaaggttaagatttacattctatatttaatctgtttagtctcatataatatgctttctctaataagatatgtcatctctctctccctctctctctctctctctctctctatatatatatatatatatgacatgtcatatgagagaaaaatattttatataagagtgaaatgattaaatttgaaccatataatcaaacatgaataatcaagatttaatcttaaaaagtcatgcgaccttttaagtagttacatcaaattaaatcttaactttatacagttcaaatttaatcttctcactcccatataagatatttcctctcataagatttggtagcggcgtcaatcatattggtggcgatgatggtgatcatggtgatgatcacattggtggtagtagtggtggtaagggtgatgacaacgacaacgaccacggtcatgtgatggtgacagtagagattaacgatcatggtagtggttgcggtggagtttatgatagttatggtggaaacagtcataacatgataatggtcatggttgtgataggtttaagatatcttaggacatcacatattaaatctttaagatatatatttttttgttttgtaaatcttatggagattttataattgaataaaattttaatttgaattttaaatgttttaatcttgatctctttcattttaatctaatcgttgaaattaattattttcattttcatataagaacataagatgattattctcatatgatagatacatcttttaaacacatacacacttgtaaattagctatgaaaaaattgaaaggataataactttattgtcttagattataaaatgtggttatgccgtgatataaaaaagtatgcaaatctataaataacatgtgttgatttaattgttaaaaagaaattaaaaaatacttaccttttgagtcaatcaaatcgttgattcaagtcgaatcaaatcgattcatgtcgaatcatatgcatatcgaatccaactaatagttgagattcaataggtgtctaattctagactaatgggttgagccgagtctaataataacatgtttggataaatggcgcttaatttcaattcaattattttttcacgtttatagtaaagaactctttgtagctttgagattttcacggtgaaaaattgatttttgtggtgccaccgcagaaccaaagataccataagtcgccaatgaattgcaattgcaaggtcaagattttttttcgtttgattctgaaatgaatatataatgatgatatgttgtaaaatttgggtccatggatacatccctttcgtatgtagcaccagctagcaagtgtgtgacagggacagaaaaagttgcacaaaacgacagttacacttgtaagggcctggaatggtttaaaacttggaactttttctctgactacactcactcactccttctctcttctctctcagtgtaccacactctctgccacctatggcccagcgtcaactctctccttttccctacccttccacctcctcttttgtcttccactttactctcccactaccactcctttcatgacttcaacttcaacaaccacctctttctctctcacttcttctcttgctcttatcaaaacaaaccactatgaataatactaacgataataatagttacaacaatacactctagcttctagcctgctacctttccttatcaccagcaccaatggcttcagacgccaaactctcctcttctgagtccctcaacggtttgaaattcggccaaaaaatctattttgaggatgttggtcttgctactccagccacctctcttacttcttcttcttcttcttcttctgctgctactgttacttcttcttcttcttcaaggaaaggaagaggtgggtctgttcaaccagctcaacctcccaggtgtcaagttgaagggtgcaaagtagatctgagtgatgcaaaagcttactattctagacacaaggtctgtggcatgcactctaaatccccttcagtcattgttgctggtcttcaacaaaggttttgtcaacagtgtagcaggtcaaaccccttctttcattattattattattattacctgaaacaaaactgaaaattctctttttcttcttcttgtgctattttaatattgaggtaagataaagtagggtagggccttgtttattttattcagtttttttgctcgttattctgcggtttggggtgtaggtttgtaggtgagatatgttatcctgttttgtcggtgcagttataggtgtgtgtgttacttgtttggagttggtttagttccttaatcgtggctttgcttttgttcaaggctttggtgtggtgtgattctttgtgtctgttcttgagatgtttgttgttcagttatgatttggctgattgtactcggtatactttacttttttttcccctcctagcgtagctttaaagtgcttatctgaacggctgtcttataacgcatgtgaaatattattgttttccttttcagcgaattgattcttgcacccccgggtgtgtttggtattaaaaataataacttcgtttgtacggaaaaaggcttgaagtgggtgtgcaatttgtagtcttaaaataacacaaacactactatgctgttttccctctagcctttgagggggccctcaattgtgtagtgaactattgatatgctgttacagttttctcattactgtttttgtgtttggattatcttttttggagaaataatatttttttttccttttatagattactagagagcctttgaaaaaaaaaaaataatgataattttccaaatataatctgttaccaaacatgaccaaactctttaggtatgtgatctgtgtaaagatacatccatgtattttactgtcatgaatcaagttgttttatagtgtgaaggcatgttatttgtactacaagagcaattccttgttgatgtgaacctaggattcttactcaaggcttgcattattatcaaaatggtagttttctgctaattggagtgagagtgactttagccttcccttgtctggtcatccataacgtgggtcctgattgttgaatttgatttatctatcttaaacagataggttgtctgattgtttatgtaatttccttgcatgaagacactagtcatgtattgcctccttgactaatgttagttttcatactgtttagtttgacttgttgatacattattgagaatgagtgcaattggtgctgctggatcatgaaatcctaaccacatgctttgcttatgtgactattcatctctattctgttttatggtgctagctatgtcagactgtcagttgtcacatgtactatccatttcaatttgactcattagtgctagaggatgctactgtgcattctaggatagtattcaatggtgtaataagcttgtattttggagacagatggccatcataattggaaagtttccaaccccaggatatggagaatatggtctatctgtcaatgaatgtggttttatatgcagtttttttttggagctgggataactttctgttggctgttctaagagtaatttgtgtctttttttttgtctcatggttcttccacagttgaaaagggttatctaagtcaaaagttacacttttccttaataatgaaaaacaatactcttctgaatagggtgacaatgaattgaaccaactcgaaaatagttcggaactcgattcaataatcagctcgttgaatttggttcatgaaccaaatgagctgaatttgagctcgttaaataaatgacccaaacttgagctgctaatagttcaactcatttgattcatgaattggctcaatatatatatatatatatatatatatatatataattgattgggttttctattattttatatatatctagggcagggtagtttttatttatgcagactttggttataattatgttttttttgtcataccttgtggccctacaaagctaatagaataagttcagttttgtcttttcacaaaaattaaacaatcattttatctaaaatctaccaataaattgaaaatttgaattgtataggatttaaaaaatttgacttttggttcatgtgatccaagctaacaagctgaattgaactgtttgtgagcttgaactgagttgagttcgagctgaaaaataaaggttcatgaaccaaaagtcaaatttttcagcaaaagatattaatgagccgaactgaactcgactcgtttccggacctacttctgaatattctaagttaaaatacatatcatgagaaagatcgtgaaaagatatttaagatggactttttagcatccaatctcccagcatttcagatgtactatcggtggatatacattgtggtttatcccctgtatttaagcttgatgacttctcttttgaattattctggtgtctttcactaaagagtaaatacaatgccatcccttttggtaatatagaagataaacaacttcattggtattgttttggtcttattttactaatacgttaaagctgaattccaagtatcatggagccgcagaatgttacttgagcaatgtttgaggattacttcagtaaaacagggctcaaggagcatcttatactgctaatatgccatctcattcaatgccaaacaagctcacatgatgtgcatcatatggaatacccttgttcttgggtttatacatattgcttatgttctgcaatctttgcaatgatgactaattcaaataagttagaaattccatctcatttgcatctgcatttaatgaaacaacaaatattttaagtgcttccaatttgtggcaacaacagcatgtcatgctaaggtaacagtagacttgtcatgttagcatttcttaagtcttggcacttgggtatgcttgtactcattcacctctctttgacataaattggaatcatttatatttgtaaaagataatatcatgtttcatagtcttgcacaggtttcatcagcttcctgagtttgatcaaggaaaaagaagttgccgtaggcgactagctggccataatgaacgtcggagaaagcccccaacaagctccctcttaacctctcgctatgccagactttcttcgtctgcttttggttagaaactatcagtaaaatatcagtttcatgtctcttccaattttttcatagggaagcatatttagtttcagcttgtgtcattcctggcatacaaaagtaaaacaaactctaatgtctggcacttttattgacagataatagtggcagaggtagcaactttctgatggaattgacttcatacccaaagcttagtctgagaaattcacttccaactcctagatcatctgagctagctcctggaaatcaaacctccacacttagctggaatggcaactcagagacatcatctgaccttttcttgcaaggttcggtgggtgggacaagcttcgccagcccgggacatcctccaggggaaagttacattggggtcaccgacacgagctgtgctctctctcttctgtcaaatcaaacatggggttctagaaacacagcaccaagtcttgggttgagtaacatgataaatttcaacgggacacccttgacacaacttgctgcatcatctcatggtgcatcaatccatcaacttccaaatacctcgtggtttttcaagggcattgattctggtaactgttcgcccgaggtggtccctgatctaggtctcggtcagatttcacagcctctcaatagccaacttcacggtgagctggacctgtcccaacagggcaggaggcattatatggatctagaacagtccagggcatatgaatctgctcattggtcactttaatgcacttgttttttttttgtcagctttgtaataacatgtagcagaaacctgtgcctacttggttacttaccagattgatgataactattgcttgttgttttgttttgttttaactgataagcaaggtgggtagccttgcctcatttgtggaactttcaggcaccgaatgaatctcacctgagagtgtgttttgttagtagtgtcttaagcacaggtatggtgttttctgccattgccgtcacctctggaattatttgtgattgggaggacagcctctttgatgtttgctaccaccgctccattttatttgatattatcttttgactatctgtcaactaagtgttgcacacattgcttgcctaagaacattttgcacagcattaagttgaatgacttggataacaaggcatgcttggttttcagttgggaggtgctcaacgtacatttcagaaggtaaaactaaaaaagctctttgtatggttttcaattgaatccaattgtaaattgggttttgtctcaaaacttagtttcaaatgaaagaaaaggtagggttgtttttgcaaaatgaagttcatataaattgaggtttgtaaactttaattaaaacatgcaacctgttcaaaaatcacttcattttttcaacacaatattagggtcacaaaacaattcagtgcatgtttacattagagtttgtaaatctaagtttgaactcagaatttcattttgctaattgagttagcaaaaagaatcttttcttctttcatttcaaattgagtatcaagacaaaattgaggtagctctcagtttataatatgatttttggtataaatcataagcatcctctagtgtaggtaatttagtttgagttagataagattactataagaagcaaagttttctctccgtagttgcatatctaggactcatttggaatctttggttaagtttgaatgatctggtgtagttgatttacgtgttggacagtagtttacaataaaactaaaactagtttttcagttttactttttgaatacatgttggggcctcctagatcaaaaagagattaattttatttattaatgaatttcggtatgcaaatcgtttagaagttacaactattggaagtgatatttctatgaataccatgataggaaagggaaaaaaacttaaaggagtctagaatagaatgaaacaaaaggggtggggatgcaggtaatcaaaattatggaaattccaaaaatgttatcatgatgagtgaagaagacatgttaaatgactttttgttgttttcagaatttttgaagatttggagtgaatattttttcgttgcaagtgtgtgcactaaggatattccctagataatttatgttgaactgaggtggacggggtgttgggagcacgtgttgaggttggttgttggatgttgagatcctctggtggttgtgacaatagtaattatttgcactttaacactcaagttaataagaattttggtagagtaataacaatattaactgatgcgtatctagagaagggagggttctgctatttataggtgttgggtggtttgaattaaatgagattggattagttaagctgtcaatctactgtcttagaatcccatacttcatctgtaaacacacatggtgtttatgcaattggtatgacttataaggtagttgcttaaaacagtagtgagaatgacactcctatcaaagtaagtgtcccaccttacaaggtatatgatgtgtagggcttggaattcgtttagaatggataatggtcgaatctttttcatgtaagcatctgaaatcgaacccttaaaagacttatttatttaccaattatgttgagttttgttggtgagtttaatttttaaaattgaaaataaaaatactcaaacatattttcttttacattttctgtttcactgaaaatataaagaaaaacagagatgatgaagcaaataaaataggcgtcggttgatggcttatttttgcttggctttcgggattataagcttgccattaataccttacaagaagtatttgaataatctatttttattatcatttttatggttggttcacatgcttccaaggaacccaacagttagtgcggaaattattgtttggtatttcaaataaaagaacccaataattagtgtgaaattattgcttggcatttcaaataaaagaggttgacttgtttagcttataaatgattttgttatcaactttgttcgaaattatttttgataaatattataggataagcaataatttttattcatattattatcattttataaattggtttggcatagcgcagca

SEQ ID NO: 298 - DNA, 8409 bp, synthetic construct
  misc_feature 1..8409: note = Synthetic polynucleotide
  source 1..8409: mol_type = other DNA
attgccgaattaagctgacaaaaaataatcaaacttaaaagtctcaaaataattacggaactactaaaccaaaggagctccttctgtgcgatgtaaacatttttttcttatggtttaaaaatataaatatataaattgaaaaaagtaattaaaattgacaatttcttaatatgcgttttctcttatttattattttcatatgtatatattattattttaaaacgtatattaattaatgttattttaaccttttaaaataaaaaatttccttatgacacgtaataaaaagaaacaaaagtaacatgttaagttctaggttttcattagtaattaataagtaaataaataataaataaagtgtttcattttcatattttgtgtaaggcgcttaccattacttaagcaacctatattaatactgacttttgtaccgtgcttttaattttaggataaaatataattttagtctttttatttttttaaatttataattttagttttttaattttttaattgagacatttcatttttcaatttaaaaagtttatgactttagtttttttattttttattgagatatttaaaaattcactattttagtctttacatttaatttcaaatattattatgtaaatattaaaggacaattatttgtttattatacatattataaatatttttttaaaattatttaattgttaacgagtttactttatttaaaataaaaaaatatacaatcaacaagtctaaaaattgatcaaagaactaaagtcacttaatttttaaaaatgagaaagaatatcccaattaaaaatagaaaaaataaaattacatagtttaaaaaagtggaagtaaaaatgttttagttaaaaaatataataaataaaatcacatatttaaaaaatagatgaagaaaaatcatattttaactttaattttataaccacgtgagagaagagaacattcaatcacagtaaacggtttattcaatatcatttaattatataagaaacgtgtgaaaaaaatgttaaatgatcataaattgactagtttcagtaggttgacatttatcttttttattttgttttttctgttgttgataagaggagcgaaagcgaaagactcgtccacgaggaatacgcaagagatcctctgccaaacacacttagcagggaagagcctatgaattaatattatcagtttcagattgttcatcgactggatccagataagtcacgaattcatcactatgatcctatttatgttttaaaaaattctaaataagtttatagctcgtaaacctctatacttaagaattaaatttaacttaaaaataaataaataattttaacatatttaaaaatataatcatgaacgtgattaaattttcagcacgctaacatattttttcagaatttaatattttatttttgatgagattcaaaattcaatatacacaatcttaaattagaatttaaaatataatcttatgaaagaaaaattaaaaaataacaataattttattatttcaaattttaatatgcaattatgtttgataaaaaaataaataaatctataaaataaaataagtataagatgtaaattaaattttgtgataagagtgatgtgatttcgtgagtaattgatttagagtctaatcgtattaaattaaattgatttatatcaagtatatgcgatccaactagtaagttaaaccggttagatatttgatttccatgtcaacaggttaatacagtcggtccaaaatcgaggccaataacaattctaagaattgcaaattttgtcttctttttcttttgattctgaaatgaatatataatgatggtatgttgtaaaatttgggtccatggatacaccccattcgtatgtagcaccagctagcaagtgtgtgacaaggacagaaaaaagttgcacaaaacgacagttatacttgttaggcctggaatggttaaaaacttggaacttttgaaccaatgagagaaattgtctgtgttgagaagaaaaaagaaaaaaagaaaaaagagagggtacaatctgtctctcgtagctggcttggctccatttctctctcagtgtacctgactctgccaccttccctacccttccaccccctcttttgtcttccactttactctcccattacaactcctttcatgaacttctatttccaacttcaaccacctctttctctctcacttaactttcccttatcaaaacaaaccacaatgaataataatactaacgataacaatacactctagacacctcaccttatcactccatcacctatggcttcagacgcaaaactctcatcttctgagtccctcaacggtttgaaattcggccaaaaaatctattttgaggatgtgggttttgctactcccgctacctctcttacttcttctgctactactgctaccactgttacttcttcttcttcttcctcttcaagaaaaggaagaggtggttctgttcaaccagctcaacctccaaggtgtcaagttgaagggtgcgaagtagatctgagtaatgcaaaaacttactattctagacacaaagtttgtggcatgcactctaaatcccctacagtcattgttgctggtcttcaacaaaggttttgtcaacagtgtagcaggtcaacccccccttctttcattattattattattatctgaagaaaaattctcttttttcttcttcttgtgctattttaatattttactatattaggtgaggtgagataaagtaggatagggtgccaaggaagattggtctagttagtaaggaacaaattcatattttcatggagttgttgctgaaaaatctgtaaatagcattgtaagtgttccgtgagtcttgaggtctatcctggtcctgttgagtctctcttttttttttttttttttttaaatttttcattaccaaaaaatgtagggtagggtcttgtttattttattaagtttttttttgttcgttattctgcggtttggggtgtaggtgagatatattatcctgttttgtcggtgcagttatagaggtgtgtgtgttacttgtttggagttggtttagttccttaatcgtggttttgcatttgttccaggctttggtgtggtgtggtgtgattctttgtgtctattcttgagatgtttttgttcagttatgatttggctgattgtgctcggtgtactttgcttttttttttttcttcctcctagcttagctttaaagtgcttatctgaatggctgtcttgtaacgcgtgtgaaatattgttttccttttcagcgaattgattcttgcagcccccaggtgtgttttggtattaaaaataataacttcgtttgtacggtaaaaaggcttgaagtgggtgtgtgcaagttgttgtagtcttaaaataacacaacactactattctgctgtttttcctccagccattgtgggggccctcaattgtgtattgatatgctgttacagttttctccttactgtttctgtgtttggattatctgttttggagaaatattttttttccttttatagattacttaagagagcctttgaaaatataaaataacgattattttccaaatacaatctgttaccaaacatgaccaaaactctataggtatgtgatctgtgtaaagatacatccaggtatttcagtttagtgttatgaatcaaattgttttattgtgcgaactgcaaaggcatgttatttgtactacatgagcaattccttgttgatctgagtgaacccaggattcttaaggcttgcattattattatcataatgatagttttctgctgattggagtttgtgagtggctgcaacctgcccttgtctggtcatccataaagtgggtcctgattattgaatattaaattttatctatcttaaacagatagtttgtctgattgttcatgtaatttccttgcatgaagacactattcatgtattgcctccttgactaatgttagtttgacttgttggcacattattgagaatgagtgcaattgatgttgctggatcatgcaatcctaaccacatgctttgctattatgactattcatctctattatggtgctagctatgtcagactgtcagttgtcacatgtactatccatttcaatttgactcatttgtgctactgtgcattctaggatagtattcaatagtgcaataagtttgtattttggagacagatggtcatcataattggaaagtttccaaccactggatatggagaatatggtctatcagtcaatgaatgtgggttttatttgcagttttttttttttttttggagctgggatggctttctgttgactgttccaagagtattttgtgtcttctttttatctcatggctcttccactgttgtctaaggcaaaagttactcttttccttaatatgaaaaacaatactcttctgaatatgctaagataaaatacatattatgagaaagatagtaaacagatatttaagatggatttcttagcatccaatctccctgcatttcagatgtactatccgatggatacacattgtagtttatctcctttatttaagcttgatgacttctcttttgaattattctgatgtctttcactaaagagtaaatacaatgctatcccttttggtaatagagaagataaagaactttattggtattgttttggtcttattttactaatatgttaaagctgaattctaagtataatggagccacaggatgttacttgagcaatgtttgagaattgcttcagtaaaacagggctaaaggaacatgttagatgctcacatgatgtgcattactgcaagatgtgagaattattactacatcatatggaataccctttttcttgggtttatacatatttgcttatgttctgtaatcttagcaatgatgacaaaatcaaatcagttagaaattccgtctcatttgcatgtgcatttaatcaaacaagagatgcgtttaagtgcttccaatttgtggcaacaacagcttggcatgctaaggaaacagtagacttgtcatgttagtatttctagtcttggcacttgggtatgcttgtactcattcacctctctttgacataaaaaattggaaccatttatatttgtaaaagctaatatcatgtttcatagccttgcacaggtttcatcagcttcctgagtttgatcaaggaaaaagaagttgccgcaggcgactagctggccataatgaacgtcggagaaagcccccacccagctccctcttgacctctcgctatgctagactttcttcatctgcttttggttagaaactcagtaaaaaatcagtttcatgtctctttcaatttttccatagggaagcatatttagtttcagcctgtgccgttcctggcatataaaacaaattctaatctctggcacttctattgacagatcatagtggcagaggtggcaactttctgatggaattggcttcatacccaaaacttagtctgagaaattcacttccaactcctaggtcatccgagctagcgcctggaaatcaaacctccaccctcagctggcacgggaactcggagacatcatctgaccttttcttgcaaggttcggtgggtgggacaagcttcgccggcccgggacatcctccaggggaaagttacactggggtcaccacctcgagctgtgctctctctcttctgtcaaatcaaacatggggttctagaaacccagcaccaagtcttgggttgaataacatggtaaatttccacgggacgcccatgacacagcctgctgcatcatctcatggtgcatcaatccatcaacttccaaataccccgtggtgtttcaagggcattgattctggtaactgttcgcccgagggggtccctgatctaggtctcggtcagatttcgcagcctctcagtagccaacttcacggtgagctggacctgtcccaacaaggcaggagacattatatggatctagaacagtccagggcatatgaatctgctcattggtcactttaatgcacttgtttttgtttttgtttttttgtcaggtttgtaataaaatgtagcacaaacctgtgcctacttggttactgaccagattgatgataactatttcttgttttggtttgttcaaactgataagcaaggtgggtagccttgcctcatttgtggaactttctggcggcgaatgaaatcacccgactgtgttttgttagtgtcttaagcataggtacggtgtcttctgccattgctgtcacctctttgctgtttgctacccctgctccaagttatttgatattatcttttgactatctgtcaacatagtgttgcacacattgctcgcctaagaatattttgcacagcattaagttgaatgacttggagaacaaggcatgtttggttttcagtaaggaagactcctatgcaggttttaataattaaaaccaaaaggggtgttcggttttcagctgaatctgattgtaaactgagttttgtctcaattttgttttgaagtttagtttgaaatgagagaaaaggtgggcttgtttttgcaaaactaaattcgtttaaatttgagtttgtaaactttaatccaaacaagcaaatttgttcaaaagtcagttaatttcgtaacacaatgttgagtttagtgaatgtttagattaagatttgtaaatgcaagttctaactcaaaatttcatttagcaaattgagtttgcaaaactagtcttttcttcttcaagttttcattttaaattgagtttcaaggaaaaatttaggtaaatctcagttcacaatcgactttaatttaaaccaaaggtttttcagttttactttttgaatagattttgggtcctcctaactgaaaactgaacaggcctttaaatgatagaagagatttatttcatttgttaatgaatgttcatatgcaacttgtttagagcttttggaagtgatatttctattttcatttttcttttccatcactctcgttctcaattttttccatcatcttcgttcaaaatttattttcctcttgttctgctgccaacctgcttgaggaaccaactgaaataaaatcattacccgtggaccaaaaataaaattgaaattccaaatccaaccttagttatgcaattaaatcccaagtcatcgtgattcatgagcactgtcctgacctttcacccttctctacaacacaattttatgcctacaaacccgccaatggaaccagtgtctactgcattgcccctgtaccattattaaagtactaccatatactattccctgcaatctacataaagatagtaaggaatgaaaccaaaattatggaaatttgaaaaatgttaccctgcaatttttatatgaataccatgataggaaagggggaatgaacttaaggagcctagtatataatgaaacaaaagggtgggggatgcaggtaaccaaaattatggaaattccaaaaatgtcatcatgatgagtgaagacatggttaaatgaccttttgttgttttcagaatttttgaaaacttggagttaatattttttcgttgcaagtgtatgcactaaggatgttccctagataattaatgttgagctggggtggatggagtgttgggagcacgtgttgaggttggttgttaggtgtggtagctaacagtgtattgtgtgacaatagtaattatttgcactttaacactcaagttaatagaaatttttggtagagtaacaacaatattgaatgatgcgtatctagagaagggagggttctgctatttataggcgttgggtggttttaattaaatgagattgaattagttaagctgtcaatcccatactgcttctgtaaacacttgtatctatgtgattcgtatgacttataaggtagttacttgaaatagtgataagaatgacactcatatctaagtgagaaggtgtcccaccttgtaaggtatatgatgtgttggattcttattgggttgaattgttggggtgtagaaacttcaaatctatttagaatggatatggatcaaatctttttcatataggtatcctaaaattcaacctttattaacatgattaaaagacttaattatttgtcaattatgttaggctttgttggcaagtttaatttttaaaattgaaaataaaaatgctctaacatattttcgttttcactttttgtttcattgaaaacataaagaaaaacagagaaatcataaagccaataaaataggccttggctgttggttttctggatggatttttgcttggcttctggattagaagcctacctttttcatttgttgaatagaagcctaccttttaataccttataagcaatatttgaataatctatttttattatcatttttctggtt

SEQ ID NO: 299 - DNA, 8996 bp, synthetic construct
  misc_feature 1..8996: note = Synthetic polynucleotide
  source 1..8996: mol_type = other DNA
aaattgattgttgttgcttcttctgcattctaagcctatccctcttaagattattgaggccacaaggtctaacacagtgaaggattctgaccagcaaggatgttgagtgaagtgtcaccgcatgaagtgtcatagtgtgagagccgcaaagaagtaaaaagttagggatgttacgccactcgttaacccctgtgatttttcactctttttctcttgtcacgtcattggaaatgaatgtcatcatgctaaattttaacataaatttatgctattcagttggataaaatgaaaataaagaaatatttttagtaagaaattatatttaacaagaaaaaatatcactatttcatatcatgctagagaacacatgttaaaaaatatgaagacaaaaagaatcaagagaccttgacaaattctaacatgtgtgactaaagtatgatagtgaggtatgacatctatttatattgtgtatcttttaacgatttcgataactttttgttttgttaacattgtctaggagtgatgtgtattatgttgttcctacttgtgtgtgtagtgatttcattgcattctactcacattaagtatctagaatcccagtattagacttaggtgtggtttgtattggtttctctcacttacgttgttagaatggttttatcgcatcttacttatgcattcaagtatctagaagcctagtattgagcctagatgtagttttatagcaccctactcaggtgttaagtatctagaatcctagtattgactttgttaacctttattgtcatttgtcaatgaattgcgtattatattgctcctatccgtgtgtagagtgattttatcacgctactcacgtgtttggtattggatctaggttcttcaaacttatgagctttgttagcttacttaggtgatccattgaataatgaatttggttaggtcatcctctcaaatttatcttttattttgaacattttggtcaacctattatgattggaggttcacaacttcttaatcgttgagcttaaaagatgtaaatcctggggtgattagtcactaactagacaatatacaacccatcaaacactgaacctaaaaataaatatttgttgtgattgatttctctaactagccagtccataaataattatttaatttctaaaaaaataaaacattttagttataactcaagaatgaaaagaataatgtatttaaatttatagttctagataaaatcattttcttattttactttaaattaaattaagtgattttttatataaaagaatattttatttgtattaaattaaaattaattttttggtatgaaattattaactatttttaaattattttaaatatatttttaaaaataattattataaaaccaaaatattattatacaaaacactttgtgatttaataaccataagtgatagcaacacattttataacactctatttaatatatttttattatagattaaaatttattaaaaattacaaatgacacttattaagaaaaattaaaaactaaatcattaagtatatgaataaactaaaataagattattttaatttaataaaaaatctcaagttcaaatcttatgttaaaactgtttaaaatacttagaaaaagaattttatcacctataatgatattttttaactcaaatataattattatttttggataatacttgtgatctaaataaaaaattaacttataatattttataatttgtaataaatttcaatcaaataattaaaaaatattttaaaagcaaataacgtttctcaaatatataaaaataaacattaaccatcaaaacgtctaactaattaattgatcagtttctatgaattattctaatattcttttaattcttataaataaataaacattaaatcattactccatttaattaaattgataaaatttcagcattaaattgatgacattgacattattattaactactagctagtagctagagggagcatcagcatcagcatcagcactgagtagcagcagcaggagcaatgagttctggttcagtcaccaaagcaccaccaccctcttcttcttcatcacccaactcttcctctgagtcccttgatggcttgaagtttggccagaaaatctactttgaggatgtgagtgttgcagcaacaacaacaactcaaggggcctacaaaacaagtcgtgtggtttcttccccttcttctgcttcaaagaagggaaggggtggttctttgcaacctcctaggtgtcaggttgagggctgcaaagtagatctgagtggtgctaaggcttactattctaggcacaaagtttgtaccatgcactccaagttccctactgttattgttgctggtttggagcaaaggttttgccaacagtgtagcaggtcaccatcctctctctctctctctctttaccttttgtttatttattattgacttcaaaaagggaaaaaatcttcttttgtttttgttactcttttgtttttgctatttttagctggcttgctatattgggtaattctacttctgctttggtttcctttcctcatcttacttccattgtttttgctatttttagtgcatttgctatactgctatgctattcacatgctcttgttttttagaagaaaaatggttttgtttttcattccaatttttacatccctttgttgtgtggtttttggctgtatcttttacatttgttgcttttcatttcaggaatttaatcatgctgattttgttccttgtgattgttctctgtagattttttttaagttgattgcgtgtaatgagtcttgcacttctcttacttcataatgaagtgtgtgcaaggtttgttttactttggtttctaatgtttctttatgattggataattgttaatgtgtctggatttgatgcatctattggtgctgtgagttgtagatgataacataaccatggtgttgtgttgtttatcttgtagttatggttagtgattggcgagggaatatgctttagaaaccaattgcttttgtgagcttgtgttgtttcttctttcctccttcatggtatcatggagtgctcatgaaagggaaaggggagttctcagcgaaagaaaaaactgtacaaatgagtgtgaaacagctgcataacatgtgaatcaatgtaaattggactctgataagtgaaaaatttagggtagtttagacataccaagccttcaagagggtcaatatataaatcaagatttggaataataagaccatgcaatactattttcccatttttaacttgttatttgctcatgaaatgatacctctctcatgtaatgtaagttcattccacaagaaaagtgcttttttccccacattattgtcatatacaaatcatgtaaggcctccttaaggtaggtagggtctaatgtcctatctttgtcttgttaacaactcaatggaaatgactaaaattaatgttactggataattcaattaacttgcttgtgactgttagatacctcaatggggcttgaatcaaagatgtaagattgtaagtgaaaagggaatagggttagaagggagaggaagagagggatatcttttggaagttagatggttaacagaagagggactgggctctcaaattccgagtgcagagagaagagtggtctgtgttctcattgttcaatatagaattatcattcttgttaccgaattctgatctagatcttaagacagtatccattttctgttcagtagtgaaagcttgattacatctgaatttactttcctcttcaatttgactcatcattgctagcaaatgttacagggcattttagggatgtgtttaatggcaaaatgttactaacaagtgggtagttttggagatggatggttatcataatttggatagttttctgaccataagatttggaatacttggcctacttgtttgccagtatttttggacaatagttatgaggtgaaataaaagagccacttaagtgacctgattggttgggcattgcataaaatgttgcagatttgtcctccactgacaaaccaattatatttttccagattaagatattttagggaattattctcttcatgcagggctaaaattttcaaaaggaattgccaacatgtgctctaagttcctattgatggttgatttcatacaccttcgttcatttgtcttgtgtattgtggtattcatatgaagatcatattagaaaataatctcttctaaaactacttaaaattgaacattcaagccattcaagatgctgtatgttctctggtgaaagataaaccttgtcgattgacaattgtgggatgttaattacttatgtggaataatggtgtttagcaactaaaaggtatagggatttttttggatgttctcctggcatggatatagtacatgttaagcactatagaataatggtgtttagcaactaaaaggtatagggattcaaatgtttttttaaacttaataacaagagtttaaacataccttgaggtagtgtgctaatgaagctttgtctgggctcttaatatgatgttatgttattatattctatggttgttgtgacttgtgaccttaaattggaatggtattatctcatggtgttaaaaatctcaaatgtcattctttttcctccaaatctcatttttcaattctttgatgctatgattatctggagaacttgtgttacaatgagagtttcaactgttttggggcttatcagcacaaatctattaatgtggtaagcttccactattgttagggagcttagatagctctctggtggctgtttcctgtgaagtattagtctattaatgtcccttttgggcttttgttccgtccaaggtcgaataggattttattgcacattttccataccctctttaaggtgcttcctattttccataatgaaattaaccagaaagagggaagggatcaataaatcgacagtgctgttatgaatcaagacagtgtttaaaaaggttttggagacatagcacagatggatgttttcaaatctactattttcagcattgtagatgagcaaacacgttaatgggcgaaacaaaatgtcatgtattcttattttggtaagaaagtgaatgtatattttaaggtctgtattctgtattcaagctcaatgcttctctttcaattatgcaagtatatattgtaccaaacatgaaatttaagttcgaagtatcttggatgggatgttccttgattgtttgttgagaggattttttgctgatgccatcacaatccttagtttaaaacatttgtgacttaggctgtatttccattatgatatactgtatccaatgcattaagatttctgataagatatgggaattgcttcttatggaaatatacctatggtattggctctatacatattagctttgttctgtaatgttgtcacaatgactaagttttactgataaattctctcatttgtaatgtgaatttaatataatgaaaggagaaatagttcaaatgcttccaattctgtggtaacaacaggtcaacagcattgcatgtaatgctaagaagcgattttgtcatgttagaattctcaaatcttgtgtcatctcatgttctcttttatagtaatgttttacttgctagaaattatatttcagaaatcttgtgtctgatcatactctttggtatcatgcttttatataaacatggaagcatttatatttcagaaaagttaatatcctctctgatggcccttgcccaggtttcatctgctttctgaatttgatgaaggaaaacgaagctgccgcaggcgacttgctggtcataatgagcgccgacgaaagcccccgccaagctccacactagcctctcactatggcagactctctacgcctatttttggttagaaattatcagtaaaacatcaattcgaaaccactctctctgcttaatgtatccctaggaagtaggaagcatcaataatttcagtttttgctattccttataaaacattcaactgtttcacccatgtttaacagataacagtggccgagccggtggctttctgatggaatttgcttcatacccaaaggttacattgcgaaatacactgccaacacaaagatcagccgagccggctctcggtaaccatgctgcaacacttacctggcaaggaagctcagacacaccatctgaatttttcctgcaagaatcagggagtggtggaacaagcttccttggttccaaacatcccttactggaaagttacactggagtgactgactcaaactgtgctctctctcttctgtcaagacaaacctggggttccagaaacacagccccagcaacaagtgttgagctgaacaacaacttgttgaatttcaatgggacatccatgacacaattttccgcggcttcttcacaagctgcagccatccatcaacttccaaatgccacctcatggttgtacctaaagggagttggttctggtgattgttcgcctgagggtgtccccgatcttggtctaggtcaaatttcgccgcctcttgatagccatcttcatggtgagcttaatgtgtcacagcagggcaggaggcattacatggatctaggggaatccagggcatgtgagtcttctcactggtctctttgacgcacttctatgctatctgttactatttgatcagttaccagactggcactaactactagcattttggtttctttgaacttagaagcaaggtgggtatatccttcttttggttgtggaactttgaaaattgaaacatccttattttggtttgtttgaacatatcagcaaggtaggtagccttgttttttttttttttttttgtggtacctttcagacattattagtttggtattggaacttagaagcaagatggggttgtcctgttttattttgtggaaatttcagacgtgtaaatcattttattgtgggtatgatcgatgttttctagcatatatgtctgtgattccactgaatgcatattccttatattgttatagctgtacttggagttatttgttaaaatcattagattaaggggatgtattggattagtattttaaagaattttaaaagattttttttataaaaaaatagaaatttaaatgttaaaaataagtcttttagtatacaattaagattttttttaccttcaaaaaagtcttatgatatttaaaagtatataatttttcattttgatgaattattttttaaaataaatttcattggattttatgtgattttttagtaggatatctaaatattttacctattaaacaatcttattcaaatcctccagatttttctggactttcttgattgattgtaacacctttctatttctcatcacacatatttccttctacttagtagaagatgttatatatacattcataactttttttcacaaagaatgttgttcaaacaaatttccaattgaacaaataggttaataccaaataatgaagacaatagttttgaaccaagagtgagaatgagaagattttaatgcatggagggttaatatagaaagatatgaaactagaatcaatagactaaaaaaaaattgttgtggaatttttttgaaaacttgtccataagcagaagccataaattaattggcttaattgtgcttttaattctctgacttttcaatttttgatgaattttatcctttaagatacttgctaattttattctatgccaatttattcctaacataactttactttttacttcaattttttatttatttttttggcaaattttacactcgacttttttttttaatcctatcattttatatttttggtaaattttaccccaacattttatatttactaatggataaatgataaggggtaaaagttgcaagtttttcaaaaattgggaataaaatgtgtcaaattaatttggaggataaaattcataaaaaaagaaaaagttaagataaaaaatgtaattaaatataattaattaaataacaattttggtgtatgaaagtagctcaaggtcaaagacattcttagagtaatatactgatattttgaataattacattaccatatatttatctaattgaaatttaatttatttgaaaaaattgaattgattttacttttttaaacattagagtttaatggttatgtccggataatgtatactaattttagacggtgattcaatcataaataattgttataactttgaaagtaattatcataaaaatctacaaacatgatgtgttttaattggattaaaaatattacattaacatatatttatctaatttaaatttaatttatttgaaattgtactaattttactttttttatttaaaccttagagtttaatattatgtactgataatgtaaattaattttagactattattcaatcataaatagttgttgatataacttttaaagtgattatcacaaaaatctacaaacatgatgtgttgtaattggattgacgataaaattattttacactattagtgcattgcctaatttctctaaatctcatattatctttatcattttcctttgacttttgtaattttgaatgttttctagaacttcgtgatctttacaaatcttttaaatttatacaatccttgttggataagtggcctcaataacttaaggagggggggggggggtgaattaagtttcaaaattttcccgttaacaaatgttaaccccattttaaatgatatatgataggctcagaatgcaaaaagaagaagaagcaatcaatttaataatgttttttttaaatgcacaagataaagtaaactataataaaataactgagataagggaagagagaattacaaacttgttttatcctcgttcggccacttcccgtgcctacgtccagttcttaagtaactcacttgatatttttcactatctctgtaaatcctttatagattttgaac

SEQ ID NO: 300 - DNA, 9441 bp, synthetic construct
  misc_feature 1..9441: note = Synthetic polynucleotide
  source 1..9441: mol_type = other DNA
caaaaaatacaaatcataaaagacactcattaaataaaaagtaagacttcaaaattttataatttgtaattatttcaaccaaacaattttaaaaaatttcagaaaaaattattaaagaagtacatataacgtttctcaaacatataaaaataaacattcacaacgttgtctaagcaagtgcatgagatgttgtaattgtatttcaattcttataataaataaaaagtttgaatagctttttagttcttgtaatttagcattttttgcttttcattcatgcaatttttttttgttttcaatccttgcaaattatgcttgttttattttcagtccttaaaatgatttagatagtttttttttctatttaaagtattatctaaactgctttaagaaaaaaaaaacaaatacaatttgttagctgtgacgaacacacatgtaaggttttggggacaattgccccacattttttttatttgtatgtgtaataaatttttttcacccccataaaaaataggtattgccccctttaagacaactttcttttaaatgaacgtataaaatgaatcaaaacaaagaataaattcatactaatttcactactttatccttttaatttttataatattgataataaaaaaaattatagaatatttttgaagtatgaaaaatactagatgaccattgtaatttgtataagtttaaacattttgaatttttttgtcataacattatgtattttgtaaaattttgtgatatttttattttgcgaaaatattataaactattggttgggttaattatgtttttggttcctaaactttttgtaaaattcccttgtagtctctaaacaatttttgtaatgtttttggtccctataatttctttcattaatagttctagtttctattatcaagcaacaatgttaatttatgacataaccaatcacattaacttgtcacaccatcaagccgtgatgacaattaagtgtaattctatgaggaatttaaaatacaatttataaaaaaaattacatatgattgttccatcaaacccttatgatgtgataagtatgctgctaacatgaatattacattatcaattaatgttgttactcaataacaagtattaaaaccatcaaaggggaagagtaaaagtataaggaccacacatactaaaaaattgtttaaagactaaaagtaaaatttgtaaaaagttttggaaaaaaatataattaatcctaaactatttcttggtcttatgaagatttttttttggattcacagttattttcaaggactaaaacaattttttttctttaaaaaattaaaaataaaaaatactaaatcacaacaactaaaaagtatttaaatctaaataaaaaaaaaggaaaaaaaagtcttttgacaccaacaaactgacaaattgagagacatagaaagaaaaatagataaaaataagagttactaataagatgtttaaaagaaaaagatgttaaaatatcaccactcaaaaaataaacattgacatcagtactccatttactaaattgataaaatttcagcattaaattgataacattgacattattaattattactcacaactaaatgaattaatgtataactagctagtactagagggagcatcagcatcagcactgattagccagcagcagcaataggttctggttcagtcaccaaagcaccaccctcttcaaggactaaataattatattctttaaaaaacaaaataaaaaaaatactaaattacaacaactaaaaagtatttaaatttaaaaaaaataaggaaaaaaaatcttttgacactaacaaattgacaaattgagagacatagaaagaaaaagaaaaaaaaaatattaatataataaataacgtgatcagttaaaaaaaatagataaaaataaaaatgactaataagatgtttaaatgaaaaagatgttaaaacatcaccacttaaaaaataaacattgacatcagtactccatttactaaattgataaaatttcagcattaaattgataacattgacattattaactattagtctcaacgaaatgaattaatgtatacacccacgctagtactaaagggagcatcagcatcagcactcagtagccagcagcagcaatgggttctgcttcagtcaccaaagcaccaccctcttcctcctcatcacccaactcttcttctactgagtcccttgatggcttgaagtttggccagaaaatctactttgaggaagtgagtgttgctgcagcagcagcaacaacaactcaagggggcaacaaaacaagaggtggggtttctgcttcaaagaagggaaggggtggctcagtgcaagctcctaggtgtcaggttgcgggctgcaaagtagatctgagtggtgctaaggcttactattctaggcacaaagtttgtgccatgcactccaagtcccctactgtcactgttgctggtttggagcaaaggttttgccaacagtgtagcaggtcacctcttatctctttctctctctctcactttctacttttttatttattattgacttcaaaaagaaataaaaaaatcttcttttgtttttttttttttttttagttggcttgttatattgggtagttctgcttctgctttggtttattttcctcatcttactccttggtttctgctatttttagtgtttttgctattttgggtgcttttggaatactgctatgctattcacactctctttttagaaaaaaaataatggtttttgtttttctttccaatttttacatcctattcttttgtggttttggctgtatcttttacattgcttgtttttcatttcagggctttaatcgtgctgattttgttctctgtagattttttttattttattttttatgttgattgcgtgtaatgagtcatgctcttctcttacttcgtaatgaagtgtgtgaaggtttgtttttctatggtttctaatgctgcttaatgattggagaatggttcacgtttctggtttgatgcatctgttggtgctgtgagttgtagatgatagcataaccatggggttgtgttgtttatcttgtagttaacttatgggtagtgatttgcgaaggaatatgctttagaaaacaatggtgctgttgtgagcttgtgttgtttcccccttcccctttcatggtatcatgaagtgctcattaaaggaaaacggagctactagggtaggaaataggtgtagaaatgagtgcaaaacaacaactaagttgcataacatttaaagcaatgtagagtggactcagataaatgaaaagtttaggattctgttttgacataccatgccttcaagagtgtcaagatttggaacaataagtccataatatacaattttttcccttttaatttgttatgtgaagttgatattgtccttttgttcatgagatgatacctctctcatgcaagttcattccataagaaaagtgctttttccccaacattatcttatgcagatcatgcaatgcccccataaggtcatagggtctgatgttgtatctttgtcttgctggacaatgcaatccatggtaacttgctattgtgactgttagataactcaatgggacttgaatcagttaattacagaaataacagttagaaactgttatatgtttaactcaagcttgattacaactgaaattactttactccaatttcattgttgctagcaaatgctacagggcattttagggatttgcttaatggcaaaatgtccctaacaagtgtgtaattttggaggtggatggttatcattttttgggtggttttctgactgtaagatgcaaaatctggcctatttgtttgcagtattttttgtgacaataagaatgaggtgaaacaaaagagccatttgagttctatgatctgttggacattgtgtaaaatgttgcagatctcttccgacaaatgtccaaaccaattactcttattgggtttgagatattttagagaatgattctcgtcctgcagagctaaaattttcaaattaagatgcaacctccctttgccaacttgttctctaagttcctattgatggtctggtcttgtgggatccatatcgtagatcattttagaaaagaatctcttctagatgacttgatattgaacattatatgttgtagacacatgtgaccttcaatccattcaagatgcaacatgttctcaggggaaagatagatctcattgactgacaattatgggatgttaattacttatgttgaataatgatgtttagcaactaaaaggtgtaggaaatttctggatgctctcccagtatggatatagtacttgttgagaactatggcataattcttgtagctaacccctttgaatgtgaaaaaaattgtttggttgttgattgctgctcttctgttttatggcatggttggaacattttttttttatcatctgatgtgtggtttattggtaattaattgaagttgaactttctttcactggtgtaaagatgtagatggaatggtgtgtaaggaaaaatttgatggctacttagtatgaaaaatttgatgaaaagtaccacaaaaatagacatggctgatttccatttgatttttttataagtaatctaatttgttaattagcagaaagcctcttctttgattttgtgcaaattcatttgtaatgttttcatatcacatagcaccggtcggtgtgtgaatgctatttacaattattagacttgcaggttcctttccacagaagctacattggtgtgtatatatatatatatatatatatatatatatataacatattctgtgtttctctactgtgcttaactcaagttcaaatgttttcttactttacaatgagtttaaacatgccttgaggtaacgtgctaattaagctttatctgggctcgtaataggatgttatgatattctatagttgttgtgaccttaaattggaatagtattaacatatggtgttaaaaaatttcaaatgttgctctcttttctccaaatctcatttgtcaattctctgaggctatgatgatctggaaaacttgtgttacaatgggagtttcaagtttcaactgctttggggtttatcggcagttcagcacaagtctatgaatgtggtaagcaaccactgttgttgtggagcttggatagctctctgatggctgtgaacaatgaagtgtaacttttatccctttttgggctttttgtcccgtcaagggttgaaaaggattttattgcacatatttcataccaatttaaggtgcatactattttccataatgaaattaatcagaaagagggatcaacaaatgcatagtgctgttatgaatgttcaagacagtggattaaaaaggttttggagagtacaaatgggcatttttcagaactactaatttccgcattgcagatgagcatatggtatttggtgaaacaaaaggtcatctattcttattttggtaacagagtgaatctatattttaaggtttgtatcttgtattcaagctcaacgacttctctttcaattatgctagtatactgtataaattgtactaaacataatgttatccctcttagcaagaagcgagcgagaaatttttggtatctatgttttagtactgatatccatattgaagttgagttcgaagtatcttggtaccatgggatgttccctgattgcatgttgagaggattttttgctgatgctatcacaacccttgtatccaatgcattaagatttctgtaagatatgggaattgcatcttatggaaatatacgtatgttcttggctttatacatattagctttgttctgtacttttgtcacagttaggaagtatgagtgataaattctctcatttgtaatgtgatcatttaatttaatgaaagtataaaatacttcaaatgcttccaattctgtggtaataacaaatcaacagcattgcatgtaatgattagaagcaattttgtcatgttagaaattctcaaatcttgtgtcatttcgtgttttcttttgtagtatacttttacttaaacttggtagcaattatatttcagacatcttgtgtctgatcatactctctttgtatataaatctggaagcatttatacttcagaaaagctaatatcctctttcatggccctttcccaggtttcatctgctttctgaatttgatcaaggaaaacgtagctgccgcaagcgacttgctggccataatgagcgccgacgaaagcccccaccaagctccatattaacctctcgctatggcagactctctgtgcctatttttggttagaaattatcagtaaaacatcaattccaaaccactctctctgctttatgtatccctaggaagcatcaataatttcagtttttgctattgcatataaaacattcaactgtttcccttatgtttaacagataacaatggtcgagacgatggctttctgatggaatttgcttcataccccaagcttactttgagaaatacattgccaactcaaagatcaactgagccggctcctgggaaccatgctgcaacactcacctggcaaggaggttcagacactccatctgaatttttcctgcaaggatcagggagtagtggtggaacaagcttccttagttcaaaacatcccttagtggaaagttacactggagtcactgactcaaactgtgctctctctcttctgtcaagtcaaacctggggttccagaaacacagccccagcaacaagttttgaactgaacaacaacttgttcaatttcaatgggacatccatgacacaatttgctgtggcgtctttgcaagctgcagccatccatcaacttccaaatgctacctcatggttgtacctaaagggagttgattctggtaattgttcgcctgagcgtgtccccaatcacggtcttgctcaaatttctccccctcttcatagccatgttcatagtgagcttgatgtgtcacagcagggcaggaggcattacatgatctaggggagtccagggcctgtgagtcttctcactggtctctttgaagccccttctttgctatcagaaagtatgaggggaatctgtgcctatttgatcacctaactactagcattttggtttctttgaacttagaagtaaggtgggtatatccttcccttgtttgtggaacattgaaaattgaaacatccttattttggcttgtttgaacttataagcaaggtaggtagccttgtttctttgtggtacttttcagacattactagtttggtgttggaacttagaagcaagatgggtagtcttgttttattttgtggaaatttcagacctgtatcatttgaatgtgggtatgattgatgttttatagcatctacgtctgtgattccactgactgcatattccttatattgctatagctgtgctagaactactttcgagtttttttgttagaatctatagattaaatgataataataagtcttttggtattaggattaggattatgatttttaaaagactttttttttcaataaaaaatatatagattaaatgataacgataagtcttttggtatttaattaagttttttaaaatgaatttccatgaatttcatgcgatcttttaatatgatatacaaatatcgcacctattaaacaatcttatccaatttagatttttttttctcttttttgttggttgtcacacctttctctatcccatcacacatgttccattctctctctctctctctctctctctctctctctctatatatatatatatatatatatatatatatatatatatatatatatatatatatatatatatatatatattgcatcttaagtttaccaaacaaataatgaagacaataattttgaatcaagagcgaggacaagaagattctaaagcatggaggattagtttagaagaagatagtactagaattaattgacttaaaaaaaaataaagaaaaaaagtgtgatggaaaaatgttcttgaaaagcttgtccataaagagaagacattaattaaaaaagtgtgatgtaaagtaattttatatttttattcaattgcaaatcatcatttgtatgatttttaaagtgattatcataaaattcagtggatttatcatacatgatagattgtggttggatgacaataaatttattttacactattggtgcattgcctagtttctctgaatcatatcgtatctttatcatttttcatggactcttgtaattttgaatgttttctaagaatcttgaatccttacaaacttttaaattcatacaatcctttcaaatattttaatttcttatgttataaattcattaaaattcaaactataaaatcttaattacaaaaatcttttaaaaagttttgataagtcataacattctcacatagttttttaaaatgcatacaattttttatgtcaaaatagtcttttaaaatttcaatccaatatatatccctaaaagaaaataaaagataaagatgaagagtaatttttttcaaacttacttttggagtaacatgatttttctctctctctctctctctctctctctctctatatatatatatatatatatatatatatatatatatatatgttttatatagtgggttttgccacccattacatcaactcccttgtggatcctcctgagcccaaaagagaaaaccacaagagagttgctaggtgcacccagcagaattgttggtgcacccaacaattatgtgaatgggcaaaattgtccttgtttaaaagtttaaaaaataaacgcgcccattcagtgttgcttcttccgtgagtgcatccatctaccatgtctgctcgtcttcttcgcgcttctccttgttctccgtgcctcggctgccatttccgttcgttttgggaggttttttggtgccatttctgtgtttccaccgtcgaggttagtacctcctatctcccctcccctttaatgtccaatggtgatttagtttaggattttttggctgtagggtagatgacttcgtttctctgcaagtttgtctgcgaaaatggtgaagttggagcgttttccgtttacggaagaacttattccgtatgaaggaaattcttctaacagaagaagtttttccgtaatttcctacggaagaaggttcttccgtatgaagtaaatctttgtatggaagaaccttcttccgtattaacttagggaagaacttctttcgtagactcttacagaagaacttctttcgtatgactcttacggaagaacttcttccgtaagaaggtttccaatttttttattttttaattaattttttttataaattaagttttatttctttgttttgatttataataggtatatttagttttttaataataaaaaattgtatgcttgcatggtt

SEQ ID NO: 301 - DNA, 23 bp, synthetic construct
  misc_feature 1..23: note = Synthetic oligonucleotide
  source 1..23: mol_type = other DNA
atttgacagaagagagagagcac

SEQ ID NO: 302 - DNA, 54 bp, Glycine max
  source 1..54: mol_type = genomic DNA
ctctcaagtctcaacgaaatgaattaatgtatatacactcactcactctcgtta

SEQ ID NO: 303 - DNA, 12 bp, Glycine max
  source 1..12: mol_type = genomic DNA
cactctagttag

SEQ ID NO: 304 - DNA, 10 bp, Glycine max
  source 1..10: mol_type = genomic DNA
aaattgtctg

SEQ ID NO: 305 - DNA, 68 bp, Glycine max
  source 1..68: mol_type = genomic DNA
attcttggtttttggacagacaagggtgtgtttggttctctgactacactcactcactccttctctct

SEQ ID NO: 306 - DNA, 127 bp, Glycine max
  source 1..127: mol_type = genomic DNA
ttgaagcaatgagatgagaggaaattgttgagttgagaagaaaaaaaaagaagagagggtacaatctgtctctcgtagctggctggctccattatttttggttttggacagacaagggtgtgtttgg

SEQ ID NO: 307 - DNA, 13 bp, Glycine max
  source 1..13: mol_type = genomic DNA
tcgagctgtgctc

SEQ ID NO: 308 - DNA, 29 bp, Glycine max
  source 1..29: mol_type = genomic DNA
tcgagctgtgctctctctcttctgtcaaa

SEQ ID NO: 309 - DNA, 11 bp, Glycine max
  source 1..11: mol_type = genomic DNA
ctgtgctctct

SEQ ID NO: 310 - DNA, 3674 bp, synthetic construct
  misc_feature 1..3674: note = Synthetic polynucleotide
  source 1..3674: mol_type = other DNA
atggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggtcgccaccccgcccccgccattgctttctcattcagttacttccttttcagtttgacagctagttgcatccgctctgcttcctgtgttttatttccgcttcttgtctagggtttatggaaacttggctaaaattaaacagttttaggttcactattatcgatgtacatcacatctatgggctatggcgatgacaattgttttttttcctgacaccttctctttctggagaatgaaggattttttttaaaaaaaaacaaagatgagaacttgctttggtatcactactatactttaatgcttggtttgaatggtgctcatgagaactttaaagttggtaggtttgatgggtgctcctaaatatgatagagtgatgtgactcagccttgcctgcctgtctgtcagtttgttctgcattagattccagtgttaacttgcctgtcagttaacaagccattactatatggaatttgccttgagcttgatcggattgtgggtggtctggtccccaaaggaacaccactgaggaatttcaaaggagcctttgccgatgtggttgagtattaatctagagttccaactttacaagttgataatagtgctcattgcttagtctgcctcaaaaatagcactagacattatttgtcccctgtacttaaccaaactacatttggggtccttcacaaaaaaaaaacctacattaggcatgatcagttggccctcagtggtccccaaggaagaacaggcttgtctttgccactgtattattcttggtgttacaactttacaggttgtatgatactattcatattcaatgtttagtgcctaaaaaaacatgctcctctattttggctactaatacttaacaaactaaattaaattcattcggttctagatctggagaaaaaagtctagtcccttcaggtggtacaccgaacttaggttgtttctggaatagcttgtttcagtaggatacacttagcatattcattcagaaatttgctactgatgcctaatgatagcaagtgtatggaacgatcgagctgtctggtgcctaatgatagtaagcgtatgggaagatcgatctgtattgtgtcttagtaccagaacaatgcagcagaggggaaggaaagaaaaaagtagaatgtagttacgatcctacagacagctggttgtttcggtaatttggttctctgtaatttgacaatggtagcccgtaggattaaggcagtgtttagttaacttcaccgtatcatattctgtaatatatagtttcaatttttgttaggttcgatccaagcttttggttttctttgtcgataaattagttacaacagaaaaacaaaacatgatatataatggaagtgtggaactacatgtcttaaacatcgatatgcgtaggggtaggacctgggcacaccaccctatcgaccacaccttttgtgctttgggactaggctaaaggggtgtcctaacgtccccagcaaagaaaaggtgccacacttctggatttgggctgtactaaaccacagcaccacatagccacaagccacttatagcaacaacacatcattagagaagctaagttggaccatccacctgctttgctgttaaggacacctggcttgacatttgcatggaaatctcattggtggatgtgcattgttgtgttaggcccggcatctcacatctctgtgaatgattacatgcactttctctttgctttcttttagtatggatgtgcaggtgtaattatgagtgacataagacagaggttaatcttttaatggatgttcagtactggcctagcccagtaggaagagaaatgttactcttgagggcttgtacgttttgctctcaatccatatagattgggtgagatcgagtggatttaaatcctaaacaagtgaaaatctttcataattttttccaatcccattcaatccacatgggataggaataaccgaacaagcccgaatgtatagcattcccactggctttgacctaaagtatggttttttatatctatgtcgcactatgcatgttgctactaagataagttgcgctattaaggtaaactagcactttgaacttactattatcccatcattcaacttgcatttcattctattgtaagtgttattatggttgatgcttgatccatgaatttaatacttcctgcttcgtttgcattatacacaaaaattatcaatctacatcaataatatatgtggatttggagaatgcaactagcttgtattcctcataaaaccctgctactgtgcctcctcgcctgtaagttgaatcaaggacgcttgagtgaccacatctctctgtccacactgatcacaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtggtacaaaaaagctgcttcatttctttctacttggtgacttgggtgaccaaagtttcagttttttcgtatctgttgctaatgttcctacgcttgatccagaagagcccggcggcaggttcagaagcttcatgctggatttctcatacccaagggttccaagcgccatgagggatgggtttccagcggttcgacctggtggtgaaagggtgcctggcagcagtatccagtggcaagcacctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctag

SEQ ID NO: 311 - protein, 405 aa, synthetic construct
  REGION 1..405: note = Synthetic polypeptide
MEAGGASGGDDGDGQLHGLKFGKKIYFEDAGGSSSSGGSGSASATAPPATQQPSPPAASPRAAPGGGRRGRAAAGGAGPSLPPAPPRCLVDGCNADLTDAKTYYCRHKVCEMHSKEPRVVVNGLELRFCQQCSRFHNLAEFDQQKKSCRKRLAGHNERRRRPPPGPLASRYGRLAAPLGEPGGRFRSFMLDFSYPRVPSAMRDGFPAVRPGGERVPGSSIQWQAPPPPHHQSAAAGYGAHSYGSPGSSSSSSRPPVFPAGPELPPGGGCLAGVPTDSSCALSLLSTQPWDAAGHSAGHGHGHGHAASLPATAGFDGNPVAMASSYIAPPSPWTDSRAHEGGRRNVPQLPPDVPLGDVHSGPGTHHGQFSGELELALQGNRPAPAPRNGQGSAGAFDQAGSTTEWS

SEQ ID NO: 312 - DNA, 3755 bp, synthetic construct
  misc_feature 1..3755: note = Synthetic polynucleotide
  source 1..3755: mol_type = other DNA
atggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggtcgccaccccgcccccgccattgctttctcattcagttacttccttttcagtttgacagctagttgcatccgctctgcttcctgtgttttatttccgcttcttgtctagggtttatggaaacttggctaaaattaaacagttttaggttcactattatcgatgtacatcacatctatgggctatggcgatgacaattgttttttttcctgacaccttctctttctggagaatgaaggattttttttaaaaaaaaacaaagatgagaacttgctttggtatcactactatactttaatgcttggtttgaatggtgctcatgagaactttaaagttggtaggtttgatgggtgctcctaaatatgatagagtgatgtgactcagccttgcctgcctgtctgtcagtttgttctgcattagattccagtgttaacttgcctgtcagttaacaagccattactatatggaatttgccttgagcttgatcggattgtgggtggtctggtccccaaaggaacaccactgaggaatttcaaaggagcctttgccgatgtggttgagtattaatctagagttccaactttacaagttgataatagtgctcattgcttagtctgcctcaaaaatagcactagacattatttgtcccctgtacttaaccaaactacatttggggtccttcacaaaaaaaaaacctacattaggcatgatcagttggccctcagtggtccccaaggaagaacaggcttgtctttgccactgtattattcttggtgttacaactttacaggttgtatgatactattcatattcaatgtttagtgcctaaaaaaacatgctcctctattttggctactaatacttaacaaactaaattaaattcattcggttctagatctggagaaaaaagtctagtcccttcaggtggtacaccgaacttaggttgtttctggaatagcttgtttcagtaggatacacttagcatattcattcagaaatttgctactgatgcctaatgatagcaagtgtatggaacgatcgagctgtctggtgcctaatgatagtaagcgtatgggaagatcgatctgtattgtgtcttagtaccagaacaatgcagcagaggggaaggaaagaaaaaagtagaatgtagttacgatcctacagacagctggttgtttcggtaatttggttctctgtaatttgacaatggtagcccgtaggattaaggcagtgtttagttaacttcaccgtatcatattctgtaatatatagtttcaatttttgttaggttcgatccaagcttttggttttctttgtcgataaattagttacaacagaaaaacaaaacatgatatataatggaagtgtggaactacatgtcttaaacatcgatatgcgtaggggtaggacctgggcacaccaccctatcgaccacaccttttgtgctttgggactaggctaaaggggtgtcctaacgtccccagcaaagaaaaggtgccacacttctggatttgggctgtactaaaccacagcaccacatagccacaagccacttatagcaacaacacatcattagagaagctaagttggaccatccacctgctttgctgttaaggacacctggcttgacatttgcatggaaatctcattggtggatgtgcattgttgtgttaggcccggcatctcacatctctgtgaatgattacatgcactttctctttgctttcttttagtatggatgtgcaggtgtaattatgagtgacataagacagaggttaatcttttaatggatgttcagtactggcctagcccagtaggaagagaaatgttactcttgagggcttgtacgttttgctctcaatccatatagattgggtgagatcgagtggatttaaatcctaaacaagtgaaaatctttcataattttttccaatcccattcaatccacatgggataggaataaccgaacaagcccgaatgtatagcattcccactggctttgacctaaagtatggttttttatatctatgtcgcactatgcatgttgctactaagataagttgcgctattaaggtaaactagcactttgaacttactattatcccatcattcaacttgcatttcattctattgtaagtgttattatggttgatgcttgatccatgaatttaatacttcctgcttcgtttgcattatacacaaaaattatcaatctacatcaataatatatgtggatttggagaatgcaactagcttgtattcctcataaaaccctgctactgtgcctcctcgcctgtaagttgaatcaaggacgcttgagtgaccacatctctctgtccacactgatcacaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtggtacaaaaaagctgcttcatttctttctacttggtgacttgggtgaccaaagtttcagttttttcgtatctgttgctaatgttcctacgcttgatccagaagagcccggcggcaggttcagaagcttcatgctggatttctcatacccaagggttccaagcgccatgagggatgggtttccagcggttcgacctggtggtgaaagggtgcctggcagcagtatccagtggcaagcagcgatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctaggaggccaaataaaagcgcggcacatcgtcgtagccgcacagcgagatggagctggaagcctggctccgctagctag

SEQ ID NO: 313 - protein, 433 aa, synthetic construct
  REGION 1..433: note = Synthetic polypeptide
MEAGGASGGDDGDGQLHGLKFGKKIYFEDAGGSSSSGGSGSASATAPPATQQPSPPAASPRAAPGGGRRGRAAAGGAGPSLPPAPPRCLVDGCNADLTDAKTYYCRHKVCEMHSKEPRVVVNGLELRFCQQCSRFHNLAEFDQQKKSCRKRLAGHNERRRRPPPGPLASRYGRLAAPLGEPGGRFRSFMLDFSYPRVPSAMRDGFPAVRPGGERVPGSSIQWQAAILLPLTIRARQQDTVPTRTGARVARRRRRGHRCSLLARSSPQAVDVSQESPRTLAVLSLFCQLSHGTLLATAPATATATATPRHCPQQQASTATPWPWRVATSRRQAPGPTPGPTKAGGGTCLNCHPTSPSATCTLAQAPITASSQVSSSLPCRETGQPRRRAMARAPRARSTRPAARRSGRSRRPNKSAAHRRSRTARWSWKPGSAS

SEQ ID NO: 314 - DNA, 3681 bp, synthetic construct
  misc_feature 1..3681: note = Synthetic polynucleotide
  source 1..3681: mol_type = other DNA
atggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggtcgccaccccgcccccgccattgctttctcattcagttacttccttttcagtttgacagctagttgcatccgctctgcttcctgtgttttatttccgcttcttgtctagggtttatggaaacttggctaaaattaaacagttttaggttcactattatcgatgtacatcacatctatgggctatggcgatgacaattgttttttttcctgacaccttctctttctggagaatgaaggattttttttaaaaaaaaacaaagatgagaacttgctttggtatcactactatactttaatgcttggtttgaatggtgctcatgagaactttaaagttggtaggtttgatgggtgctcctaaatatgatagagtgatgtgactcagccttgcctgcctgtctgtcagtttgttctgcattagattccagtgttaacttgcctgtcagttaacaagccattactatatggaatttgccttgagcttgatcggattgtgggtggtctggtccccaaaggaacaccactgaggaatttcaaaggagcctttgccgatgtggttgagtattaatctagagttccaactttacaagttgataatagtgctcattgcttagtctgcctcaaaaatagcactagacattatttgtcccctgtacttaaccaaactacatttggggtccttcacaaaaaaaaaacctacattaggcatgatcagttggccctcagtggtccccaaggaagaacaggcttgtctttgccactgtattattcttggtgttacaactttacaggttgtatgatactattcatattcaatgtttagtgcctaaaaaaacatgctcctctattttggctactaatacttaacaaactaaattaaattcattcggttctagatctggagaaaaaagtctagtcccttcaggtggtacaccgaacttaggttgtttctggaatagcttgtttcagtaggatacacttagcatattcattcagaaatttgctactgatgcctaatgatagcaagtgtatggaacgatcgagctgtctggtgcctaatgatagtaagcgtatgggaagatcgatctgtattgtgtcttagtaccagaacaatgcagcagaggggaaggaaagaaaaaagtagaatgtagttacgatcctacagacagctggttgtttcggtaatttggttctctgtaatttgacaatggtagcccgtaggattaaggcagtgtttagttaacttcaccgtatcatattctgtaatatatagtttcaatttttgttaggttcgatccaagcttttggttttctttgtcgataaattagttacaacagaaaaacaaaacatgatatataatggaagtgtggaactacatgtcttaaacatcgatatgcgtaggggtaggacctgggcacaccaccctatcgaccacaccttttgtgctttgggactaggctaaaggggtgtcctaacgtccccagcaaagaaaaggtgccacacttctggatttgggctgtactaaaccacagcaccacatagccacaagccacttatagcaacaacacatcattagagaagctaagttggaccatccacctgctttgctgttaaggacacctggcttgacatttgcatggaaatctcattggtggatgtgcattgttgtgttaggcccggcatctcacatctctgtgaatgattacatgcactttctctttgctttcttttagtatggatgtgcaggtgtaattatgagtgacataagacagaggttaatcttttaatggatgttcagtactggcctagcccagtaggaagagaaatgttactcttgagggcttgtacgttttgctctcaatccatatagattgggtgagatcgagtggatttaaatcctaaacaagtgaaaatctttcataattttttccaatcccattcaatccacatgggataggaataaccgaacaagcccgaatgtatagcattcccactggctttgacctaaagtatggttttttatatctatgtcgcactatgcatgttgctactaagataagttgcgctattaaggtaaactagcactttgaacttactattatcccatcattcaacttgcatttcattctattgtaagtgttattatggttgatgcttgatccatgaatttaatacttcctgcttcgtttgcattatacacaaaaattatcaatctacatcaataatatatgtggatttggagaatgcaactagcttgtattcctcataaaaccctgctactgtgcctcctcgcctgtaagttgaatcaaggacgcttgagtgaccacatctctctgtccacactgatcacaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtggtacaaaaaagctgcttcatttctttctacttggtgacttgggtgaccaaagtttcagttttttcgtatctgttgctaatgttcctacgcttgatccagaagagcccggcggcaggttcagaagcttcatgctggatttctcatacccaagggttccaagcgccatgagggatgggtttccagcggttcgacctggtggtgaaagggtgcctggcagcagtatccagtggcaagcagctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctag

SEQ ID NO: 315 - protein, 248 aa, synthetic construct
  REGION 1..248: note = Synthetic polypeptide
MEAGGASGGDDGDGQLHGLKFGKKIYFEDAGGSSSSGGSGSASATAPPATQQPSPPAASPRAAPGGGRRGRAAAGGAGPSLPPAPPRCLVDGCNADLTDAKTYYCRHKVCEMHSKEPRVVVNGLELRFCQQCSRFHNLAEFDQQKKSCRKRLAGHNERRRRPPPGPLASRYGRLAAPLGEPGGRFRSFMLDFSYPRVPSAMRDGFPAVRPGGERVPGSSIQWQAARSSSPSPSERGSRIRCPLVREPG

SEQ ID NO: 316 - DNA, 3677 bp, synthetic construct
  misc_feature 1..3677: note = Synthetic polynucleotide
  source 1..3677: mol_type = other DNA
atggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggtcgccaccccgcccccgccattgctttctcattcagttacttccttttcagtttgacagctagttgcatccgctctgcttcctgtgttttatttccgcttcttgtctagggtttatggaaacttggctaaaattaaacagttttaggttcactattatcgatgtacatcacatctatgggctatggcgatgacaattgttttttttcctgacaccttctctttctggagaatgaaggattttttttaaaaaaaaacaaagatgagaacttgctttggtatcactactatactttaatgcttggtttgaatggtgctcatgagaactttaaagttggtaggtttgatgggtgctcctaaatatgatagagtgatgtgactcagccttgcctgcctgtctgtcagtttgttctgcattagattccagtgttaacttgcctgtcagttaacaagccattactatatggaatttgccttgagcttgatcggattgtgggtggtctggtccccaaaggaacaccactgaggaatttcaaaggagcctttgccgatgtggttgagtattaatctagagttccaactttacaagttgataatagtgctcattgcttagtctgcctcaaaaatagcactagacattatttgtcccctgtacttaaccaaactacatttggggtccttcacaaaaaaaaaacctacattaggcatgatcagttggccctcagtggtccccaaggaagaacaggcttgtctttgccactgtattattcttggtgttacaactttacaggttgtatgatactattcatattcaatgtttagtgcctaaaaaaacatgctcctctattttggctactaatacttaacaaactaaattaaattcattcggttctagatctggagaaaaaagtctagtcccttcaggtggtacaccgaacttaggttgtttctggaatagcttgtttcagtaggatacacttagcatattcattcagaaatttgctactgatgcctaatgatagcaagtgtatggaacgatcgagctgtctggtgcctaatgatagtaagcgtatgggaagatcgatctgtattgtgtcttagtaccagaacaatgcagcagaggggaaggaaagaaaaaagtagaatgtagttacgatcctacagacagctggttgtttcggtaatttggttctctgtaatttgacaatggtagcccgtaggattaaggcagtgtttagttaacttcaccgtatcatattctgtaatatatagtttcaatttttgttaggttcgatccaagcttttggttttctttgtcgataaattagttacaacagaaaaacaaaacatgatatataatggaagtgtggaactacatgtcttaaacatcgatatgcgtaggggtaggacctgggcacaccaccctatcgaccacaccttttgtgctttgggactaggctaaaggggtgtcctaacgtccccagcaaagaaaaggtgccacacttctggatttgggctgtactaaaccacagcaccacatagccacaagccacttatagcaacaacacatcattagagaagctaagttggaccatccacctgctttgctgttaaggacacctggcttgacatttgcatggaaatctcattggtggatgtgcattgttgtgttaggcccggcatctcacatctctgtgaatgattacatgcactttctctttgctttcttttagtatggatgtgcaggtgtaattatgagtgacataagacagaggttaatcttttaatggatgttcagtactggcctagcccagtaggaagagaaatgttactcttgagggcttgtacgttttgctctcaatccatatagattgggtgagatcgagtggatttaaatcctaaacaagtgaaaatctttcataattttttccaatcccattcaatccacatgggataggaataaccgaacaagcccgaatgtatagcattcccactggctttgacctaaagtatggttttttatatctatgtcgcactatgcatgttgctactaagataagttgcgctattaaggtaaactagcactttgaacttactattatcccatcattcaacttgcatttcattctattgtaagtgttattatggttgatgcttgatccatgaatttaatacttcctgcttcgtttgcattatacacaaaaattatcaatctacatcaataatatatgtggatttggagaatgcaactagcttgtattcctcataaaaccctgctactgtgcctcctcgcctgtaagttgaatcaaggacgcttgagtgaccacatctctctgtccacactgatcacaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtggtacaaaaaagctgcttcatttctttctacttggtgacttgggtgaccaaagtttcagttttttcgtatctgttgctaatgttcctacgcttgatccagaagagcccggcggcaggttcagaagcttcatgctggatttctcatacccaagggttccaagcgccatgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctag

SEQ ID NO: 317 - protein, 407 aa, synthetic construct
  REGION 1..407: note = Synthetic polypeptidfe
MEAGGASGGDDGDGQLHGLKFGKKIYFEDAGGSSSSGGSGSASATAPPATQQPSPPAASPRAAPGGGRRGRAAAGGAGPSLPPAPPRCLVDGCNADLTDAKTYYCRHKVCEMHSKEPRVVVNGLELRFCQQCSRFHNLAEFDQQKKSCRKRLAGHNERRRRPPPGPLASRYGRLAAPLGEPGGRFRSFMLDFSYPRVPSAMRDGFPAVRPGGERVPGSSIQWQAASLDPPPPHHQSAAAGYGAHSYGSPGSSSSSSRPPVFPAGPELPPGGGCLAGVPTDSSCLLSTQPWDAAGHSAGHGHGHGHAASLPATAGFDGNPVAMASSYIAPPSPWTDSRAHEGGRRNVPQLPPDVPLGDVHSGPGTHHGQFSGELELALQGNRPAPAPRNGQGSAGAFDQAGSTTEWSL

SEQ ID NO: 318 - DNA, 3749 bp, synthetic construct
  misc_feature 1..3749: note = Synthetic polynucleotide
  source 1..3749: mol_type = other DNA
atggaggccggcggcgccagcggcggcgacgacggggacggccagctgcacggcctcaagttcggtaagaagatctacttcgaggacgccggcggctccagtagcagcggcggcagtggcagtgccagcgcgaccgcgcctccagcgacgcagcagccgtcgccgccggccgcttcgcctagggccgcgcccggtggcggcaggaggggcagggccgcggctggcggcgcgggcccctccttgccgcccgcgcccccgcgctgcctggtcgacggctgcaacgccgacctcaccgacgccaagacctactactgccgccacaaggtgtgcgaaatgcactccaaggagccccgcgtcgtcgtcaacggtctcgagctacgcttctgccagcagtgcagcaggtcgccaccccgcccccgccattgctttctcattcagttacttccttttcagtttgacagctagttgcatccgctctgcttcctgtgttttatttccgcttcttgtctagggtttatggaaacttggctaaaattaaacagttttaggttcactattatcgatgtacatcacatctatgggctatggcgatgacaattgttttttttcctgacaccttctctttctggagaatgaaggattttttttaaaaaaaaacaaagatgagaacttgctttggtatcactactatactttaatgcttggtttgaatggtgctcatgagaactttaaagttggtaggtttgatgggtgctcctaaatatgatagagtgatgtgactcagccttgcctgcctgtctgtcagtttgttctgcattagattccagtgttaacttgcctgtcagttaacaagccattactatatggaatttgccttgagcttgatcggattgtgggtggtctggtccccaaaggaacaccactgaggaatttcaaaggagcctttgccgatgtggttgagtattaatctagagttccaactttacaagttgataatagtgctcattgcttagtctgcctcaaaaatagcactagacattatttgtcccctgtacttaaccaaactacatttggggtccttcacaaaaaaaaaacctacattaggcatgatcagttggccctcagtggtccccaaggaagaacaggcttgtctttgccactgtattattcttggtgttacaactttacaggttgtatgatactattcatattcaatgtttagtgcctaaaaaaacatgctcctctattttggctactaatacttaacaaactaaattaaattcattcggttctagatctggagaaaaaagtctagtcccttcaggtggtacaccgaacttaggttgtttctggaatagcttgtttcagtaggatacacttagcatattcattcagaaatttgctactgatgcctaatgatagcaagtgtatggaacgatcgagctgtctggtgcctaatgatagtaagcgtatgggaagatcgatctgtattgtgtcttagtaccagaacaatgcagcagaggggaaggaaagaaaaaagtagaatgtagttacgatcctacagacagctggttgtttcggtaatttggttctctgtaatttgacaatggtagcccgtaggattaaggcagtgtttagttaacttcaccgtatcatattctgtaatatatagtttcaatttttgttaggttcgatccaagcttttggttttctttgtcgataaattagttacaacagaaaaacaaaacatgatatataatggaagtgtggaactacatgtcttaaacatcgatatgcgtaggggtaggacctgggcacaccaccctatcgaccacaccttttgtgctttgggactaggctaaaggggtgtcctaacgtccccagcaaagaaaaggtgccacacttctggatttgggctgtactaaaccacagcaccacatagccacaagccacttatagcaacaacacatcattagagaagctaagttggaccatccacctgctttgctgttaaggacacctggcttgacatttgcatggaaatctcattggtggatgtgcattgttgtgttaggcccggcatctcacatctctgtgaatgattacatgcactttctctttgctttcttttagtatggatgtgcaggtgtaattatgagtgacataagacagaggttaatcttttaatggatgttcagtactggcctagcccagtaggaagagaaatgttactcttgagggcttgtacgttttgctctcaatccatatagattgggtgagatcgagtggatttaaatcctaaacaagtgaaaatctttcataattttttccaatcccattcaatccacatgggataggaataaccgaacaagcccgaatgtatagcattcccactggctttgacctaaagtatggttttttatatctatgtcgcactatgcatgttgctactaagataagttgcgctattaaggtaaactagcactttgaacttactattatcccatcattcaacttgcatttcattctattgtaagtgttattatggttgatgcttgatccatgaatttaatacttcctgcttcgtttgcattatacacaaaaattatcaatctacatcaataatatatgtggatttggagaatgcaactagcttgtattcctcataaaaccctgctactgtgcctcctcgcctgtaagttgaatcaaggacgcttgagtgaccacatctctctgtccacactgatcacaggttccacaacctggccgaatttgaccagcaaaagaaaagctgtcgcaagcgtctagcaggccacaacgaacgccggagacggccacctcctggaccccttgcgtcacgatatggccgtctcgctgcaccgctcggtggtacaaaaaagctgcttcatttctttctacttggtgacttgggtgaccaaagtttcagttttttcgtatctgttgctaatgttcctacgcttgatccagaagagcccggcggcaggttcagaagcttcatgctggatttctcatacccaagggttccaagcgccatgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagcttctgtcaactcagccatgggacgctgctggccacagcgccggccacggccacggccacggccacgccgcgtcactgcccgcaacagcaggcttcgacggcaaccccgtggccatggcgagtagctacatcgcgccgccaagcccctggaccgactcccgggcccacgaaggcgggcggcggaacgtgcctcaactgccacccgacgtccccctcggcgacgtgcactctggcccaggcacccatcacggccagttctcaggtgagctcgagcttgccctgcagggaaacaggccagccccggcgccgcgcaatggccagggctccgcgggcgcgttcgaccaggccggcagcacgacggagtggtcgctctaggaggccaaataaaagcgcggcacatcgtcgtagccgcacagcgagatggagctggaagcctggctccgctagctag

SEQ ID NO: 319 - protein, 431 aa, synthetic construct
  REGION 1..431: note = Synthetic polypeptide
MEAGGASGGDDGDGQLHGLKFGKKIYFEDAGGSSSSGGSGSASATAPPATQQPSPPAASPRAAPGGGRRGRAAAGGAGPSLPPAPPRCLVDGCNADLTDAKTYYCRHKVCEMHSKEPRVVVNGLELRFCQQCSRFHNLAEFDQQKKSCRKRLAGHNERRRRPPPGPLASRYGRLAAPLGEPGGRFRSFMLDFSYPRVPSAMRDGFPAVRPGGERVPGSSIQWQAASLDPPPPHHQSAAAGYGAHSYGSPGSSSSSSRPPVFPAGPELPPGGGCLAGVPTDSSFCQLSHGTLLATAPATATATATPRHCPQQQASTATPWPWRVATSRRQAPGPTPGPTKAGGGTCLNCHPTSPSATCTLAQAPITASSQVSSSLPCRETGQPRRRAMARAPRARSTRPAARRSGRSRRPNKSAAHRRSRTARWSWKPGSAS

SEQ ID NO: 320 - DNA, 3346 bp, synthetic construct
  misc_feature 1..3346: note = Synthetic polynucleotide
  source 1..3346: mol_type = other DNA
atggagtccggcggtggcggggacgaccagctgcacggcctcaagttcggcaagaagatctacttcgaggacgccgccggctccagcagcggcagcagcagcggcggtggcagcgcgcccgcgcctccagcgacgcagcagccgtcgccgccggccgcttcgcctagggccccggccggcggcggcaggaggggcagggccgcggccggcggcgcgggcccctcgacggcgcccgcgcccgcgcgctgccaggtcgacggctgcaacgttgacctcaccgacgtcaagcccgcctactactgccgccacaaggtgtgcaaaatgcactccaaggagccccgcgtcctcgtcaacggcctcgagcagcgcttctgccagcagtgcagcaggtcaccacccagccccgccccgccattgctgcacagttgcactccccccctcttccacctccacctctcattcaattgattgtttccagttgtcacttgttggttgcattcgctctgcagtctgctcccccgtgttttttttttccagtcccactcccttcattcacagtctagggttttagggaggctacctagacgaagaccagacggtttcaggtccatcataccacggacacgctacaaaactatagatgcgctaaacagtgggagctagggaaagaaacttggctcggtttgacggagcttccatcccatttcatctatggcgacggcttctcctcctgaagctgaacaagaacgaaggaggaaaaaaacaaaagctgctttggcattactgctacgctttaatgtttcgcttgaatggtgctcatgagcacaggtaggtctgatggatgctcctcctaaaaatatgatagagtgatgtgactcagcctgcccctgccctgcctgcctgtctgtcagttcgctttgcattagattccaccgctaatactactcgggtatcccagtgaagcctgtcagctaacaagccattacaataagcgattagtagccttgagcctggttatacgtgtggtccccaaaaaaggaacagcaacagtgaggaattttcaaaggagtctttgctttgccgatgcagtagtattttaatttagaggagtcccaactttgcacaggttgacactatccattgttttagctagctagcctgcctcaaatatagcattatacattgtttgtccctgtacttaaccatactacatcgggcatccttcgcccccgccccccccccccccccccctttttttttttgcattatgcaaatggtcccccgaggaagaacaagttgtagcctttgcaatgtgttgttcttggcgctgcaactttacaggttgtacgatcctgttcatccggtgtaagatctggaggatttctagtcccttcaggtgctacaccaaactagactgttcctggaacagctttgtttcctttggatacaattagcattttcattcataaatcttaattcagtcatctatctaatttgacatacaatagagagagagagagaaactaaacatgatggtggcgagctgcatctaatttgatatgggtagggcctggacacattaccctagcggcgaccgcaggctttgtgctttaggactaggctacaggagtgtcctcatgtccccagcaaaagagaagggcgccacagttctggatttggggctctctactaaaccacactgccacttatagcaacaaggcatatcattagagaagagctaagttggaccagccaccagtaccacttggcgttattagatggaaacctcattattggtgggtgtgtactgttgtgttaagcccggcatcacacatctctatgaaatgattacacacacactttctctttgcttttcttttctttttagtatggatgtgcaggtgtcattatgagcggcataagacagaggttaatgttaatctttaatgggggatgtgcagtagtacaaccctaacctagggagaagagaaatgttactctctttgatgattgtactggcattcccactggctttgacctaaagtgtggtttcctatatccatgtcgcgctaagcgctatgcatgttgctagctactactgagataaactagtgccttcaacttgctacgtactgtcccaccattcagcttgcactttattctactgtaagagttactactatggtaaatggtttatgcttggtccatggaactttttactgcctgatttgtttgcattctacacaaaagctatgaatctacagcaatgtatatgtggatttatagaatgcaacttccattacttataaaaattctgttgctgtgccaccaacttccattacctgtaataaactgggatcaagggcgcgtgaatgaacacacctctgtttgtcgcatcaatcgcaggttccaccagctgcctgaattcgaccagctaaagaagagctgccgcaaacgcctcgcaggccacaacgagcgccggaggaggccgccgcctggaccccttgcgtcacgatacggccgtcacgctgcgtcgctcggcggtaaccaaaagctggtttttaccctgtgatgtcgtctcggcgaccaaagttcccgtcttttttttgtaaccgctaatgttcatgtgcttggtccagaagagcccggcaggctcagaagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagcttctgtcaactcagccatgggatgctgcccacagccacagccacagccacgctgcgccaacagcgggtttcgacggcggcagccctgtggcgccctccctcatggcggcgagtagctacatcgcgccgagcccctggaccgagaccgactcctggggccacgaaggcgggcggagcgtgcctcagctgccacctgacgacgtccccctcggcgaggtgcactccggctcgagcagccaccacggccagttctcaggtgagctcgagctcgccctgcagggaaacaggccagcgccagggtcggcggcaccgccagcgccgcgcaataatcagggctccgcgggcacgttcgaccaggctggcaacacgatggactggtcgctctaggccgggggccgaaataaagcgcagcagatcctag

SEQ ID NO: 321 - protein, 410 aa, synthetic construct
  REGION 1..410: note = Syntthetic polypeptide
MESGGGGDDQLHGLKFGKKIYFEDAAGSSSGSSSGGGSAPAPPATQQPSPPAASPRAPAGGGRRGRAAAGGAGPSTAPAPARCQVDGCNVDLTDVKPAYYCRHKVCKMHSKEPRVLVNGLEQRFCQQCSRFHQLPEFDQLKKSCRKRLAGHNERRRRPPPGPLASRYGRHAASLGEEPGRLRSFMLDFSYPRVSSAMRGGFPAVRPGGERVPGGIQWQAGLDPRHHQGAVAGYGAHYGSEGGSSSSARPPVFPGPELPPGGCLAGVPADSSFCQLSHGMLPTATATATLRQQRVSTAAALWRPPSWRRVATSRRAPGPRPTPGATKAGGACLSCHLTTSPSARCTPARAATTASSQVSSSSPCRETGQRQGRRHRQRRAIIRAPRARSTRLATRWTGRSRPGAEIKRSRS

SEQ ID NO: 322 - DNA, 3320 bp, synthetic construct
  misc_feature 1..3320: note = Synthetic polynucleotide
  source 1..3320: mol_type = other DNA
atggagtccggcggtggcggggacgaccagctgcacggcctcaagttcggcaagaagatctacttcgaggacgccgccggctccagcagcggcagcagcagcggcggtggcagcgcgcccgcgcctccagcgacgcagcagccgtcgccgccggccgcttcgcctagggccccggccggcggcggcaggaggggcagggccgcggccggcggcgcgggcccctcgacggcgcccgcgcccgcgcgctgccaggtcgacggctgcaacgttgacctcaccgacgtcaagcccgcctactactgccgccacaaggtgtgcaaaatgcactccaaggagccccgcgtcctcgtcaacggcctcgagcagcgcttctgccagcagtgcagcaggtcaccacccagccccgccccgccattgctgcacagttgcactccccccctcttccacctccacctctcattcaattgattgtttccagttgtcacttgttggttgcattcgctctgcagtctgctcccccgtgttttttttttccagtcccactcccttcattcacagtctagggttttagggaggctacctagacgaagaccagacggtttcaggtccatcataccacggacacgctacaaaactatagatgcgctaaacagtgggagctagggaaagaaacttggctcggtttgacggagcttccatcccatttcatctatggcgacggcttctcctcctgaagctgaacaagaacgaaggaggaaaaaaacaaaagctgctttggcattactgctacgctttaatgtttcgcttgaatggtgctcatgagcacaggtaggtctgatggatgctcctcctaaaaatatgatagagtgatgtgactcagcctgcccctgccctgcctgcctgtctgtcagttcgctttgcattagattccaccgctaatactactcgggtatcccagtgaagcctgtcagctaacaagccattacaataagcgattagtagccttgagcctggttatacgtgtggtccccaaaaaaggaacagcaacagtgaggaattttcaaaggagtctttgctttgccgatgcagtagtattttaatttagaggagtcccaactttgcacaggttgacactatccattgttttagctagctagcctgcctcaaatatagcattatacattgtttgtccctgtacttaaccatactacatcgggcatccttcgcccccgccccccccccccccccccctttttttttttgcattatgcaaatggtcccccgaggaagaacaagttgtagcctttgcaatgtgttgttcttggcgctgcaactttacaggttgtacgatcctgttcatccggtgtaagatctggaggatttctagtcccttcaggtgctacaccaaactagactgttcctggaacagctttgtttcctttggatacaattagcattttcattcataaatcttaattcagtcatctatctaatttgacatacaatagagagagagagagaaactaaacatgatggtggcgagctgcatctaatttgatatgggtagggcctggacacattaccctagcggcgaccgcaggctttgtgctttaggactaggctacaggagtgtcctcatgtccccagcaaaagagaagggcgccacagttctggatttggggctctctactaaaccacactgccacttatagcaacaaggcatatcattagagaagagctaagttggaccagccaccagtaccacttggcgttattagatggaaacctcattattggtgggtgtgtactgttgtgttaagcccggcatcacacatctctatgaaatgattacacacacactttctctttgcttttcttttctttttagtatggatgtgcaggtgtcattatgagcggcataagacagaggttaatgttaatctttaatgggggatgtgcagtagtacaaccctaacctagggagaagagaaatgttactctctttgatgattgtactggcattcccactggctttgacctaaagtgtggtttcctatatccatgtcgcgctaagcgctatgcatgttgctagctactactgagataaactagtgccttcaacttgctacgtactgtcccaccattcagcttgcactttattctactgtaagagttactactatggtaaatggtttatgcttggtccatggaactttttactgcctgatttgtttgcattctacacaaaagctatgaatctacagcaatgtatatgtggatttatagaatgcaacttccattacttataaaaattctgttgctgtgccaccaacttccattacctgtaataaactgggatcaagggcgcgtgaatgaacacacctctgtttgtcgcatcaatcgcaggttccaccagctgcctgaattcgaccagctaaagaagagctgccgcaaacgcctcgcaggccacaacgagcgccggaggaggccgccgcctggaccccttgcgtcacgatacggccgtcacgctgcgtcgctcggcggtaaccaaaagctggtttttaccctgtgatgtcgtctcggcgaccaaagttcccgtcttttttttgtaaccgctaatgttcatgtgcttggtccagaagagcccggcaggctcagaagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctctctctcttctgtcaactcagccatgggatgctgcccacagccacagccacagccacgctgcgccaacagcgggtttcgacggcggcagccctgtggcgccctccctcatggcggcgagtagctacatcgcgccgagcccctggaccgagaccgactcctggggccacgaaggcgggcggagcgtgcctcagctgccacctgacgacgtccccctcggcgaggtgcactccggctcgagcagccaccacggccagttctcaggtgagctcgagctcgccctgcagggaaacaggccagcgccagggtcggcggcaccgccagcgccgcgcaataatcagggctccgcgggcacgttcgaccaggctggcaacacgatggactggtcgctctag

SEQ ID NO: 323 - protein, 311 aa, synthetic construct
  REGION 1..311: note = Synthetic polypeptide
MESGGGGDDQLHGLKFGKKIYFEDAAGSSSGSSSGGGSAPAPPATQQPSPPAASPRAPAGGGRRGRAAAGGAGPSTAPAPARCQVDGCNVDLTDVKPAYYCRHKVCKMHSKEPRVLVNGLEQRFCQQCSRFHQLPEFDQLKKSCRKRLAGHNERRRRPPPGPLASRYGRHAASLGEEPGRLRSFMLDFSYPRVSSAMRGGFPAVRPGGERVPGGIQWQAGLDPRHHQGAVAGYGAHYGSEGGSSSSARPPVFPGPELPPGGCLAGVPADSSSLSSVNSAMGCCPQPQPQPRCANSGFRRRQPCGALPHGGE

SEQ ID NO: 324 - DNA, 3316 bp, synthetic construct
  misc_feature 1..3316: note = Synthetic polynucleotide
  source 1..3316: mol_type = other DNA
atggagtccggcggtggcggggacgaccagctgcacggcctcaagttcggcaagaagatctacttcgaggacgccgccggctccagcagcggcagcagcagcggcggtggcagcgcgcccgcgcctccagcgacgcagcagccgtcgccgccggccgcttcgcctagggccccggccggcggcggcaggaggggcagggccgcggccggcggcgcgggcccctcgacggcgcccgcgcccgcgcgctgccaggtcgacggctgcaacgttgacctcaccgacgtcaagcccgcctactactgccgccacaaggtgtgcaaaatgcactccaaggagccccgcgtcctcgtcaacggcctcgagcagcgcttctgccagcagtgcagcaggtcaccacccagccccgccccgccattgctgcacagttgcactccccccctcttccacctccacctctcattcaattgattgtttccagttgtcacttgttggttgcattcgctctgcagtctgctcccccgtgttttttttttccagtcccactcccttcattcacagtctagggttttagggaggctacctagacgaagaccagacggtttcaggtccatcataccacggacacgctacaaaactatagatgcgctaaacagtgggagctagggaaagaaacttggctcggtttgacggagcttccatcccatttcatctatggcgacggcttctcctcctgaagctgaacaagaacgaaggaggaaaaaaacaaaagctgctttggcattactgctacgctttaatgtttcgcttgaatggtgctcatgagcacaggtaggtctgatggatgctcctcctaaaaatatgatagagtgatgtgactcagcctgcccctgccctgcctgcctgtctgtcagttcgctttgcattagattccaccgctaatactactcgggtatcccagtgaagcctgtcagctaacaagccattacaataagcgattagtagccttgagcctggttatacgtgtggtccccaaaaaaggaacagcaacagtgaggaattttcaaaggagtctttgctttgccgatgcagtagtattttaatttagaggagtcccaactttgcacaggttgacactatccattgttttagctagctagcctgcctcaaatatagcattatacattgtttgtccctgtacttaaccatactacatcgggcatccttcgcccccgccccccccccccccccccctttttttttttgcattatgcaaatggtcccccgaggaagaacaagttgtagcctttgcaatgtgttgttcttggcgctgcaactttacaggttgtacgatcctgttcatccggtgtaagatctggaggatttctagtcccttcaggtgctacaccaaactagactgttcctggaacagctttgtttcctttggatacaattagcattttcattcataaatcttaattcagtcatctatctaatttgacatacaatagagagagagagagaaactaaacatgatggtggcgagctgcatctaatttgatatgggtagggcctggacacattaccctagcggcgaccgcaggctttgtgctttaggactaggctacaggagtgtcctcatgtccccagcaaaagagaagggcgccacagttctggatttggggctctctactaaaccacactgccacttatagcaacaaggcatatcattagagaagagctaagttggaccagccaccagtaccacttggcgttattagatggaaacctcattattggtgggtgtgtactgttgtgttaagcccggcatcacacatctctatgaaatgattacacacacactttctctttgcttttcttttctttttagtatggatgtgcaggtgtcattatgagcggcataagacagaggttaatgttaatctttaatgggggatgtgcagtagtacaaccctaacctagggagaagagaaatgttactctctttgatgattgtactggcattcccactggctttgacctaaagtgtggtttcctatatccatgtcgcgctaagcgctatgcatgttgctagctactactgagataaactagtgccttcaacttgctacgtactgtcccaccattcagcttgcactttattctactgtaagagttactactatggtaaatggtttatgcttggtccatggaactttttactgcctgatttgtttgcattctacacaaaagctatgaatctacagcaatgtatatgtggatttatagaatgcaacttccattacttataaaaattctgttgctgtgccaccaacttccattacctgtaataaactgggatcaagggcgcgtgaatgaacacacctctgtttgtcgcatcaatcgcaggttccaccagctgcctgaattcgaccagctaaagaagagctgccgcaaacgcctcgcaggccacaacgagcgccggaggaggccgccgcctggaccccttgcgtcacgatacggccgtcacgctgcgtcgctcggcggtaaccaaaagctggtttttaccctgtgatgtcgtctcggcgaccaaagttcccgtcttttttttgtaaccgctaatgttcatgtgcttggtccagaagagcccggcaggctcagaagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctctcttctgtcaactcagccatgggatgctgcccacagccacagccacagccacgctgcgccaacagcgggtttcgacggcggcagccctgtggcgccctccctcatggcggcgagtagctacatcgcgccgagcccctggaccgagaccgactcctggggccacgaaggcgggcggagcgtgcctcagctgccacctgacgacgtccccctcggcgaggtgcactccggctcgagcagccaccacggccagttctcaggtgagctcgagctcgccctgcagggaaacaggccagcgccagggtcggcggcaccgccagcgccgcgcaataatcagggctccgcgggcacgttcgaccaggctggcaacacgatggactggtcgctctag

SEQ ID NO: 325 - protein, 400 aa, synthetic construct
  REGION 1..400: note = Synthetic polypeptide
MESGGGGDDQLHGLKFGKKIYFEDAAGSSSGSSSGGGSAPAPPATQQPSPPAASPRAPAGGGRRGRAAAGGAGPSTAPAPARCQVDGCNVDLTDVKPAYYCRHKVCKMHSKEPRVLVNGLEQRFCQQCSRFHQLPEFDQLKKSCRKRLAGHNERRRRPPPGPLASRYGRHAASLGEEPGRLRSFMLDFSYPRVSSAMRGGFPAVRPGGERVPGGIQWQAGLDPRHHQGAVAGYGAHYGSEGGSSSSARPPVFPGPELPPGGCLAGVPADSSSLLSTQPWDAAHSHSHSHAAPTAGFDGGSPVAPSLMAASSYIAPSPWTETDSWGHEGGRSVPQLPPDDVPLGEVHSGSSSHHGQFSGELELALQGNRPAPGSAAPPAPRNNQGSAGTFDQAGNTMDWSL

SEQ ID NO: 326 - DNA, 23 bp, synthetic construct
  misc_feature 1..23: note = Synthetic oligonucleotide
  source 1..23: mol_type = other DNA
cagtggcaagcgggcttggatcc

SEQ ID NO: 327 - DNA, 23 bp, synthetic construct
  misc_feature 1..23: note = Synthetic oligonucleotide
  source 1..23: mol_type = other DNA
cagtggcaagcagcgagcctaga

SEQ ID NO: 328 - DNA, 12 bp, Zea mays
  source 1..12: mol_type = genomic DNA
gcgagcctagat

SEQ ID NO: 329 - DNA, 13 bp, Zea mays
  source 1..13: mol_type = genomic DNA
tgtgctctctctc

SEQ ID NO: 330 - DNA, 13 bp, Zea mays
  source 1..13: mol_type = genomic DNA
gtgctctctctct

SEQ ID NO: 331 - DNA, 18 bp, Zea mays
  source 1..18: mol_type = genomic DNA
gttgtttaagtaaagcga

SEQ ID NO: 332 - DNA, 381 bp, Zea mays
  source 1..381: mol_type = genomic DNA
gggaatccccacaaggcagcagatggaagcgaaaggtggcggtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggaggagagagaagagagggagccagtccagctccagccagccggacgcccggaccaacaccaccc

SEQ ID NO: 333 - DNA, 301 bp, Zea mays
  source 1..301: mol_type = genomic DNA
ggtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggaggagagagaagagagggagccag

SEQ ID NO: 334 - DNA, 261 bp, Zea mays
  source 1..261: mol_type = genomic DNA
gcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggagga

SEQ ID NO: 335 - DNA, 221 bp, Zea mays
  source 1..221: mol_type = genomic DNA
gccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctctt

SEQ ID NO: 336 - DNA, 181 bp, Zea mays
  source 1..181: mol_type = genomic DNA
gaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgcca

SEQ ID NO: 337 - DNA, 126 bp, Zea mays
  source 1..126: mol_type = genomic DNA
gaggagacagccacagccatacagggatgctgcctgcttcgtgaccgtgctggtggtggtgggctgcagcctgcagctcgtatcacacgcccacagtgcaggaaggagggagtggaagaagagaga

SEQ ID NO: 338 - DNA, 140 bp, Zea mays
  source 1..140: mol_type = genomic DNA
tgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgcca

SEQ ID NO: 339 - DNA, 64 bp, Zea mays
  source 1..64: mol_type = genomic DNA
gaggagacagccacagccatacagggatgctgcctgcttcgtgaccgtgctggtggtggtgggc

SEQ ID NO: 340 - DNA, 85 bp, Zea mays
  source 1..85: mol_type = genomic DNA
tgaccgtgctggtggtggtgggctgcagcctgcagctcgtatcacacgcccacagtgcaggaaggagggagtggaagaagagaga

SEQ ID NO: 341 - DNA, 78 bp, Zea mays
  source 1..78: mol_type = genomic DNA
aggagggagtggaagaagagagaggaagttgtttttaaaggcaacagcgagtcccgctgctgtccctctccttcgcca

SEQ ID NO: 342 - DNA, 223 bp, Zea mays
  source 1..223: mol_type = genomic DNA
gggaatccccacaaggcagcagatggaagcgaaaggtggcggtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagag

SEQ ID NO: 343 - DNA, 143 bp, Zea mays
  source 1..143: mol_type = genomic DNA
ggtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtat

SEQ ID NO: 344 - DNA, 104 bp, Zea mays
  source 1..104: mol_type = genomic DNA
gcgcgggcgcggctagctaggccggctcgtgtgcgtgcgcgaggagacagccacagccatacagggatgctgcctgcttcgtgaccgtgctggtggtggtgggc

SEQ ID NO: 345 - DNA, 63 bp, Zea mays
  source 1..63: mol_type = genomic DNA
gccggctcgtgtgcgtgcgcgaggagacagccacagccatacagggatgctgcctgcttcgtg

SEQ ID NO: 346 - DNA, 223 bp, Zea mays
  source 1..223: mol_type = genomic DNA
gtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggaggagagagaagagagggagccagtccagctccagccagccggacgcccggaccaacaccaccc

SEQ ID NO: 347 - DNA, 143 bp, Zea mays
  source 1..143: mol_type = genomic DNA
caggaaggagggagtggaagaagagagaggaagttgtttttaaaggcaacagcgagtcccgctgctgtccctctccttcgccacctccctccctcactctcttccctccctctcggaggaggagagagaagagagggagccag

SEQ ID NO: 348 - DNA, 103 bp, Zea mays
  source 1..103: mol_type = genomic DNA
aagagagaggaagttgtttttaaaggcaacagcgagtcccgctgctgtccctctccttcgccacctccctccctcactctcttccctccctctcggaggagga

SEQ ID NO: 349 - DNA, 63 bp, Zea mays
  source 1..63: mol_type = genomic DNA
taaaggcaacagcgagtcccgctgctgtccctctccttcgccacctccctccctcactctctt

SEQ ID NO: 350 - DNA, 223 bp, Zea mays
  source 1..223: mol_type = genomic DNA
gtggcgcggcgtggcgcgggcgcgggcgcggctagctaggccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgct

SEQ ID NO: 351 - DNA, 143 bp, Zea mays
  source 1..143: mol_type = genomic DNA
ccggctcgtgtgcgtgcgcgaggagacagccacagccatacagggatgctgcctgcttcgtgaccgtgctggtggtggtgggctgcagcctgcagctcgtatcacacgcccacagtgcaggaaggagggagtggaagaagaga

SEQ ID NO: 352 - DNA, 104 bp, Zea mays
  source 1..104: mol_type = genomic DNA
aggagacagccacagccatacagggatgctgcctgcttcgtgaccgtgctggtggtggtgggctgcagcctgcagctcgtatcacacgcccacagtgcaggaag

SEQ ID NO: 353 - DNA, 63 bp, Zea mays
  source 1..63: mol_type = genomic DNA
cagggatgctgcctgcttcgtgaccgtgctggtggtggtgggctgcagcctgcagctcgtatc

SEQ ID NO: 354 - DNA, 223 bp, Zea mays
  source 1..223: mol_type = genomic DNA
gagacagccacagccatacagggatgctgcctgcttcgtgaccgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctccctccctcactctcttccctccctctcggaggaggagagag

SEQ ID NO: 355 - DNA, 143 bp, Zea mays
  source 1..143: mol_type = genomic DNA
accgtgctggtggtggtgggctgcagcctgcagctcgtatcacacgcccacagtgcaggaaggagggagtggaagaagagagaggaagttgtttttaaaggcaacagcgagtcccgctgctgtccctctccttcgccacctcc

SEQ ID NO: 356 - DNA, 103 bp, Zea mays
  source 1..103: mol_type = genomic DNA
ctgcagcctgcagctcgtatcacacgcccacagtgcaggaaggagggagtggaagaagagagaggaagttgtttttaaaggcaacagcgagtcccgctgctgt

SEQ ID NO: 357 - DNA, 63 bp, Zea mays
  source 1..63: mol_type = genomic DNA
cacacgcccacagtgcaggaaggagggagtggaagaagagagaggaagttgtttttaaaggca

SEQ ID NO: 358 - DNA, 378 bp, Zea mays
  source 1..378: mol_type = genomic DNA
aagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagccacagccacagccacgctgcgccaacagcgggtttcgacggcggcagccctgtggcgccctc

SEQ ID NO: 359 - DNA, 298 bp, Zea mays
  source 1..298: mol_type = genomic DNA
gccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagccacagccacagccacgctgcgc

SEQ ID NO: 360 - DNA, 257 bp, Zea mays
  source 1..257: mol_type = genomic DNA
gcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagcca

SEQ ID NO: 361 - DNA, 218 bp, Zea mays
  source 1..218: mol_type = genomic DNA
aaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagcca

SEQ ID NO: 362 - DNA, 178 bp, Zea mays
  source 1..178: mol_type = genomic DNA
cagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctct

SEQ ID NO: 363 - DNA, 118 bp, Zea mays
  source 1..118: mol_type = genomic DNA
cagtggcaagcgggcttggatcctcgtcaccaccaaggcgcggtcgcgggatacggcgcccactatgggagcgagggtggtagctcgtcgtcggcgaggccgccggtgttccctggcc

SEQ ID NO: 364 - DNA, 83 bp, Zea mays
  source 1..83: mol_type = genomic DNA
gaggccgccggtgttccctggcccggagctgcccccaggtggatgccttgcaggagtccccgcggactccagctgtgctctct

SEQ ID NO: 365 - DNA, 224 bp, Zea mays
  source 1..224: mol_type = genomic DNA
aagcttcatgctggatttctcgtacccgagggtctcaagcgccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtagctcgtcgtcggcgaggccgccggtgttccctggcccggagc

SEQ ID NO: 366 - DNA, 143 bp, Zea mays
  source 1..143: mol_type = genomic DNA
gccatgaggggtgggtttccggcggtgaggcccggtggtgaaagggtgcctggcggtatccagtggcaagcgggcttggatcctcgtcaccaccaaggcgcggtcgcgggatacggcgcccactatgggagcgagggtggtag

SEQ ID NO: 367 - DNA, 103 bp, Zea mays
  source 1..103: mol_type = genomic DNA
ggcggtgaggcccggtggtgaaagggtgcctggcggtatccagtggcaagcgggcttggatcctcgtcaccaccaaggcgcggtcgcgggatacggcgcccac

SEQ ID NO: 368 - DNA, 63 bp, Zea mays
  source 1..63: mol_type = genomic DNA
aaagggtgcctggcggtatccagtggcaagcgggcttggatcctcgtcaccaccaaggcgcgg

SEQ ID NO: 369 - DNA, 223 bp, Zea mays
  source 1..223: mol_type = genomic DNA
gtatccagtggcaagcgggcttggatcctcgtcaccaccaaggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagcca

SEQ ID NO: 370 - DNA, 143 bp, Zea mays
  source 1..143: mol_type = genomic DNA
aggcgcggtcgcgggatacggcgcccactatgggagcgagggtggtagctcgtcgtcggcgaggccgccggtgttccctggcccggagctgcccccaggtggatgccttgcaggagtccccgcggactccagctgtgctctct

SEQ ID NO: 371 - DNA, 103 bp, Zea mays
  source 1..103: mol_type = genomic DNA
gcgcccactatgggagcgagggtggtagctcgtcgtcggcgaggccgccggtgttccctggcccggagctgcccccaggtggatgccttgcaggagtccccgc

SEQ ID NO: 372 - DNA, 63 bp, Zea mays
  source 1..63: mol_type = genomic DNA
ggtggtagctcgtcgtcggcgaggccgccggtgttccctggcccggagctgcccccaggtgga

SEQ ID NO: 373 - DNA, 223 bp, Zea mays
  source 1..223: mol_type = genomic DNA
gcgcccactatgggagcgagggtggtagctcgtcgtcggcgaggccgccggtgttccctggcccggagctgcccccaggtggatgccttgcaggagtccccgcggactccagctgtgctctctctcttctgtcaactcagccatgggatgctgcccacagccacagccacagccacgctgcgccaacagcgggtttcgacggcggcagccctgtggcgccctc

SEQ ID NO: 374 - DNA, 143 bp, Zea mays
  source 1..143: mol_type = genomic DNA
gaggccgccggtgttccctggcccggagctgcccccaggtggatgccttgcaggagtccccgcggactccagctgtgctctctctcttctgtcaactcagccatgggatgctgcccacagccacagccacagccacgctgcgc

SEQ ID NO: 375 - DNA, 103 bp, Zea mays
  source 1..103: mol_type = genomic DNA
gcccggagctgcccccaggtggatgccttgcaggagtccccgcggactccagctgtgctctctctcttctgtcaactcagccatgggatgctgcccacagcca

SEQ ID NO: 376 - DNA, 63 bp, Zea mays
  source 1..63: mol_type = genomic DNA
ggatgccttgcaggagtccccgcggactccagctgtgctctctctcttctgtcaactcagcca

SEQ ID NO: 377 - DNA, 361 bp, Zea mays
  source 1..361: mol_type = genomic DNA
catcaccatggctccgcctccatgcatcatcatttctactgaataataatgtgcttgtaccctccaacgttgccgcctggcattgccaagaatcggttccaagggggtgttgtttaagtaaagcgagaccacaggcacagcgcggctttgcagcatgctcttggcaccagtggtgtggtgtggtgttgctgctgatagactgaccgacggcagaatgagatccacgcagcagcttctttcaaatccacaaaattttctcagtggagaacaccagcacctttcctgcgaaagattttttccccttcccttgcgaattcatttcccttttgcccaaccacacagtctgctgcgcaggtgtttc

SEQ ID NO: 378 - DNA, 280 bp, Zea mays
  source 1..280: mol_type = genomic DNA
gaataataatgtgcttgtaccctccaacgttgccgcctggcattgccaagaatcggttccaagggggtgttgtttaagtaaagcgagaccacaggcacagcgcggctttgcagcatgctcttggcaccagtggtgtggtgtggtgttgctgctgatagactgaccgacggcagaatgagatccacgcagcagcttctttcaaatccacaaaattttctcagtggagaacaccagcacctttcctgcgaaagattttttccccttcccttgcgaattcatt

SEQ ID NO: 379 - DNA, 241 bp, Zea mays
  source 1..241: mol_type = genomic DNA
cctccaacgttgccgcctggcattgccaagaatcggttccaagggggtgttgtttaagtaaagcgagaccacaggcacagcgcggctttgcagcatgctcttggcaccagtggtgtggtgtggtgttgctgctgatagactgaccgacggcagaatgagatccacgcagcagcttctttcaaatccacaaaattttctcagtggagaacaccagcacctttcctgcgaaagattttttccc

SEQ ID NO: 380 - DNA, 201 bp, Zea mays
  source 1..201: mol_type = genomic DNA
cattgccaagaatcggttccaagggggtgttgtttaagtaaagcgagaccacaggcacagcgcggctttgcagcatgctcttggcaccagtggtgtggtgtggtgttgctgctgatagactgaccgacggcagaatgagatccacgcagcagcttctttcaaatccacaaaattttctcagtggagaacaccagcaccttt

SEQ ID NO: 381 - DNA, 161 bp, Zea mays
  source 1..161: mol_type = genomic DNA
aagggggtgttgtttaagtaaagcgagaccacaggcacagcgcggctttgcagcatgctcttggcaccagtggtgtggtgtggtgttgctgctgatagactgaccgacggcagaatgagatccacgcagcagcttctttcaaatccacaaaattttctcag

SEQ ID NO: 382 - DNA, 223 bp, Zea mays
  source 1..223: mol_type = genomic DNA
catcaccatggctccgcctccatgcatcatcatttctactgaataataatgtgcttgtaccctccaacgttgccgcctggcattgccaagaatcggttccaagggggtgttgtttaagtaaagcgagaccacaggcacagcgcggctttgcagcatgctcttggcaccagtggtgtggtgtggtgttgctgctgatagactgaccgacggcagaatgagatcc

SEQ ID NO: 383 - DNA, 143 bp, Zea mays
  source 1..143: mol_type = genomic DNA
gaataataatgtgcttgtaccctccaacgttgccgcctggcattgccaagaatcggttccaagggggtgttgtttaagtaaagcgagaccacaggcacagcgcggctttgcagcatgctcttggcaccagtggtgtggtgtgg

SEQ ID NO: 384 - DNA, 102 bp, Zea mays
  source 1..102: mol_type = genomic DNA
cctccaacgttgccgcctggcattgccaagaatcggttccaagggggtgttgtttaagtaaagcgagaccacaggcacagcgcggctttgcagcatgctctt

SEQ ID NO: 385 - DNA, 63 bp, Zea mays
  source 1..63: mol_type = genomic DNA
cattgccaagaatcggttccaagggggtgttgtttaagtaaagcgagaccacaggcacagcgc

SEQ ID NO: 386 - DNA, 223 bp, Zea mays
  source 1..223: mol_type = genomic DNA
gagaccacaggcacagcgcggctttgcagcatgctcttggcaccagtggtgtggtgtggtgttgctgctgatagactgaccgacggcagaatgagatccacgcagcagcttctttcaaatccacaaaattttctcagtggagaacaccagcacctttcctgcgaaagattttttccccttcccttgcgaattcatttcccttttgcccaaccacacagtctgc

SEQ ID NO: 387 - DNA, 143 bp, Zea mays
  source 1..143: mol_type = genomic DNA
caccagtggtgtggtgtggtgttgctgctgatagactgaccgacggcagaatgagatccacgcagcagcttctttcaaatccacaaaattttctcagtggagaacaccagcacctttcctgcgaaagattttttccccttccc

SEQ ID NO: 388 - DNA, 103 bp, Zea mays
  source 1..103: mol_type = genomic DNA
gttgctgctgatagactgaccgacggcagaatgagatccacgcagcagcttctttcaaatccacaaaattttctcagtggagaacaccagcacctttcctgcg

SEQ ID NO: 389 - DNA, 63 bp, Zea mays
  source 1..63: mol_type = genomic DNA
cgacggcagaatgagatccacgcagcagcttctttcaaatccacaaaattttctcagtggaga

SEQ ID NO: 390 - DNA, 222 bp, Zea mays
  source 1..222: mol_type = genomic DNA
agcgcggctttgcagcatgctcttggcaccagtggtgtggtgtggtgttgctgctgatagactgaccgacggcagaatgagatccacgcagcagcttctttcaaatccacaaaattttctcagtggagaacaccagcacctttcctgcgaaagattttttccccttcccttgcgaattcatttcccttttgcccaaccacacagtctgctgcgcaggtgttt

SEQ ID NO: 391 - DNA, 143 bp, Zea mays
  source 1..143: mol_type = genomic DNA
tgtggtgttgctgctgatagactgaccgacggcagaatgagatccacgcagcagcttctttcaaatccacaaaattttctcagtggagaacaccagcacctttcctgcgaaagattttttccccttcccttgcgaattcattt

SEQ ID NO: 392 - DNA, 103 bp, Zea mays
  source 1..103: mol_type = genomic DNA
actgaccgacggcagaatgagatccacgcagcagcttctttcaaatccacaaaattttctcagtggagaacaccagcacctttcctgcgaaagattttttccc

SEQ ID NO: 393 - DNA, 62 bp, Zea mays
  source 1..62: mol_type = genomic DNA
atccacgcagcagcttctttcaaatccacaaaattttctcagtggagaacaccagcaccttt

SEQ ID NO: 394 - DNA, 418 bp, Zea mays
  source 1..418: mol_type = genomic DNA
ggaatcacaccattcaccaagcagcagatggaagcgaaaggtggcgccgcggcgtggctagctaggccggctcgtgcgcacgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctccctctctttccttcctattagaggagagagaagagggggagagccagtcagccagccagcccaagaacagcgtacgtacgttgtcaagtcccgtcgccgc

SEQ ID NO: 395 - DNA, 338 bp, Zea mays
  source 1..338: mol_type = genomic DNA
gtggcgccgcggcgtggctagctaggccggctcgtgcgcacgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctccctctctttccttcctattagaggagagagaagagggggagagccagtcagccagccagc

SEQ ID NO: 396 - DNA, 298 bp, Zea mays
  source 1..298: mol_type = genomic DNA
gctaggccggctcgtgcgcacgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctccctctctttccttcctattagaggagagagaagaggggga

SEQ ID NO: 397 - DNA, 258 bp, Zea mays
  source 1..258: mol_type = genomic DNA
cgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctccctctctttccttcctatta

SEQ ID NO: 398 - DNA, 218 bp, Zea mays
  source 1..218: mol_type = genomic DNA
tgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctc

SEQ ID NO: 399 - DNA, 167 bp, Zea mays
  source 1..167: mol_type = genomic DNA
tgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttt

SEQ ID NO: 400 - DNA, 76 bp, Zea mays
  source 1..76: mol_type = genomic DNA
tgcgagcgcgcggagacagacacgggactggttgcgtgaccgagctctggtctggtggctggtgctgagagagaga

SEQ ID NO: 401 - DNA, 165 bp, Zea mays
  source 1..165: mol_type = genomic DNA
ggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctc

SEQ ID NO: 402 - DNA, 113 bp, Zea mays
  source 1..113: mol_type = genomic DNA
ggtggctggtgctgagagagagagggagacagacgacctgcagctcgcatcacagtcccacagtgcaggagggagtggaagagagtagagagagagagagagagagagttgtt

SEQ ID NO: 403 - DNA, 74 bp, Zea mays
  source 1..74: mol_type = genomic DNA
gagagagagagagagagttgttttgaaggcaacagcgagccctgctgtccctctgcttcgccacctcctccctc

SEQ ID NO: 404 - DNA, 223 bp, Zea mays
  source 1..223: mol_type = genomic DNA
ggaatcacaccattcaccaagcagcagatggaagcgaaaggtggcgccgcggcgtggctagctaggccggctcgtgcgcacgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcagga

SEQ ID NO: 405 - DNA, 223 bp, Zea mays
  source 1..223: mol_type = genomic DNA
gtggctagctaggccggctcgtgcgcacgcgtgagagaccgtgctcgtgcgagcgcgcggagacagacacgggactggttgcgtgaccgagctctggtctggtggctggtgctgagagagagagggagacagacgacctgcagctcgcatcacagtcccacagtgcaggagggagtggaagagagtagagagagagagagagagagagttgttttgaaggcaa

SEQ ID NO: 406 - DNA, 223 bp, Zea mays
  source 1..223: mol_type = genomic DNA
ctctggtctggtggctggtgctgagagagagagggagacagacgacctgcagctcgcatcacagtcccacagtgcaggagggagtggaagagagtagagagagagagagagagagagttgttttgaaggcaacagcgagccctgctgtccctctgcttcgccacctcctccctccctctctttccttcctattagaggagagagaagagggggagagccagtc

SEQ ID NO: 407 - DNA, 223 bp, Zea mays
  source 1..223: mol_type = genomic DNA
gctcgcatcacagtcccacagtgcaggagggagtggaagagagtagagagagagagagagagagagttgttttgaaggcaacagcgagccctgctgtccctctgcttcgccacctcctccctccctctctttccttcctattagaggagagagaagagggggagagccagtcagccagccagcccaagaacagcgtacgtacgttgtcaagtcccgtcgccgc

SEQ ID NO: 408 - DNA, 396 bp, Zea mays
  source 1..396: mol_type = genomic DNA
cttcatgctggatttctcatacccaagggttccaagcgccatgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcga

SEQ ID NO: 409 - DNA, 316 bp, Zea mays
  source 1..316: mol_type = genomic DNA
atgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggcc

SEQ ID NO: 410 - DNA, 276 bp, Zea mays
  source 1..276: mol_type = genomic DNA
ggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacag

SEQ ID NO: 411 - DNA, 236 bp, Zea mays
  source 1..236: mol_type = genomic DNA
gggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagcca

SEQ ID NO: 412 - DNA, 196 bp, Zea mays
  source 1..196: mol_type = genomic DNA
cagtggcaagcagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctct

SEQ ID NO: 413 - DNA, 133 bp, Zea mays
  source 1..133: mol_type = genomic DNA
cagtggcaagcagcgagcctagatcctcctccccctcaccatcagagcgcggcagcaggatacggtgcccactcgtacgggagcccgggtagctcgtcgtcgtcgtcgaggccaccggtgttccctgctggcc

SEQ ID NO: 414 - DNA, 86 bp, Zea mays
  source 1..86: mol_type = genomic DNA
gccaccggtgttccctgctggcccggagctccccccaggcggtggatgtctcgcaggagtccccacggactctagctgtgctctct

SEQ ID NO: 415 - DNA, 223 bp, Zea mays
  source 1..223: mol_type = genomic DNA
cttcatgctggatttctcatacccaagggttccaagcgccatgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagcccgggtagctcgtcgtcgtcgtcgaggccaccggtgttc

SEQ ID NO: 416 - DNA, 144 bp, Zea mays
  source 1..144: mol_type = genomic DNA
atgagggatgggtttccagcggttcgacctggtggtgaaagggtgcctggcagcagtatccagtggcaagcagcgagcctagatcctcctccccctcaccatcagagcgcggcagcaggatacggtgcccactcgtacgggagc

SEQ ID NO: 417 - DNA, 103 bp, Zea mays
  source 1..103: mol_type = genomic DNA
ggttcgacctggtggtgaaagggtgcctggcagcagtatccagtggcaagcagcgagcctagatcctcctccccctcaccatcagagcgcggcagcaggatac

SEQ ID NO: 418 - DNA, 63 bp, Zea mays
  source 1..63: mol_type = genomic DNA
gggtgcctggcagcagtatccagtggcaagcagcgagcctagatcctcctccccctcaccatc

SEQ ID NO: 419 - DNA, 223 bp, Zea mays
  source 1..223: mol_type = genomic DNA
cagcgagcctagatcctcctccccctcaccatcagagcgcggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggcca

SEQ ID NO: 420 - DNA, 143 bp, Zea mays
  source 1..143: mol_type = genomic DNA
ggcagcaggatacggtgcccactcgtacgggagcccgggtagctcgtcgtcgtcgtcgaggccaccggtgttccctgctggcccggagctccccccaggcggtggatgtctcgcaggagtccccacggactctagctgtgctc

SEQ ID NO: 421 - DNA, 104 bp, Zea mays
  source 1..104: mol_type = genomic DNA
actcgtacgggagcccgggtagctcgtcgtcgtcgtcgaggccaccggtgttccctgctggcccggagctccccccaggcggtggatgtctcgcaggagtcccc

SEQ ID NO: 422 - DNA, 63 bp, Zea mays
  source 1..63: mol_type = genomic DNA
agctcgtcgtcgtcgtcgaggccaccggtgttccctgctggcccggagctccccccaggcggt

SEQ ID NO: 423 - DNA, 223 bp, Zea mays
  source 1..223: mol_type = genomic DNA
cgtacgggagcccgggtagctcgtcgtcgtcgtcgaggccaccggtgttccctgctggcccggagctccccccaggcggtggatgtctcgcaggagtccccacggactctagctgtgctctctctcttctgtcaactcagccatgggacgctgctggccacagcgccggccacggccacggccacggccacgccgcgtcactgcccgcaacagcaggcttcga

SEQ ID NO: 424 - DNA, 143 bp, Zea mays
  source 1..143: mol_type = genomic DNA
accggtgttccctgctggcccggagctccccccaggcggtggatgtctcgcaggagtccccacggactctagctgtgctctctctcttctgtcaactcagccatgggacgctgctggccacagcgccggccacggccacggcc

SEQ ID NO: 425 - DNA, 103 bp, Zea mays
  source 1..103: mol_type = genomic DNA
cggagctccccccaggcggtggatgtctcgcaggagtccccacggactctagctgtgctctctctcttctgtcaactcagccatgggacgctgctggccacag

SEQ ID NO: 426 - DNA, 63 bp, Zea mays
  source 1..63: mol_type = genomic DNA
ggatgtctcgcaggagtccccacggactctagctgtgctctctctcttctgtcaactcagcca

SEQ ID NO: 427 - DNA, 362 bp, Zea mays
  source 1..362: mol_type = genomic DNA
tggtttgaactcgtcggggactttgctcgatgtgttgttattaatctcatgctcatccttccgctaatgttgcctggcggcatcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcggcacctttcctgtgaaaaaagagattttattgtttccttcctcctgtctcgaatcacagcttgtcgtgcagctagctgttc

SEQ ID NO: 428 - DNA, 282 bp, Zea mays
  source 1..282: mol_type = genomic DNA
ttaatctcatgctcatccttccgctaatgttgcctggcggcatcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcggcacctttcctgtgaaaaaagagattttattgtttccttcc

SEQ ID NO: 429 - DNA, 242 bp, Zea mays
  source 1..242: mol_type = genomic DNA
ccgctaatgttgcctggcggcatcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcggcacctttcctgtgaaaaaag

SEQ ID NO: 430 - DNA, 202 bp, Zea mays
  source 1..202: mol_type = genomic DNA
catcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcgg

SEQ ID NO: 431 - DNA, 162 bp, Zea mays
  source 1..162: mol_type = genomic DNA
aaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcag

SEQ ID NO: 432 - DNA, 91 bp, Zea mays
  source 1..91: mol_type = genomic DNA
aaggcagcgttgttcatgtaaagcgagaccgcaggctttgcagcgtgctctcagcaccagtggtgtgggtgccgatagacaggctgactga

SEQ ID NO: 433 - DNA, 94 bp, Zea mays
  source 1..94: mol_type = genomic DNA
gtgccgatagacaggctgactgacggagggcagaatgagatccatcgtccatgcagcagagcagcttcctccaaatctacagaaattttctcag

SEQ ID NO: 434 - DNA, 223 bp, Zea mays
  source 1..223: mol_type = genomic DNA
tggtttgaactcgtcggggactttgctcgatgtgttgttattaatctcatgctcatccttccgctaatgttgcctggcggcatcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgca

SEQ ID NO: 435 - DNA, 143 bp, Zea mays
  source 1..143: mol_type = genomic DNA
ttaatctcatgctcatccttccgctaatgttgcctggcggcatcgccaggaatcggttccaaggcagcgttgttcatgtaaagcgagaccgcaggctttgcagcgtgctctcagcaccagtggtgtgggtgccgatagacagg

SEQ ID NO: 436 - DNA, 103 bp, Zea mays
  source 1..103: mol_type = genomic DNA
ccgctaatgttgcctggcggcatcgccaggaatcggttccaaggcagcgttgttcatgtaaagcgagaccgcaggctttgcagcgtgctctcagcaccagtgg

SEQ ID NO: 437 - DNA, 63 bp, Zea mays
  source 1..63: mol_type = genomic DNA
catcgccaggaatcggttccaaggcagcgttgttcatgtaaagcgagaccgcaggctttgcag

SEQ ID NO: 438 - DNA, 223 bp, Zea mays
  source 1..223: mol_type = genomic DNA
gttgcctggcggcatcgccaggaatcggttccaaggcagcgttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcggcacctttcc

SEQ ID NO: 439 - DNA, 142 bp, Zea mays
  source 1..142: mol_type = genomic DNA
ttgttcatgtaaagcgagaccgcaggctttgcagcgtgctctcagcaccagtggtgtgggtgccgatagacaggctgactgacggagggcagaatgagatccatcgtccatgcagcagagcagcttcctccaaatctacaga

SEQ ID NO: 440 - DNA, 103 bp, Zea mays
  source 1..103: mol_type = genomic DNA
ccgcaggctttgcagcgtgctctcagcaccagtggtgtgggtgccgatagacaggctgactgacggagggcagaatgagatccatcgtccatgcagcagagca

SEQ ID NO: 441 - DNA, 63 bp, Zea mays
  source 1..63: mol_type = genomic DNA
tctcagcaccagtggtgtgggtgccgatagacaggctgactgacggagggcagaatgagatcc

SEQ ID NO: 442 - DNA, 223 bp, Zea mays
  source 1..223: mol_type = genomic DNA
gcagcgtgctctcagcaccagtggtgtgggtgccgatagacaggctgactgacggagggcagaatgagatccatcgtccatgcagcagagcagcttcctccaaatctacagaaattttctcagtggatgagaacaaacagcggcacctttcctgtgaaaaaagagattttattgtttccttcctcctgtctcgaatcacagcttgtcgtgcagctagctgttc

SEQ ID NO: 443 - DNA, 143 bp, Zea mays
  source 1..143: mol_type = genomic DNA
caggctgactgacggagggcagaatgagatccatcgtccatgcagcagagcagcttcctccaaatctacagaaattttctcagtggatgagaacaaacagcggcacctttcctgtgaaaaaagagattttattgtttccttcc

SEQ ID NO: 444 - DNA, 103 bp, Zea mays
  source 1..103: mol_type = genomic DNA
agaatgagatccatcgtccatgcagcagagcagcttcctccaaatctacagaaattttctcagtggatgagaacaaacagcggcacctttcctgtgaaaaaag

SEQ ID NO: 445 - DNA, 63 bp, Zea mays
  source 1..63: mol_type = genomic DNA
tgcagcagagcagcttcctccaaatctacagaaattttctcagtggatgagaacaaacagcgg